(19) United States
(12) Patent Application Publication
(10) Pub. No.: US 2002/0042055 A1
(43) Pub. Date: Apr. 11, 2002

(54) Alteration of hydrolase genes and screening of the resulting libraries for the ability to catalyze specific reactions

(76) Inventor: Joseph A. Affholter, Zephyr Cove, NV (US)

Correspondence Address: LAW OFFICES OF JONATHAN ALAN QUINE, P O BOX 458, ALAMEDA, CA, 94501

(21) Appl. No.: 09/747,277

(22) Filed: Dec. 22, 2000

Related U.S. Application Data
(60) Provisional application No. 60/171,875, filed on Dec. 23, 1999.
(60) Provisional application No. 60/226,238, filed on Aug. 17, 2000.

(51) Int. Cl. C12Q 1/68; G01N 33/53; C12P 19/34

(52) U.S. Cl. 435/6; 435/7.1; 435/91.2

(57) ABSTRACT

The present invention relates to halocarbon and halohydrocarbon chemistry, including methods of dehalogenating halocarbons and halohydrocarbons to provide, inter alia, alcohols, polyols, and epoxides. In general, the methods involve reaction pathways catalyzed by altered hydrolase enzymes that can provide stereoselective or stereospecific reaction products. The invention also includes methods of providing altered nucleic acids that encode altered dehalogenase or other hydrolase enzymes. Additionally, the invention includes various reaction formats and kits.

### BACKGROUND OF THE INVENTION

Halocarbons and halohydrocarbons are ubiquitous chemical compounds derived from various naturally occurring processes and certain chemical manufacturing operations. Many of these chemical species constitute recalcitrant compounds that contaminate surface and subsurface environments. Limited recovery, in the form of heat, from certain waste halohydrocarbons has been achieved by simply burning waste products. Remediation methods for subterranean environments (e.g., for groundwater contamination) have included vacuum extraction of contaminants from shallow zones and air sparging. However, these methods merely result in the transfer of the contaminants from the subsurface environments to either the atmosphere or to an activated carbon form which often ends up in landfills. Contaminated soils, sludges, and activated carbon have also been incinerated but only at high cost. Alternative remedial approaches have included various bioremediation methods that attempt to exploit the ability of some microorganisms to degrade certain recalcitrant compounds. For example, many have sought to develop dehalogenating enzyme systems that utilize either whole-cell microbial catalysts or ex vivo enzymes for bioremediation.

Methods of converting recalcitrant halohydrocarbon compounds as well as other halohydrocarbons to useful products including various carboxylic acids, alcohols, haloalcohols, polyols, halopolyols, epoxides, and/or haloepoxides would be desirable. The present invention provides new methods to enantioselectively or enantiospecifically produce these and other compounds and altered enzymes capable of catalyzing single and/or multiple step reaction pathways. These, as well as a variety of additional features, will become apparent upon review of the following description. SUMMARY OF THE INVENTION

The present invention relates to various reaction pathways (e.g., halocarbon or halohydrocarbon reaction pathways, intermediate isomer reaction pathways, ring opening reaction pathways, and the like) in which altered hydrolase enzymes (e.g., altered dehalogenases) catalyze single or multiple steps in those pathways. In certain cases, these altered enzymes can be involved, e.g., in fewer than all steps (e.g., one step) in a multiple step reaction pathway. In other embodiments, an altered hydrolase catalyzes more than one reaction in multiple step reaction pathways.

In general, the methods include converting one or more reagents (e.g., halocarbons, halohydrocarbons, or the like) to one or more products by incubating the reagent or reagents with an altered hydrolase (e.g., an artificially evolved hydrolase, a recursively recombined hydrolase, a recombinant hydrolase, a chimeric hydrolase, a mutant hydrolase, and the like). Reaction pathway steps can be catalyzed by altered enzymes, e.g., expressed in a heterologous host organism, immobilized on a substrate, free in solution, and the like. Additionally, each step in various multiple step reaction pathways can occur sequentially in a single reaction vessel (e.g., a bioreactor) in which the pathway lacks intervening processing steps, such as separation or purification steps. Alternatively, one or more steps in certain multiple step reaction pathways can optionally occur in distinct reaction vessels.

In one aspect, the invention provides methods of generating diversity in one or more populations of hydrolase nucleic acids. The methods optionally include altering at least one nucleotide of one or more members of the one or more populations of hydrolase nucleic acids using at least one modification technique in which encoded polypeptides of each of the one or more members include abilities to catalyze at least two different reactions or at least two steps in a multiple step reaction pathway. An additional option includes recombining two or more members of the one or more populations of hydrolase nucleic acids in which encoded polypeptides of each of the two or more members include abilities to catalyze at least one different reaction than other members of the one or more populations of hydrolase nucleic acids. A further option includes recombining one or more members from at least a first population of hydrolase nucleic acids, which one or more members encode one or more polypeptides comprising abilities to catalyze at least one step in a multiple step reaction pathway and recombining one or more members from at least a second population of hydrolase nucleic acids, which one or more members encode one or more polypeptides comprising abilities to catalyze at least one different step in the multiple step reaction pathway. Irrespective of the option utilized, the methods also include selecting or screening at least one altered or recombined member of the one or more populations of hydrolase nucleic acids to generate diversity in the one or more populations of hydrolase nucleic acids and repeating the altering step, or at least one of the recombining steps, and the selecting or screening step at least once.

The methods of the present invention can employ myriad different halocarbon and halohydrocarbon reagents (e.g., molecules, molecular appendages or substituent groups, etc.) that typically include from about one to about 100 carbon atoms. The carbon atoms or one or more subsets of the carbon atoms can include, e.g., a straight chain structure, a branched structure, a ring structure, a double bond, a triple bond, and the like. For example, a preferred general class of reactants can include essentially any halohydrocarbon whether cyclic or acyclic (e.g., haloalkanes, haloalkenes, haloalkynes, haloalkyl nitrites, haloalkyl amides, haloalkyl carboxylic acids (e.g., α-haloacids, β-haloacids, and the like), haloalkyl carboxylic acid esters, haloalcohols, halopolyols, haloepoxides, and the like). Typically, the α-haloacid or β-haloacid is a pharmaceutical precursor. A reactant can be, e.g., a xenobiotic or a naturally occurring compound which can also be a component of a mixture derived from various chemical manufacturing operations or from other processes. Additionally, reaction pathways can involve various intermediates, and reactants (e.g., with at least one prochiral or chiral center) can be enantioselectively or enantiospecifically converted to products. The general types of products produced by these methods can include other hydrocarbons or halohydrocarbons (e.g., nitrites, amides, carboxylic acids (e.g., α-hydroxy acids, β-hydroxy acids, and the like), esters, alcohols, haloalcohols, polyols, halopolyols, epoxides, haloepoxides, polyethers, and the like). Specific reactants, intermediates, and products are discussed, infra.

The general types of altered or recombined hydrolase enzymes provided by the invention include, e.g., an altered or recombined halocarbon dehalogenase; an altered or recombined halohydrocarbon dehalogenase; an altered or recombined haloalcohol dehalogenase, an altered or recombined halohydrocarbon-haloalcohol dehalogenase, an altered or recombined halopolyol dehalogenase, an altered or recombined haloalcohol-halopolyol dehalogenase, an altered or recombined halohydrocarbon-haloalcohol-halopolyol dehalogenase, an altered or recombined first haloalcohol isomer providing halohydrocarbon-second haloalcohol isomer providing halohydrocarbon dehalogenase, an altered or recombined epoxide providing halohydrocarbon-haloalcohol isomer dehalogenase, an altered or recombined epoxide providing first haloalcohol isomer-epoxide providing second haloalcohol isomer dehalogenase, an altered or recombined haloepoxide dehalogenase, an altered or recombined epoxide hydrolase, an altered or recombined haloepoxide dehalogenase-epoxide hydrolase, and the like. Specific altered or recombined dehalogenases or hydrolases are discussed, infra.

The various dehalogenases and other hydrolases of the present invention can be altered using many different techniques. A preferred method involves recursive sequence recombination (e.g., DNA shuffling). Recombination methods can be conducted, e.g., in vitro, in vivo, in silico, synthetically, or using whole genomes. Chimeric enzymes including identifiable components derived from two or more parental sequences can also be used. Altered enzymes can additionally be developed using many well-known mutagenic approaches, such as cassette mutagenesis, site-directed mutagenesis, chemical mutagenesis, error-prone PCR, site saturation mutagenesis, ensemble mutagenesis, recursive ensemble mutagenesis, and the like. Other techniques suitable for creating diversity can also be utilized.

As mentioned, preferred methods of providing a population of altered hydrolase nucleic acids include recursive sequence recombination. These methods include hybridizing sets of hydrolase nucleic acid fragments (e.g., synthesized oligonucleotides and nuclease digested nucleic acids). Additionally, these nucleic acid fragments can overlap. The set of hybridized hydrolase nucleic acid fragments can be elongated or ligated to provide the population of altered hydrolase nucleic acids. Thereafter, the population of altered hydrolase nucleic acids can be expressed to provide an altered hydrolase. Optionally, the altered hydrolase nucleic acids can be introduced into a cell (e.g., an organism), in which the introduced altered hydrolase nucleic acids can be expressed to provide an altered hydrolase.

The methods of providing a population of altered hydrolase nucleic acids can further include denaturing the population of altered hydrolase nucleic acids and rehybridizing the denatured population of altered hydrolase nucleic acids. The rehybridized population of altered hydrolase nucleic acids can be extended to provide a population of further altered hydrolase nucleic acids and, optionally, the denaturing, rehybridizing, and extending steps can be repeated at least once. Additionally, the population of further altered hydrolase nucleic acids can optionally be selected or screened for an efficient catalyzation of a reaction of interest (e.g., the conversion of a reactant to an enantiomerically-enriched product) by an encoded altered hydrolase. The population of further altered hydrolase nucleic acids can also be expressed to provide a further altered hydrolase.

The present invention also relates to an integrated system that includes a computer or computer readable medium that includes a data set corresponding to a set of character strings corresponding to a set of dehalogenase or other hydrolase nucleic acid fragments. The integrated system can optionally include a sequence comparison instruction set for optimizing alignment of homologous nucleic acid sequences. The system can also include an automatic synthesizer coupled to an output of the computer or computer readable medium. The automatic synthesizer can accept instructions from the computer or computer readable medium and those instructions can direct synthesis of a set of dehalogenase or other hydrolase nucleic acid fragments.

The integrated system can additionally include one or more robotic control element(s) for incubating, denaturing, hybridizing, elongating and/or ligating the set of dehalogenase or other hydrolase nucleic acid fragments. The integrated system can further include a detector for detecting and/or characterizing a nucleic acid produced by elongation and/or ligation of the set of dehalogenase or other hydrolase nucleic acid fragments, or an encoded product thereof.

The present invention also includes a kit and an assay system for performing one or more of the altered enzyme catalyzed reactions described herein. For example, the kit optionally includes a set of instructions for practicing the methods described herein; assay components that include altered enzymes, or cells that include altered enzymes, or both, and one or more reagents; and a container for packaging the set of instructions and the assay components. The assay system typically includes a set of instructions for practicing the methods disclosed herein, and a reaction vessel (e.g., a bioreactor) that includes altered enzymes, or cells that include the altered enzymes, or both. The reaction vessel optionally also includes, e.g., a reagent inlet, a reagent outlet, a sparging line, an impeller, or the like. The altered enzymes of the assay system typically include immobilized altered enzymes, or altered enzymes free in solution, or both. As further alternatives, the cells of the assay system optionally include immobilized cells, or cells free in solution, or both. Any altered enzyme disclosed herein is optionally utilized in the kit or the assay system of the present invention.

Definitions

Unless otherwise indicated, the following definitions supplement those in the art.

An “organic” chemical compound or substituent group is one that includes at least one carbon atom, but which also typically includes other substituents, such as amino, alkoxy, cyano, hydroxy, carboxy, halo, and/or other groups. Organic compounds or groups can include any number of carbon atoms, but typically fall in the range of about one to about 100 carbon atoms or in the range of about one to about 50 carbon atoms. General classes of organic compounds or groups include, inter alia, halocarbons, halohydrocarbons, haloalcohols, alcohols, halopolyols, polyols, haloepoxides, and epoxides.

The term “halocarbons” refers to organic chemical compounds or substituents that are completely substituted. Halocarbons have at least one carbon atom and at least one halogen atom, but can also include other substituents, such as amino, alkoxy, cyano, hydroxy, carboxy, and/or other groups. For example, specific halocarbon compounds or groups include CCl₄, CBr₄, C₂Cl₄, CCl₂F₂, CCl₃F, —CF₃, or the like, and are typically at least partially dehalogenated in reactions catalyzed by the various halocarbon dehalogenases disclosed herein.

The term “halohydrocarbons” refers to organic chemical compounds or species (e.g., molecules, substituent groups, etc.) that are composed of at least one carbon, hydrogen, and halogen atom, but which can also include other substituents. For example, suitable halohydrocarbons can include essentially any functional group or groups, such as amino, alkoxy, cyano, hydroxy, carboxy, and/or other groups. Preferred halohydrocarbons include two or more halogen atoms in which the halogens are on adjacent carbon atoms, or compounds with a single halogen atom on a carbon adjacent to a carboxylic acid carbon. A “halogen” atom can include chlorine, bromine, iodine, astatine, and fluorine atoms. Preferred enzymes of the invention remove, e.g., bromine, chlorine, and fluorine.

Halohydrocarbons can be “acyclic” in which case the compounds include chains of carbon atoms that do not involve cyclic structures. These acyclic structures can occur, e.g., as free compounds, as side-chains, or as other functional appendages or groups of larger molecules, such as functional groups of aromatic or other cyclic compounds. Halohydrocarbons can also be “cyclic” molecules or groups in which case the carbon atoms are arranged in a ring or rings, e.g., halocyclopropane, 1,2-dihalocyclopentane, 1,2,3-trihalohexane, 1,3-dihalocyclohexene, 1,3,4,6-tetrahalo-1,4-cyclohexadiene, and the like. Additionally, halohydrocarbons can include “aromatic” molecules or groups. Aromatic halohydrocarbons are cyclic compounds that include completely conjugated ring structures, such as halobenzene, dihalobenzene, and the like.

Examples of preferred general classes of halohydrocarbons which can be acted upon by the enzymes of the invention include α-haloacid, β-haloacid, α-haloamide, β-haloamide, α-halonitrile, β-halonitrile, α-haloester, β-haloester, 1,2-dihaloethane, trihalomethane, trihaloethane, 2-haloethanoic acid, 2-haloethanamide, 2-haloethanenitrile, alkyl 2-haloethanoate, 2-halopropanoic acid, 2-halopropanamide, 2-halopropanenitrile, alkyl 2-halopropanoate, 3-halopropanoic acid, 3-halopropanamide, 3-halopropanenitrile, alkyl 3-halopropanoate, 2-halobutanoic acid, 2-halobutanamide, 2-halobutanenitrile, alkyl 2-halobutanoate, 3-halobutanoic acid, 3-halobutanamide, 3-halobutanenitrile, alkyl 3-halobutanoate, 4-halobutanoic acid, 4-halobutanamide, 4-halobutanenitrile, alkyl 4-halobutanoate, 2,3-dihalopropanenitrile, 3,4-dihalobutanenitrile, 2,3-dihalopropanamide, 3,4-dihalobutanamide, 2,3-dihalopropanoic acid, 3,4-dihalobutanoic acid, alkyl 2,3-dihalopropanoate, alkyl 3,4-dihalobutanoate, trihaloethene, trans-1,2-dihaloethene, 1,1-dihaloethene, haloethene, 1,2-dihalopropane, 1,2-dihalobutane, 1,2,3-trihalopropane, and the like.

Halohydrocarbon molecules and groups which can be acted upon by the enzymes of the invention include, e.g., 1,2,3-trichloropropane, 1,2-dichloropropane, 1,2-dichloroethane, 1,2-dibromoethane, 3-chloro-1-propene, 1,2-dibromo-3-chloropropane, 1,2-dichlorobutane, chloroethene, 1-chloropropane, 1,3-dichloropropane, 1,2,3,4-tetrachlorobutane, 1,2,4,5-tetrachloropentane, 1,2,5,6-tetrachlorohexane, 1,2,6,7-tetrachloroheptane, 1,2,7,8-tetrachlorooctane, 1,3,4,6-tetrachloro-1,4-cyclohexadiene, tetrachloroethene, trichloroethene, trans-1,2-dichloroethene, cis-1,2-dichloroethene, 1,1-dichloroethene, chloroethene, 3-chloropropene, 2,3-dichloropropanenitrile, 2,3-dibromopropanenitrile, (R)-2,3-dichloropropanenitrile, (R)-2,3-dibromopropanenitrile, (S)-2,3-dichloropropanenitrile, (S)-2,3-dibromopropanenitrile, 3,4-dichlorobutanenitrile, 3,4-dibromobutanenitrile, (R)-3,4-dichlorobutanenitrile, (R)-3,4-dibromobutanenitrile, (S)-3,4-dichlorobutanenitrile, (S)-3,4-dibromobutanenitrile, 2,3-dichloropropanamide, 2,3-dibromopropanamide, (R)-2,3-dichloropropanamide, (R)-2,3-dibromopropanamide, (S)-2,3-dichloropropanamide, (S)-2,3-dibromopropanamide, 3,4-dichlorobutanamide, 3,4-dibromobutanamide, (R)-3,4-dichlorobutanamide, (R)-3,4-dibromobutanamide, (S)-3,4-dichlorobutanamide, (S)-3,4-dibromobutanamide, 2,3-dichloropropanoic acid, 2,3-dibromopropanoic acid, (R)-2,3-dichloropropanoic acid, (R)-2,3-dibromopropanoic acid, (S)-2,3-dichloropropanoic acid, (S)-2,3-dibromopropanoic acid, 3,4-dichlorobutanoic acid, 3,4-dibromobutanoic acid, (R)-3,4-dichlorobutanoic acid, (R)-3,4-dibromobutanoic acid, (S)-3,4-dichlorobutanoic acid, (S)-3,4-dibromobutanoic acid, alkyl 2,3-dichloropropanoate, alkyl 2,3-dibromopropanoate, alkyl 3,4-dichlorobutanoate, alkyl 3,4-dibromobutanoate, (R)-alkyl 2,3-dichloropropanoate, (R)-alkyl 2,3-dibromopropanoate, (R)-alkyl 3,4-dichlorobutanoate, (R)-alkyl 3,4-dibromobutanoate, (S)-alkyl 2,3-dichloropropanoate, (S)-alkyl 2,3-dibromopropanoate, (S)-alkyl 3,4-dichlorobutanoate, (S)-alkyl 3,4-dibromobutanoate, 2-chloroethanoic acid, 2-bromoethanoic acid, 2-chloroethanamide, 2-bromoethanamide, 2-chloroethanenitrile, 2-bromoethanenitrile, alkyl 2-chloroethanoate, alkyl 2-bromoethanoate, 2-chloropropanoic acid, 2-bromopropanoic acid, 2-chloropropanamide, 2-bromopropanamide, 2-chloropropanenitrile, 2-bromopropanenitrile, alkyl 2-chloropropanoate, alkyl 2-bromopropanoate, 3-chloropropanoic acid, 3-bromopropanoic acid, 3-chloropropanamide, 3-bromopropanamide, 3-chloropropanenitrile, 3-bromopropanenitrile, alkyl 3-chloropropanoate, alkyl 3-bromopropanoate, 2-chlorobutanoic acid, 2-bromobutanoic acid, 2-chlorobutanamide, 2-bromobutanamide, 2-chlorobutanenitrile, 2-bromobutanenitrile, alkyl 2-chlorobutanoate, alkyl 2-bromobutanoate, 3-chlorobutanoic acid, 3-bromobutanoic acid, 3-chlorobutanamide, 3-bromobutanamide, 3-chlorobutanenitrile, 3-bromobutanenitrile, alkyl 3-chlorobutanoate, alkyl 3-bromobutanoate, 4-chlorobutanoic acid, 4-bromobutanoic acid, 4-chlorobutanamide, 4-bromobutanamide, 4-chlorobutanenitrile, 4-bromobutanenitrile, alkyl 4-chlorobutanoate, alkyl 4-bromobutanoate, (R)-2-chloropropanoic acid, (R)-2-bromopropanoic acid, (S)-2-chloropropanoic acid, (S)-2-bromopropanoic acid, (R)-2-chloropropanamide, (R)-2-bromopropanamide, (S)-2-chloropropanamide, (S)-2-bromopropanamide, (R)-2-chloropropanenitrile, (R)-2-bromopropanenitrile, (S)-2-chloropropanenitrile, (S)-2-bromopropanenitrile, (R)-alkyl 2-chloropropanoate, (R)-alkyl 2-bromopropanoate, (S)-alkyl 2-chloropropanoate, (S)-alkyl 2-bromopropanoate, (R)-2-chlorobutanoic acid, (R)-2-bromobutanoic acid, (S)-2-chlorobutanoic acid, (S)-2-bromobutanoic acid, (R)-2-chlorobutanamide, (R)-2-bromobutanamide, (S)-2-chlorobutanamide, (S)-2-bromobutanamide, (R)-2-chlorobutanenitrile, (R)-2-bromobutanenitrile, (S)-2-chlorobutanenitrile, (S)-2-bromobutanenitrile, (R)-alkyl 2-chlorobutanoate, (R)-alkyl 2-bromobutanoate, (S)-alkyl 2-chlorobutanoate, (S)-alkyl 2-bromobutanoate, (R)-3-chlorobutanoic acid, (R)-3-bromobutanoic acid, (S)-3-chlorobutanoic acid, (S)-3-bromobutanoic acid, (R)-3-chlorobutanamide, (R)-3-bromobutanamide, (S)-3-chlorobutanamide, (S)-3-bromobutanamide, (R)-3-chlorobutanenitrile, (R)-3-bromobutanenitrile, (S)-3-chlorobutanenitrile, (S)-3-bromobutanenitrile, (R)-alkyl 3-chlorobutanoate, (R)-alkyl 3-bromobutanoate, (S)-alkyl 3-chlorobutanoate, (S)-alkyl 3-bromobutanoate, 2,3-dichlorobutanenitrile, 2,3-dibromobutanenitrile, 2,3-dichlorobutanamide, 2,3-dibromobutanamide, 2,3-dichlorobutanoic acid, 2,3-dibromobutanoic acid, alkyl 2,3-dichlorobutanoate, alkyl 2,3-dibromobutanoate, (R,R)-2,3-dichlorobutanenitrile, (R,R)-2,3-dibromobutanenitrile, (R,R)-2,3-dichlorobutanamide, (R,R)-2,3-dibromobutanamide, (R,R)-2,3-dichlorobutanoic acid, (R,R)-2,3-dibromobutanoic acid, (R,R)-alkyl 2,3-dichlorobutanoate, (R,R)-alkyl 2,3-dibromobutanoate, (S,S)-2,3-dichlorobutanenitrile, (S,S)-2,3-dibromobutanenitrile, (S,S)-2,3-dichlorobutanamide, (S,S)-2,3-dibromobutanamide, (S,S)-2,3-dichlorobutanoic acid, (S,S)-2,3-dibromobutanoic acid, (S,S)-alkyl 2,3-dichlorobutanoate, (S,S)-alkyl 2,3-dibromobutanoate, (R,S)-2,3-dichlorobutanenitrile, (R,S)-2,3-dibromobutanenitrile, (R,S)-2,3-dichlorobutanamide, (R,S)-2,3-dibromobutanamide, (R,S)-2,3-dichlorobutanoic acid, (R,S)-2,3-dibromobutanoic acid, (R,S)-alkyl 2,3-dichlorobutanoate, (R,S)-alkyl 2,3-dibromobutanoate, (S,R)-2,3-dichlorobutanenitrile, (S,R)-2,3-dibromobutanenitrile, (S,R)-2,3-dichlorobutanamide, (S,R)-2,3-dibromobutanamide, (S,R)-2,3-dichlorobutanoic acid, (S,R)-2,3-dibromobutanoic acid, (S,R)-alkyl 2,3-dichlorobutanoate, (S,R)-alkyl 2,3-dibromobutanoate, 2,3,4-trichlorobutanenitrile, 2,3,4-tribromobutanenitrile, 2,3,4-trichlorobutanamide, 2,3,4-tribromobutanamide, 2,3,4-trichlorobutanoic acid, 2,3,4-tribromobutanoic acid, alkyl 2,3,4-trichlorobutanoate, alkyl 2,3,4-tribromobutanoate, (R,R)-2,3,4-trichlorobutanenitrile, (R,R)-2,3,4-tribromobutanenitrile, (R,R)-2,3,4-trichlorobutanamide, (R,R)-2,3,4-tribromobutanamide, (R,R)-2,3,4-trichlorobutanoic acid, (R,R)-2,3,4-tribromobutanoic acid, (R,R)-alkyl 2,3,4-trichlorobutanoate, (R,R)-alkyl 2,3,4-tribromobutanoate, (S,S)-2,3,4-trichlorobutanenitrile, (S,S)-2,3,4-tribromobutanenitrile, (S,S)-2,3,4-trichlorobutanamide, (S,S)-2,3,4-tribromobutanamide, (S,S)-2,3,4-trichlorobutanoic acid, (S,S)-2,3,4-tribromobutanoic acid, (S,S)-alkyl 2,3,4-trichlorobutanoate, (S,S)-alkyl 2,3,4-tribromobutanoate, (R,S)-2,3,4-trichlorobutanenitrile, (R,S)-2,3,4-tribromobutanenitrile, (R,S)-2,3,4-trichlorobutanamide, (R,S)-2,3,4-tribromobutanamide, (R,S)-2,3,4-trichlorobutanoic acid, (R,S)-2,3,4-tribromobutanoic acid, (R,S)-alkyl 2,3,4-trichlorobutanoate, (R,S)-alkyl 2,3,4-tribromobutanoate, (S,R)-2,3,4-trichlorobutanenitrile, (S,R)-2,3,4-tribromobutanenitrile, (S,R)-2,3,4-trichlorobutanamide, (S,R)-2,3,4-tribromobutanamide, (S,R)-2,3,4-trichlorobutanoic acid, (S,R)-2,3,4-tribromobutanoic acid, (S,R)-alkyl 2,3,4-trichlorobutanoate, (S,R)-alkyl 2,3,4-tribromobutanoate, and the like.

The term “alcohol” refers to an organic molecule or group that includes at least one hydroxy group, but which can also include other substituents, such as amino, alkoxy, cyano, carboxy, halo, and/or other groups. Examples of preferred alcohols that can function as reactants and/or products of the invention include 2-propen-1-ol, 1-propanol, α-hydroxy acids, β-hydroxy acids, α-hydroxyamides, β-hydroxyamides, α-hydroxynitriles, β-hydroxynitriles, α-hydroxyesters, β-hydroxyesters, 2-hydroxyethanoic acid, 2-hydroxyethanamide, 2-hydroxyethanenitrile, alkyl 2-hydroxyethanoate, 2-hydroxypropanoic acid, 2-hydroxypropanamide, 2-hydroxypropanenitrile, alkyl 2-hydroxypropanoate, 3-hydroxypropanoic acid, 3-hydroxypropanamide, 3-hydroxypropanenitrile, alkyl 3-hydroxypropanoate, 2-hydroxybutanoic acid, 2-hydroxybutanamide, 2-hydroxybutanenitrile, alkyl 2-hydroxybutanoate, 3-hydroxybutanoic acid, 3-hydroxybutanamide, 3-hydroxybutanenitrile, alkyl 3-hydroxybutanoate, 4-hydroxybutanoic acid, 4-hydroxybutanamide, 4-hydroxybutanenitrile, alkyl 4-hydroxybutanoate, and the like.

Alcohol enantiomers that can function as reactants and/or products of the invention include, e.g., (R)-2-hydroxypropanoic acid, (S)-2-hydroxypropanoic acid, (R)-2-hydroxypropanamide, (S)-2-hydroxypropanamide, (R)-2-hydroxypropanenitrile, (S)-2-hydroxypropanenitrile, (R)-alkyl 2-hydroxypropanoate, (S)-alkyl 2-hydroxypropanoate, (R)-2-hydroxybutanoic acid, (S)-2-hydroxybutanoic acid, (R)-2-hydroxybutanamide, (S)-2-hydroxybutanamide, (R)-2-hydroxybutanenitrile, (S)-2-hydroxybutanenitrile, (R)-alkyl 2-hydroxybutanoate, (S)-alkyl 2-hydroxybutanoate, (R)-3-hydroxybutanoic acid, (S)-3-hydroxybutanoic acid, (R)-3-hydroxybutanamide, (S)-3-hydroxybutanamide, (R)-3-hydroxybutanenitrile, (S)-3-hydroxybutanenitrile, (R)-alkyl 3-hydroxybutanoate, (S)-alkyl 3-hydroxybutanoate, and the like.

A “haloalcohol” is a halo-substituted alcohol. The term “halohydrin” or “vicinal halohydrin” refers to an organic molecule or substituent group that contains both a hydroxyl substituent and a halogen substituent on adjacent carbon atoms of the molecule or group. Haloalcohols and/or intermediate haloalcohols that can function as reactants and/or products of the invention include, e.g., 2-halo-3-hydroxypropanenitrile, 3-halo-2-hydroxypropanenitrile, 3-halo-4-hydroxybutanenitrile, 4-halo-3-hydroxybutanenitrile, 2-halo-3-hydroxypropanamide, 3-halo-2-hydroxypropanamide, 3-halo-4-hydroxybutanamide, 4-halo-3-hydroxybutanamide, 2-halo-3-hydroxypropanoic acid, 3-halo-2-hydroxypropanoic acid, 3-halo-4-hydroxybutanoic acid, 4-halo-3-hydroxybutanoic acid, alkyl 2-halo-3-hydroxypropanoate, alkyl 3-halo-2-hydroxypropanoate, alkyl 3-halo-4-hydroxybutanoate, alkyl 4-halo-3-hydroxybutanoate, 2,3-dichloro-1-propanol, 2-chloro-1-propanol, 1-chloro-2-propanol, 2-chloro-1-ethanol, 3-chloro-1-propanol, 1,3-dichloro-2-propanol, 2,3,4-trichloro-1-butanol, 2,4,5-trichloro-1-pentanol, 2,5,6-trichloro-1-hexanol, 2,6,7-trichloro-1-heptanol, 2,7,8-trichloro-1-octanol, 2-chloro-3-hydroxypropanenitrile, 3-chloro-2-hydroxypropanenitrile, 2-bromo-3-hydroxypropanenitrile, 3-bromo-2-hydroxypropanenitrile, 3-chloro-4-hydroxybutanenitrile, 4-chloro-3-hydroxybutanenitrile, 3-bromo-4-hydroxybutanenitrile, 4-bromo-3-hydroxybutanenitrile, 2-chloro-3-hydroxypropanamide, 3-chloro-2-hydroxypropanamide, 2-bromo-3-hydroxypropanamide, 3-bromo-2-hydroxypropanamide, 3-chloro-4-hydroxybutanamide, 4-chloro-3-hydroxybutanamide, 3-bromo-4-hydroxybutanamide, 4-bromo-3-hydroxybutanamide, 2-chloro-3-hydroxypropanoic acid, 3-chloro-2-hydroxypropanoic acid, 2-bromo-3-hydroxypropanoic acid, 3-bromo-2-hydroxypropanoic acid, 3-chloro-4-hydroxybutanoic acid, 4-chloro-3-hydroxybutanoic acid, 3-bromo-4-hydroxybutanoic acid, 4-bromo-3-hydroxybutanoic acid, alkyl 2-chloro-3-hydroxypropanoate, alkyl 3-chloro-2-hydroxypropanoate, alkyl 2-bromo -3-hydroxypropanoate , alkyl 3-bromo-2-hydroxypropanoate, alkyl 3-chloro-4-hydroxybutanoate, alkyl 4-chloro -3-hydroxybutanoate, alkyl 3-bromo-4-hydroxybutanoate, alkyl 4-bromo-3-hydroxybutanoate, 2-chloro-3-hydroxybutanenitrile, 2-bromo-3-hydroxybutanenitrile, 3-chloro-2-hydroxybutanenitrile, 3-bromo-2-hydroxybutanenitrile, 2-chloro-3-hydroxybutanamide, 2-bromo-3-hydroxybutanamide, 3-chloro-2-hydroxybutanamide, 3-bromo-2-hydroxybutanamide, 2-chloro-3-hydroxybutanoic acid, 2-bromo-3-hydroxybutanoic acid, 3-chloro-2-hydroxybutanoic acid, 3-bromo-2-hydroxybutanoic acid, alkyl 2-chloro-3-hydroxybutanoate, alkyl 2-bromo-3-hydroxybutanoate, alkyl 3-chloro-2-hydroxybutanoate, alkyl 2-bromo-3-hydroxybutanoate, 2,3-dichloro-4-hydroxybutanenitrile, 2,3-dibromo-4-hydroxybutanenitrile, 2,3-dichloro-4-hydroxybutanamide, 2,3-dibromo-4-hydroxybutanamide, 2,3-dichloro-4-hydroxybutanoic acid, 2,3-dibromo-4-hydroxybutanoic acid, alkyl 2,3-dichloro-4-hydroxybutanoate, alkyl 2,3-dibromo-4-hydroxybutanoate, 2,4-dichloro-3-hydroxybutanenitrile, 2,4-dibromo-3-hydroxybutanenitrile, 2,4-dichloro-3-hydroxybutanamide, 2,4-dibromo-3-hydroxybutanamide, 2,4-dichloro-3-hydroxybutanoic acid, 2,4-dibromo-3-hydroxybutanoic acid, alkyl 2,4-dichloro-3-hydroxybutanoate, alkyl 2,4-dibromo-3-hydroxybutanoate, 3,4-dichloro-2-hydroxybutanenitrile, 3,4-dibromo-2-hydroxybutanenitrile, 3,4-dichloro-2-hydroxybutanamide, 3,4-dibromo-2-hydroxybutanamide, 3,4-dichloro-2-hydroxybutanoic acid, 3,4-dibromo-2-hydroxybutanoic acid, alkyl 3,4-dichloro-2-hydroxybutanoate, alkyl 3,4-dibromo-2-hydroxybutanoate, and the like.

Haloalcohol enantiomers that can function as reactants and/or products of the invention include, e.g., (R)-2,3-dichloro-1-propanol, (R)-2-chloro-1-propanol, (R)-1-chloro-2-propanol, (S)-2,3-dichloro-1-propanol, (S)-2-chloro-1-propanol, (S)-1-chloro-2-propanol, (R,R)-2,3,4-trichloro-1-butanol, (S,S)-2,3,4-trichloro-1-butanol, (R,S)-2,3,4-trichloro-1-butanol, (R,S)-2,3,4-trichloro-1-butanol, (R,R)-2,4,5-trichloro-1-pentanol, (S,S)-2,4,5-trichloro-1-pentanol, (R,S)-2,4,5-trichloro-1-pentanol, (S,R)-2,4,5-trichloro-1-pentanol, (R,R)-2,5,6-trichloro-1-hexanol, (S,S)-2,5,6-trichloro-1-hexanol, (R,S)-2,5,6-trichloro-1-hexanol, (S,R)-2,5,6-trichloro-1-hexanol, (R,R)-2,6,7-trichloro-1-heptanol, (S,S)-2,6,7-trichloro-1-heptanol, (R,S)-2,6,7-trichloro-1-heptanol, (S,R)-2,6,7-trichloro-1-heptanol, (R,R)-2,7,8-trichloro-1-octanol, (S,S)-2,7,8-trichloro-1-octanol, (R,S)-2,7,8-trichloro-1-octanol, (S,R)-2,7,8-trichloro-1-octanol, (R)-2-chloro-3-hydroxypropanenitrile, (R)-3-chloro-2-hydroxypropanenitrile, (R)-2-bromo-3-hydroxypropanenitrile, (R)-3-bromo-2-hydroxypropanenitrile, (S)-2-chloro-3-hydroxypropanenitrile, (S)-3-chloro-2-hydroxypropanenitrile, (S)-2-bromo-3-hydroxypropanenitrile, (S)-3-bromo-2-hydroxypropanenitrile, (R)-3-chloro-4-hydroxybutanenitrile, (R)-4-chloro-3-hydroxybutanenitrile, (R)-3-bromo-4-hydroxybutanenitrile, (R)-4-bromo-3-hydroxybutanenitrile, (S)-3-chloro-4-hydroxybutanenitrile, (S)-4-chloro-3-hydroxybutanenitrile, (S)-3-bromo-4-hydroxybutanenitrile, (S)-4-bromo-3-hydroxybutanenitrile, (R)-2-chloro-3-hydroxypropanamide, (R)-3-chloro-2-hydroxypropanamide, (R)-2-bromo-3-hydroxypropanamide, (R)-3-bromo-2-hydroxypropanamide, (S)-2-chloro-3-hydroxypropanamide, (S)-3-chloro-2-hydroxypropanamide, (S)-2-bromo-3-hydroxypropanamide, (S)-3-bromo-2-hydroxypropanamide, (R)-3-chloro-4-hydroxybutanamide, (R)-4-chloro-3-hydroxybutanamide, (R)-3-bromo-4-hydroxybutanamide, (R)-4-bromo-3-hydroxybutanamide, (S)-3-chloro-4-hydroxybutanamide, (S)-4-chloro-3-hydroxybutanamide, (S)-3-bromo-4-hydroxybutanamide, (S)-4-bromo-3-hydroxybutanamide, (R)-2-chloro-3-hydroxypropanoic acid, (R)-3-chloro-2-hydroxypropanoic acid, (R)-2-bromo-3-hydroxypropanoic acid, (R)-3-bromo-2-hydroxypropanoic acid, (S)-2-chloro-3-hydroxypropanoic acid, (S)-3-chloro-2-hydroxypropanoic acid, (S)-2-bromo-3-hydroxypropanoic acid, (S)-3-bromo-2-hydroxypropanoic acid, (R)-3-chloro-4-hydroxybutanoic acid, (R)-4-chloro-3-hydroxybutanoic acid, (R)-3-bromo-4-hydroxybutanoic acid, (R)-4-bromo-3-hydroxybutanoic acid, (S)-3-chloro-4-hydroxybutanoic acid, (S)-4-chloro-3-hydroxybutanoic acid, (S)-3-bromo-4-hydroxybutanoic acid, (S)-4-bromo-3-hydroxybutanoic acid (R)-alkyl 2-chloro-3-hydroxypropanoate, (R)-alkyl 3-chloro-2-hydroxypropanoate, (R)-alkyl 2-bromo-3-hydroxypropanoate, (R)-alkyl 3-bromo-2-hydroxypropanoate, (R)-alkyl 3-chloro-4-hydroxybutanoate, (R)-alkyl 4-chloro-3-hydroxybutanoate,)-alkyl 3-bromo-4-hydroxybutanoate, (R)-alkyl 4-bromo-3-hydroxybutanoate, (R)-alkyl 2-chloro-3-hydroxypropanoate, (R)-alkyl 3-chloro-2-hydroxypropanoate, (S)-alkyl 2-bromo-3-hydroxypropanoate, (S)-alkyl 3-bromo-2-hydroxypropanoate, (S)-alkyl 3-chloro-4-hydroxybutanoate, (S)-alkyl 4-chloro-3-hydroxybutanoate, (S)-alkyl 3-bromo-4-hydroxybutanoate, (S)-alkyl 4-bromo-3-hydroxybutanoate, , (R,R)-2-chloro-3-hydroxybutanenitrile, (R,R)-2-bromo-3-hydroxybutanenitrile, (R,R)-3-chloro-2-hydroxybutanenitrile, (R,R)-3-bromo-2-hydroxybutanenitrile, (R,R)-2-chloro-3-hydroxybutanamide, (R,R)-2-bromo-3-hydroxybutanamide, (R,R)-3-chloro-2-hydroxybutanamide, (R,R)-3-bromo-2-hydroxybutanamide, (R,R)-2-chloro-3-hydroxybutanoic acid, (R,R)-2-bromo-3-hydroxybutanoic acid, (R,R)-3-chloro-2-hydroxybutanoic acid, (R,R)-3-bromo-2-hydroxybutanoic acid, (R,R)-alkyl 2-chloro-3-hydroxybutanoate, (R,R)-alkyl 2-bromo-3-hydroxybutanoate, (R,R)-alkyl 3-chloro-2-hydroxybutanoate, (R,R)-alkyl 3-bromo-2-hydroxybutanoate, (S,S)-2-chloro-3-hydroxybutanenitrile, (S,S)-2-bromo-3-hydroxybutanenitrile, (S,S)-3-chloro-2-hydroxybutanenitrile, (S,S)-3-bromo-2-hydroxybutanenitrile, (S,S)-2-chloro-3-hydroxybutanamide, (S,S)-2-bromo-3-hydroxybutanamide, (S,S)-3-chloro-2-hydroxybutanamide, (S,S)-3-bromo-2-hydroxybutanamide, (S,S)-2-chloro-3-hydroxybutanoic acid, (S,S)-2-bromo-3-hydroxybutanoic acid, (S,S)-3-chloro-2-hydroxybutanoic acid, (S,S)-3-bromo-2-hydroxybutanoic acid, (S,S)-alkyl 2-chloro-3-hydroxybutanoate, (S,S)-alkyl 2-bromo-3-hydroxybutanoate, (S,S)-alkyl 3-chloro-2-hydroxybutanoate, (S,S)-alkyl 3-bromo-2-hydroxybutanoate, (R,S)-2-chloro-3-hydroxybutanenitrile, (R,S)-2-bromo-3-hydroxybutanenitrile, (R,S)-3-chloro-2-hydroxybutanenitrile, (R,S)-3-bromo-2-hydroxybutanenitrile, (R,S)-2-chloro-3-hydroxybutanamide, (R,S)-2-bromo-3-hydroxybutanamide, (R,S)-3-chloro-2-hydroxybutanamide, (R,S)-3-bromo-2-hydroxybutanamide, (R,S)-2-chloro-3-hydroxybutanoic acid, (R,S)-2-bromo-3-hydroxybutanoic acid, (R,S)-3-chloro-2-hydroxybutanoic acid, (R,S)-3-bromo-2-hydroxybutanoic acid, (R,S)-alkyl 2-chloro-3-hydroxybutanoate, (R,S)-alkyl 2-bromo-3-hydroxybutanoate, (R,S)-alkyl 3-chloro-2-hydroxybutanoate, (R,S)-alkyl 3-bromo-2-hydroxybutanoate, (S,R)-2-chloro-3-hydroxybutanenitrile, (S,R)-2-bromo-3-hydroxybutanenitrile, (S,R)-3-chloro-2-hydroxybutanenitrile, (S,R)-3-bromo-2-hydroxybutanenitrile, (S,R)-2-chloro-3-hydroxybutanamide, (S,R)-2-bromo-3-hydroxybutanamide, (S,R)-3-chloro-2-hydroxybutanamide, (S,R)-3-bromo-2-hydroxybutanamide, (S,R)-2-chloro-3-hydroxybutanoic acid, (S,R)-2-bromo-3-hydroxybutanoic acid, (S,R)-3-chloro-2-hydroxybutanoic acid, (S,R)-3-bromo-2-hydroxybutanoic acid, (S,R)-alkyl 2-chloro-3-hydroxybutanoate, (S,R)-alkyl 2-bromo-3-hydroxybutanoate, (S,R)-alkyl 3-chloro-2-hydroxybutanoate, (S,R)-alkyl 3-bromo-2-hydroxybutanoate, (R,R)-2,3-dichloro-4-hydroxybutanenitrile, (R,R)-2,3-dibromo-4-hydroxybutanenitrile, (R,R)-2,3-dichloro-4-hydroxybutanamide, (S,R)-2,3-dibromo-4-hydroxybutanamide, (R,R)-2,3-dichloro-4-hydroxybutanoic acid, (R,R)-2,3-dibromo-4-hydroxybutanoic acid, (R,R)-alkyl 2,3-dichloro-4-hydroxybutanoate , (R,R)-alkyl 2,3-dibromo-4-hydroxybutanoate, (R,R)-2,4-dichloro-3-hydroxybutanenitrile, (R,R)-2,4-dibromo-3-hydroxybutanenitrile, (R,R)-2,4-dichloro-3-hydroxybutanamide, (R,R)-2,4-dibromo-3-hydroxybutanamide, (R,R)-2,4-dichloro-3-hydroxybutanoic acid, (R,R)-2,4-dibromo-3-hydroxybutanoic acid, (R,R)-alkyl 2,4-dichloro-3-hydroxybutanoate, (R,R)-alkyl 2,4-dibromo-3-hydroxybutanoate, (R,R)-3,4-dichloro-2-hydroxybutanenitrile, (R,R)-3,4-dibromo-2-hydroxybutanenitrile, (R,R)-3,4-dichloro-2-hydroxybutanamide, (R,R)-3,4-dibromo-2-hydroxybutanamide, (R,R)-3,4-dichloro-2-hydroxybutanoic acid, (R,R)-3,4-dibromo-2-hydroxybutanoic acid, (R,R)-alkyl 3,4-dichloro-2-hydroxybutanoate, (R,R)-alkyl 3,4-dibromo-2-hydroxybutanoate, (S,S)-2,3-dichloro-4-hydroxybutanenitrile, (S,S)-2,3-dibromo-4-hydroxybutanenitrile, (S,S)-2,3-dichloro-4-hydroxybutanamide, (S,S)-2,3-dibromo-4-hydroxybutanamide, (S,S)-2,3-dichloro-4-hydroxybutanoic acid, (S,S)-2,3-dibromo-4-hydroxybutanoic acid, (S,S)-alkyl 2,3-dichloro-4-hydroxybutanoate, (S,S)-alkyl 2,3-dibromo-4-hydroxybutanoate, (S,S)-2,4-dichloro-3-hydroxybutanenitrile, (S,S)-2,4-dibromo-3-hydroxybutanenitrile, (S,S)-2,4-dichloro-3-hydroxybutanamide, (S,S)-2,4-dibromo-3-hydroxybutanamide, (S,S)-2,4-dichloro-3-hydroxybutanoic acid, (S,S)-2,4-dibromo-3-hydroxybutanoic acid, (S,S)-alkyl 2,4-dichloro-3-hydroxybutanoate, (S,S)-alkyl 2,4-dibromo-3-hydroxybutanoate, (S,S)-3,4-dichloro-2-hydroxybutanenitrile, (S,S)-3,4-dibromo-2-hydroxybutanenitrile, (S,S)-3,4-dichloro-2-hydroxybutanamide, (S,S)-3,4-dibromo-2-hydroxybutanamide, (S,S)-3,4-dichloro-2-hydroxybutanoic acid, (S,S)-3,4-dibromo-2-hydroxybutanoic acid, (S,S)-alkyl 3,4-dichloro-2-hydroxybutanoate, (S,S)-alkyl 3,4-dibromo-2-hydroxybutanoate, (R,S)-2,3-dichloro-4-hydroxybutanenitrile, (R,S)-2,3-dibromo-4-hydroxybutanenitrile, (S,S)-2,3-dichloro-4-hydroxybutanamide, (R,S)-2,3-dibromo-4-hydroxybutanamide, (R,S)-2,3-dichloro-4-hydroxybutanoic acid, (R,S)-2,3-dibromo-4-hydroxybutanoic acid, (R,S)-alkyl 2,3-dichloro-4-hydroxybutanoate, (R,S)-alkyl 2,3-dibromo-4-hydroxybutanoate, (R,S)-2,4-dichloro-3-hydroxybutanenitrile, (R,S)-2,4-dibromo-3-hydroxybutanenitrile, (R,S)-2,4-dichloro-3-hydroxybutanamide, (R,S)-2,4-dibromo-3,4hydroxybutanamide, (R,S)-2,4-dichloro-3-hydroxybutanoic acid, (R,S)-2,4-dibromo-3-hydroxybutanoic acid, (R,S)-alkyl 2,4-dichloro-3-hydroxybutanoate , (R,S)-alkyl 2,4-dibromo-3-hydroxybutanoate , (R,S)-3,4-dichloro-2-hydroxybutanenitrile, (R,S)-3,4-dibromo-2-hydroxybutanenitrile, (R,S)-3,4-dichloro-2-hydroxybutanamide, (R,S)-3,4-dibromo-2-hydroxybutanamide, (R,S)-3,4-dichloro-2-hydroxybutanoic acid, (R,S)-3,4-dibromo-2-hydroxybutanoic acid , (R,S)-alkyl 3,4-dichloro-2-hydroxybutanoate, (R,S)-alkyl 3,4-dibromo-2-hydroxybutanoate, (S,R)-2,3-dichloro-4-hydroxybutanenitrile, (S,R)-2,3-dibromo-4-hydroxybutanenitrile, (S,R)-2,3-dichloro-4-hydroxybutanamide, (S,R)-2,3-dibromo-4-hydroxybutanamide, (S,R)-2,3-dichloro-4-hydroxybutanoic acid, (S,R)-2,3-dibromo-4-hydroxybutanoic acid, (S,R)-alkyl 2,3-dichloro-4-hydroxybutanoate, (S,R)-alkyl 2,3-dibromo-4-hydroxybutanoate, (S,R)-2,4-dichloro-3-hydroxybutanenitrile, (S,R)-2,4-dibromo-3-hydroxybutanenitrile, (S,R)-2,4-dichloro-3-hydroxybutanamide, (S,R)-2,4-dibromo-3-hydroxybutanamide, (S,R)-2,4-dichloro-3-hydroxybutanoic acid, (S,R)-2,4-dibromo-3-hydroxybutanoic acid, (S,R)-alkyl 2,4-dichloro-3-hydroxybutanoate, (S,R)-alkyl 2,4-dibromo-3-hydroxybutanoate, (S,R)-3,4-dichloro-2-hydroxybutanenitrile, (S,R)-3,4-dibromo-2-hydroxybutanenitrile, (S,R)-3,4-dichloro-2-hydroxybutanamide, (S,R)-3,4-dibromo-2-hydroxybutanamide, (S,R)-3,4-dichloro-2-hydroxybutanoic acid, (S,R)-3,4-dibromo-2-hydroxybutanoic acid, (S,R)-alkyl 3,4-dichloro-2-hydroxybutanoate, (S,R)-alkyl 3,⁴-dibromo-2-hydroxybutanoate, and the like.

The term “polyol” refers to an organic molecule or group that includes two or more hydroxy groups, but which can also include other substituents, such as amino, alkoxy, cyano, carboxy, halo, and/or other groups. They can include diols, triols, and the like. Polyols that can function as reactants and/or products of the invention include, e.g., 1,3-propanediol, 1,2-ethanediol, 1,2-propanediol, 1,2,3-propanetriol, 1,2-butanediol, 2,3-dihydoxypropanenitrile, 3,4-dihydoxybutanenitrile, 2,3-dihydoxypropanamide, 3,4-dihydoxybutanamide, 2,3-dihydoxypropanoic acid, 3,4-dihydoxybutanoic acid, alkyl 2,3-dihydroxypropanoate, alkyl 3,4-dihydroxybutanoate, 2,3-dihydroxybutanenitrile, 2,3-dihydroxybutanamide, 2,3-dihydroxybutanoic acid, alkyl 2,3-dihydroxybutanoate, and the like.

Polyol enantiomers that can function as reactants and/or products of the invention include, e.g., (R)-1,2-propanediol, (R)-1,2-butanediol, (S)-1,2-propanediol, (S)-1,2-butanediol, (R)-2,3-dihydoxypropanenitrile, (S)-2,3-dihydoxypropanenitrile, (R)-3,4-dihydoxybutanenitrile, (S)-3,4-dihydoxybutanenitrile, (R)-2,3-dihydoxypropanamide, (S)-2,3-dihydoxypropanamide, (R)-3,4-dihydoxybutanamide, (S)-3,4-dihydoxybutanamide, (R)-2,3-dihydoxypropanoic acid, (S)-2,3-dihydoxypropanoic acid, (R)-3,4-dihydoxybutanoic acid, (S)-3,4-dihydoxybutanoic acid, (R)-alkyl 2,3-dihydroxypropanoate, (R)-alkyl 3,4-dihydroxybutanoate, (S)-alkyl 2,3-dihydroxypropanoate, (S)-alkyl 3,4-dihydroxybutanoate, (R,R)-2,3-dihydroxybutanenitrile, (R,R)-2,3-dihydroxybutanamide, (R,R)-2,3-dihydroxybutanoic acid, (R,R)-alkyl 2,3-dihydroxybutanoate, (S,S)-2,3-dihydroxybutanenitrile, (S,S)-2,3-dihydroxybutanamide, (S,S)-2,3-dihydroxybutanoic acid, (S,S)-alkyl 2,3-dihydroxybutanoate, (R,S)-2,3-dihydroxybutanenitrile, (R,S)-2,3-dihydroxybutanamide, (R,S)-2,3-dihydroxybutanoic acid, (R,S)-alkyl 2,3-dihydroxybutanoate, (S,R)-2,3-dihydroxybutanenitrile, (S,R)-2,3-dihydroxybutanamide, (S,R)-2,3-dihydroxybutanoic acid, (S,R)-alkyl 2,3-dihydroxybutanoate, and the like.

The term “halopolyol” is a halo-substituted polyol. Examples of halopolyols and/or intermediate halopolyols that can function as reactants and/or products of the invention include 2-chloro-1,3-propanediol, 2,3-dichloro-1,4-butanediol, 2,4-dichloro-1,5-pentanediol, 2,5-dichloro-1,6-hexanediol, 2,6-dichloro-1,7-heptanediol, 2,7-dichloro-1,8-octanediol, 2-halo-3,4-dihydroxybutanenitrile, 2-halo-3,4-dihydroxybutanamide, 2-halo-3,4-dihydroxybutanoic acid, alkyl 2-halo-3,4-dihydroxybutanoate, 3-halo-2,4-dihydroxybutanenitrile, 3-halo-2,4-dihydroxybutanamide, 3-halo-2,4-dihydroxybutanoic acid, alkyl 3-halo-2,4-dihydroxybutanoate, 4-halo-2,3-dihydroxybutanenitrile, 4-halo-2,3-dihydroxybutanamide, 4-halo-2,3-dihydroxybutanoic acid, alkyl 4-halo-2,3-dihydroxybutanoate, 2-chloro-3,4-dihydroxybutanenitrile, 2-bromo-3,4-dihydroxybutanenitrile, 2-chloro-3,4-dihydroxybutanamide, 2-bromo-3,4-dihydroxybutanamide, 2-chloro-3,4-dihydroxybutanoic acid, 2-bromo-3,4-dihydroxybutanoic acid, alkyl 2-chloro-3,4-dihydroxybutanoate, alkyl 2-bromo-3,4-dihydroxybutanoate, 3-chloro-2,4-dihydroxybutanenitrile, 3-bromo-2,4-dihydroxybutanenitrile, 3-chloro-2,4-dihydroxybutanamide, 3-bromo-2,4-dihydroxybutanamide, 3-chloro-2,4-dihydroxybutanoic acid, 3-bromo-2,4-dihydroxybutanoic acid, alkyl 3-chloro-2,4-dihydroxybutanoate, alkyl 3-bromo-2,4-dihydroxybutanoate, 4-chloro-2,3-dihydroxybutanenitrile, 4-bromo-2,3-dihydroxybutanenitrile, 4-chloro-2,3-dihydroxybutanamide, 4-bromo-2,3-dihydroxybutanamide, 4-chloro-2,3-dihydroxybutanoic acid, 4-bromo-2,3-dihydroxybutanoic acid, alkyl 4-chloro-2,3-dihydroxybutanoate, alkyl 4-bromo-2,3-dihydroxybutanoate, and the like.

Halopolyol enantiomers that can function as reactants and/or products of the invention include, e.g., (R,R)-2,3-dichloro-1,4-butanediol, (S,S)-2,3-dichloro-1,4-butanediol, (R,S)-2,3-dichloro-1,4-butanediol, (R,R)-2,4-dichloro-1,5-pentanediol, (S,S)-2,4-dichloro-1,5-pentanediol, (R,S)-2,4-dichloro-1,5-pentanediol, (R,R)-2,5-dichloro-1,6-hexanediol, (S,S)-2,5-dichloro-1,6-hexanediol, (R,S)-2,5-dichloro-1,6-hexanediol, (R,R)-2,6-dichloro-1,7-heptanediol, (S,S)-2,6-dichloro-1,7-heptanediol, (R,S)-2,6-dichloro-1,7-heptanediol, (R,R)-2,7-dichloro-1,8-octanediol, (S,S)-2,7-dichloro-1,8-octanediol, (R,S)-2,7-dichloro-1,8-octanediol, (R,R)-2-chloro-3,4-dihydroxybutanenitrile, (R,R)-2-bromo-3,4-dihydroxybutanenitrile, (R,R)-2-chloro-3,4-dihydroxybutanamide, (R,R)-2-bromo-3,4-dihydroxybutanamide, (R,R)-2-chloro-3,4-dihydroxybutanoic acid, (R,R)-2-bromo-3,4-dihydroxybutanoic acid, (R,R)-alkyl 2-chloro-3,4-dihydroxybutanoate, (R,R)-alkyl 2-bromo-3,4-dihydroxybutanoate, (R,R)-3-chloro-2,4-dihydroxybutanenitrile, (R,R)-3-bromo-2,4-dihydroxybutanenitrile, (R,R)-3-chloro-2,4-dihydroxybutanamide, (R,R)-3-bromo-2,4-dihydroxybutanamide, (R,R)-3-chloro-2,4-dihydroxybutanoic acid, (R,R)-3-bromo-2,4-dihydroxybutanoic acid, (R,R)-alkyl 3-chloro-2,4-dihydroxybutanoate, (R,R)-alkyl 3-bromo-2,4-dihydroxybutanoate, (R,R)-4-chloro-2,3-dihydroxybutanenitrile, (R,R)-4-bromo-2,3-dihydroxybutanenitrile, (R,R)-4-chloro-2,3-dihydroxybutanamide, (R,R)-4-bromo-2,3-dihydroxybutanamide, (R,R)-4-chloro-2,3-dihydroxybutanoic acid, (R,R)-4-bromo-2,3-dihydroxybutanoic acid, (R,R)-alkyl 4-chloro-2,3-dihydroxybutanoate, (R,R)-alkyl 4-bromo-2,3-dihydroxybutanoate, (S,S)-2-chloro-3,4-dihydroxybutanenitrile, (S,S)-2-bromo-3,4-dihydroxybutanenitrile, (S,S)-2-chloro-3,4-dihydroxybutanamide, (S,S)-2-bromo-3,4-dihydroxybutanamide, (S,S)-2-chloro-3,4-dihydroxybutanoic acid, (S,S)-2-bromo-3,4-dihydroxybutanoic acid, (S,S)-alkyl 2-chloro-3,4-dihydroxybutanoate, (S,S)-alkyl 2-bromo-3,4-dihydroxybutanoate, (S,S)-3-chloro-2,4-dihydroxybutanenitrile, (S,S)-3-bromo-2,4-dihydroxybutanenitrile, (S,S)-3-chloro-2,4-dihydroxybutanamide, (S,S)-3-bromo-2,4-dihydroxybutanamide, (S,S)-3-chloro-2,4-dihydroxybutanoic acid, (S,S)-3-bromo-2,4-dihydroxybutanoic acid, (S,S)-alkyl 3-chloro-2,4-dihydroxybutanoate, (S,S)-alkyl 3-bromo-2,4-dihydroxybutanoate, (S,S)-4-chloro-2,3-dihydroxybutanenitrile, (S,S)-4-bromo-2,3-dihydroxybutanenitrile, (S,S)-4-chloro-2,3-dihydroxybutanamide, (S,S)-4-bromo-2,3-dihydroxybutanamide, (S,S)-4-chloro-2,3-dihydroxybutanoic acid, (S,S)-4-bromo-2,3-dihydroxybutanoic acid, (S,S)-alkyl 4-chloro-2,3-dihydroxybutanoate, (S,S)-alkyl 4-bromo-2,3-dihydroxybutanoate, (R,S)-2-chloro-3,4-dihydroxybutanenitrile, (R,S)-2-bromo-3,4-dihydroxybutanenitrile, (R,S)-2-chloro-3,4-dihydroxybutanamide, (R,S)-2-bromo-3,4-dihydroxybutanamide, (R,S)-2-chloro-3,4-dihydroxybutanoic acid, (R,S)-2-bromo-3,4-dihydroxybutanoic acid, (R,S)-alkyl 2-chloro-3,4-dihydroxybutanoate, (R,S)-alkyl 2-bromo-3,4-dihydroxybutanoate, (R,S)-3-chloro-2,4-dihydroxybutanenitrile, (R,S)-3-bromo-2,4-dihydroxybutanenitrile, (R,S)-3-chloro-2,4-dihydroxybutanamide, (R,S)-3-bromo-2,4-dihydroxybutanamide, (R,S)-3-chloro-2,4-dihydroxybutanoic acid, (R,S)-3-bromo-2,4-dihydroxybutanoic acid, (R,S)-alkyl 3-chloro-2,4-dihydroxybutanoate, (R,S)-alkyl 3-bromo-2,4-dihydroxybutanoate, (R,S)-4-chloro-2,3-dihydroxybutanenitrile, (R,S)-4-bromo-2,3-dihydroxybutanenitrile, (R,S)-4-chloro-2,3-dihydroxybutanamide, (R,S)-4-bromo-2,3-dihydroxybutanamide, (R,S)-4-chloro-2,3-dihydroxybutanoic acid, (R,S)-4-bromo-2,3-dihydroxybutanoic acid, (R,S)-alkyl 4-chloro-2,3-dihydroxybutanoate, (R,S)-alkyl 4-bromo-2,3-dihydroxybutanoate, (S,R)-2-chloro-3,4-dihydroxybutanenitrile, (S,R)-2-bromo-3,4-dihydroxybutanenitrile, (S,R)-2-chloro-3,4-dihydroxybutanamide, (S,R)-2-bromo-3,4-dihydroxybutanamide, (S,R)-2-chloro-3,4-dihydroxybutanoic acid, (S,R)-2-bromo-3,4-dihydroxybutanoic acid, (S,R)-alkyl 2-chloro-3,4-dihydroxybutanoate, (S,R)-alkyl 2-bromo-3,4-dihydroxybutanoate, (S,R)-3-chloro-2,4-dihydroxybutanenitrile, (S,R)-3-bromo-2,4-dihydroxybutanenitrile, (S,R)-3-chloro-2,4-dihydroxybutanamide, (S,R)-3-bromo-2,4-dihydroxybutanamide, (S,R)-3-chloro-2,4-dihydroxybutanoic acid, (S,R)-3-bromo-2,4-dihydroxybutanoic acid, (S,R)-alkyl 3-chloro-2,4-dihydroxybutanoate, (S,R)-alkyl 3-bromo-2,4-dihydroxybutanoate, (S,R)-4-chloro-2,3-dihydroxybutanenitrile, (S,R)-4-bromo-2,3-dihydroxybutanenitrile, (S,R)-4-chloro-2,3-dihydroxybutanamide, (S,R)-4-bromo-2,3-dihydroxybutanamide, (S,R)-4-chloro-2,3-dihydroxybutanoic acid, (S,R)-4-bromo-2,3-dihydroxybutanoic acid, (S,R)-alkyl 4-chloro-2,3-dihydroxybutanoate, (S,R)-alkyl 4-bromo-2,3-dihydroxybutanoate, and the like.

The term “epoxide” refers to an organic molecule or group that includes at least an oxygen atom in a three-membered ring (i.e., a cyclic ether), but which can also include other substituent groups. A “haloepoxide” is a halo-substituted epoxide. Examples of epoxides and haloepoxides that can function as reactants and/or products of the invention include 1-halo-2,3-epoxypropane, 1,2-dihalo-3,4-epoxybutane, 1,2-dihalo-4,5-epoxypentane, 1,2-dihalo-5,6-epoxyhexane, 1,2-dihalo-6,7-epoxyheptane, 1,2-dihalo-7,8-epoxyoctane, 4-halo-2,3-epoxybutanenitrile, 4-halo-2,3-epoxybutanamide, 4-halo-2,3-epoxybutanoic acid, alkyl 4-halo-2,3-epoxybutanoate, 2-halo-3,4-epoxybutanenitrile, 2-halo-3,4-epoxybutanamide, 2-halo-3,4-epoxybutanoic acid, alkyl 2-halo-3,4-epoxybutanoate, 1-chloro-2,3-epoxypropane, 2,3-epoxy-1-propanol, 1,2-epoxypropane, ethylene oxide, 1,2-dichloro-3,4-epoxybutane, 1,2-dichloro-4,5-epoxypentane, 1,2-dichloro-5,6-epoxyhexane, 1,2-dichloro-6,7-epoxyheptane, 1,2-dichloro-7,8-epoxyoctane, 1,2,3,4-diepoxybutane, 1,2,4,5-diepoxypentane, 1,2,5,6-diepoxyhexane, 1,2,6,7-diepoxyheptane, 1,2,7,8-diepoxyoctane, 2,3-epoxy-1-propanenitrile, 3,4-epoxy-1-butanenitrile, 2,3-epoxy-1-propanamide, 3,4-epoxy-1-butanamide, 2,3-epoxy-1-propanoic acid, 3,4-epoxy-1-butanoic acid, alkyl 2,3-epoxy-1-propanoate, alkyl 3,4-epoxy-1-butanoate, 2,3-epoxy-1-butanenitrile, 2,3-epoxy-1-butanamide, 2,3-epoxy-1-butanoic acid, alkyl 2,3-epoxy-1-butanoate, 4-hydroxy-2,3-epoxybutanenitrile, 4-hydroxy-2,3-epoxybutanamide, 4-hydroxy-2,3-epoxybutanoic acid, alkyl 4-hydroxy-2,3-epoxybutanoate, 2-hydroxy-3,4-epoxybutanenitrile, 2-hydroxy-3,4-epoxybutanamide, 2-hydroxy-3,4-epoxybutanoic acid, alkyl 2-hydroxy-3,4-epoxybutanoate, 4-chloro-2,3-epoxybutanenitrile, 4-bromo-2,3-epoxybutanenitrile, 4-chloro-2,3-epoxybutanamide, 4-bromo-2,3-epoxybutanamide, 4-chloro-2,3-epoxybutanoic acid, 4-bromo-2,3-epoxybutanoic acid, alkyl 4-chloro-2,3-epoxybutanoate, alkyl 4-bromo-2,3-epoxybutanoate, 2-chloro-3,4-epoxybutanenitrile, 2-bromo-3,4-epoxybutanenitrile, 2-chloro-3,4-epoxybutanamide, 2-bromo-3,4-epoxybutanamide, 2-chloro-3,4-epoxybutanoic acid, 2-bromo-3,4-epoxybutanoic acid, alkyl 2-chloro-3,4-epoxybutanoate, alkyl 2-bromo-3,4-epoxybutanoate, and the like.

Enantiomers of epoxides and haloepoxides that can function as reactants and/or products of the invention include, e.g., (R)-1-chloro-2,3-epoxypropane, (R)-2,3-epoxy-1-propanol, (R)-1,2-epoxypropane, (S)-1-chloro-2,3-epoxypropane, (S)-2,3-epoxy-1-propanol, (S)-1,2-epoxypropane, (R,R)-1,2-dichloro-3,4-epoxybutane, (S,S)1,2-dichloro-3,4-epoxybutane, (R,S)-1,2-dichloro-3,4-epoxybutane, (S,R)-1,2-dichloro-3,4-epoxybutane, (R,R)-1,2-dichloro-4,5-epoxypentane, (S,S)-1,2-dichloro-4,5-epoxypentane, (R,S)-1,2-dichloro-4,5-epoxypentane, (S,R)-1,2-dichloro-4,5-epoxypentane, (R,R)-1,2-dichloro-5,6-epoxyhexane, (S,R)-1,2-dichloro-5,6-epoxyhexane, (R,S)-1,2-dichloro-5,6-epoxyhexane, (S,R)-1,2-dichloro-5,6-epoxyhexane, (R,R)-1,2-dichloro-6,7-epoxyheptane, (S,S)-1,2-dichloro-6,7-epoxyheptane, (R,S)-1,2-dichloro-6,7-epoxyheptane, (S,R)-1,2-dichloro-6,7-epoxyheptane, (R,R)-1,2-dichloro-7,8-epoxyoctane, (S,S)-1,2-dichloro-7,8-epoxyoctane, (R,S)-1,2-dichloro-7,8-epoxyoctane, (S,R)-1,2-dichloro-7,8-epoxyoctane, (R,R)-1,2,3,4-diepoxybutane, (S,S)-1,2,3,4-diepoxybutane, (R,S)-1,2,3,4-diepoxybutane, (R,R)-1,2,4,5-diepoxypentane, (S,S)-1,2,4,5-diepoxypentane, (R,S)-1,2,4,5-diepoxypentane, (R,R)-1,2,5,6-diepoxyhexane, (S,S)-1,2,5,6-diepoxyhexane, (R,S)-1,2,5,6-diepoxyhexane, (R,R)-1,2,6,7-diepoxyheptane, (S,S)-1,2,6,7-diepoxyheptane, (R,S)-1,2,6,7-diepoxyheptane, (R,R)-1,2,7,8-diepoxyoctane, (S,S)-1,2,7,8-diepoxyoctane, (R,S)-1,2,7,8-diepoxyoctane, (R)-2,3-epoxy-1-propanenitrile, (S)-2,3-epoxy-1-propanenitrile, (R)-3,4-epoxy-1-butanenitrile, (S)-3,4-epoxy-1-butanenitrile, (R)-2,3-epoxy-1-propanamide, (S)-2,3-epoxy-1-propanamide, (R)-3,4-epoxy-1-butanamide, (S)-3,4-epoxy-1-butanamide, (R)-2,3-epoxy-1-propanoic acid, (S)-2,3-epoxy-1-propanoic acid, (R)-3,4-epoxy-1-butanoic acid, (S)-3,4-epoxy-1-butanoic acid, (R)-alkyl 2,3-epoxy-1-propanoate, (R)-alkyl 3,4-epoxy-1-butanoate, (S)-alkyl 2,3-epoxy-1-propanoate, (S)-alkyl 3,4-epoxy-1-butanoate, (R,R)-2,3-epoxy-1-butanenitrile, (R,R)-2,3-epoxy-1-butanamide, (R,R)-2,3-epoxy-1-butanoic acid, (R,R)-alkyl 2,3-epoxy-1-butanoate, (S,S)-2,3-epoxy-1-butanenitrile, (S,S)-2,3-epoxy-1-butanamide, (S,S)-2,3-epoxy-1-butanoic acid, (S,S)-alkyl 2,3-epoxy-1-butanoate, (R,S)-2,3-epoxy-1-butanenitrile, (R,S)-2,3-epoxy-1-butanamide, (R,S)-2,3-epoxy-1-butanoic acid, (R,S)-alkyl 2,3-epoxy-1-butanoate, (S,R)-2,3-epoxy-1-butanenitrile, (S,R)-2,3-epoxy-1-butanamide, (S,R)-2,3-epoxy-1-butanoic acid, (S,R)-alkyl 2,3-epoxy-1-butanoate, (R,R)-4-hydroxy-2,3-epoxybutanenitrile, (R,R)-4-hydroxy-2,3-epoxybutanamide, (R,R)-4-hydroxy-2,3-epoxybutanoic acid, (R,R)-alkyl 4-hydroxy-2,3-epoxybutanoate, (R,R)-2-hydroxy-3,4-epoxybutanenitrile, (R,R)-2-hydroxy-3,4-epoxybutanamide, (R,R)-2-hydroxy-3,4-epoxybutanoic acid, (R,R)-alkyl 2-hydroxy-3,4-epoxybutanoate, (S,S)-4-hydroxy-2,3-epoxybutanenitrile, (S,S)-4-hydroxy-2,3-epoxybutanamide, (S,S)-4-hydroxy-2,3-epoxybutanoic acid, (S,S)-alkyl 4-hydroxy-2,3-epoxybutanoate, (S,S)-2-hydroxy-3,4-epoxybutanenitrile, (S,S)-2-hydroxy-3,4-epoxybutanamide, (S,S)-2-hydroxy-3,4-epoxybutanoic acid, (S,S)-alkyl 2-hydroxy-3,4-epoxybutanoate, (R,S)-4-hydroxy-2,3-epoxybutanenitrile, (R,S)-4-hydroxy-2,3-epoxybutanamide, (R,S)-4-hydroxy-2,3-epoxybutanoic acid, (R,S)-alkyl 4-hydroxy-2,3-epoxybutanoate, (R,S)-2-hydroxy-3,4-epoxybutanenitrile, (R,S)-2-hydroxy-3,4-epoxybutanamide, (R,S)-2-hydroxy-3,4-epoxybutanoic acid, (R,S)-alkyl 2-hydroxy-3,4-epoxybutanoate, (S,R)-4-hydroxy-2,3-epoxybutanenitrile, (S,R)-4-hydroxy-2,3-epoxybutanamide, (S,R)-4-hydroxy-2,3-epoxybutanoic acid, (S,R)-alkyl 4-hydroxy-2,3-epoxybutanoate, (S,R)-2-hydroxy-3,4-epoxybutanenitrile, (S,R)-2-hydroxy-3,4-epoxybutanamide, (S,R)-2-hydroxy-3,4-epoxybutanoic acid, (S,R)-alkyl 2-hydroxy-3,4-epoxybutanoate, (R,R)-4-chloro-2,3-epoxybutanenitrile, (R,R)-4-bromo-2,3-epoxybutanenitrile, (R,R)-4-chloro-2,3-epoxybutanamide, (R,R)-4-bromo-2,3-epoxybutanamide, (R,R)-4-chloro-2,3-epoxybutanoic acid, (R,R)-4-bromo-2,3-epoxybutanoic acid, (R,R)-alkyl 4-chloro-2,3-epoxybutanoate, (R,R)-alkyl 4-bromo-2,3-epoxybutanoate, (R,R)-2-chloro-3,4-epoxybutanenitrile, (R,R)-2-bromo-3,4-epoxybutanenitrile, (R,R)-2-chloro-3,4-epoxybutanamide, (R,R)-2-bromo-3,4-epoxybutanamide, (R,R)-2-chloro-3,4-epoxybutanoic acid, (R,R)-2-bromo-3,4-epoxybutanoic acid, (R,R)-alkyl 2-chloro-3,4-epoxybutanoate, (R,R)-alkyl 2-bromo-3,4-epoxybutanoate, (S,S)-4-chloro-2,3-epoxybutanenitrile, (S,S)-4-bromo-2,3-epoxybutanenitrile, (S,S)-4-chloro-2,3-epoxybutanamide, (S,S)-4-bromo-2,3-epoxybutanamide, (S,S)-4-chloro-2,3-epoxybutanoic acid, (S,S)-4-bromo-2,3-epoxybutanoic acid, (S,S)-alkyl 4-chloro-2,3-epoxybutanoate, (S,S)-alkyl 4-bromo-2,3-epoxybutanoate, (S,S)-2-chloro-3,4-epoxybutanenitrile, (S,S)-2-bromo-3,4-epoxybutanenitrile, (S,S)-2-chloro-3,4-epoxybutanamide, (S,S)-2-bromo-3,4-epoxybutanamide, (S,S)-2-chloro-3,4-epoxybutanoic acid, (S,S)-2-bromo-3,4-epoxybutanoic acid, (S,S)-alkyl 2-chloro-3,4-epoxybutanoate, (S,S)-alkyl 2-bromo-3,4-epoxybutanoate, (R,S)-4-chloro-2,3-epoxybutanenitrile, (R,S)-4-bromo-2,3-epoxybutanenitrile, (R,S)-4-chloro-2,3-epoxybutanamide, (R,S)-4-bromo-2,3-epoxybutanamide, (R,S)-4-chloro-2,3-epoxybutanoic acid, (R,S)-4-bromo-2,3-epoxybutanoic acid, (R,S)-alkyl 4-chloro-2,3-epoxybutanoate, (R,S)-alkyl 4-bromo-2,3-epoxybutanoate, (R,S)-2-chloro-3,4-epoxybutanenitrile, (R,S)-2-bromo-3,4-epoxybutanenitrile, (R,S)-2-chloro-3,4-epoxybutanamide, (R,S)-2-bromo-3,4-epoxybutanamide, (R,S)-2-chloro-3,4-epoxybutanoic acid, (R,S)-2-bromo-3,4-epoxybutanoic acid, (R,S)-alkyl 2-chloro-3,4-epoxybutanoate, (R,S)-alkyl 2-bromo-3,4-epoxybutanoate, (S,R)-4-chloro-2,3-epoxybutanenitrile, (S,R)-4-bromo-2,3-epoxybutanenitrile, (S,R)-4-chloro-2,3-epoxybutanamide, (S,R)-4-bromo-2,3-epoxybutanamide, (S,R)-4-chloro-2,3-epoxybutanoic acid, (S,R)-4-bromo-2,3-epoxybutanoic acid, (S,R)-alkyl 4-chloro -2,3-epoxybutanoate, (S,R)-alkyl 4-bromo-2,3-epoxybutanoate, (S,R)-2-chloro-3,4-epoxybutanenitrile, (S,R)-2-bromo-3,4-epoxybutanenitrile, (S,R)-2-chloro-3,4-epoxybutanamide, (S,R)-2-bromo-3,4-epoxybutanamide, (S,R)-2-chloro-3,4-epoxybutanoic acid, (S,R)-2-bromo-3,4-epoxybutanoic acid, (S,R)-alkyl 2-chloro-3,4-epoxybutanoate, (S,R)-alkyl 2-bromo-3,4-epoxybutanoate, and the like.

A chemical process that forms unequal quantities of product stereoisomers is “stereoselective.” By comparison, a “stereospecific” reaction produces only one product stereoisomer. Similarly, a process is “enantioselective” if it yields an excess of one enantiomer (i.e., more than 50% but less than 100%), whereas an “enantiospecific” process yields one enantiomer exclusively. A “chiral center,” as used herein, refers to a carbon atom with four different point ligands attached thereto. If a chiral center is generated when a point ligand is replaced by a new point ligand, the original center is a “prochiral center.”

An “artificially evolved hydrolase,” “altered hydrolase,” or “altered dehalogenase” refers to a protein- or nucleic acid-based catalyst or enzyme, such as a dehalogenase or the like, created using one or more diversity generating techniques. For example, the artificially evolved hydrolases of the invention are optionally produced by recombining (e.g., via recursive recombination, whole genome recombination, synthetic recombination, in silico recombination, or the like) two or more nucleic acids encoding one or more parental enzymes, or by mutating one or more nucleic acids that encode hydrolases, e.g., using site directed mutagenesis, cassette mutagenesis, random mutagenesis, recursive ensemble mutagenesis, in vivo mutagenesis, or the like. A nucleic acid encoding a parental enzyme includes a polynucleotide or gene that, through the mechanisms of transcription and translation, produces an amino acid sequence corresponding to a parental enzyme, e.g., an unevolved or naturally-occurring hydrolase. Artificially evolved hydrolases also relate to chimeric enzymes that include identifiable component sequences (e.g., functional domains, etc.) derived from two or more parents. Additionally, when a particular product includes stereoisomers (e.g., enantiomeric isomers), artificially evolved hydrolases typically yield the product in a stereoselective (e.g., enantioselective) manner. They are also optionally evolved to yield those products stereospecifically (e.g., enantiospecifically). Diversity generating methodologies that are optionally used to produce the artificially evolved hydrolases of the present invention are discussed in greater detail below.

Hydrolases, which include dehalogenases, can catalyze the hydrolysis of a wide variety of bonds including, e.g., carbon-oxygen bonds of epoxide rings. Dehalogenases can catalyze the removal of one or more halogen atoms from a molecule or substituent group. Both dehalogenases and hydrolases are classified by the Enzyme Commission (EC) number 3. Altered dehalogenases can convert various cyclic and acyclic halocarbon and/or halohydrocarbon reactants (e.g., haloalcohols, halohydrins, halopolyols, haloepoxides, haloalkanes, haloalkenes, haloalkynes, haloalkyl nitriles, haloalkyl amides, haloalkyl carboxylic acids (e.g., α-haloacids, β-haloacids, etc.), haloalkyl carboxylic acid esters, and the like) to one or more other halocarbon, halohydrocarbon or hydrocarbon products (e.g., alcohols, haloalcohols, halohydrins, polyols, halopolyols, epoxides, haloepoxides, and the like).

As used herein, the terms “altered hydrolase” and “altered dehalogenase” can be further characterized by the reactant upon which a particular enzyme acts and/or the particular products which a particular enzyme produces. For example, an “altered 2,3-dichloro-1-propanol providing halohydrocarbon dehalogenase” is an altered dehalogenase that converts a halohydrocarbon (e.g., 1,2,3-trichloropropane) to 2,3-dichloro-1-propanol. Aside from catalyzing one reaction or a single step in a reaction pathway, an altered dehalogenase or an altered hydrolase can also include enzymes (e.g., a single enzyme) that catalyze multiple reactions or steps in a particular reaction pathway. For example, an “altered 1-chloro-2,3-epoxypropane providing halohydrocarbon-haloalcohol dehalogenase” is an altered dehalogenase that can catalyze the conversion of a halohydrocarbon (e.g., 1,2,3-trichloropropane) to 1-chloro-2,3-epoxypropane through an intermediate haloalcohol (e.g., 2,3-dichloro-1-propanol). As an additional example, an “altered at least predominantly (R)-2,3-dichloro-1-propanol providing halohydrocarbon dehalogenase” is an altered dehalogenase that converts a halohydrocarbon (e.g., 1,2,3-trichloropropane) at least enantioselectively to (R)-2,3-dichloro-1-propanol. However, this mode of enzyme characterization also includes dehalogenases or other hydrolases that catalyze enantiospecific reactions.

The present invention provides new pathways that employ various classes or groups of altered hydrolase enzymes. In general, these classes or groups include, e.g., altered halohydrocarbon dehalogenases, altered haloalcohol dehalogenases, altered halopolyol dehalogenases, altered epoxide providing halohydrocarbon-haloalcohol isomer dehalogenases, altered halohydrocarbon-haloalcohol dehalogenases, altered haloalcohol-halopolyol dehalogenases, altered halohydrocarbon-haloalcohol-halopolyol dehalogenases, altered first haloalcohol isomer providing halohydrocarbon-second haloalcohol isomer providing halohydrocarbon dehalogenases, altered epoxide providing first haloalcohol isomer-epoxide providing second haloalcohol isomer dehalogenases, altered haloepoxide dehalogenases, altered epoxide hydrolases, altered haloepoxide dehalogenase-epoxide hydrolases, and the like.

An “altered halohydrocarbon dehalogenase” can include, e.g., an altered 2,3-dihalo-1-propanol providing halohydrocarbon dehalogenase, an altered 2-halo-1-propanol providing halohydrocarbon dehalogenase, an altered 1-halo-2-propanol providing halohydrocarbon dehalogenase, an altered 2-halo-1-ethanol providing halohydrocarbon dehalogenase, an altered 3-halo-1-propanol providing halohydrocarbon dehalogenase, an altered 1,3-dihalo-2-propanol providing halohydrocarbon dehalogenase, an altered 2,3,4-trihalo-1-butanol providing halohydrocarbon dehalogenase, an altered 2,4,5-trihalo-1-pentanol providing halohydrocarbon dehalogenase, an altered 2,5,6-trihalo-1-hexanol providing halohydrocarbon dehalogenase, an altered 2,6,7-trihalo-1-heptanol providing halohydrocarbon dehalogenase, an altered 2,7,8-trihalo-1-octanol providing halohydrocarbon dehalogenase, an altered 2-halo-3-hydroxypropanenitrile providing halohydrocarbon dehalogenase, an altered 3-halo-2-hydroxypropanenitrile providing halohydrocarbon dehalogenase, an altered 3-halo-4-hydroxybutanenitrile providing halohydrocarbon dehalogenase, an altered 4-halo-3-hydroxybutanenitrile providing halohydrocarbon dehalogenase, an altered 2-halo-3-hydroxypropanamide providing halohydrocarbon dehalogenase, an altered 3-halo-2-hydroxypropanamide providing halohydrocarbon dehalogenase, an altered 3-halo-4-hydroxybutanamide providing halohydrocarbon dehalogenase, an altered 4-halo-3-hydroxybutanamide providing halohydrocarbon dehalogenase, an altered 2-halo-3-hydroxypropanoic acid providing halohydrocarbon dehalogenase, an altered 3-halo-2-hydroxypropanoic acid providing halohydrocarbon dehalogenase, an altered 3-halo-4-hydroxybutanoic acid providing halohydrocarbon dehalogenase, an altered 4-halo-3-hydroxybutanoic acid providing halohydrocarbon dehalogenase, an altered alkyl 2,3-dihalopropanoate providing halohydrocarbon dehalogenase, an altered alkyl 3,4-dihalobutanoate providing halohydrocarbon dehalogenase, an altered alkyl 2-halo-3-hydroxypropanoate providing halohydrocarbon dehalogenase, an altered alkyl 3-halo-2-hydroxypropanoate providing halohydrocarbon dehalogenase, an altered alkyl 3-halo-4-hydroxybutanoate providing halohydrocarbon dehalogenase, an altered alkyl 4-halo-3-hydroxybutanoate providing halohydrocarbon dehalogenase, an altered 2-halo-3-hydroxybutanenitrile providing halohydrocarbon dehalogenase, an altered 3-halo-2-hydroxybutanenitrile providing halohydrocarbon dehalogenase, an altered 2-halo-3-hydroxybutanamide providing halohydrocarbon dehalogenase, an altered 3-halo-2-hydroxybutanamide providing halohydrocarbon dehalogenase, an altered 2-halo-3-hydroxybutanoic acid providing halohydrocarbon dehalogenase, an altered 3-halo-2-hydroxybutanoic acid providing halohydrocarbon dehalogenase, an altered alkyl 2-halo-3-hydroxybutanoate providing halohydrocarbon dehalogenase, an altered alkyl 3-halo-2-hydroxybutanoate providing halohydrocarbon dehalogenase, an altered 2,3-dihalo-4-hydroxybutanenitrile providing halohydrocarbon dehalogenase, an altered 2,3-dihalo-4-hydroxybutanamide providing halohydrocarbon dehalogenase, an altered 2,3-dihalo-4-hydroxybutanoic acid providing halohydrocarbon dehalogenase, an altered alkyl 2,3-dihalo-4-hydroxybutanoate providing halohydrocarbon dehalogenase, an altered 2,4-dihalo-3-hydroxybutanenitrile providing halohydrocarbon dehalogenase, an altered 2,4-dihalo-3-hydroxybutanamide providing halohydrocarbon dehalogenase, an altered 2,4-dihalo-3-hydroxybutanoic acid providing halohydrocarbon dehalogenase, an altered alkyl 2,4-dihalo-3-hydroxybutanoate providing halohydrocarbon dehalogenase, an altered 3,4-dihalo-2-hydroxybutanenitrile providing halohydrocarbon dehalogenase, an altered 3,4-dihalo-2-hydroxybutanamide providing halohydrocarbon dehalogenase, an altered 3,4-dihalo-2-hydroxybutanoic acid providing halohydrocarbon dehalogenase, an altered alkyl 3,4-dihalo-2-hydroxybutanoate providing halohydrocarbon dehalogenase, an altered 2-halo-3,4-dihydroxybutanenitrile providing halohydrocarbon dehalogenase, an altered 2-halo-3,4-dihydroxybutanamide providing halohydrocarbon dehalogenase, an altered 2-halo-3,4-dihydroxybutanoic acid providing halohydrocarbon dehalogenase, an altered alkyl 2-halo -3,4-dihydroxybutanoate providing halohydrocarbon dehalogenase, an altered 3-halo-2,4-dihydroxybutanenitrile providing halohydrocarbon dehalogenase, an altered 3-halo-2,4-dihydroxybutanamide providing halohydrocarbon dehalogenase, an altered 3-halo-2,4-dihydroxybutanoic acid providing halohydrocarbon dehalogenase, an altered alkyl 3-halo -2,4-dihydroxybutanoate providing halohydrocarbon dehalogenase, an altered 4-halo-2,3-dihydroxybutanenitrile providing halohydrocarbon dehalogenase, an altered 4-halo-2,3-dihydroxybutanamide providing halohydrocarbon dehalogenase, an altered 4-halo-2,3-dihydroxybutanoic acid providing halohydrocarbon dehalogenase, an altered alkyl 4-halo-2,3-dihydroxybutanoate providing halohydrocarbon dehalogenase, an altered 4-halo-2,3-epoxybutanenitrile providing halohydrocarbon dehalogenase, an altered 4-halo -2,3-epoxybutanamide providing halohydrocarbon dehalogenase, an altered 4-halo-2,3-epoxybutanoic acid providing halohydrocarbon dehalogenase, an altered alkyl 4-halo-2,3-epoxybutanoate providing halohydrocarbon dehalogenase, an altered 2-halo-3,4-epoxybutanenitrile providing halohydrocarbon dehalogenase, an altered 2-halo-3,4-epoxybutanamide providing halohydrocarbon dehalogenase, an altered 2-halo-3,4-epoxybutanoic acid providing halohydrocarbon dehalogenase, an altered alkyl 2-halo-3,4-epoxybutanoate providing halohydrocarbon dehalogenase, an altered 2,3-dichloro-1-propanol providing halohydrocarbon dehalogenase, an altered 2-chloro-1-propanol providing halohydrocarbon dehalogenase, an altered 1-chloro-2-propanol providing halohydrocarbon dehalogenase, an altered 2-chloro-1-ethanol providing halohydrocarbon dehalogenase, an altered 1,2-ethanediol providing halohydrocarbon dehalogenase, an altered 1,2-propanediol providing halohydrocarbon dehalogenase, an altered 1,2,3-propanetriol providing halohydrocarbon dehalogenase, an altered 1,3-propanediol providing halohydrocarbon dehalogenase, an altered 1,2-butanediol providing halohydrocarbon dehalogenase, an altered 1-propanol providing halohydrocarbon dehalogenase, an altered 3-chloro-1-propanol providing halohydrocarbon dehalogenase, an altered 2-propen-1-ol providing halohydrocarbon dehalogenase, an altered 1,3-dichloro-2-propanol providing halohydrocarbon dehalogenase, an altered 2,3,4-trichloro-1-butanol providing halohydrocarbon dehalogenase, an altered 2,4,5-trichloro-1-pentanol providing halohydrocarbon dehalogenase, an altered 2,5,6trichloro-1-hexanol providing halohydrocarbon dehalogenase, an altered 2,6,7-trichloro-1-heptanol providing halohydrocarbon dehalogenase, an altered 2,7,8-trichloro-1-octanol providing halohydrocarbon dehalogenase, an altered at least predominantly (R)-2,3-dichloro-1-propanol providing halohydrocarbon dehalogenase, an altered at least predominantly (S)-2,3-dichloro-1-propanol providing halohydrocarbon dehalogenase, an altered at least predominantly (R)-2-chloro-1-propanol providing halohydrocarbon dehalogenase, an altered at least pre dominantly (R)-1-chloro-2-propanol providing halohydrocarbon dehalogenase, an altered at least predominantly (S)-2-chloro -1-propanol providing halohydrocarbon dehalogenase, an altered at least predominantly (S)-1-chloro-2-propanol providing halohydrocarbon dehalogenase, an altered at least predominantly (R)-1,2-propanediol providing halohydrocarbon dehalogenase, an altered at least predominantly (R)-1,2-butanediol providing halohydrocarbon dehalogenase, an altered at least predominantly (S)-1,2-propanediol providing halohydrocarbon dehalogenase, an altered at least predominantly (S)-1,2-butanediol providing halohydrocarbon dehalogenase, an altered at least predominantly (R,R)-2,3,4-trichloro-1-butanol providing halohydrocarbon dehalogenase, an altered at least predominantly (S,S)-2,3,4-trichloro-1-butanol providing halohydrocarbon dehalogenase, an altered at least predominantly (R,S)-2,3,4-trichloro-1-butanol providing halohydrocarbon dehalogenase, an altered at least predominantly (S,R)-2,3,4-trichloro-1-butanol providing halohydrocarbon dehalogenase, an altered at least predominantly (R,R)-2,4,5-trichloro-1-pentanol providing halohydrocarbon dehalogenase, an altered at least predominantly (S,S)-2,4,5-trichloro-1-pentanol providing halohydrocarbon dehalogenase, an altered at least predominantly (R,S)-2,4,5-trichloro-1-pentanol providing halohydrocarbon dehalogenase, an altered at least predominantly (S,R)-2,4,5-trichloro-1-pentanol providing halohydrocarbon dehalogenase, an altered at least predominantly (R,R)-2,5,6-trichloro-1-hexanol providing halohydrocarbon dehalogenase, an altered at least predominantly (S,S)-2,5,6-trichloro-1-hexanol providing halohydrocarbon dehalogenase, an altered at least predominantly (R,S)-2,5,6-trichloro-1-hexanol providing halohydrocarbon dehalogenase, an altered at least predominantly (S,R)-2,5,6-trichloro-1-hexanol providing halohydrocarbon dehalogenase, an altered at least predominantly (R,R)-2,6,7-trichloro-1-heptanol providing halohydrocarbon dehalogenase, an altered at least predominantly (S,S)-2,6,7-trichloro-1-heptanol providing halohydrocarbon dehalogenase, an altered at least predominantly (R,S)-2,6,7-trichloro-1-heptanol providing halohydrocarbon dehalogenase, an altered at least predominantly (S,R)-2,6,7-trichloro-1-heptanol providing halohydrocarbon dehalogenase, an altered at least predominantly (R,R)-2,7,8-trichloro-1-octanol providing halohydrocarbon dehalogenase, an altered at least predominantly (S,S)-2,7,8-trichloro-1-octanol providing halohydrocarbon dehalogenase, an altered at least predominantly (R,S)-2,7,8-trichloro-1-octanol providing halohydrocarbon dehalogenase, an altered at least predominantly (S,R)-2,7,8-trichloro-1-octanol providing halohydrocarbon dehalogenase, an altered 2-chloro-3-hydroxypropanenitrile providing halohydrocarbon dehalogenase, an altered 3-chloro-2-hydroxypropanenitrile providing halohydrocarbon dehalogenase, an altered 2-bromo-3-hydroxypropanenitrile providing halohydrocarbon dehalogenase, an altered 3-bromo-2-hydroxypropanenitrile providing halohydrocarbon dehalogenase, an altered 3-chloro-4-hydroxybutanenitrile providing halohydrocarbon dehalogenase, an altered 4-chloro-3-hydroxybutanenitrile providing halohydrocarbon dehalogenase, an altered 3-bromo-4-hydroxybutanenitrile providing halohydrocarbon dehalogenase, an altered 4-bromo-3-hydroxybutanenitrile providing halohydrocarbon dehalogenase, an altered 2-chloro-3-hydroxypropanamide providing halohydrocarbon dehalogenase, an altered 3-chloro-2-hydroxypropanamide providing halohydrocarbon dehalogenase, an altered 2-bromo-3-hydroxypropanamide providing halohydrocarbon dehalogenase, an altered 3-bromo-2-hydroxypropanamide providing halohydrocarbon dehalogenase, an altered 3-chloro-4-hydroxybutanamide providing halohydrocarbon dehalogenase, an altered 4-chloro-3-hydroxybutanamide providing halohydrocarbon dehalogenase, an altered 3-bromo-4-hydroxybutanamide providing halohydrocarbon dehalogenase, an altered 4-bromo-3-hydroxybutanamide providing halohydrocarbon dehalogenase, an altered 2-chloro-3-hydroxypropanoic acid providing halohydrocarbon dehalogenase, an altered 3-chloro-2-hydroxypropanoic acid providing halohydrocarbon dehalogenase, an altered 2-bromo-3-hydroxypropanoic acid providing halohydrocarbon dehalogenase, an altered 3-bromo-2-hydroxypropanoic acid providing halohydrocarbon dehalogenase, an altered 3-chloro-4-hydroxybutanoic acid providing halohydrocarbon dehalogenase, an altered 4-chloro-3-hydroxybutanoic acid providing halohydrocarbon dehalogenase, an altered 3-bromo-4-hydroxybutanoic acid providing halohydrocarbon dehalogenase, an altered 4-bromo-3-hydroxybutanoic acid providing halohydrocarbon dehalogenase, an altered at least predominantly (R)-2-chloro-3-hydroxypropanenitrile providing halohydrocarbon dehalogenase, an altered at least predominantly (R)-3-chloro-2-hydroxypropanenitrile providing halohydrocarbon dehalogenase, an altered at least predominantly (R)-2-bromo-3-hydroxypropanenitrile providing halohydrocarbon dehalogenase, an altered at least predominantly (R)-3-bromo-2-hydroxypropanenitrile providing halohydrocarbon dehalogenase, an altered at least predominantly (S)-2-chloro-3-hydroxypropanenitrile providing halohydrocarbon dehalogenase, an altered at least predominantly (S)-3-chloro-2-hydroxypropanenitrile providing halohydrocarbon dehalogenase, an altered at least predominantly (S)-2-bromo-3-hydroxypropanenitrile providing halohydrocarbon dehalogenase, an altered at least predominantly (S)-3-bromo-2-hydroxypropanenitrile providing halohydrocarbon dehalogenase, an altered at least predominantly (R)-3-chloro-4-hydroxybutanenitrile providing halohydrocarbon dehalogenase, an altered at least predominantly (R)-4-chloro-3-hydroxybutanenitrile providing halohydrocarbon dehalogenase, an altered at least predominantly (R)-3-bromo-4-hydroxybutanenitrile providing halohydrocarbon dehalogenase, an altered at least predominantly (R)-4-bromo-3-hydroxybutanenitrile providing halohydrocarbon dehalogenase, an altered at least predominantly (S)-3-chloro-4-hydroxybutanenitrile providing halohydrocarbon dehalogenase, an altered at least predominantly (S)-4-chloro-3-hydroxybutanenitrile providing halohydrocarbon dehalogenase, an altered at least predominantly (S)-3-bromo-4-hydroxybutanenitrile providing halohydrocarbon dehalogenase, an altered at least predominantly (S)-4-bromo-3-hydroxybutanenitrile providing halohydrocarbon dehalogenase, an altered at least predominantly (R)-2-chloro-3-hydroxypropanamide providing halohydrocarbon dehalogenase, an altered at least predominantly (R)-3-chloro-2-hydroxypropanamide providing halohydrocarbon dehalogenase, an altered at least predominantly (R)-2-bromo-3-hydroxypropanamide providing halohydrocarbon dehalogenase, an altered at least predominantly (R)-3-bromo-2-hydroxypropanamide providing halohydrocarbon dehalogenase, an altered at least predominantly (S)-2-chloro-3-hydroxypropanamide providing halohydrocarbon dehalogenase, an altered at least predominantly (S)-3-chloro-2-hydroxypropanamide providing halohydrocarbon dehalogenase, an altered at least predominantly (S)-2-bromo-3-hydroxypropanamide providing halohydrocarbon dehalogenase, an altered at least predominantly (S)-3-bromo-2-hydroxypropanamide providing halohydrocarbon dehalogenase, an altered at least predominantly (R)-3-chloro-4-hydroxybutanamide providing halohydrocarbon dehalogenase, an altered at least predominantly (R)-4-chloro-3-hydroxybutanamide providing halohydrocarbon dehalogenase, an altered at least predominantly (R)-3-bromo-4-hydroxybutanamide providing halohydrocarbon dehalogenase, an altered at least predominantly (R)-4-bromo-3-hydroxybutanamide providing halohydrocarbon dehalogenase, an altered at least predominantly (S)-3-chloro-4-hydroxybutanamide providing halohydrocarbon dehalogenase, an altered at least predominantly (S)-4-chloro-3-hydroxybutanamide providing halohydrocarbon dehalogenase, an altered at least predominantly (S)-3-bromo-4-hydroxybutanamide providing halohydrocarbon dehalogenase, an altered at least predominantly (S)-4-bromo-3-hydroxybutanamide providing halohydrocarbon dehalogenase, an altered at least predominantly (R)-2-chloro-3-hydroxypropanoic acid providing halohydrocarbon dehalogenase, an altered at least predominantly (R)-3-chloro-2-hydroxypropanoic acid providing halohydrocarbon dehalogenase, an altered at least predominantly (R)-2-bromo-3-hydroxypropanoic acid providing halohydrocarbon dehalogenase, an altered at least predominantly (R)-3-bromo-2-hydroxypropanoic acid providing halohydrocarbon dehalogenase, an altered at least predominantly (S)-2-chloro-3-hydroxypropanoic acid providing halohydrocarbon dehalogenase, an altered at least predominantly (S)-3-chloro-2-hydroxypropanoic acid providing halohydrocarbon dehalogenase, an altered at least predominantly (S)-2-bromo-3-hydroxypropanoic acid providing halohydrocarbon dehalogenase, an altered at least predominantly (S)-3-bromo-2-hydroxypropanoic acid providing halohydrocarbon dehalogenase, an altered at least predominantly (R)-3-chloro-4-hydroxybutanoic acid providing halohydrocarbon dehalogenase, an altered at least predominantly (R)-4-chloro-3-hydroxybutanoic acid providing halohydrocarbon dehalogenase, an altered at least predominantly providing (R)-3-bromo-4-hydroxybutanoic acid halohydrocarbon dehalogenase, an altered at least predominantly (R)-4-bromo-3-hydroxybutanoic acid providing halohydrocarbon dehalogenase, an altered at least predominantly (S)-3-chloro-4-hydroxybutanoic acid providing halohydrocarbon dehalogenase, an altered at least predominantly (S)-4-chloro-3-hydroxybutanoic acid providing halohydrocarbon dehalogenase, an altered at least predominantly (S)-3-bromo-4-hydroxybutanoic acid providing halohydrocarbon dehalogenase, an altered at least predominantly (S)-4-bromo-3-hydroxybutanoic acid providing halohydrocarbon dehalogenase, an altered 2,3-dihydoxypropanenitrile providing halohydrocarbon dehalogenase, an altered 3,4-dihydoxybutanenitrile providing halohydrocarbon dehalogenase, an altered 2,3-dihydoxypropanamide providing halohydrocarbon dehalogenase, an altered 3,4-dihydoxybutanamide providing halohydrocarbon dehalogenase, an altered 2,3-dihydoxypropanoic acid providing halohydrocarbon dehalogenase, an altered 3,4-dihydoxybutanoic acid providing halohydrocarbon dehalogenase, an altered at least predominantly (R)-2,3-dihydoxypropanenitrile providing halohydrocarbon dehalogenase, an altered at least predominantly (S)-2,3-dihydoxypropanenitrile providing halohydrocarbon dehalogenase, an altered at least predominantly (R)-3,4-dihydoxybutanenitrile providing halohydrocarbon dehalogenase, an altered at least predominantly (S)-3,4-dihydoxybutanenitrile providing halohydrocarbon dehalogenase, an altered at least predominantly (R)-2,3-dihydoxypropanamide providing halohydrocarbon dehalogenase, an altered at least predominantly (S)-2,3-dihydoxypropanamide providing halohydrocarbon dehalogenase, an altered at least predominantly (R)-3,4-dihydoxybutanamide providing halohydrocarbon dehalogenase, an altered at least predominantly (S)-3,4-dihydoxybutanamide providing halohydrocarbon dehalogenase, an altered at least predominantly (R)-2,3-dihydoxypropanoic acid providing halohydrocarbon dehalogenase, an altered at least predominantly (S)-2,3-dihydoxypropanoic acid providing halohydrocarbon dehalogenase, an altered at least predominantly (R)-3,4-dihydoxybutanoic acid providing halohydrocarbon dehalogenase, an altered at least predominantly (S)-3,4-dihydoxybutanoic acid providing halohydrocarbon dehalogenase, an altered 2,3-epoxy-1-propanenitrile providing halohydrocarbon dehalogenase, an altered 3,4-epoxy-1-butanenitrile providing halohydrocarbon dehalogenase, an altered 2,3-epoxy-1-propanamide providing halohydrocarbon dehalogenase, an altered 3,4-epoxy-1-butanamide providing halohydrocarbon dehalogenase, an altered 2,3-epoxy-1-propanoic acid providing halohydrocarbon dehalogenase, an altered 3,4-epoxy-1-butanoic acid providing halohydrocarbon dehalogenase, an altered at least predominantly (R)-2,3-epoxy-1-propanenitrile providing halohydrocarbon dehalogenase, an altered at least predominantly (S)-2,3-epoxy-1-propanenitrile providing halohydrocarbon dehalogenase, an altered at least predominantly (R)-3,4-epoxy-1-butanenitrile providing halohydrocarbon dehalogenase, an altered at least predominantly (S)-3,4-epoxy-1-butanenitrile providing halohydrocarbon dehalogenase, an altered at least predominantly (R)-2,3-epoxy-1-propanamide providing halohydrocarbon dehalogenase, an altered at least predominantly (S)-2,3-epoxy-1-propanamide providing halohydrocarbon dehalogenase, an altered at least predominantly (R)-3,4-epoxy-1-butanamide providing halohydrocarbon dehalogenase, an altered at least predominantly (S)-3,4-epoxy-1-butanamide providing halohydrocarbon dehalogenase, an altered at least predominantly (R)-2,3-epoxy-1-propanoic acid providing halohydrocarbon dehalogenase, an altered at least predominantly (S)-2,3-epoxy-1-propanoic acid providing halohydrocarbon dehalogenase, an altered at least predominantly (R)-3,4-epoxy-1-butanoic acid providing halohydrocarbon dehalogenase, an altered at least predominantly (S)-3,4-epoxy-1-butanoic acid providing halohydrocarbon dehalogenase, an altered alkyl 2,3-dichloropropanoate providing halohydrocarbon dehalogenase, an altered alkyl 2,3-dibromopropanoate providing halohydrocarbon dehalogenase, an altered alkyl 3,4-dichlorobutanoate providing halohydrocarbon dehalogenase, an altered alkyl 3,4-dibromobutanoate providing halohydrocarbon dehalogenase, an altered alkyl 2-chloro-3-hydroxypropanoate providing halohydrocarbon dehalogenase, an altered alkyl 3-chloro-2-hydroxypropanoate providing halohydrocarbon dehalogenase, an altered alkyl 2-bromo-3-hydroxypropanoate providing halohydrocarbon dehalogenase, an altered alkyl 3-bromo-2-hydroxypropanoate providing halohydrocarbon dehalogenase, an altered alkyl 3-chloro-4-hydroxybutanoate providing halohydrocarbon dehalogenase, an altered alkyl 4-chloro-3-hydroxybutanoate providing halohydrocarbon dehalogenase, an altered alkyl 3-bromo-4-hydroxybutanoate providing halohydrocarbon dehalogenase, an altered alkyl 4-bromo-3-hydroxy butanoate providing halohydrocarbon dehalogenase, an altered alkyl 2,3-dihydroxypropanoate providing halohydrocarbon dehalogenase, an altered alkyl 3,4-dihydroxybutanoate providing halohydrocarbon dehalogenase, an altered alkyl 2,3-epoxy-1-propanoate providing halohydrocarbon dehalogenase, an altered alkyl 3,4-epoxy-1-butanoate providing halohydrocarbon dehalogenase, an altered at least predominantly (R)-alkyl 2,3-dichloropropanoate providing halohydrocarbon dehalogenase, an altered at least predominantly (R)-alkyl 2,3-dibromopropanoate providing halohydrocarbon dehalogenase, an altered at least predominantly (R)-alkyl 3,4-dichlorobutanoate providing halohydrocarbon dehalogenase, an altered at least predominantly (R)-alkyl 3,4-dibromobutanoate providing halohydrocarbon dehalogenase, an altered at least predominantly (S)-alkyl 2,3-dichloropropanoate providing halohydrocarbon dehalogenase, an altered at least predominantly (S)-alkyl 2,3-dibromopropanoate providing halohydrocarbon dehalogenase, an altered at least predominantly (S)-alkyl 3,4-dichlorobutanoate providing halohydrocarbon dehalogenase, an altered at least predominantly (S)-alkyl 3,4-dibromobutanoate providing halohydrocarbon dehalogenase, an altered at least predominantly (R)-alkyl 2-chloro-3-hydroxypropanoate providing halohydrocarbon dehalogenase, an altered at least predominantly (R)-alkyl 3-chloro-2-hydroxypropanoate providing halohydrocarbon dehalogenase, an altered at least predominantly (R)-alkyl 2-bromo-3-hydroxypropanoate providing halohydrocarbon dehalogenase, an altered at least predominantly (R)-alkyl 3-bromo-2-hydroxypropanoate providing halohydrocarbon dehalogenase, an altered at least predominantly (R)-alkyl 3-chloro-4-hydroxybutanoate providing halohydrocarbon dehalogenase, an altered at least predominantly (R)-alkyl 4-chloro-3-hydroxybutanoate providing halohydrocarbon dehalogenase, an altered at least predominantly (R)-alkyl 3-bromo-4-hydroxybutanoate providing halohydrocarbon dehalogenase, an altered at least predominantly (R)-alkyl 4-bromo-3-hydroxybutanoate providing halohydrocarbon dehalogenase, an altered at least predominantly (S)-alkyl 2-chloro-3-hydroxypropanoate providing halohydrocarbon dehalogenase, an altered at least predominantly (S)-alkyl 3-chloro-2-hydroxypropanoate providing halohydrocarbon dehalogenase, an altered at least predominantly (S)-alkyl 2-bromo-3-hydroxypropanoate providing halohydrocarbon dehalogenase, an altered at least predominantly (S)-alkyl 3-bromo-2-hydroxypropanoate providing halohydrocarbon dehalogenase, an altered at least predominantly (S)-alkyl 3-chloro-4-hydroxybutanoate providing halohydrocarbon dehalogenase, an altered at least predominantly (S)-alkyl 4-chloro-3-hydroxybutanoate providing halohydrocarbon dehalogenase, an altered at least predominantly (S)-alkyl 3-bromo-4-hydroxybutanoate providing halohydrocarbon dehalogenase, an altered at least predominantly (S)-alkyl 4-bromo-3-hydroxybutanoate providing halohydrocarbon dehalogenase, an altered at least predominantly (R)-alkyl 2,3-dihydroxypropanoate providing halohydrocarbon dehalogenase, an altered at least predominantly (R)-alkyl 3,4-dihydroxybutanoate providing halohydrocarbon dehalogenase, an altered at least predominantly (S)-alkyl 2,3-dihydroxypropanoate providing halohydrocarbon dehalogenase, an altered at least predominantly (S)-alkyl 3,4-dihydroxybutanoate providing halohydrocarbon dehalogenase, an altered at least predominantly (R)-alkyl 2,3-epoxy-1-propanoate providing halohydrocarbon dehalogenase, an altered at least predominantly (R)-alkyl 3,4-epoxy-1-butanoate providing halohydrocarbon dehalogenase, an altered at least predominantly (S)-alkyl 2,3-epoxy-1-propanoate providing halohydrocarbon dehalogenase, an altered at least predominantly (S)-alkyl 3,4-epoxy-1-butanoate providing halohydrocarbon dehalogenase, an altered 2-hydroxyethanoic acid providing halohydrocarbon dehalogenase, an altered 2-hydroxyethanamide providing halohydrocarbon dehalogenase, an altered 2-hydroxyethanenitrile providing halohydrocarbon dehalogenase, an altered alkyl 2-hydroxyethanoate providing halohydrocarbon dehalogenase, an altered 2-hydroxypropanoic acid providing halohydrocarbon dehalogenase, an altered 2-hydroxypropanamide providing halohydrocarbon dehalogenase, an altered 2-hydroxypropanenitrile providing halohydrocarbon dehalogenase, an altered alkyl 2-hydroxypropanoate providing halohydrocarbon dehalogenase, an altered 3-hydroxypropanoic acid providing halohydrocarbon dehalogenase, an altered 3-hydroxypropanamide providing halohydrocarbon dehalogenase, an altered 3-hydroxypropanenitrile providing halohydrocarbon dehalogenase, an altered alkyl 3-hydroxypropanoate providing halohydrocarbon dehalogenase, an altered 2-hydroxybutanoic acid providing halohydrocarbon dehalogenase, an altered 2-hydroxybutanamide providing halohydrocarbon dehalogenase, an altered 2-hydroxybutanenitrile providing halohydrocarbon dehalogenase, an altered alkyl 2-hydroxybutanoate providing halohydrocarbon dehalogenase, an altered 3-hydroxybutanoic acid providing halohydrocarbon dehalogenase, an altered 3-hydroxybutanamide providing halohydrocarbon dehalogenase, an altered 3-hydroxybutanenitrile providing halohydrocarbon dehalogenase, an altered alkyl 3-hydroxybutanoate providing halohydrocarbon dehalogenase, an altered 4-hydroxybutanoic acid providing halohydrocarbon dehalogenase, an altered 4-hydroxybutanamide providing halohydrocarbon dehalogenase, an altered 4-hydroxybutanenitrile providing halohydrocarbon dehalogenase, an altered alkyl 4-hydroxybutanoate providing halohydrocarbon dehalogenase, an altered at least predominantly (R)-2-hydroxypropanoic acid providing halohydrocarbon dehalogenase, an altered at least predominantly (S)-2-hydroxypropanoic acid providing halohydrocarbon dehalogenase, an altered at least predominantly (R)-2-hydroxypropanamide providing halohydrocarbon dehalogenase, an altered at least predominantly (S)-2-hydroxypropanamide providing halohydrocarbon dehalogenase, an altered at least predominantly (R)-2-hydroxypropanenitrile providing halohydrocarbon dehalogenase, an altered at least predominantly (S)-2-hydroxypropanenitrile providing halohydrocarbon dehalogenase, an altered at least predominantly (R)-alkyl 2-hydroxypropanoate providing halohydrocarbon dehalogenase, an altered at least predominantly (S)-alkyl 2-hydroxypropanoate providing halohydrocarbon dehalogenase, an altered at least predominantly (R)-2-hydroxybutanoic acid providing halohydrocarbon dehalogenase, an altered at least predominantly (S)-2-hydroxybutanoic acid providing halohydrocarbon dehalogenase, an altered at least predominantly (R)-2-hydroxybutanamide providing halohydrocarbon dehalogenase, an altered at least predominantly (S)-2-hydroxybutanamide providing halohydrocarbon dehalogenase, an altered at least predominantly (R)-2-hydroxybutanenitrile providing halohydrocarbon dehalogenase, an altered at least predominantly (S)-2-hydroxybutanenitrile providing halohydrocarbon dehalogenase, an altered at least predominantly (R)-alkyl 2-hydroxybutanoate providing halohydrocarbon dehalogenase, an altered at least predominantly (S)-alkyl 2-hydroxybutanoate providing halohydrocarbon dehalogenase, an altered at least predominantly (R)-3-hydroxybutanoic acid providing halohydrocarbon dehalogenase, an altered at least predominantly (S)-3-hydroxybutanoic acid providing halohydrocarbon dehalogenase, an altered at least predominantly (R)-3-hydroxybutanamide providing halohydrocarbon dehalogenase, an altered at least predominantly (S)-3-hydroxybutanamide providing halohydrocarbon dehalogenase, an altered at least predominantly (R)-3-hydroxybutanenitrile providing halohydrocarbon dehalogenase, an altered at least predominantly (S)-3-hydroxybutanenitrile providing halohydrocarbon dehalogenase, an altered at least predominantly (R)-alkyl 3-hydroxybutanoate providing halohydrocarbon dehalogenase, an altered at least predominantly (S)-alkyl 3-hydroxybutanoate providing halohydrocarbon dehalogenase, an altered 2-chloro-3-hydroxybutanenitrile providing halohydrocarbon dehalogenase, an altered 2-bromo-3-hydroxybutanenitrile providing halohydrocarbon dehalogenase, an altered 3-chloro-2-hydroxybutanenitrile providing halohydrocarbon dehalogenase, an altered 3-bromo-2-hydroxybutanenitrile providing halohydrocarbon dehalogenase, an altered 2-chloro-3-hydroxybutanamide providing halohydrocarbon dehalogenase, an altered 2-bromo-3-hydroxybutanamide providing halohydrocarbon dehalogenase, an altered 3-chloro-2-hydroxybutanamide providing halohydrocarbon dehalogenase, an altered 3-bromo-2-hydroxybutanamide providing halohydrocarbon dehalogenase, an altered 2-chloro-3-hydroxybutanoic acid providing halohydrocarbon dehalogenase, an altered 2-bromo-3-hydroxybutanoic acid providing halohydrocarbon dehalogenase, an altered 3-chloro-2-hydroxybutanoic acid providing halohydrocarbon dehalogenase, an altered 3-bromo-2-hydroxybutanoic acid providing halohydrocarbon dehalogenase, an altered alkyl 2-chloro-3-hydroxybutanoate providing halohydrocarbon dehalogenase, an altered alkyl 2-bromo-3-hydroxybutanoate providing halohydrocarbon dehalogenase, an altered alkyl 3-chloro-2-hydroxybutanoate providing halohydrocarbon dehalogenase, an altered alkyl 3-bromo-2-hydroxybutanoate providing halohydrocarbon dehalogenase, an altered 2,3-dihydroxybutanenitrile providing halohydrocarbon dehalogenase, an altered 2,3-dihydroxybutanamide providing halohydrocarbon dehalogenase, an altered 2,3-dihydroxybutanoic acid providing halohydrocarbon dehalogenase, an altered alkyl 2,3-dihydroxybutanoate providing halohydrocarbon dehalogenase, an altered 2,3-epoxy-1-butanenitrile providing halohydrocarbon dehalogenase, an altered 2,3-epoxy-1-butanamide providing halohydrocarbon dehalogenase, an altered 2,3-epoxy-1-butanoic acid providing halohydrocarbon dehalogenase, an altered alkyl 2,3-epoxy-1-butanoate providing halohydrocarbon dehalogenase, an altered at least predominantly (R,R)-2,3-epoxy-1-butanenitrile providing halohydrocarbon dehalogenase, an altered at least predominantly (R,R)-2,3-epoxy-1-butanamide providing halohydrocarbon dehalogenase, an altered at least predominantly (R,R)-2,3-epoxy-1-butanoic acid providing halohydrocarbon dehalogenase, an altered at least predominantly (R,R)-alkyl 2,3-epoxy-1-butanoate providing halohydrocarbon dehalogenase, an altered at least predominantly (S,S)-2,3-epoxy-1-butanenitrile providing halohydrocarbon dehalogenase, an altered at least predominantly (S,S)-2,3-epoxy-1-butanamide providing halohydrocarbon dehalogenase, an altered at least predominantly (S,S)-2,3-epoxy-1-butanoic acid providing halohydrocarbon dehalogenase, an altered at least predominantly (S,S)-alkyl 2,3-epoxy-1-butanoate providing halohydrocarbon dehalogenase, an altered at least predominantly (R,S)-2,3-epoxy-1-butanenitrile providing halohydrocarbon dehalogenase, an altered at least predominantly (R,S)-2,3-epoxy-1-butanamide providing halohydrocarbon dehalogenase, an altered at least predominantly (R,S)-2,3-epoxy-1-butanoic acid providing halohydrocarbon dehalogenase, an altered at least predominantly (R,S)-alkyl 2,3-epoxy-1-butanoate providing halohydrocarbon dehalogenase, an altered at least predominantly (S,R)-2,3-epoxy-1-butanenitrile providing halohydrocarbon dehalogenase, an altered at least predominantly (S,R)-2,3-epoxy-1-butanamide providing halohydrocarbon dehalogenase, an altered at least predominantly (S,R)-2,3-epoxy-1-butanoic acid providing halohydrocarbon dehalogenase, an altered at least predominantly (S,R)-alkyl 2,3-epoxy-1-butanoate providing halohydrocarbon dehalogenase, an altered at least predominantly (R,R)-2,3-dihydroxybutanenitrile providing halohydrocarbon dehalogenase, an altered at least predominantly (R,R)-2,3-dihydroxybutanamide providing halohydrocarbon dehalogenase, an altered at least predominantly (R,R)-2,3-dihydroxybutanoic acid providing halohydrocarbon dehalogenase, an altered at least predominantly (R,R)-alkyl 2,3-dihydroxybutanoate providing halohydrocarbon dehalogenase, an altered at least predominantly (S,S)-2,3-dihydroxybutanenitrile providing halohydrocarbon dehalogenase, an altered at least predominantly (S,S)-2,3-dihydroxybutanamide providing halohydrocarbon dehalogenase, an altered at least predominantly (S,S)-2,3-dihydroxybutanoic acid providing halohydrocarbon dehalogenase, an altered at least predominantly (S,S)-alkyl 2,3-dihydroxybutanoate providing halohydrocarbon dehalogenase, an altered at least predominantly (R,S)-2,3-dihydroxybutanenitrile providing halohydrocarbon dehalogenase, an altered at least predominantly (R,S)-2,3-dihydroxybutanamide providing halohydrocarbon dehalogenase, an altered at least predominantly (R,S)-2,3-dihydroxybutanoic acid providing halohydrocarbon dehalogenase, an altered at least predominantly (R,S)-alkyl 2,3-dihydroxybutanoate providing halohydrocarbon dehalogenase, an altered at least predominantly (S,R)-2,3-dihydroxybutanenitrile providing halohydrocarbon dehalogenase, an altered at least predominantly (S,R)-2,3-dihydroxybutanamide providing halohydrocarbon dehalogenase, an altered at least predominantly (S,R)-2,3-dihydroxybutanoic acid providing halohydrocarbon dehalogenase, an altered at least predominantly (S,R)-alkyl 2,3-dihydroxybutanoate providing halohydrocarbon dehalogenase, an altered at least predominantly (R,R)-2-chloro-3-hydroxybutanenitrile providing halohydrocarbon dehalogenase, an altered at least predominantly (R,R)-2-bromo-3-hydroxybutanenitrile providing halohydrocarbon dehalogenase, an altered at least predominantly (R,R)-3-chloro-2-hydroxybutanenitrile providing halohydrocarbon dehalogenase, an altered at least predominantly (R,R)-3-bromo-2-hydroxybutanenitrile providing halohydrocarbon dehalogenase, an altered at least predominantly (R,R)-2-chloro-3-hydroxybutanamide providing halohydrocarbon dehalogenase, an altered at least predominantly (R,R)-2-bromo-3-hydroxybutanamide providing halohydrocarbon dehalogenase, an altered at least predominantly (R,R)-3-chloro-2-hydroxybutanamide providing halohydrocarbon dehalogenase, an altered at least predominantly (R,R)-3-bromo-2-hydroxybutanamide providing halohydrocarbon dehalogenase, an altered at least predominantly (R,R)-2-chloro-3-hydroxybutanoic acid providing halohydrocarbon dehalogenase, an altered at least predominantly (R,R)-2-bromo-3-hydroxybutanoic acid providing halohydrocarbon dehalogenase, an altered at least predominantly (R,R)-3-chloro-2-hydroxybutanoic acid providing halohydrocarbon dehalogenase, an altered at least predominantly (R,R)-3-bromo-2-hydroxybutanoic acid providing halohydrocarbon dehalogenase, an altered at least predominantly (R,R)-alkyl 2-chloro-3-hydroxybutanoate providing halohydrocarbon dehalogenase, an altered at least predominantly (R,R)-alkyl 2-bromo-3-hydroxybutanoate providing halohydrocarbon dehalogenase, an altered at least predominantly (R,R)-alkyl 3-chloro-2-hydroxybutanoate providing halohydrocarbon dehalogenase, an altered at least predominantly (R,R)-alkyl 3-bromo-2-hydroxybutanoate providing halohydrocarbon dehalogenase, an altered at least predominantly (S,S)-2-chloro-3-hydroxybutanenitrile providing halohydrocarbon dehalogenase, an altered at least predominantly (S,S)-2-bromo-3-hydroxybutanenitrile providing halohydrocarbon dehalogenase, an altered at least predominantly (S,S)-3-chloro-2-hydroxybutanenitrile providing halohydrocarbon dehalogenase, an altered at least predominantly (S,S)-3-bromo-2-hydroxybutanenitrile providing halohydrocarbon dehalogenase, an altered at least predominantly (S,S)-2-chloro-3-hydroxybutanamide providing halohydrocarbon dehalogenase, an altered at least predominantly (S,S)-2-bromo-3-hydroxybutanamide providing halohydrocarbon dehalogenase, an altered at least predominantly (S,S)-3-chloro-2-hydroxybutanamide providing halohydrocarbon dehalogenase, an altered at least predominantly (S,S)-3-bromo-2-hydroxybutanamide providing halohydrocarbon dehalogenase, an altered at least predominantly (S,S)-2-chloro-3-hydroxybutanoic acid providing halohydrocarbon dehalogenase, an altered at least predominantly (S,S)-2-bromo-3-hydroxybutanoic acid providing halohydrocarbon dehalogenase, an altered at least predominantly (S,S)-3-chloro-2-hydroxybutanoic acid providing halohydrocarbon dehalogenase, an altered at least predominantly (S,S)-3-bromo-2-hydroxybutanoic acid providing halohydrocarbon dehalogenase, an altered at least predominantly (S,S)-alkyl 2-chloro-3-hydroxybutanoate providing halohydrocarbon dehalogenase, an altered at least predominantly (S,S)-alkyl 2-bromo-3-hydroxybutanoate providing halohydrocarbon dehalogenase, an altered at least predominantly (S,S)-alkyl 3-chloro-2-hydroxybutanoate providing halohydrocarbon dehalogenase, an altered at least predominantly (S,S)-alkyl 3-bromo-2-hydroxybutanoate providing halohydrocarbon dehalogenase, an altered at least predominantly (R,S)-2-chloro-3-hydroxybutanenitrile providing halohydrocarbon dehalogenase, an altered at least predominantly (R,S)-2-bromo-3-hydroxybutanenitrile providing halohydrocarbon dehalogenase, an altered at least predominantly (R,S)-3-chloro-2-hydroxybutanenitrile providing halohydrocarbon dehalogenase, an altered at least predominantly (R,S)-3-bromo-2-hydroxybutanenitrile providing halohydrocarbon dehalogenase, an altered at least predominantly (R,S)-2-chloro-3-hydroxybutanamide providing halohydrocarbon dehalogenase, an altered at least predominantly (R,S)-2-bromo-3-hydroxybutanamide providing halohydrocarbon dehalogenase, an altered at least predominantly (R,S)-3-chloro-2-hydroxybutanamide providing halohydrocarbon dehalogenase, an altered at least predominantly (R,S)-3-bromo-2-hydroxybutanamide providing halohydrocarbon dehalogenase, an altered at least predominantly (R,S)-2-chloro-3-hydroxybutanoic acid providing halohydrocarbon dehalogenase, an altered at least predominantly (R,S)-2-bromo-3-hydroxybutanoic acid providing halohydrocarbon dehalogenase, an altered at least predominantly (R,S)-3-chloro-2-hydroxybutanoic acid providing halohydrocarbon dehalogenase, an altered at least predominantly (R,S)-3-bromo-2-hydroxybutanoic acid providing halohydrocarbon dehalogenase, an altered at least predominantly (R,S)-alkyl 2-chloro-3-hydroxybutanoate providing halohydrocarbon dehalogenase, an altered at least predominantly (R,S)-alkyl 2-bromo-3-hydroxybutanoate providing halohydrocarbon dehalogenase, an altered at least predominantly (R,S)-alkyl 3-chloro-2-hydroxybutanoate providing halohydrocarbon dehalogenase, an altered at least predominantly (R,S)-alkyl 3-bromo-2-hydroxybutanoate providing halohydrocarbon dehalogenase, an altered at least predominantly (S,R)-2-chloro-3-hydroxybutanenitrile providing halohydrocarbon dehalogenase, an altered at least predominantly (S,R)-2-bromo-3-hydroxybutanenitrile providing halohydrocarbon dehalogenase, an altered at least predominantly (S,R)-3-chloro-2-hydroxybutanenitrile providing halohydrocarbon dehalogenase, an altered at least predominantly (S,R)-3-bromo-2-hydroxybutanenitrile providing halohydrocarbon dehalogenase, an altered at least predominantly (S,R)-2-chloro-3-hydroxybutanamide providing halohydrocarbon dehalogenase, an altered at least predominantly (S,R)-2-bromo-3-hydroxybutanamide providing halohydrocarbon dehalogenase, an altered at least predominantly (S,R)-3-chloro-2-hydroxybutanamide providing halohydrocarbon dehalogenase, an altered at least predominantly (S,R)-3-bromo-2-hydroxybutanamide providing halohydrocarbon dehalogenase, an altered at least predominantly (S,R)-2-chloro-3-hydroxybutanoic acid providing halohydrocarbon dehalogenase, an altered at least predominantly (S,R)-2-bromo-3-hydroxybutanoic acid providing halohydrocarbon dehalogenase, an altered at least predominantly (S,R)-3-chloro-2-hydroxybutanoic acid providing halohydrocarbon dehalogenase, an altered at least predominantly (S,R)-3-bromo-2-hydroxybutanoic acid providing halohydrocarbon dehalogenase, an altered at least predominantly (S,R)-alkyl 2-chloro-3-hydroxybutanoate providing halohydrocarbon dehalogenase, an altered at least predominantly (S,R)-alkyl 2-bromo-3-hydroxybutanoate providing halohydrocarbon dehalogenase, an altered at least predominantly (S,R)-alkyl 3-chloro-2-hydroxybutanoate providing halohydrocarbon dehalogenase, an altered at least predominantly (S,R)-alkyl 3-bromo-2-hydroxybutanoate providing halohydrocarbon dehalogenase, an altered 2,3-dichloro-4-hydroxybutanenitrile providing halohydrocarbon dehalogenase, an altered 2,3-dibromo-4-hydroxybutanenitrile providing halohydrocarbon dehalogenase, an altered 2,3-dichloro-4-hydroxybutanamide providing halohydrocarbon dehalogenase, an altered 2,3-dibromo-4-hydroxybutanamide providing halohydrocarbon dehalogenase, an altered 2,3-dichloro-4-hydroxybutanoic acid providing halohydrocarbon dehalogenase, an altered 2,3-dibromo-4-hydroxybutanoic acid providing halohydrocarbon dehalogenase, an altered alkyl 2,3-dichloro-4-hydroxybutanoate providing halohydrocarbon dehalogenase, an altered alkyl 2,3-dibromo-4-hydroxybutanoate providing halohydrocarbon dehalogenase, an altered 2,4-dichloro-3-hydroxybutanenitrile providing halohydrocarbon dehalogenase, an altered 2,4-dibromo-3-hydroxybutanenitrile providing halohydrocarbon dehalogenase, an altered 2,4-dichloro-3-hydroxybutanamide providing halohydrocarbon dehalogenase, an altered 2,4-dibromo-3-hydroxybutanamide providing halohydrocarbon dehalogenase, an altered 2,4-dichloro-3-hydroxybutanoic acid providing halohydrocarbon dehalogenase, an altered 2,4-dibromo-3-hydroxybutanoic acid providing halohydrocarbon dehalogenase, an altered alkyl 2,4-dichloro-3-hydroxybutanoate providing halohydrocarbon dehalogenase, an altered alkyl 2,4-dibromo-3-hydroxybutanoate providing halohydrocarbon dehalogenase, an altered 3,4-dichloro-2-hydroxybutanenitrile providing halohydrocarbon dehalogenase, an altered 3,4-dibromo-2-hydroxybutanenitrile providing halohydrocarbon dehalogenase, an altered 3,4-dichloro-2-hydroxybutanamide providing halohydrocarbon dehalogenase, an altered 3,4-dibromo-2-hydroxybutanamide providing halohydrocarbon dehalogenase, an altered 3,4-dichloro-2-hydroxybutanoic acid providing halohydrocarbon dehalogenase, an altered 3,4-dibromo-2-hydroxybutanoic acid providing halohydrocarbon dehalogenase, an altered alkyl 3,4-dichloro-2-hydroxybutanoate providing halohydrocarbon dehalogenase, an altered alkyl 3,4-dibromo-2-hydroxybutanoate providing halohydrocarbon dehalogenase, an altered at least predominantly (R,R)-2,3-dichloro-4-hydroxybutanenitrile providing halohydrocarbon dehalogenase, an altered at least predominantly (R,R)-2,3-dibromo-4-hydroxybutanenitrile providing halohydrocarbon dehalogenase, an altered at least predominantly (R,R)-2,3-dichloro-4-hydroxybutanamide providing halohydrocarbon dehalogenase, an altered at least predominantly (R,R)-2,3-dibromo-4-hydroxybutanamide providing halohydrocarbon dehalogenase, an altered at least predominantly (R,R)-2,3-dichloro-4-hydroxybutanoic acid providing halohydrocarbon dehalogenase, an altered at least predominantly (R,R)-2,3-dibromo-4-hydroxybutanoic acid providing halohydrocarbon dehalogenase, an altered at least predominantly (R,R)-alkyl 2,3-dichloro-4-hydroxybutanoate providing halohydrocarbon dehalogenase, an altered at least predominantly (R,R)-alkyl 2,3-dibromo-4-hydroxybutanoate providing halohydrocarbon dehalogenase, an altered at least predominantly (R,R)-2,4-dichloro-3-hydroxybutanenitrile providing halohydrocarbon dehalogenase, an altered at least predominantly (R,R)-2,4-dibromo-3-hydroxybutanenitrile providing halohydrocarbon dehalogenase, an altered at least predominantly (R,R)-2,4-dichloro-3-hydroxybutanamide providing halohydrocarbon dehalogenase, an altered at least predominantly (R,R)-2,4-dibromo-3-hydroxybutanamide providing halohydrocarbon dehalogenase, an altered at least predominantly (R,R)-2,4-dichloro-3-hydroxybutanoic acid providing halohydrocarbon dehalogenase, an altered at least predominantly (R,R)-2,4-dibromo-3-hydroxybutanoic acid providing halohydrocarbon dehalogenase, an altered at least predominantly (R,R)-alkyl 2,4-dichloro-3-hydroxybutanoate providing halohydrocarbon dehalogenase, an altered at least predominantly (R,R)-alkyl 2,4-dibromo-3-hydroxybutanoate providing halohydrocarbon dehalogenase, an altered at least predominantly (R,R)-3,4-dichloro-2-hydroxybutanenitrile providing halohydrocarbon dehalogenase, an altered at least predominantly (R,R)-3,4-dibromo-2-hydroxybutanenitrile providing halohydrocarbon dehalogenase, an altered at least predominantly (R,R)-3,4-dichloro-2-hydroxybutanamide providing halohydrocarbon dehalogenase, an altered at least predominantly (R,R)-3,4-dibromo-2-hydroxybutanamide providing halohydrocarbon dehalogenase, an altered at least predominantly (R,R)-3,4-dichloro-2-hydroxybutanoic acid providing halohydrocarbon dehalogenase, an altered at least predominantly (R,R)-3,4-dibromo-2-hydroxybutanoic acid providing halohydrocarbon dehalogenase, an altered at least predominantly (R,R)-alkyl 3,4-dichloro-2-hydroxybutanoate providing halohydrocarbon dehalogenase, an altered at least predominantly (R,R)-alkyl 3,4-dibromo-2-hydroxybutanoate providing halohydrocarbon dehalogenase, an altered at least predominantly (S,S)-2,3-dichloro-4-hydroxybutanenitrile providing halohydrocarbon dehalogenase, an altered at least predominantly (S,S)-2,3-dibromo-4-hydroxybutanenitrile providing halohydrocarbon dehalogenase, an altered at least predominantly (S,S)-2,3-dichloro-4-hydroxybutanamide providing halohydrocarbon dehalogenase, an altered at least predominantly (S,S)-2,3-dibromo-4-hydroxybutanamide providing halohydrocarbon dehalogenase, an altered at least predominantly (S,S)-2,3-dichloro-4-hydroxybutanoic acid providing halohydrocarbon dehalogenase, an altered at least predominantly (S,S)-2,3-dibromo-4-hydroxybutanoic acid providing halohydrocarbon dehalogenase, an altered at least predominantly (S,S)-alkyl 2,3-dichloro-4-hydroxybutanoate providing halohydrocarbon dehalogenase, an altered at least predominantly (S,S)-alkyl 2,3-dibromo-4-hydroxybutanoate providing halohydrocarbon dehalogenase, an altered at least predominantly (S,S)-2,4-dichloro-3-hydroxybutanenitrile providing halohydrocarbon dehalogenase, an altered at least predominantly (S,S)-2,4-dibromo-3-hydroxybutanenitrile providing halohydrocarbon dehalogenase, an altered at least predominantly (S,S)-2,4-dichloro-3-hydroxybutanamide providing halohydrocarbon dehalogenase, an altered at least predominantly (S,S)-2,4-dibromo-3-hydroxybutanamide providing halohydrocarbon dehalogenase, an altered at least predominantly (S,S)-2,4-dichloro-3-hydroxybutanoic acid providing halohydrocarbon dehalogenase, an altered at least predominantly (S,S)-2,4-dibromo-3-hydroxybutanoic acid providing halohydrocarbon dehalogenase, an altered at least predominantly (S,S)-alkyl 2,4-dichloro-3-hydroxybutanoate providing halohydrocarbon dehalogenase, an altered at least predominantly (S,S)-alkyl 2,4-dibromo-3-hydroxybutanoate providing halohydrocarbon dehalogenase, an altered at least predominantly (S,S)-3,4-dichloro-2-hydroxybutanenitrile providing halohydrocarbon dehalogenase, an altered at least predominantly (S,S)-3,4-dibromo-2-hydroxybutanenitrile providing halohydrocarbon dehalogenase, an altered at least predominantly (S,S)-3,4-dichloro-2-hydroxybutanamide providing halohydrocarbon dehalogenase, an altered at least predominantly (S,S)-3,4-dibromo-2-hydroxybutanamide providing halohydrocarbon dehalogenase, an altered at least predominantly (S,S)-3,4-dichloro-2-hydroxybutanoic acid providing halohydrocarbon dehalogenase, an altered at least predominantly (S,S)-3,4-dibromo-2-hydroxybutanoic acid providing halohydrocarbon dehalogenase, an altered at least predominantly (S,S)-alkyl 3,4-dichloro-2-hydroxybutanoate providing halohydrocarbon dehalogenase, an altered at least predominantly (S,S)-alkyl 3,4-dibromo-2-hydroxybutanoate providing halohydrocarbon dehalogenase, an altered at least predominantly (R,S)-2,3-dichloro-4-hydroxybutanenitrile providing halohydrocarbon dehalogenase, an altered at least predominantly (R,S)-2,3-dibromo-4-hydroxybutanenitrile providing halohydrocarbon dehalogenase, an altered at least predominantly (R,S)-2,3-dichloro-4-hydroxybutanamide providing halohydrocarbon dehalogenase, an altered at least predominantly (R,S)-2,3-dibromo-4-hydroxybutanamide providing halohydrocarbon dehalogenase, an altered at least predominantly (R,S)-2,3-dichloro-4-hydroxybutanoic acid providing halohydrocarbon dehalogenase, an altered at least predominantly (R,S)-2,3-dibromo-4-hydroxybutanoic acid providing halohydrocarbon dehalogenase, an altered at least predominantly (R,S)-alkyl 2,3-dichloro-4-hydroxybutanoate providing halohydrocarbon dehalogenase, an altered at least predominantly (R,S)-alkyl 2,3-dibromo-4-hydroxybutanoate providing halohydrocarbon dehalogenase, an altered at least predominantly (R,S)-2,4-dichloro-3-hydroxybutanenitrile providing halohydrocarbon dehalogenase, an altered at least predominantly (R,S)-2,4-dibromo-3-hydroxybutanenitrile providing halohydrocarbon dehalogenase, an altered at least predominantly (R,S)-2,4-dichloro-3-hydroxybutanamide providing halohydrocarbon dehalogenase, an altered at least predominantly (R,S)-2,4-dibromo-3-hydroxybutanamide providing halohydrocarbon dehalogenase, an altered at least predominantly (R,S)-2,4-dichloro-3-hydroxybutanoic acid providing halohydrocarbon dehalogenase, an altered at least predominantly (R,S)-2,4-dibromo-3-hydroxybutanoic acid providing halohydrocarbon dehalogenase, an altered at least predominantly (R,S)-alkyl 2,4-dichloro-3-hydroxybutanoate providing halohydrocarbon dehalogenase, an altered at least predominantly (R,S)-alkyl 2,4-dibromo-3-hydroxybutanoate providing halohydrocarbon dehalogenase, an altered at least predominantly (R,S)-3,4-dichloro-2-hydroxybutanenitrile providing halohydrocarbon dehalogenase, an altered at least predominantly (R,S)-3,4-dibromo-2-hydroxybutanenitrile providing halohydrocarbon dehalogenase, an altered at least predominantly (R,S)-3,4-dichloro-2-hydroxybutanamide providing halohydrocarbon dehalogenase, an altered at least predominantly (R,S)-3,4-dibromo-2-hydroxybutanamide providing halohydrocarbon dehalogenase, an altered at least predominantly (R,S)-3,4-dichloro-2-hydroxybutanoic acid providing halohydrocarbon dehalogenase, an altered at least predominantly (R,S)-3,4-dibromo-2-hydroxybutanoic acid providing halohydrocarbon dehalogenase, an altered at least predominantly (R,S)-alkyl 3,4-dichloro-2-hydroxybutanoate providing halohydrocarbon dehalogenase, an altered at least predominantly (R,S)-alkyl 3,4-dibromo-2-hydroxybutanoate providing halohydrocarbon dehalogenase, an altered at least predominantly (S,R)-2,3-dichloro-4-hydroxybutanenitrile providing halohydrocarbon dehalogenase, an altered at least predominantly (S,R)-2,3-dibromo-4-hydroxybutanenitrile providing halohydrocarbon dehalogenase, an altered at least predominantly (S,R)-2,3-dichloro-4-hydroxybutanamide providing halohydrocarbon dehalogenase, an altered at least predominantly (S,R)-2,3-dibromo-4-hydroxybutanamide providing halohydrocarbon dehalogenase, an altered at least predominantly (S,R)-2,3-dichloro-4-hydroxybutanoic acid providing halohydrocarbon dehalogenase, an altered at least predominantly (S,R)-2,3-dibromo-4-hydroxybutanoic acid providing halohydrocarbon dehalogenase, an altered at least predominantly (S,R)-alkyl 2,3-dichloro-4-hydroxybutanoate providing halohydrocarbon dehalogenase, an altered at least predominantly (S,R)-alkyl 2,3-dibromo-4-hydroxybutanoate providing halohydrocarbon dehalogenase, an altered at least predominantly (S,R)-2,4-dichloro-3-hydroxybutanenitrile providing halohydrocarbon dehalogenase, an altered at least predominantly (S,R)-2,4-dibromo-3-hydroxybutanenitrile providing halohydrocarbon dehalogenase, an altered at least predominantly (S,R)-2,4-dichloro-3-hydroxybutanamide providing halohydrocarbon dehalogenase, an altered at least predominantly (S,R)-2,4-dibromo-3-hydroxybutanamide providing halohydrocarbon dehalogenase, an altered at least predominantly (S,R)-2,4-dichloro-3-hydroxybutanoic acid providing halohydrocarbon dehalogenase, an altered at least predominantly (S,R)-2,4-dibromo-3-hydroxybutanoic acid providing halohydrocarbon dehalogenase, an altered at least predominantly (S,R)-alkyl 2,4-dichloro-3-hydroxybutanoate providing halohydrocarbon dehalogenase, an altered at least predominantly (S,R)-alkyl 2,4-dibromo-3-hydroxybutanoate providing halohydrocarbon dehalogenase, an altered at least predominantly (S,R)-3,4-dichloro-2-hydroxybutanenitrile providing halohydrocarbon dehalogenase, an altered at least predominantly (S,R)-3,4-dibromo-2-hydroxybutanenitrile providing halohydrocarbon dehalogenase, an altered at least predominantly (S,R)-3,4-dichloro-2-hydroxybutanamide providing halohydrocarbon dehalogenase, an altered at least predominantly (S,R)-3,4-dibromo-2-hydroxybutanamide providing halohydrocarbon dehalogenase, an altered at least predominantly (S,R)-3,4-dichloro-2-hydroxybutanoic acid providing halohydrocarbon dehalogenase, an altered at least predominantly (S,R)-3,4-dibromo-2-hydroxybutanoic acid providing halohydrocarbon dehalogenase, an altered at least predominantly (S,R)-alkyl 3,4-dichloro-2-hydroxybutanoate providing halohydrocarbon dehalogenase, an altered at least predominantly (S,R)-alkyl 3,4-dibromo-2-hydroxybutanoate providing halohydrocarbon dehalogenase, an altered 2-chloro-3,4-dihydroxybutanenitrile providing halohydrocarbon dehalogenase, an altered 2-bromo-3,4-dihydroxybutanenitrile providing halohydrocarbon dehalogenase, an altered 2-chloro-3,4-dihydroxybutanamide providing halohydrocarbon dehalogenase, an altered 2-bromo-3,4-dihydroxybutanamide providing halohydrocarbon dehalogenase, an altered 2-chloro-3,4-dihydroxybutanoic acid providing halohydrocarbon dehalogenase, an altered 2-bromo-3,4-dihydroxybutanoic acid providing halohydrocarbon dehalogenase, an altered alkyl 2-chloro-3,4-dihydroxybutanoate providing halohydrocarbon dehalogenase, an altered alkyl 2-bromo-3,4-dihydroxybutanoate providing halohydrocarbon dehalogenase, an altered 3-chloro-2,4-dihydroxybutanenitrile providing halohydrocarbon dehalogenase, an altered 3-bromo-2,4-dihydroxybutanenitrile providing halohydrocarbon dehalogenase, an altered 3-chloro-2,4-dihydroxybutanamide providing halohydrocarbon dehalogenase, an altered 3-bromo-2,4-dihydroxybutanamide providing halohydrocarbon dehalogenase, an altered 3-chloro-2,4-dihydroxybutanoic acid providing halohydrocarbon dehalogenase, an altered 3-bromo-2,4-dihydroxybutanoic acid providing halohydrocarbon dehalogenase, an altered alkyl 3-chloro-2,4-dihydroxybutanoate providing halohydrocarbon dehalogenase, an altered alkyl 3-bromo-2,4-dihydroxybutanoate providing halohydrocarbon dehalogenase, an altered 4-chloro-2,3-dihydroxybutanenitrile providing halohydrocarbon dehalogenase, an altered 4-bromo-2,3-dihydroxybutanenitrile providing halohydrocarbon dehalogenase, an altered 4-chloro-2,3-dihydroxybutanamide providing halohydrocarbon dehalogenase, an altered 4-bromo-2,3-dihydroxybutanamide providing halohydrocarbon dehalogenase, an altered 4-chloro-2,3-dihydroxybutanoic acid providing halohydrocarbon dehalogenase, an altered 4-bromo-2,3-dihydroxybutanoic acid providing halohydrocarbon dehalogenase, an altered alkyl 4-chloro-2,3-dihydroxybutanoate providing halohydrocarbon dehalogenase, an altered alkyl 4-bromo-2,3-dihydroxybutanoate providing halohydrocarbon dehalogenase, an altered at least predominantly (R,R)-2-chloro-3,4-dihydroxybutanenitrile providing halohydrocarbon dehalogenase, an altered at least predominantly (R,R)-2-bromo-3,4-dihydroxybutanenitrile providing halohydrocarbon dehalogenase, an altered at least predominantly (R,R)-2-chloro-3,4-dihydroxybutanamide providing halohydrocarbon dehalogenase, an altered at least predominantly (R,R)-2-bromo-3,4-dihydroxybutanamide providing halohydrocarbon dehalogenase, an altered at least predominantly (R,R)-2-chloro-3,4-dihydroxybutanoic acid providing halohydrocarbon dehalogenase, an altered at least predominantly (R,R)-2-bromo-3,4-dihydroxybutanoic acid providing halohydrocarbon dehalogenase, an altered at least predominantly (R,R)-alkyl 2-chloro-3,4-dihydroxybutanoate providing halohydrocarbon dehalogenase, an altered at least predominantly (R,R)-alkyl 2-bromo-3,4-dihydroxybutanoate providing halohydrocarbon dehalogenase, an altered at least predominantly (R,R)-3-chloro-2,4-dihydroxybutanenitrile providing halohydrocarbon dehalogenase, an altered at least predominantly (R,R)-3-bromo-2,4-dihydroxybutanenitrile providing halohydrocarbon dehalogenase, an altered at least predominantly (R,R)-3-chloro-2,4-dihydroxybutanamide providing halohydrocarbon dehalogenase, an altered at least predominantly (R,R)-3-bromo-2,4-dihydroxybutanamide providing halohydrocarbon dehalogenase, an altered at least predominantly (R,R)-3-chloro-2,4-dihydroxybutanoic acid providing halohydrocarbon dehalogenase, an altered at least predominantly (R,R)-3-bromo-2,4-dihydroxybutanoic acid providing halohydrocarbon dehalogenase, an altered at least predominantly (R,R)-alkyl 3-chloro-2,4-dihydroxybutanoate providing halohydrocarbon dehalogenase, an altered at least predominantly (R,R)-alkyl 3-bromo-2,4-dihydroxybutanoate providing halohydrocarbon dehalogenase, an altered at least predominantly (R,R)-4-chloro-2,3-dihydroxybutanenitrile providing halohydrocarbon dehalogenase, an altered at least predominantly (R,R)-4-bromo-2,3-dihydroxybutanenitrile providing halohydrocarbon dehalogenase, an altered at least predominantly (R,R)-4-chloro-2,3-dihydroxybutanamide providing halohydrocarbon dehalogenase, an altered at least predominantly (R,R)-4-bromo-2,3-dihydroxybutanamide providing halohydrocarbon dehalogenase, an altered at least predominantly (R,R)-4-chloro-2,3-dihydroxybutanoic acid providing halohydrocarbon dehalogenase, an altered at least predominantly (R,R)-4-bromo-2,3-dihydroxybutanoic acid providing halohydrocarbon dehalogenase, an altered at least predominantly (R,R)-alkyl 4-chloro-2,3-dihydroxybutanoate providing halohydrocarbon dehalogenase, an altered at least predominantly (R,R)-alkyl 4-bromo-2,3-dihydroxybutanoate providing halohydrocarbon dehalogenase, an altered at least predominantly (S,S)-2-chloro-3,4-dihydroxybutanenitrile providing halohydrocarbon dehalogenase, an altered at least predominantly (S,S)-2-bromo-3,4-dihydroxybutanenitrile providing halohydrocarbon dehalogenase, an altered at least predominantly (S,S)-2-chloro-3,4-dihydroxybutanamide providing halohydrocarbon dehalogenase, an altered at least predominantly (S,S)-2-bromo-3,4-dihydroxybutanamide providing halohydrocarbon dehalogenase, an altered at least predominantly (S,S)-2-chloro-3,4-dihydroxybutanoic acid providing halohydrocarbon dehalogenase, an altered at least predominantly (S,S)-2-bromo-3,4-dihydroxybutanoic acid providing halohydrocarbon dehalogenase, an altered at least predominantly (S,S)-alkyl 2-chloro-3,4-dihydroxybutanoate providing halohydrocarbon dehalogenase, an altered at least predominantly (S,S)-alkyl 2-bromo-3,4-dihydroxybutanoate providing halohydrocarbon dehalogenase, an altered at least predominantly (S,S)-3-chloro-2,4-dihydroxybutanenitrile providing halohydrocarbon dehalogenase, an altered at least predominantly (S,S)-3-bromo-2,4-dihydroxybutanenitrile providing halohydrocarbon dehalogenase, an altered at least predominantly (S,S)-3-chloro-2,4-dihydroxybutanamide providing halohydrocarbon dehalogenase, an altered at least predominantly (S,S)-3-bromo-2,4-dihydroxybutanamide providing halohydrocarbon dehalogenase, an altered at least predominantly (S,S)-3-chloro-2,4-dihydroxybutanoic acid providing halohydrocarbon dehalogenase, an altered at least predominantly (S,S)-3-bromo-2,4-dihydroxybutanoic acid providing halohydrocarbon dehalogenase, an altered at least predominantly (S,S)-alkyl 3-chloro-2,4-dihydroxybutanoate providing halohydrocarbon dehalogenase, an altered at least predominantly (S,S)-alkyl 3-bromo-2,4-dihydroxybutanoate providing halohydrocarbon dehalogenase, an altered at least predominantly (S,S)-4-chloro-2,3-dihydroxybutanenitrile providing halohydrocarbon dehalogenase, an altered at least predominantly (S,S)-4-bromo-2,3-dihydroxybutanenitrile providing halohydrocarbon dehalogenase, an altered at least predominantly (S,S)-4-chloro-2,3-dihydroxybutanamide providing halohydrocarbon dehalogenase, an altered at least predominantly (S,S)-4-bromo-2,3-dihydroxybutanamide providing halohydrocarbon dehalogenase, an altered at least predominantly (S,S)-4-chloro-2,3-dihydroxybutanoic acid providing halohydrocarbon dehalogenase, an altered at least predominantly (S,S)-4-bromo-2,3-dihydroxybutanoic acid providing halohydrocarbon dehalogenase, an altered at least predominantly (S,S)-alkyl 4-chloro-2,3-dihydroxybutanoate providing halohydrocarbon dehalogenase, an altered at least predominantly (S,S)-alkyl 4-bromo-2,3-dihydroxybutanoate providing halohydrocarbon dehalogenase, an altered at least predominantly (R,S)-2-chloro-3,4-dihydroxybutanenitrile providing halohydrocarbon dehalogenase, an altered at least predominantly (R,S)-2-bromo-3,4-dihydroxybutanenitrile providing halohydrocarbon dehalogenase, an altered at least predominantly (R,S)-2-chloro-3,4-dihydroxybutanamide providing halohydrocarbon dehalogenase, an altered at least predominantly (R,S)-2-bromo-3,4-dihydroxybutanamide providing halohydrocarbon dehalogenase, an altered at least predominantly (R,S)-2-chloro-3,4-dihydroxybutanoic acid providing halohydrocarbon dehalogenase, an altered at least predominantly (R,S)-2-bromo-3,4-dihydroxybutanoic acid providing halohydrocarbon dehalogenase, an altered at least predominantly (R,S)-alkyl 2-chloro-3,4-dihydroxybutanoate providing halohydrocarbon dehalogenase, an altered at least predominantly (R,S)-alkyl 2-bromo-3,4-dihydroxybutanoate providing halohydrocarbon dehalogenase, an altered at least predominantly (R,S)-3-chloro-2,4-dihydroxybutanenitrile providing halohydrocarbon dehalogenase, an altered at least predominantly (R,S)-3-bromo-2,4-dihydroxybutanenitrile providing halohydrocarbon dehalogenase, an altered at least predominantly (R,S)-3-chloro-2,4-dihydroxybutanamide providing halohydrocarbon dehalogenase, an altered at least predominantly (R,S)-3-bromo-2,4-dihydroxybutanamide providing halohydrocarbon dehalogenase, an altered at least predominantly (R,S)-3-chloro-2,4-dihydroxybutanoic acid providing halohydrocarbon dehalogenase, an altered at least predominantly (R,S)-3-bromo-2,4-dihydroxybutanoic acid providing halohydrocarbon dehalogenase, an altered at least predominantly (R,S)-alkyl 3-chloro-2,4-dihydroxybutanoate providing halohydrocarbon dehalogenase, an altered at least predominantly (R,S)-alkyl 3-bromo-2,4-dihydroxybutanoate providing halohydrocarbon dehalogenase, an altered at least predominantly (R,S)-4-chloro-2,3-dihydroxybutanenitrile providing halohydrocarbon dehalogenase, an altered at least predominantly (R,S)-4-bromo-2,3-dihydroxybutanenitrile providing halohydrocarbon dehalogenase, an altered at least predominantly (R,S)-4-chloro-2,3-dihydroxybutanamide providing halohydrocarbon dehalogenase, an altered at least predominantly (R,S)-4-bromo-2,3-dihydroxybutanamide providing halohydrocarbon dehalogenase, an altered at least predominantly (R,S)-4-chloro-2,3-dihydroxybutanoic acid providing halohydrocarbon dehalogenase, an altered at least predominantly (R,S)-4-bromo-2,3-dihydroxybutanoic acid providing halohydrocarbon dehalogenase, an altered at least predominantly (R,S)-alkyl 4-chloro-2,3-dihydroxybutanoate providing halohydrocarbon dehalogenase, an altered at least predominantly (R,S)-alkyl 4-bromo-2,3-dihydroxybutanoate providing halohydrocarbon dehalogenase, an altered at least predominantly (S,R)-2-chloro-3,4-dihydroxybutanenitrile providing halohydrocarbon dehalogenase, an altered at least predominantly (S,R)-2-bromo-3,4-dihydroxybutanenitrile providing halohydrocarbon dehalogenase, an altered at least predominantly (S,R)-2-chloro-3,4-dihydroxybutanamide providing halohydrocarbon dehalogenase, an altered at least predominantly (S,R)-2-bromo-3,4-dihydroxybutanamide providing halohydrocarbon dehalogenase, an altered at least predominantly (S,R)-2-chloro-3,4-dihydroxybutanoic acid providing halohydrocarbon dehalogenase, an altered at least predominantly (S,R)-2-bromo-3,4-dihydroxybutanoic acid providing halohydrocarbon dehalogenase, an altered at least predominantly (S,R)-alkyl 2-chloro-3,4-dihydroxybutanoate providing halohydrocarbon dehalogenase, an altered at least predominantly (S,R)-alkyl 2-bromo-3,4-dihydroxybutanoate providing halohydrocarbon dehalogenase, an altered at least predominantly (S,R)-3-chloro-2,4-dihydroxybutanenitrile providing halohydrocarbon dehalogenase, an altered at least predominantly (S,R)-3-bromo-2,4-dihydroxybutanenitrile providing halohydrocarbon dehalogenase, an altered at least predominantly (S,R)-3-chloro-2,4-dihydroxybutanamide providing halohydrocarbon dehalogenase, an altered at least predominantly (S,R)-3-bromo-2,4-dihydroxybutanamide providing halohydrocarbon dehalogenase, an altered at least predominantly (S,R)-3-chloro-2,4-dihydroxybutanoic acid providing halohydrocarbon dehalogenase, an altered at least predominantly (S,R)-3-bromo-2,4-dihydroxybutanoic acid providing halohydrocarbon dehalogenase, an altered at least predominantly (S,R)-alkyl 3-chloro-2,4-dihydroxybutanoate providing halohydrocarbon dehalogenase, an altered at least predominantly (S,R)-alkyl 3-bromo-2,4-dihydroxybutanoate providing halohydrocarbon dehalogenase, an altered at least predominantly (S,R)-4-chloro-2,3-dihydroxybutanenitrile providing halohydrocarbon dehalogenase, an altered at least predominantly (S,R)-4-bromo-2,3-dihydroxybutanenitrile providing halohydrocarbon dehalogenase, an altered at least predominantly (S,R)-4-chloro-2,3-dihydroxybutanamide providing halohydrocarbon dehalogenase, an altered at least predominantly (S,R)-4-bromo-2,3-dihydroxybutanamide providing halohydrocarbon dehalogenase, an altered at least predominantly (S,R)-4-chloro-2,3-dihydroxybutanoic acid providing halohydrocarbon dehalogenase, an altered at least predominantly (S,R)-4-bromo-2,3-dihydroxybutanoic acid providing halohydrocarbon dehalogenase, an altered at least predominantly (S,R)-alkyl 4-chloro-2,3-dihydroxybutanoate providing halohydrocarbon dehalogenase, an altered at least predominantly (S,R)-alkyl 4-bromo-2,3-dihydroxybutanoate providing halohydrocarbon dehalogenase, an altered 4-hydroxy-2,3-epoxybutanenitrile providing halohydrocarbon dehalogenase, an altered 4-hydroxy-2,3-epoxybutanamide providing halohydrocarbon dehalogenase, an altered 4-hydroxy-2,3-epoxybutanoic acid providing halohydrocarbon dehalogenase, an altered alkyl 4-hydroxy-2,3-epoxybutanoate providing halohydrocarbon dehalogenase, an altered 2-hydroxy-3,4-epoxybutanenitrile providing halohydrocarbon dehalogenase, an altered 2-hydroxy-3,4-epoxybutanamide providing halohydrocarbon dehalogenase, an altered 2-hydroxy-3,4-epoxybutanoic acid providing halohydrocarbon dehalogenase, an altered alkyl 2-hydroxy-3,4-epoxybutanoate providing halohydrocarbon dehalogenase, an altered at least predominantly (R,R)-4-hydroxy-2,3-epoxybutanenitrile providing halohydrocarbon dehalogenase, an altered at least predominantly (R,R)-4-hydroxy-2,3-epoxybutanamide providing halohydrocarbon dehalogenase, an altered at least predominantly (R,R)-4-hydroxy-2,3-epoxybutanoic acid providing halohydrocarbon dehalogenase, an altered at least predominantly (R,R)-alkyl 4-hydroxy-2,3-epoxybutanoate providing halohydrocarbon dehalogenase, an altered at least predominantly (R,R)-2-hydroxy-3,4-epoxybutanenitrile providing halohydrocarbon dehalogenase, an altered at least predominantly (R,R)-2-hydroxy-3,4-epoxybutanamide providing halohydrocarbon dehalogenase, an altered at least predominantly (R,R)-2-hydroxy-3,4-epoxybutanoic acid providing halohydrocarbon dehalogenase, an altered at least predominantly (R,R)-alkyl 2-hydroxy-3,4-epoxybutanoate providing halohydrocarbon dehalogenase, an altered at least predominantly (S,S)-4-hydroxy-2,3-epoxybutanenitrile providing halohydrocarbon dehalogenase, an altered at least predominantly (S,S)-4-hydroxy-2,3-epoxybutanamide providing halohydrocarbon dehalogenase, an altered at least predominantly (S,S)-4-hydroxy-2,3-epoxybutanoic acid providing halohydrocarbon dehalogenase, an altered at least predominantly (S,S)-alkyl 4-hydroxy-2,3-epoxybutanoate providing halohydrocarbon dehalogenase, an altered at least predominantly (S,S)-2-hydroxy-3,4-epoxybutanenitrile providing halohydrocarbon dehalogenase, an altered at least predominantly (S,S)-2-hydroxy-3,4-epoxybutanamide providing halohydrocarbon dehalogenase, an altered at least predominantly (S,S)-2-hydroxy-3,4-epoxybutanoic acid providing halohydrocarbon dehalogenase, an altered at least predominantly (S,S)-alkyl 2-hydroxy-3,4-epoxybutanoate providing halohydrocarbon dehalogenase, an altered at least predominantly (R,S)-4-hydroxy-2,3-epoxybutanenitrile providing halohydrocarbon dehalogenase, an altered at least predominantly (R,S)-4-hydroxy-2,3-epoxybutanamide providing halohydrocarbon dehalogenase, an altered at least predominantly (R,S)-4-hydroxy-2,3-epoxybutanoic acid providing halohydrocarbon dehalogenase, an altered at least predominantly (R,S)-alkyl 4-hydroxy-2,3-epoxybutanoate providing halohydrocarbon dehalogenase, an altered at least predominantly (R,S)-2-hydroxy-3,4-epoxybutanenitrile providing halohydrocarbon dehalogenase, an altered at least predominantly (R,S)-2-hydroxy-3,4-epoxybutanamide providing halohydrocarbon dehalogenase, an altered at least predominantly (R,S)-2-hydroxy-3,4-epoxybutanoic acid providing halohydrocarbon dehalogenase, an altered at least predominantly (R,S)-alkyl 2-hydroxy-3,4-epoxybutanoate providing halohydrocarbon dehalogenase, an altered at least predominantly (S,R)-4-hydroxy-2,3-epoxybutanenitrile providing halohydrocarbon dehalogenase, an altered at least predominantly (S,R)-4-hydroxy-2,3-epoxybutanamide providing halohydrocarbon dehalogenase, an altered at least predominantly (S,R)-4-hydroxy-2,3-epoxybutanoic acid providing halohydrocarbon dehalogenase, an altered at least predominantly (S,R)-alkyl 4-hydroxy-2,3-epoxybutanoate providing halohydrocarbon dehalogenase, an altered at least predominantly (S,R)-2-hydroxy-3,4-epoxybutanenitrile providing halohydrocarbon dehalogenase, an altered at least predominantly (S,R)-2-hydroxy-3,4-epoxybutanamide providing halohydrocarbon dehalogenase, an altered at least predominantly (S,R)-2-hydroxy-3,4-epoxybutanoic acid providing halohydrocarbon dehalogenase, an altered at least predominantly (S,R)-alkyl 2-hydroxy-3,4-epoxybutanoate providing halohydrocarbon dehalogenase, an altered 4-chloro-2,3-epoxybutanenitrile providing halohydrocarbon dehalogenase, an altered 4-bromo-2,3-epoxybutanenitrile providing halohydrocarbon dehalogenase, an altered 4-chloro-2,3-epoxybutanamide providing halohydrocarbon dehalogenase, an altered 4-bromo-2,3-epoxybutanamide providing halohydrocarbon dehalogenase, an altered 4-chloro-2,3-epoxybutanoic acid providing halohydrocarbon dehalogenase, an altered 4-bromo-2,3-epoxybutanoic acid providing halohydrocarbon dehalogenase, an altered alkyl 4-chloro-2,3-epoxybutanoate providing halohydrocarbon dehalogenase, an altered alkyl 4-bromo-2,3-epoxybutanoate providing halohydrocarbon dehalogenase, an altered 2-chloro-3,4-epoxybutanenitrile providing halohydrocarbon dehalogenase, an altered 2-bromo-3,4-epoxybutanenitrile providing halohydrocarbon dehalogenase, an altered 2-chloro-3,4-epoxybutanamide providing halohydrocarbon dehalogenase, an altered 2-bromo-3,4-epoxybutanamide providing halohydrocarbon dehalogenase, an altered 2-chloro-3,4-epoxybutanoic acid providing halohydrocarbon dehalogenase, an altered 2-bromo-3,4-epoxybutanoic acid providing halohydrocarbon dehalogenase, an altered alkyl 2-chloro-3,4-epoxybutanoate providing halohydrocarbon dehalogenase, an altered alkyl 2-bromo-3,4-epoxybutanoate providing halohydrocarbon dehalogenase, an altered at least predominantly (R,R)-4-chloro-2,3-epoxybutanenitrile providing halohydrocarbon dehalogenase, an altered at least predominantly (R,R)-4-bromo-2,3-epoxybutanenitrile providing halohydrocarbon dehalogenase, an altered at least predominantly (R,R)-4-chloro-2,3-epoxybutanamide providing halohydrocarbon dehalogenase, an altered at least predominantly (R,R)-4-bromo-2,3-epoxybutanamide providing halohydrocarbon dehalogenase, an altered at least predominantly (R,R)-4-chloro-2,3-epoxybutanoic acid providing halohydrocarbon dehalogenase, an altered at least predominantly (R,R)-4-bromo-2,3-epoxybutanoic acid providing halohydrocarbon dehalogenase, an altered at least predominantly (R,R)-alkyl 4-chloro-2,3-epoxybutanoate providing halohydrocarbon dehalogenase, an altered at least predominantly (R,R)-alkyl 4-bromo-2,3-epoxybutanoate providing halohydrocarbon dehalogenase, an altered at least predominantly (R,R)-2-chloro-3,4-epoxybutanenitrile providing halohydrocarbon dehalogenase, an altered at least predominantly (R,R)-2-bromo-3,4-epoxybutanenitrile providing halohydrocarbon dehalogenase, an altered at least predominantly (R,R)-2-chloro-3,4-epoxybutanamide providing halohydrocarbon dehalogenase, an altered at least predominantly (R,R)-2-bromo-3,4-epoxybutanamide providing halohydrocarbon dehalogenase, an altered at least predominantly (R,R)-2-chloro-3,4-epoxybutanoic acid providing halohydrocarbon dehalogenase, an altered at least predominantly (R,R)-2-bromo-3,4-epoxybutanoic acid providing halohydrocarbon dehalogenase, an altered at least predominantly (R,R)-alkyl 2-chloro-3,4-epoxybutanoate providing halohydrocarbon dehalogenase, an altered at least predominantly (R,R)-alkyl 2-bromo-3,4-epoxybutanoate providing halohydrocarbon dehalogenase, an altered at least predominantly (S,S)-4-chloro-2,3-epoxybutanenitrile providing halohydrocarbon dehalogenase, an altered at least predominantly (S,S)-4-bromo-2,3-epoxybutanenitrile providing halohydrocarbon dehalogenase, an altered at least predominantly (S,S)-4-chloro-2,3-epoxybutanamide providing halohydrocarbon dehalogenase, an altered at least predominantly (S,S)-4-bromo-2,3-epoxybutanamide providing halohydrocarbon dehalogenase, an altered at least predominantly (S,S)-4-chloro-2,3-epoxybutanoic acid providing halohydrocarbon dehalogenase, an altered at least predominantly (S,S)-4-bromo-2,3-epoxybutanoic acid providing halohydrocarbon dehalogenase, an altered at least predominantly (S,S)-alkyl 4-chloro-2,3-epoxybutanoate providing halohydrocarbon dehalogenase, an altered at least predominantly (S,S)-alkyl 4-bromo-2,3-epoxybutanoate providing halohydrocarbon dehalogenase, an altered at least predominantly (S,S)-2-chloro-3,4-epoxybutanenitrile providing halohydrocarbon dehalogenase, an altered at least predominantly (S,S)-2-bromo-3,4-epoxybutanenitrile providing halohydrocarbon dehalogenase, an altered at least predominantly (S,S)-2-chloro-3,4-epoxybutanamide providing halohydrocarbon dehalogenase, an altered at least predominantly (S,S)-2-bromo-3,4-epoxybutanamide providing halohydrocarbon dehalogenase, an altered at least predominantly (S,S)-2-chloro-3,4-epoxybutanoic acid providing halohydrocarbon dehalogenase, an altered at least predominantly (S,S)-2-bromo-3,4-epoxybutanoic acid providing halohydrocarbon dehalogenase, an altered at least predominantly (S,S)-alkyl 2-chloro-3,4-epoxybutanoate providing halohydrocarbon dehalogenase, an altered at least predominantly (S,S)-alkyl 2-bromo-3,4-epoxybutanoate providing halohydrocarbon dehalogenase, an altered at least predominantly (R,S)-4-chloro-2,3-epoxybutanenitrile providing halohydrocarbon dehalogenase, an altered at least predominantly (R,S)-4-bromo-2,3-epoxybutanenitrile providing halohydrocarbon dehalogenase, an altered at least predominantly (R,S)-4-chloro-2,3-epoxybutanamide providing halohydrocarbon dehalogenase, an altered at least predominantly (R,S)-4-bromo-2,3-epoxybutanamide providing halohydrocarbon dehalogenase, an altered at least predominantly (R,S)-4-chloro-2,3-epoxybutanoic acid providing halohydrocarbon dehalogenase, an altered at least predominantly (R,S)-4-bromo-2,3-epoxybutanoic acid providing halohydrocarbon dehalogenase, an altered at least predominantly (R,S)-alkyl 4-chloro-2,3-epoxybutanoate providing halohydrocarbon dehalogenase, an altered at least predominantly (R,S)-alkyl 4-bromo-2,3-epoxybutanoate providing halohydrocarbon dehalogenase, an altered at least predominantly (R,S)-2-chloro-3,4-epoxybutanenitrile providing halohydrocarbon dehalogenase, an altered at least predominantly (R,S)-2-bromo-3,4-epoxybutanenitrile providing halohydrocarbon dehalogenase, an altered at least predominantly (R,S)-2-chloro-3,4-epoxybutanamide providing halohydrocarbon dehalogenase, an altered at least predominantly (R,S)-2-bromo-3,4-epoxybutanamide providing halohydrocarbon dehalogenase, an altered at least predominantly (R,S)-2-chloro-3,4-epoxybutanoic acid providing halohydrocarbon dehalogenase, an altered at least predominantly (R,S)-2-bromo-3,4-epoxybutanoic acid providing halohydrocarbon dehalogenase, an altered at least predominantly (R,S)-alkyl 2-chloro-3,4-epoxybutanoate providing halohydrocarbon dehalogenase, an altered at least predominantly (R,S)-alkyl 2-bromo-3,4-epoxybutanoate providing halohydrocarbon dehalogenase, an altered at least predominantly (S,R)-4-chloro-2,3-epoxybutanenitrile providing halohydrocarbon dehalogenase, an altered at least predominantly (S,R)-4-bromo-2,3-epoxybutanenitrile providing halohydrocarbon dehalogenase, an altered at least predominantly (S,R)-4-chloro-2,3-epoxybutanamide providing halohydrocarbon dehalogenase, an altered at least predominantly (S,R)-4-bromo-2,3-epoxybutanamide providing halohydrocarbon dehalogenase, an altered at least predominantly (S,R)-4-chloro-2,3-epoxybutanoic acid providing halohydrocarbon dehalogenase, an altered at least predominantly (S,R)-4-bromo-2,3-epoxybutanoic acid providing halohydrocarbon dehalogenase, an altered at least predominantly (S,R)-alkyl 4-chloro-2,3-epoxybutanoate providing halohydrocarbon dehalogenase, an altered at least predominantly (S,R)-alkyl 4-bromo-2,3-epoxybutanoate providing halohydrocarbon dehalogenase, an altered at least predominantly (S,R)-2-chloro-3,4-epoxybutanenitrile providing halohydrocarbon dehalogenase, an altered at least predominantly (S,R)-2-bromo-3,4-epoxybutanenitrile providing halohydrocarbon dehalogenase, an altered at least predominantly (S,R)-2-chloro-3,4-epoxybutanamide providing halohydrocarbon dehalogenase, an altered at least predominantly (S,R)-2-bromo-3,4-epoxybutanamide providing halohydrocarbon dehalogenase, an altered at least predominantly (S,R)-2-chloro-3,4-epoxybutanoic acid providing halohydrocarbon dehalogenase, an altered at least predominantly (S,R)-2-bromo-3,4-epoxybutanoic acid providing halohydrocarbon dehalogenase, an altered at least predominantly (S,R)-alkyl 2-chloro-3,4-epoxybutanoate providing halohydrocarbon dehalogenase, an altered at least predominantly (S,R)-alkyl 2-bromo-3,4-epoxybutanoate providing halohydrocarbon dehalogenase, and the like.

An “altered haloalcohol dehalogenase” can include, e.g., an altered 1-halo-2,3-epoxypropane providing haloalcohol dehalogenase, an altered 1,2-dihalo-3,4-epoxybutane providing haloalcohol dehalogenase, an altered 1,2-dihalo-4,5-epoxypentane providing haloalcohol dehalogenase, an altered 1,2-dihalo-5,6-epoxyhexane providing haloalcohol dehalogenase, an altered 1,2-dihalo-6,7-epoxyheptane providing haloalcohol dehalogenase, an altered 1,2-dihalo-7,8-epoxyoctane providing haloalcohol dehalogenase, an altered 2-halo-1,3-propanediol providing haloalcohol dehalogenase, an altered 2,3-dihalo-1,4-butanediol providing haloalcohol dehalogenase, an altered 2,4-dihalo-1,5-pentanediol providing haloalcohol dehalogenase, an altered 2,5-dihalo-1,6-hexanediol providing haloalcohol dehalogenase, an altered 2,6-dihalo-1,7-heptanediol providing haloalcohol dehalogenase, an altered 2,7-dihalo-1,8-octanediol providing haloalcohol dehalogenase, an altered 4-halo-2,3-dihydroxybutanenitrile providing haloalcohol dehalogenase, an altered 4-halo-2,3-dihydroxybutanamide providing haloalcohol dehalogenase, an altered 4-halo-2,3-dihydroxybutanoic acid providing haloalcohol dehalogenase, an altered alkyl 4-halo-2,3-dihydroxybutanoate providing haloalcohol dehalogenase, an altered 3-halo-2,4-dihydroxybutanenitrile providing haloalcohol dehalogenase, an altered 3-halo-2,4-dihydroxybutanamide providing haloalcohol dehalogenase, an altered 3-halo-2,4-dihydroxybutanoic acid providing haloalcohol dehalogenase, an altered alkyl 3-halo-2,4-dihydroxybutanoate providing haloalcohol dehalogenase, an altered 2-halo-3,4-dihydroxybutanenitrile providing haloalcohol dehalogenase, an altered 2-halo-3,4-dihydroxybutanamide providing haloalcohol dehalogenase, an altered 2-halo-3,4-dihydroxybutanoic acid providing haloalcohol dehalogenase, an altered alkyl 2-halo-3,4-dihydroxybutanoate providing haloalcohol dehalogenase, an altered 2-halo-3,4-epoxybutanenitrile providing haloalcohol dehalogenase, an altered 2-halo-3,4-epoxybutanamide providing haloalcohol dehalogenase, an altered 2-halo-3,4-epoxybutanoic acid providing haloalcohol dehalogenase, an altered alkyl 2-halo-3,4-epoxybutanoate providing haloalcohol dehalogenase, an altered 4-halo-2,3-epoxybutanenitrile providing haloalcohol dehalogenase, an altered 4-halo-2,3-epoxybutanamide providing haloalcohol dehalogenase, an altered 4-halo-2,3-epoxybutanoic acid providing haloalcohol dehalogenase, an altered alkyl 4-halo-2,3-epoxybutanoate providing haloalcohol dehalogenase, an altered 1-chloro-2,3-epoxypropane providing haloalcohol dehalogenase, an altered 1,2-epoxypropane providing haloalcohol dehalogenase, an altered ethylene oxide providing haloalcohol dehalogenase, an altered 1,2-dichloro-3,4-epoxybutane providing haloalcohol dehalogenase, an altered 1,2-dichloro-4,5-epoxypentane providing haloalcohol dehalogenase, an altered 1,2-dichloro-5,6-epoxyhexane providing haloalcohol dehalogenase, an altered 1,2-dichloro-6,7-epoxyheptane providing haloalcohol dehalogenase, an altered 1,2-dichloro-7,8-epoxyoctane providing haloalcohol dehalogenase, an altered 2-chloro-1,3-propanediol providing haloalcohol dehalogenase, an altered 1,2-ethanediol providing haloalcohol dehalogenase, an altered 1,3-propanediol providing haloalcohol dehalogenase, an altered 2,3-dichloro-1,4-butanediol providing haloalcohol dehalogenase, an altered 2,4-dichloro-1,5-pentanediol providing haloalcohol dehalogenase, an altered 2,5-dichloro-1,6-hexanediol providing haloalcohol dehalogenase, an altered 2,6-dichloro-1,7-heptanediol providing haloalcohol dehalogenase, an altered 2,7-dichloro-1,8-octanediol providing haloalcohol dehalogenase, an altered at least predominantly (R)-1-chloro-2,3-epoxypropane providing haloalcohol dehalogenase, an altered at least predominantly (S)-1-chloro-2,3-epoxypropane providing haloalcohol dehalogenase, an altered at least predominantly (R)-1,2-epoxypropane providing haloalcohol dehalogenase, an altered at least predominantly (S)-1,2-epoxypropane providing haloalcohol dehalogenase, an altered at least predominantly (R,R)-1,2-dichloro-3,4-epoxybutane providing haloalcohol dehalogenase, an altered at least predominantly (S,S)-1,2-dichloro-3,4-epoxybutane providing haloalcohol dehalogenase, an altered at least predominantly (R,S)-1,2-dichloro-3,4-epoxybutane providing haloalcohol dehalogenase, an altered at least predominantly (S,R)-1,2-dichloro-3,4-epoxybutane providing haloalcohol dehalogenase, an altered at least predominantly (R,R)-1,2-dichloro-4,5-epoxypentane providing haloalcohol dehalogenase, an altered at least predominantly (S,S)-1,2-dichloro-4,5-epoxypentane providing haloalcohol dehalogenase, an altered at least predominantly (R,S)-1,2-dichloro-4,5-epoxypentane providing haloalcohol dehalogenase, an altered at least predominantly (S,R)-1,2-dichloro-4,5-epoxypentane providing haloalcohol dehalogenase, an altered at least predominantly (R,R)-1,2-dichloro-5,6-epoxyhexane providing haloalcohol dehalogenase, an altered at least predominantly (S,S)-1,2-dichloro-5,6-epoxyhexane providing haloalcohol dehalogenase, an altered at least predominantly (R,S)-1,2-dichloro-5,6-epoxyhexane providing haloalcohol dehalogenase, an altered at least predominantly (S,R)-1,2-dichloro-5,6-epoxyhexane providing haloalcohol dehalogenase, an altered at least predominantly (R,R)-1,2-dichloro-6,7-epoxyheptane providing haloalcohol dehalogenase, an altered at least predominantly (S,S)-1,2-dichloro-6,7-epoxyheptane providing haloalcohol dehalogenase, an altered at least predominantly (R,S)-1,2-dichloro-6,7-epoxyheptane providing haloalcohol dehalogenase, an altered at least predominantly (S,R)-1,2-dichloro-6,7-epoxyheptane providing haloalcohol dehalogenase, an altered at least predominantly (R,R)-1,2-dichloro-7,8-epoxyoctane providing haloalcohol dehalogenase, an altered at least predominantly (S,S)-1,2-dichloro-7,8-epoxyoctane providing haloalcohol dehalogenase, an altered at least predominantly (R,S)-1,2-dichloro-7,8-epoxyoctane providing haloalcohol dehalogenase, an altered at least predominantly (S,R)-1,2-dichloro-7,8-epoxyoctane providing haloalcohol dehalogenase, an altered at least predominantly (R,R)-2,3-dichloro-1,4-butanediol providing haloalcohol dehalogenase, an altered at least predominantly (S,S)-2,3-dichloro-1,4-butanediol providing haloalcohol dehalogenase, an altered at least predominantly (R,S)-2,3-dichloro-1,4-butanediol providing haloalcohol dehalogenase, an altered at least predominantly (R,R)-2,4-dichloro-1,5-pentanediol providing haloalcohol dehalogenase, an altered at least predominantly (S,S)-2,4-dichloro-1,5-pentanediol providing haloalcohol dehalogenase, an altered at least predominantly (R,S)-2,4-dichloro-1,5-pentanediol providing haloalcohol dehalogenase, an altered at least predominantly (R,R)-2,5-dichloro-1,6-hexanediol providing haloalcohol dehalogenase, an altered at least predominantly (S,S)-2,5-dichloro-1,6-hexanediol providing haloalcohol dehalogenase, an altered at least predominantly (R,S)-2,5-dichloro-1,6-hexanediol providing haloalcohol dehalogenase, an altered at least predominantly (R,R)-2,6-dichloro-1,7-heptanediol providing haloalcohol dehalogenase, an altered at least predominantly (S,S)-2,6-dichloro-1,7-heptanediol providing haloalcohol dehalogenase, an altered at least predominantly (R,S)-2,6-dichloro-1,7-heptanediol providing haloalcohol dehalogenase, an altered at least predominantly (R,R)-2,7-dichloro-1,8-octanediol providing haloalcohol dehalogenase, an altered at least predominantly (S,S)-2,7-dichloro-1,8-octanediol providing haloalcohol dehalogenase, an altered at least predominantly (R,S)-2,7-dichloro-1,8-octanediol providing haloalcohol dehalogenase, an altered 2,3-dihydoxypropanenitrile providing haloalcohol dehalogenase, an altered 3,4-dihydoxybutanenitrile providing haloalcohol dehalogenase, an altered 2,3-dihydoxypropanamide providing haloalcohol dehalogenase, an altered 3,4-dihydoxybutanamide providing haloalcohol dehalogenase, an altered 2,3-dihydoxypropanoic acid providing haloalcohol dehalogenase, an altered 3,4-dihydoxybutanoic acid providing haloalcohol dehalogenase, an altered at least predominantly (R)-2,3-dihydoxypropanenitrile providing haloalcohol dehalogenase, an altered at least predominantly (S)-2,3-dihydoxypropanenitrile providing haloalcohol dehalogenase, an altered at least predominantly (R)-3,4-dihydoxybutanenitrile providing haloalcohol dehalogenase, an altered at least predominantly (S)-3,4-dihydoxybutanenitrile providing haloalcohol dehalogenase, an altered at least predominantly (R)-2,3-dihydoxypropanamide providing haloalcohol dehalogenase, an altered at least predominantly (S)-2,3-dihydoxypropanamide providing haloalcohol dehalogenase, an altered at least predominantly (R)-3,4-dihydoxybutanamide providing haloalcohol dehalogenase, an altered at least predominantly (S)-3,4-dihydoxybutanamide providing haloalcohol dehalogenase, an altered at least predominantly (R)-2,3-dihydoxypropanoic acid providing haloalcohol dehalogenase, an altered at least predominantly (S)-2,3-dihydoxypropanoic acid providing haloalcohol dehalogenase, an altered at least predominantly (R)-3,4-dihydoxybutanoic acid providing haloalcohol dehalogenase, an altered at least predominantly (S)-3,4-dihydoxybutanoic acid providing haloalcohol dehalogenase, an altered 2,3-epoxy-1-propanenitrile providing haloalcohol dehalogenase, an altered 3,4-epoxy-1-butanenitrile providing haloalcohol dehalogenase, an altered 2,3-epoxy-1-propanamide providing haloalcohol dehalogenase, an altered 3,4-epoxy-1-butanamide providing haloalcohol dehalogenase, an altered 2,3-epoxy-1-propanoic acid providing haloalcohol dehalogenase, an altered 3,4-epoxy-1-butanoic acid providing haloalcohol dehalogenase, an altered at least predominantly (R)-2,3-epoxy-1-propanenitrile providing haloalcohol dehalogenase, an altered at least predominantly (S)-2,3-epoxy-1-propanenitrile providing haloalcohol dehalogenase, an altered at least predominantly (R)-3,4-epoxy-1-butanenitrile providing haloalcohol dehalogenase, an altered at least predominantly (S)-3,4-epoxy-1-butanenitrile providing haloalcohol dehalogenase, an altered at least predominantly (R)-2,3-epoxy-1-propanamide providing haloalcohol dehalogenase, an altered at least predominantly (S)-2,3-epoxy-1-propanamide providing haloalcohol dehalogenase, an altered at least predominantly (R)-3,4-epoxy-1-butanamide providing haloalcohol dehalogenase, an altered at least predominantly (S)-3,4-epoxy-1-butanamide providing haloalcohol dehalogenase, an altered at least predominantly (R)-2,3-epoxy-1-propanoic acid providing haloalcohol dehalogenase, an altered at least predominantly (S)-2,3-epoxy-1-propanoic acid providing haloalcohol dehalogenase, an altered at least predominantly (R)-3,4-epoxy-1-butanoic acid providing haloalcohol dehalogenase, an altered at least predominantly (S)-3,4-epoxy-1-butanoic acid providing haloalcohol dehalogenase, an altered alkyl 2,3-dihydroxypropanoate providing haloalcohol dehalogenase, an altered alkyl 3,4-dihydroxybutanoate providing haloalcohol dehalogenase, an altered alkyl 2,3-epoxy-1-propanoate providing haloalcohol dehalogenase, an altered alkyl 3,4-epoxy-1-butanoate providing haloalcohol dehalogenase, an altered at least predominantly (R)-alkyl 2,3-dihydroxypropanoate providing haloalcohol dehalogenase, an altered at least predominantly (R)-alkyl 3,4-dihydroxybutanoate providing haloalcohol dehalogenase, an altered at least predominantly (S)-alkyl 2,3-dihydroxypropanoate providing haloalcohol dehalogenase, an altered at least predominantly (S)-alkyl 3,4-dihydroxybutanoate providing haloalcohol dehalogenase, an altered at least predominantly (R)-alkyl 2,3-epoxy-1-propanoate providing haloalcohol dehalogenase, an altered at least predominantly (R)-alkyl 3,4-epoxy-1-butanoate providing haloalcohol dehalogenase, an altered at least predominantly (S)-alkyl 2,3-epoxy-1-propanoate providing haloalcohol dehalogenase, an altered at least predominantly (S)-alkyl 3,4-epoxy-1-butanoate providing haloalcohol dehalogenase, an altered 2,3-dihydroxybutanenitrile providing haloalcohol dehalogenase, an altered 2,3-dihydroxybutanamide providing haloalcohol dehalogenase, an altered 2,3-dihydroxybutanoic acid providing haloalcohol dehalogenase, an altered alkyl 2,3-dihydroxybutanoate providing haloalcohol dehalogenase, an altered 2,3-epoxy-1-butanenitrile providing haloalcohol dehalogenase, an altered 2,3-epoxy-1-butanamide providing haloalcohol dehalogenase, an altered 2,3-epoxy-1-butanoic acid providing haloalcohol dehalogenase, an altered alkyl 2,3-epoxy-1-butanoate providing haloalcohol dehalogenase, an altered at least predominantly (R,R)-2,3-dihydroxybutanenitrile providing haloalcohol dehalogenase, an altered at least predominantly (R,R)-2,3-dihydroxybutanamide providing haloalcohol dehalogenase, an altered at least predominantly (R,R)-2,3-dihydroxybutanoic acid providing haloalcohol dehalogenase, an altered at least predominantly (R,R)-alkyl 2,3-dihydroxybutanoate providing haloalcohol dehalogenase, an altered at least predominantly (S,S)-2,3-dihydroxybutanenitrile providing haloalcohol dehalogenase, an altered at least predominantly (S,S)-2,3-dihydroxybutanamide providing haloalcohol dehalogenase, an altered at least predominantly (S,S)-2,3-dihydroxybutanoic acid providing haloalcohol dehalogenase, an altered at least predominantly (S,S)-alkyl 2,3-dihydroxybutanoate providing haloalcohol dehalogenase, an altered at least predominantly (R,S)-2,3-dihydroxybutanenitrile providing haloalcohol dehalogenase, an altered at least predominantly (R,S)-2,3-dihydroxybutanamide providing haloalcohol dehalogenase, an altered at least predominantly (R,S)-2,3-dihydroxybutanoic acid providing haloalcohol dehalogenase, an altered at least predominantly (R,S)-alkyl 2,3-dihydroxybutanoate providing haloalcohol dehalogenase, an altered at least predominantly (S,R)-2,3-dihydroxybutanenitrile providing haloalcohol dehalogenase, an altered at least predominantly (S,R)-2,3-dihydroxybutanamide providing haloalcohol dehalogenase, an altered at least predominantly (S,R)-2,3-dihydroxybutanoic acid providing haloalcohol dehalogenase, an altered at least predominantly (S,R)-alkyl 2,3-dihydroxybutanoate providing haloalcohol dehalogenase, an altered at least predominantly (R,R)-2,3-epoxy-1-butanenitrile providing haloalcohol dehalogenase, an altered at least predominantly (R,R)-2,3-epoxy-1-butanamide providing haloalcohol dehalogenase, an altered at least predominantly (R,R)-2,3-epoxy-1-butanoic acid providing haloalcohol dehalogenase, an altered at least predominantly (R,R)-alkyl 2,3-epoxy-1-butanoate providing haloalcohol dehalogenase, an altered at least predominantly (S,S)-2,3-epoxy-1-butanenitrile providing haloalcohol dehalogenase, an altered at least predominantly (S,S)-2,3-epoxy-1-butanamide providing haloalcohol dehalogenase, an altered at least predominantly (S,S)-2,3-epoxy-1-butanoic acid providing haloalcohol dehalogenase, an altered at least predominantly (S,S)-alkyl 2,3-epoxy-1-butanoate providing haloalcohol dehalogenase, an altered at least predominantly (R,S)-2,3-epoxy-1-butanenitrile providing haloalcohol dehalogenase, an altered at least predominantly (R,S)-2,3-epoxy-1-butanamide providing haloalcohol dehalogenase, an altered at least predominantly (R,S)-2,3-epoxy-butanoic acid providing haloalcohol dehalogenase, an altered at least predominantly (R,S)-alkyl 2,3-epoxy-1-butanoate providing haloalcohol dehalogenase, an altered at least predominantly (S,R)-2,3-epoxy -1-butanenitrile providing haloalcohol dehalogenase, an altered at least predominantly (S,R)-2,3-epoxy-1-butanamide providing haloalcohol dehalogenase, an altered at least predominantly (S,R)-2,3-epoxy-1-butanoic acid providing haloalcohol dehalogenase, an altered at least predominantly (S,R)-alkyl 2,3-epoxy-1-butanoate providing haloalcohol dehalogenase, an altered at least predominantly (S,R)-4-chloro-2,3-dihydroxybutanenitrile providing haloalcohol dehalogenase, an altered at least predominantly (S,R)-4-bromo-2,3-dihydroxybutanenitrile providing haloalcohol dehalogenase, an altered at least predominantly (S,R)-4-chloro-2,3-dihydroxybutanamide providing haloalcohol dehalogenase, an altered at least predominantly (S,R)-4-bromo-2,3-dihydroxybutanamide providing haloalcohol dehalogenase, an altered at least predominantly (S,R)-4-chloro-2,3-dihydroxybutanoic acid providing haloalcohol dehalogenase, an altered at least predominantly (S,R)-4-bromo-2,3-dihydroxybutanoic acid providing haloalcohol dehalogenase, an altered at least predominantly (S,R)-alkyl 4-chloro-2,3-dihydroxybutanoate providing haloalcohol dehalogenase, an altered at least predominantly (S,R)-alkyl 4-bromo-2,3-dihydroxybutanoate providing haloalcohol dehalogenase, an altered at least predominantly (S,R)-3-chloro-2,4-dihydroxybutanenitrile providing haloalcohol dehalogenase, an altered at least predominantly (S,R)-3-bromo-2,4-dihydroxybutanenitrile providing haloalcohol dehalogenase, an altered at least predominantly (S,R)-3-chloro-2,4-dihydroxybutanamide providing haloalcohol dehalogenase, an altered at least predominantly (S,R)-3-bromo-2,4-dihydroxybutanamide providing haloalcohol dehalogenase, an altered at least predominantly (S,R)-3-chloro-2,4-dihydroxybutanoic acid providing haloalcohol dehalogenase, an altered at least predominantly (S,R)-3-bromo-2,4-dihydroxybutanoic acid providing haloalcohol dehalogenase, an altered at least predominantly (S,R)-alkyl 3-chloro-2,4-dihydroxybutanoate providing haloalcohol dehalogenase, an altered at least predominantly (S,R)-alkyl 3-bromo-2,4-dihydroxybutanoate providing haloalcohol dehalogenase, an altered at least predominantly (S,R)-2-chloro-3,4-dihydroxybutanenitrile providing haloalcohol dehalogenase, an altered at least predominantly (S,R)-2-bromo-3,4-dihydroxybutanenitrile providing haloalcohol dehalogenase, an altered at least predominantly (S,R)-2-chloro-3,4-dihydroxybutanamide providing haloalcohol dehalogenase, an altered at least predominantly (S,R)-2-bromo-3,4-dihydroxybutanamide providing haloalcohol dehalogenase, an altered at least predominantly (S,R)-2-chloro-3,4-dihydroxybutanoic acid providing haloalcohol dehalogenase, an altered at least predominantly (S,R)-2-bromo-3,4-dihydroxybutanoic acid providing haloalcohol dehalogenase, an altered at least predominantly (S,R)-alkyl 2-chloro-3,4-dihydroxybutanoate providing haloalcohol dehalogenase, an altered at least predominantly (S,R)-alkyl 2-bromo-3,4-dihydroxybutanoate providing haloalcohol dehalogenase, an altered at least predominantly (R,S)-4-chloro-2,3-dihydroxybutanenitrile providing haloalcohol dehalogenase, an altered at least predominantly (R,S)-4-bromo-2,3-dihydroxybutanenitrile providing haloalcohol dehalogenase, an altered at least predominantly (R,S)-4-chloro-2,3-dihydroxybutanamide providing haloalcohol dehalogenase, an altered at least predominantly (R,S)-4-bromo-2,3-dihydroxybutanamide providing haloalcohol dehalogenase, an altered at least predominantly (R,S)-4-chloro-2,3-dihydroxybutanoic acid providing haloalcohol dehalogenase, an altered at least predominantly (R,S)-4-bromo-2,3-dihydroxybutanoic acid providing haloalcohol dehalogenase, an altered at least predominantly (R,S)-alkyl 4-chloro-2,3-dihydroxybutanoate providing haloalcohol dehalogenase, an altered at least predominantly (R,S)-alkyl 4-bromo-2,3-dihydroxybutanoate providing haloalcohol dehalogenase, an altered at least predominantly (R,S)-3-chloro-2,4-dihydroxybutanenitrile providing haloalcohol dehalogenase, an altered at least predominantly (R,S)-3-bromo-2,4-dihydroxybutanenitrile providing haloalcohol dehalogenase, an altered at least predominantly (R,S)-3-chloro-2,4-dihydroxybutanamide providing haloalcohol dehalogenase, an altered at least predominantly (R,S)-3-bromo-2,4-dihydroxybutanamide providing haloalcohol dehalogenase, an altered at least predominantly (R,S)-3-chloro-2,4-dihydroxybutanoic acid providing haloalcohol dehalogenase, an altered at least predominantly (R,S)-3-bromo-2,4-dihydroxybutanoic acid providing haloalcohol dehalogenase, an altered at least predominantly (R,S)-alkyl 3-chloro-2,4-dihydroxybutanoate providing haloalcohol dehalogenase, an altered at least predominantly (R,S)-alkyl 3-bromo-2,4-dihydroxybutanoate providing haloalcohol dehalogenase, an altered at least predominantly (R,S)-2-chloro-3,4-dihydroxybutanenitrile providing haloalcohol dehalogenase, an altered at least predominantly (R,S)-2-bromo-3,4-dihydroxybutanenitrile providing haloalcohol dehalogenase, an altered at least predominantly (R,S)-2-chloro-3,4-dihydroxybutanamide providing haloalcohol dehalogenase, an altered at least predominantly (R,S)-2-bromo-3,4-dihydroxybutanamide providing haloalcohol dehalogenase, an altered at least predominantly (R,S)-2-chloro-3,4-dihydroxybutanoic acid providing haloalcohol dehalogenase, an altered at least predominantly (R,S)-2-bromo-3,4-dihydroxybutanoic acid providing haloalcohol dehalogenase, an altered at least predominantly (R,S)-alkyl 2-chloro-3,4-dihydroxybutanoate providing haloalcohol dehalogenase, an altered at least predominantly (R,S)-alkyl 2-bromo-3,4-dihydroxybutanoate providing haloalcohol dehalogenase, an altered at least predominantly (S,S)-4-chloro-2,3-dihydroxybutanenitrile providing haloalcohol dehalogenase, an altered at least predominantly (S,S)-4-bromo-2,3-dihydroxybutanenitrile providing haloalcohol dehalogenase, an altered at least predominantly (S,S)-4-chloro-2,3-dihydroxybutanamide providing haloalcohol dehalogenase, an altered at least predominantly (S,S)-4-bromo-2,3-dihydroxybutanamide providing haloalcohol dehalogenase, an altered at least predominantly (S,S)-4-chloro-2,3-dihydroxybutanoic acid providing haloalcohol dehalogenase, an altered at least predominantly (S,S)-4-bromo-2,3-dihydroxybutanoic acid providing haloalcohol dehalogenase, an altered at least predominantly (S,S)-alkyl 4-chloro-2,3-dihydroxybutanoate providing haloalcohol dehalogenase, an altered at least predominantly (S,S)-alkyl 4-bromo-2,3-dihydroxybutanoate providing haloalcohol dehalogenase, an altered at least predominantly (S,S)-3-chloro-2,4-dihydroxybutanenitrile providing haloalcohol dehalogenase, an altered at least predominantly (S,S)-3-bromo-2,4-dihydroxybutanenitrile providing haloalcohol dehalogenase, an altered at least predominantly (S,S)-3-chloro-2,4-dihydroxybutanamide providing haloalcohol dehalogenase, an altered at least predominantly (S,S)-3-bromo-2,4-dihydroxybutanamide providing haloalcohol dehalogenase, an altered at least predominantly (S,S)-3-chloro-2,4-dihydroxybutanoic acid providing haloalcohol dehalogenase, an altered at least predominantly (S,S)-3-bromo-2,4-dihydroxybutanoic acid providing haloalcohol dehalogenase, an altered at least predominantly (S,S)-alkyl 3-chloro-2,4-dihydroxybutanoate providing haloalcohol dehalogenase, an altered at least predominantly (S,S)-alkyl 3-bromo-2,4-dihydroxybutanoate providing haloalcohol dehalogenase, an altered at least predominantly (S,S)-2-chloro-3,4-dihydroxybutanenitrile providing haloalcohol dehalogenase, an altered at least predominantly (S,S)-2-bromo-3,4-dihydroxybutanenitrile providing haloalcohol dehalogenase, an altered at least predominantly (S,S)-2-chloro-3,4-dihydroxybutanamide providing haloalcohol dehalogenase, an altered at least predominantly (S,S)-2-bromo-3,4-dihydroxybutanamide providing haloalcohol dehalogenase, an altered at least predominantly (S,S)-2-chloro-3,4-dihydroxybutanoic acid providing haloalcohol dehalogenase, an altered at least predominantly (S,S)-2-bromo-3,4-dihydroxybutanoic acid providing haloalcohol dehalogenase, an altered at least predominantly (S,S)-alkyl 2-chloro-3,4-dihydroxybutanoate providing haloalcohol dehalogenase, an altered at least predominantly (S,S)-alkyl 2-bromo-3,4-dihydroxybutanoate providing haloalcohol dehalogenase, an altered at least predominantly (R,R)-4-chloro-2,3-dihydroxybutanenitrile providing haloalcohol dehalogenase, an altered at least predominantly (R,R)-4-bromo-2,3-dihydroxybutanenitrile providing haloalcohol dehalogenase, an altered at least predominantly (R,R)-4-chloro-2,3-dihydroxybutanamide providing haloalcohol dehalogenase, an altered at least predominantly (R,R)-4-bromo-2,3-dihydroxybutanamide providing haloalcohol dehalogenase, an altered at least predominantly (R,R)-4-chloro-2,3-dihydroxybutanoic acid providing haloalcohol dehalogenase, an altered at least predominantly (R,R)-4-bromo-2,3-dihydroxybutanoic acid providing haloalcohol dehalogenase, an altered at least predominantly (R,R)-alkyl 4-chloro-2,3-dihydroxybutanoate providing haloalcohol dehalogenase, an altered at least predominantly (R,R)-alkyl 4-bromo-2,3-dihydroxybutanoate providing haloalcohol dehalogenase, an altered at least predominantly (R,R)-3-chloro-2,4-dihydroxybutanenitrile providing haloalcohol dehalogenase, an altered at least predominantly (R,R)-3-bromo-2,4-dihydroxybutanenitrile providing haloalcohol dehalogenase, an altered at least predominantly (R,R)-3-chloro-2,4-dihydroxybutanamide providing haloalcohol dehalogenase, an altered at least predominantly (R,R)-3-bromo-2,4-dihydroxybutanamide providing haloalcohol dehalogenase, an altered at least predominantly (R,R)-3-chloro-2,4-dihydroxybutanoic acid providing haloalcohol dehalogenase, an altered at least predominantly (R,R)-3-bromo-2,4-dihydroxybutanoic acid providing haloalcohol dehalogenase, an altered at least predominantly (R,R)-alkyl 3-chloro-2,4-dihydroxybutanoate providing haloalcohol dehalogenase, an altered at least predominantly (R,R)-alkyl 3-bromo-2,4-dihydroxybutanoate providing haloalcohol dehalogenase, an altered at least predominantly (R,R)-2-chloro-3,4-dihydroxybutanenitrile providing haloalcohol dehalogenase, an altered at least predominantly (R,R)-2-bromo-3,4-dihydroxybutanenitrile providing haloalcohol dehalogenase, an altered at least predominantly (R,R)-2-chloro-3,4-dihydroxybutanamide providing haloalcohol dehalogenase, an altered at least predominantly (R,R)-2-bromo-3,4-dihydroxybutanamide providing haloalcohol dehalogenase, an altered at least predominantly (R,R)-2-chloro-3,4-dihydroxybutanoic acid providing haloalcohol dehalogenase, an altered at least predominantly (R,R)-2-bromo-3,4-dihydroxybutanoic acid providing haloalcohol dehalogenase, an altered at least predominantly (R,R)-alkyl 2-chloro-3,4-dihydroxybutanoate providing haloalcohol dehalogenase, an altered at least predominantly (R,R)-alkyl 2-bromo-3,4-dihydroxybutanoate providing haloalcohol dehalogenase, an altered 4-chloro-2,3-dihydroxybutanenitrile providing haloalcohol dehalogenase, an altered 4-bromo-2,3-dihydroxybutanenitrile providing haloalcohol dehalogenase, an altered 4-chloro-2,3-dihydroxybutanamide providing haloalcohol dehalogenase, an altered 4-bromo-2,3-dihydroxybutanamide providing haloalcohol dehalogenase, an altered 4-chloro-2,3-dihydroxybutanoic acid providing haloalcohol dehalogenase, an altered 4-bromo-2,3-dihydroxybutanoic acid providing haloalcohol dehalogenase, an altered alkyl 4-chloro-2,3-dihydroxybutanoate providing haloalcohol dehalogenase, an altered alkyl 4-bromo-2,3-dihydroxybutanoate providing haloalcohol dehalogenase, an altered 3-chloro-2,4-dihydroxybutanenitrile providing haloalcohol dehalogenase, an altered 3-bromo-2,4-dihydroxybutanenitrile providing haloalcohol dehalogenase, an altered 3-chloro-2,4-dihydroxybutanamide providing haloalcohol dehalogenase, an altered 3-bromo-2,4-dihydroxybutanamide providing haloalcohol dehalogenase, an altered 3-chloro-2,4-dihydroxybutanoic acid providing haloalcohol dehalogenase, an altered 3-bromo-2,4-dihydroxybutanoic acid providing haloalcohol dehalogenase, an altered alkyl 3-chloro-2,4-dihydroxybutanoate providing haloalcohol dehalogenase, an altered alkyl 3-bromo-2,4-dihydroxybutanoate providing haloalcohol dehalogenase, an altered 2-chloro-3,4-dihydroxybutanenitrile providing haloalcohol dehalogenase, an altered 2-bromo-3,4-dihydroxybutanenitrile providing haloalcohol dehalogenase, an altered 2-chloro-3,4-dihydroxybutanamide providing haloalcohol dehalogenase, an altered 2-bromo-3,4-dihydroxybutanamide providing haloalcohol dehalogenase, an altered 2-chloro-3,4-dihydroxybutanoic acid providing haloalcohol dehalogenase, an altered 2-bromo-3,4-dihydroxybutanoic acid providing haloalcohol dehalogenase, an altered alkyl 2-chloro-3,4-dihydroxybutanoate providing haloalcohol dehalogenase, an altered alkyl 2-bromo-3,4-dihydroxybutanoate providing haloalcohol dehalogenase, an altered at least predominantly (S,R)-2-chloro-3,4-epoxybutanenitrile providing haloalcohol dehalogenase, an altered at least predominantly (S,R)-2-bromo-3,4-epoxybutanenitrile providing haloalcohol dehalogenase, an altered at least predominantly (S,R)-2-chloro-3,4-epoxybutanamide providing haloalcohol dehalogenase, an altered at least predominantly (S,R)-2-bromo-3,4-epoxybutanamide providing haloalcohol dehalogenase, an altered at least predominantly (S,R)-2-chloro-3,4-epoxybutanoic acid providing haloalcohol dehalogenase, an altered at least predominantly (S,R)-2-bromo-3,4-epoxybutanoic acid providing haloalcohol dehalogenase, an altered at least predominantly (S,R)-alkyl 2-chloro-3,4-epoxybutanoate providing haloalcohol dehalogenase, an altered at least predominantly (S,R)-alkyl 2-bromo-3,4-epoxybutanoate providing haloalcohol dehalogenase, an altered at least predominantly (S,R)-4-chloro-2,3-epoxybutanenitrile providing haloalcohol dehalogenase, an altered at least predominantly (S,R)-4-bromo-2,3-epoxybutanenitrile providing haloalcohol dehalogenase, an altered at least predominantly (S,R)-4-chloro-2,3-epoxybutanamide providing haloalcohol dehalogenase, an altered at least predominantly (S,R)-4-bromo-2,3-epoxybutanamide providing haloalcohol dehalogenase, an altered at least predominantly (S,R)-4-chloro-2,3-epoxybutanoic acid providing haloalcohol dehalogenase, an altered at least predominantly (S,R)-4-bromo-2,3-epoxybutanoic acid providing haloalcohol dehalogenase, an altered at least predominantly (S,R)-alkyl 4-chloro-2,3-epoxybutanoate providing haloalcohol dehalogenase, an altered at least predominantly (S,R)-alkyl 4-bromo-2,3-epoxybutanoate providing haloalcohol dehalogenase, an altered at least predominantly (R,S)-2-chloro-3,4-epoxybutanenitrile providing haloalcohol dehalogenase, an altered at least predominantly (R,S)-2-bromo-3,4-epoxybutanenitrile providing haloalcohol dehalogenase, an altered at least predominantly (R,S)-2-chloro-3,4-epoxybutanamide providing haloalcohol dehalogenase, an altered at least predominantly (R,S)-2-bromo-3,4-epoxybutanamide providing haloalcohol dehalogenase, an altered at least predominantly (R,S)-2-chloro-3,4-epoxybutanoic acid providing haloalcohol dehalogenase, an altered at least predominantly (R,S)-2-bromo-3,4-epoxybutanoic acid providing haloalcohol dehalogenase, an altered at least predominantly (R,S)-alkyl 2-chloro-3,4-epoxybutanoate providing haloalcohol dehalogenase, an altered at least predominantly (R,S)-alkyl 2-bromo-3,4-epoxybutanoate providing haloalcohol dehalogenase, an altered at least predominantly (R,S)-4-chloro-2,3-epoxybutanenitrile providing haloalcohol dehalogenase, an altered at least predominantly (R,S)-4-bromo-2,3-epoxybutanenitrile providing haloalcohol dehalogenase, an altered at least predominantly (R,S)-4-chloro-2,3-epoxybutanamide providing haloalcohol dehalogenase, an altered at least predominantly (R,S)-4-bromo-2,3-epoxybutanamide providing haloalcohol dehalogenase, an altered at least predominantly (R,S)-4-chloro-2,3-epoxybutanoic acid providing haloalcohol dehalogenase, an altered at least predominantly (R,S)-4-bromo-2,3-epoxybutanoic acid providing haloalcohol dehalogenase, an altered at least predominantly (R,S)-alkyl 4-chloro-2,3-epoxybutanoate providing haloalcohol dehalogenase, an altered at least predominantly (R,S)-alkyl 4-bromo-2,3-epoxybutanoate providing haloalcohol dehalogenase, an altered at least predominantly (S,S)-2-chloro-3,4-epoxybutanenitrile providing haloalcohol dehalogenase, an altered at least predominantly (S,S)-2-bromo-3,4-epoxybutanenitrile providing haloalcohol dehalogenase, an altered at least predominantly (S,S)-2-chloro-3,4-epoxybutanamide providing haloalcohol dehalogenase, an altered at least predominantly (S,S)-2-bromo-3,4-epoxybutanamide providing haloalcohol dehalogenase, an altered at least predominantly (S,S)-2-chloro-3,4-epoxybutanoic acid providing haloalcohol dehalogenase, an altered at least predominantly (S,S)-2-bromo-3,4-epoxybutanoic acid providing haloalcohol dehalogenase, an altered at least predominantly (S,S)-alkyl 2-chloro-3,4-epoxybutanoate providing haloalcohol dehalogenase, an altered at least predominantly (S,S)-alkyl 2-bromo-3,4-epoxybutanoate providing haloalcohol dehalogenase, an altered at least predominantly (S,S)-4-chloro-2,3-epoxybutanenitrile providing haloalcohol dehalogenase, an altered at least predominantly (S,S)-4-bromo-2,3-epoxybutanenitrile providing haloalcohol dehalogenase, an altered at least predominantly (S,S)-4-chloro-2,3-epoxybutanamide providing haloalcohol dehalogenase, an altered at least predominantly (S,S)-4-bromo-2,3-epoxybutanamide providing haloalcohol dehalogenase, an altered at least predominantly (S,S)-4-chloro-2,3-epoxybutanoic acid providing haloalcohol dehalogenase, an altered at least predominantly (S,S)-4-bromo-2,3-epoxybutanoic acid providing haloalcohol dehalogenase, an altered at least predominantly (S,S)-alkyl 4-chloro-2,3-epoxybutanoate providing haloalcohol dehalogenase, an altered at least predominantly (S,S)-alkyl 4-bromo-2,3-epoxybutanoate providing haloalcohol dehalogenase, an altered at least predominantly (R,R)-2-chloro-3,4-epoxybutanenitrile providing haloalcohol dehalogenase, an altered at least predominantly (R,R)-2-bromo-3,4-epoxybutanenitrile providing haloalcohol dehalogenase, an altered at least predominantly (R,R)-2-chloro-3,4-epoxybutanamide providing haloalcohol dehalogenase, an altered at least predominantly (R,R)-2-bromo-3,4-epoxybutanamide providing haloalcohol dehalogenase, an altered at least predominantly (R,R)-2-chloro-3,4-epoxybutanoic acid providing haloalcohol dehalogenase, an altered at least predominantly (R,R)-2-bromo-3,4-epoxybutanoic acid providing haloalcohol dehalogenase, an altered at least predominantly (R,R)-alkyl 2-chloro-3,4-epoxybutanoate providing haloalcohol dehalogenase, an altered at least predominantly providing haloalcohol dehalogenase, an altered at least predominantly (R,R)-4-chloro-2,3-epoxybutanenitrile providing haloalcohol dehalogenase, an altered at least predominantly (R,R)-4-bromo-2,3-epoxybutanenitrile providing haloalcohol dehalogenase, an altered at least predominantly (R,R)-4-chloro-2,3-epoxybutanamide providing haloalcohol dehalogenase, an altered at least predominantly (R,R)-4-bromo-2,3-epoxybutanamide providing haloalcohol dehalogenase, an altered at least predominantly (R,R)-4-chloro-2,3-epoxybutanoic acid providing haloalcohol dehalogenase, an altered at least predominantly (R,R)-4-bromo-2,3-epoxybutanoic acid providing haloalcohol dehalogenase, an altered at least predominantly (R,R)-alkyl 4-chloro-2,3-epoxybutanoate providing haloalcohol dehalogenase, an altered at least predominantly (R,R)-alkyl 4-bromo-2,3-epoxybutanoate providing haloalcohol dehalogenase, an altered 2-chloro-3,4-epoxybutanenitrile providing haloalcohol dehalogenase, an altered 2-bromo-3,4-epoxybutanenitrile providing haloalcohol dehalogenase, an altered 2-chloro-3,4-epoxybutanamide providing haloalcohol dehalogenase, an altered 2-bromo-3,4-epoxybutanamide providing haloalcohol dehalogenase, an altered 2-chloro-3,4-epoxybutanoic acid providing haloalcohol dehalogenase, an altered 2-bromo-3,4-epoxybutanoic acid providing haloalcohol dehalogenase, an altered alkyl 2-chloro-3,4-epoxybutanoate providing haloalcohol dehalogenase, an altered alkyl 2-bromo-3,4-epoxybutanoate providing haloalcohol dehalogenase, an altered 4-chloro-2,3-epoxybutanenitrile providing haloalcohol dehalogenase, an altered 4-bromo-2,3-epoxybutanenitrile providing haloalcohol dehalogenase, an altered 4-chloro-2,3-epoxybutanamide providing haloalcohol dehalogenase, an altered 4-bromo-2,3-epoxybutanamide providing haloalcohol dehalogenase, an altered 4-chloro-2,3-epoxybutanoic acid providing haloalcohol dehalogenase, an altered 4-bromo-2,3-epoxybutanoic acid providing haloalcohol dehalogenase, an altered alkyl 4-chloro-2,3-epoxybutanoate providing haloalcohol dehalogenase, an altered alkyl 4-bromo-2,3-epoxybutanoate providing haloalcohol dehalogenase, an altered at least predominantly (S,R)-4-hydroxy-2,3-epoxybutanenitrile providing haloalcohol dehalogenase, an altered at least predominantly (S,R)-4-hydroxy-2,3-epoxybutanamide providing haloalcohol dehalogenase, an altered at least predominantly (S,R)-4-hydroxy-2,3-epoxybutanoic acid providing haloalcohol dehalogenase, an altered at least predominantly (S,R)-alkyl 4-hydroxy-2,3-epoxybutanoate providing haloalcohol dehalogenase, an altered at least predominantly (S,R)-2-hydroxy-3,4-epoxybutanenitrile providing haloalcohol dehalogenase, an altered at least predominantly (S,R)-2-hydroxy-3,4-epoxybutanamide providing haloalcohol dehalogenase, an altered at least predominantly (S,R)-2-hydroxy-3,4-epoxybutanoic acid providing haloalcohol dehalogenase, an altered at least predominantly (S,R)-alkyl 2-hydroxy-3,4-epoxybutanoate providing haloalcohol dehalogenase, an altered at least predominantly (R,S)-4-hydroxy-2,3-epoxybutanenitrile providing haloalcohol dehalogenase, an altered at least predominantly (R,S)-4-hydroxy-2,3-epoxybutanamide providing haloalcohol dehalogenase, an altered at least predominantly (R,S)-4-hydroxy-2,3-epoxybutanoic acid providing haloalcohol dehalogenase, an altered at least predominantly (R,S)-alkyl 4-hydroxy-2,3-epoxybutanoate providing haloalcohol dehalogenase, an altered at least predominantly (R,S)-2-hydroxy-3,4-epoxybutanenitrile providing haloalcohol dehalogenase, an altered at least predominantly (R,S)-2-hydroxy-3,4-epoxybutanamide providing haloalcohol dehalogenase, an altered at least predominantly (R,S)-2-hydroxy-3,4-epoxybutanoic acid providing haloalcohol dehalogenase, an altered at least predominantly (R,S)-alkyl 2-hydroxy-3,4-epoxybutanoate providing haloalcohol dehalogenase, an altered at least predominantly (S,S)-4-hydroxy-2,3-epoxybutanenitrile providing haloalcohol dehalogenase, an altered at least predominantly (S,S)-4-hydroxy-2,3-epoxybutanamide providing haloalcohol dehalogenase, an altered at least predominantly (S,S)-4-hydroxy-2,3-epoxybutanoic acid providing haloalcohol dehalogenase, an altered at least predominantly (S,S)-alkyl 4-hydroxy-2,3-epoxybutanoate providing haloalcohol dehalogenase, an altered at least predominantly (S,S)-2-hydroxy-3,4-epoxybutanenitrile providing haloalcohol dehalogenase, an altered at least predominantly (S,S)-2-hydroxy-3,4-epoxybutanamide providing haloalcohol dehalogenase, an altered at least predominantly (S,S)-2-hydroxy-3,4-epoxybutanoic acid providing haloalcohol dehalogenase, an altered at least predominantly (S,S)-alkyl 2-hydroxy-3,4-epoxybutanoate providing haloalcohol dehalogenase, an altered at least predominantly (R,R)-4-hydroxy-2,3-epoxybutanenitrile providing haloalcohol dehalogenase, an altered at least predominantly (R,R)-4-hydroxy-2,3-epoxybutanamide providing haloalcohol dehalogenase, an altered at least predominantly (R,R)-4-hydroxy-2,3-epoxybutanoic acid providing haloalcohol dehalogenase, an altered at least predominantly (R,R)-alkyl 4-hydroxy-2,3-epoxybutanoate providing haloalcohol dehalogenase, an altered at least predominantly (R,R)-2-hydroxy-3,4-epoxybutanenitrile providing haloalcohol dehalogenase, an altered at least predominantly (R,R)-2-hydroxy-3,4-epoxybutanamide providing haloalcohol dehalogenase, an altered at least predominantly (R,R)-2-hydroxy-3,4-epoxybutanoic acid providing haloalcohol dehalogenase, an altered at least predominantly (R,R)-alkyl 2-hydroxy-3,4-epoxybutanoate providing haloalcohol dehalogenase, an altered 4-hydroxy-2,3-epoxybutanenitrile providing haloalcohol dehalogenase, an altered 4-hydroxy-2,3-epoxybutanamide providing haloalcohol dehalogenase, an altered 4-hydroxy-2,3-epoxybutanoic acid providing haloalcohol dehalogenase, an altered alkyl 4-hydroxy-2,3-epoxybutanoate providing haloalcohol dehalogenase, an altered 2-hydroxy-3,4-epoxybutanenitrile providing haloalcohol dehalogenase, an altered 2-hydroxy-3,4-epoxybutanamide providing haloalcohol dehalogenase, an altered 2-hydroxy-3,4-epoxybutanoic acid providing haloalcohol dehalogenase, an altered alkyl 2-hydroxy-3,4-epoxybutanoate providing haloalcohol dehalogenase, and the like.

An “altered halopolyol dehalogenase” can include, e.g., an altered 2,3-epoxy-1-propanol providing halopolyol dehalogenase, an altered 1,2,3,4-diepoxybutane providing halopolyol dehalogenase, an altered 1,2,4,5-diepoxypentane providing halopolyol dehalogenase, an altered 1,2,5,6-diepoxyhexane providing halopolyol dehalogenase, an altered 1,2,6,7-diepoxyheptane providing halopolyol dehalogenase, an altered 1,2,7,8-diepoxyoctane providing halopolyol dehalogenase, an altered (R)-2,3-epoxy—propanol providing halopolyol dehalogenase, an altered (S)-2,3-epoxy-1-propanol providing halopolyol dehalogenase, an altered (R,R)-1,2,3,4-diepoxybutane providing halopolyol dehalogenase, an altered (S,S)-1,2,3,4-diepoxybutane providing halopolyol dehalogenase, an altered (R,S)-1,2,3,4-diepoxybutane providing halopolyol dehalogenase, an altered (R,R)-1,2,4,5-diepoxypentane providing halopolyol dehalogenase, an altered (S,S)-1,2,4,5-diepoxypentane providing halopolyol dehalogenase, an altered (R,S)-1,2,4,5-diepoxypentane providing halopolyol dehalogenase, an altered (R,R)-1,2,5,6-diepoxyhexane providing halopolyol dehalogenase, an altered (S,S)-1,2,5,6-diepoxyhexane providing halopolyol dehalogenase, an altered (R,S)-1,2,5,6-diepoxyhexane providing halopolyol dehalogenase, an altered (R,R)-1,2,6,7-diepoxyheptane providing halopolyol dehalogenase, an altered (R,S)-1,2,6,7-diepoxyheptane providing halopolyol dehalogenase, an altered (R,S)-1,2,6,7-diepoxyheptane providing halopolyol dehalogenase, an altered (R,R)-1,2,7,8-diepoxyoctane providing halopolyol dehalogenase, an altered (S,S)-1,2,7,8-diepoxyoctane providing halopolyol dehalogenase, an altered (R,S)-1,2,7,8-diepoxyoctane providing halopolyol dehalogenase, an altered at least predominantly (S,R)-4-hydroxy-2,3-epoxybutanenitrile providing halopolyol dehalogenase, an altered at least predominantly (S,R)-4-hydroxy-2,3-epoxybutanamide providing halopolyol dehalogenase, an altered at least predominantly (S,R)-4-hydroxy-2,3-epoxybutanoic acid providing halopolyol dehalogenase, an altered at least predominantly (S,R)-alkyl 4-hydroxy-2,3-epoxybutanoate providing halopolyol dehalogenase, an altered at least predominantly (S,R)-2-hydroxy-3,4-epoxybutanenitrile providing halopolyol dehalogenase, an altered at least predominantly (S,R)-2-hydroxy-3,4-epoxybutanamide providing halopolyol dehalogenase, an altered at least predominantly (S,R)-2-hydroxy-3,4-epoxybutanoic acid providing halopolyol dehalogenase, an altered at least predominantly (S,R)-alkyl 2-hydroxy-3,4-epoxybutanoate providing halopolyol dehalogenase, an altered at least predominantly (R,S)-4-hydroxy-2,3-epoxybutanenitrile providing halopolyol dehalogenase, an altered at least predominantly (R,S)-4-hydroxy-2,3-epoxybutanamide providing halopolyol dehalogenase, an altered at least predominantly (R,S)-4-hydroxy-2,3-epoxybutanoic acid providing halopolyol dehalogenase, an altered at least predominantly (R,S)-alkyl 4-hydroxy-2,3-epoxybutanoate providing halopolyol dehalogenase, an altered at least predominantly (R,S)-2-hydroxy-3,4-epoxybutanenitrile providing halopolyol dehalogenase, an altered at least predominantly (R,S)-2-hydroxy-3,4-epoxybutanamide providing halopolyol dehalogenase, an altered at least predominantly (R,S)-2-hydroxy-3,4-epoxybutanoic acid providing halopolyol dehalogenase, an altered at least predominantly (R,S)-alkyl 2-hydroxy-3,4-epoxybutanoate providing halopolyol dehalogenase, an altered 4-hydroxy-2,3-epoxybutanenitrile providing halopolyol dehalogenase, an altered 4-hydroxy-2,3-epoxybutanamide providing halopolyol dehalogenase, an altered 4-hydroxy-2,3-epoxybutanoic acid providing halopolyol dehalogenase, an altered alkyl 4-hydroxy-2,3-epoxybutanoate providing halopolyol dehalogenase, an altered 2-hydroxy-3,4-epoxybutanenitrile providing halopolyol dehalogenase, an altered 2-hydroxy-3,4-epoxybutanamide providing halopolyol dehalogenase, an altered 2-hydroxy-3,4-epoxybutanoic acid providing halopolyol dehalogenase, an altered alkyl 2-hydroxy-3,4-epoxybutanoate providing halopolyol dehalogenase, an altered at least predominantly (R,R)-4-hydroxy-2,3-epoxybutanenitrile providing halopolyol dehalogenase, an altered at least predominantly (R,R)-4-hydroxy-2,3-epoxybutanamide providing halopolyol dehalogenase, an altered at least predominantly (R,R)-4-hydroxy-2,3-epoxybutanoic acid providing halopolyol dehalogenase, an altered at least predominantly (R,R)-alkyl 4-hydroxy-2,3-epoxybutanoate providing halopolyol dehalogenase, an altered at least predominantly (R,R)-2-hydroxy-3,4-epoxybutanenitrile providing halopolyol dehalogenase, an altered at least predominantly (R,R)-2-hydroxy-3,4-epoxybutanamide providing halopolyol dehalogenase, an altered at least predominantly (R,R)-2-hydroxy-3,4-epoxybutanoic acid providing halopolyol dehalogenase, an altered at least predominantly (R,R)-alkyl 2-hydroxy-3,4-epoxybutanoate providing halopolyol dehalogenase, an altered at least predominantly (S,S)-4-hydroxy-2,3-epoxybutanenitrile providing halopolyol dehalogenase, an altered at least predominantly (S,S)-4-hydroxy-2,3-epoxybutanamide providing halopolyol dehalogenase, an altered at least predominantly (S,S)-4-hydroxy-2,3-epoxybutanoic acid providing halopolyol dehalogenase, an altered at least predominantly (S,S)-alkyl 4-hydroxy-2,3-epoxybutanoate providing halopolyol dehalogenase, an altered at least predominantly (S,S)-2-hydroxy-3,4-epoxybutanenitrile providing halopolyol dehalogenase, an altered at least predominantly (S,S)-2-hydroxy-3,4-epoxybutanamide providing halopolyol dehalogenase, an altered at least predominantly (S,S)-2-hydroxy-3,4-epoxybutanoic acid providing halopolyol dehalogenase, an altered at least predominantly (S,S)-alkyl 2-hydroxy-3,4-epoxybutanoate providing halopolyol dehalogenase, and the like.

An “altered halohydrocarbon-haloalcohol dehalogenase” can include, e.g., an altered 1-halo-2,3-epoxypropane providing halohydrocarbon-haloalcohol dehalogenase, an altered 2-halo-1,3-propanediol providing halohydrocarbon-haloalcohol dehalogenase, an altered 1,2-dihalo-3,4-epoxybutane providing halohydrocarbon-haloalcohol dehalogenase, an altered 2,3-dihalo-1,4-butanediol providing halohydrocarbon-haloalcohol dehalogenase, an altered 1,2-dihalo-4,5-epoxypentane providing halohydrocarbon-haloalcohol dehalogenase, an altered 2,4-dihalo-1,5-pentanediol providing halohydrocarbon-haloalcohol dehalogenase, an altered 1,2-dihalo-5,6-epoxyhexane providing halohydrocarbon-haloalcohol dehalogenase, an altered 2,5-dihalo-1,6-hexanediol providing halohydrocarbon-haloalcohol dehalogenase, an altered 1,2-dihalo-6,7-epoxyheptane providing halohydrocarbon-haloalcohol dehalogenase, an altered 2,6-dihalo-1,7-heptanediol providing halohydrocarbon-haloalcohol dehalogenase, an altered 1,2-dihalo-7,8-epoxyoctane providing halohydrocarbon-haloalcohol dehalogenase, an altered 2,7-dihalo-1,8-octanediol providing halohydrocarbon-haloalcohol dehalogenase, an altered 2-halo-3,4-dihydroxybutanenitrile providing halohydrocarbon-haloalcohol dehalogenase, an altered 2-halo-3,4-dihydroxybutanamide providing halohydrocarbon-haloalcohol dehalogenase, an altered 2-halo-3,4-dihydroxybutanoic acid providing halohydrocarbon-haloalcohol dehalogenase, an altered alkyl 2-halo-3,4-dihydroxybutanoate providing halohydrocarbon-haloalcohol dehalogenase, an altered 3-halo-2,4-dihydroxybutanenitrile providing halohydrocarbon-haloalcohol dehalogenase, an altered 3-halo-2,4-dihydroxybutanamide providing halohydrocarbon-haloalcohol dehalogenase, an altered 3-halo-2,4-dihydroxybutanoic acid providing halohydrocarbon-haloalcohol dehalogenase, an altered alkyl 3-halo-2,4-dihydroxybutanoate providing halohydrocarbon-haloalcohol dehalogenase, an altered 4-halo-2,3-dihydroxybutanenitrile providing halohydrocarbon-haloalcohol dehalogenase, an altered 4-halo-2,3-dihydroxybutanamide providing halohydrocarbon-haloalcohol dehalogenase, an altered 4-halo-2,3-dihydroxybutanoic acid providing halohydrocarbon-haloalcohol dehalogenase, an altered alkyl 4-halo-2,3-dihydroxybutanoate providing halohydrocarbon-haloalcohol dehalogenase, an altered 4-halo-2,3-epoxybutanenitrile providing halohydrocarbon-haloalcohol dehalogenase, an altered 4-halo-2,3-epoxybutanamide providing halohydrocarbon-haloalcohol dehalogenase, an altered 4-halo-2,3-epoxybutanoic acid providing halohydrocarbon-haloalcohol dehalogenase, an altered alkyl 4-halo-2,3-epoxybutanoate providing halohydrocarbon-haloalcohol dehalogenase, an altered 2-halo-3,4-epoxybutanenitrile providing halohydrocarbon-haloalcohol dehalogenase, an altered 2-halo-3,4-epoxybutanamide providing halohydrocarbon-haloalcohol dehalogenase, an altered 2-halo-3,4-epoxybutanoic acid providing halohydrocarbon-haloalcohol dehalogenase, an altered alkyl 2-halo-3,4-epoxybutanoate providing halohydrocarbon-haloalcohol dehalogenase, an altered 1-chloro-2,3-epoxypropane providing halohydrocarbon-haloalcohol dehalogenase, an altered 1,2-epoxypropane providing halohydrocarbon-haloalcohol dehalogenase, an altered ethylene oxide providing halohydrocarbon-haloalcohol dehalogenase, an altered 2-chloro-1,3-propanediol providing halohydrocarbon-haloalcohol dehalogenase, an altered 1,2-ethanediol providing halohydrocarbon-haloalcohol dehalogenase, an altered 1,3-propanediol providing halohydrocarbon-haloalcohol dehalogenase, an altered 1,2-dichloro-3,4-epoxybutane providing halohydrocarbon-haloalcohol dehalogenase, an altered 2,3-dichloro-1,4-butanediol providing halohydrocarbon-haloalcohol dehalogenase, an altered 1,2-dichloro-4,5-epoxypentane providing halohydrocarbon-haloalcohol dehalogenase, an altered 2,4-dichloro-1,5-pentanediol providing halohydrocarbon-haloalcohol dehalogenase, an altered 1,2-dichloro-5,6-epoxyhexane providing halohydrocarbon-haloalcohol dehalogenase, an altered 2,5-dichloro-1,6-hexanediol providing halohydrocarbon-haloalcohol dehalogenase, an altered 1,2-dichloro-6,7-epoxyheptane providing halohydrocarbon-haloalcohol dehalogenase, an altered 2,6-dichloro-1,7-heptanediol providing halohydrocarbon-haloalcohol dehalogenase, an altered 1,2-dichloro-7,8-epoxyoctane providing halohydrocarbon-haloalcohol dehalogenase, an altered 2,7-dichloro-1,8-octanediol providing halohydrocarbon-haloalcohol dehalogenase, an altered at least predominantly (R)-1-chloro-2,3-epoxypropane providing halohydrocarbon-haloalcohol dehalogenase, an altered at least predominantly (R)-1,2-epoxypropane providing halohydrocarbon-haloalcohol dehalogenase, an altered at least predominantly (R,R)-1,2-dichloro-3,4-epoxybutane providing halohydrocarbon-haloalcohol dehalogenase, an altered at least predominantly (S,S)-1,2-dichloro-3,4-epoxybutane providing halohydrocarbon-haloalcohol dehalogenase, an altered at least predominantly (R,S)-1,2-dichloro-3,4-epoxybutane providing halohydrocarbon-haloalcohol dehalogenase, an altered at least predominantly (S,R)-1,2-dichloro-3,4-epoxybutane providing halohydrocarbon-haloalcohol dehalogenase, an altered at least predominantly (R,R)-1,2-dichloro-4,5-epoxypentane providing halohydrocarbon-haloalcohol dehalogenase, an altered at least predominantly (S,S)-1,2-dichloro-4,5-epoxypentane providing halohydrocarbon-haloalcohol dehalogenase, an altered at least predominantly (R,S)-1,2-dichloro-4,5-epoxypentane providing halohydrocarbon-haloalcohol dehalogenase, an altered at least predominantly (S,R)-1,2-dichloro-4,5-epoxypentane providing halohydrocarbon-haloalcohol dehalogenase, an altered at least predominantly (R,R)-1,2-dichloro-5,6-epoxyhexane providing halohydrocarbon-haloalcohol dehalogenase, an altered at least predominantly (S,S)-1,2-dichloro-5,6-epoxyhexane providing halohydrocarbon-haloalcohol dehalogenase, an altered at least predominantly (R,S)-1,2-dichloro-5,6-epoxyhexane providing halohydrocarbon-haloalcohol dehalogenase, an altered at least predominantly (S,R)-1,2-dichloro-5,6-epoxyhexane providing halohydrocarbon-haloalcohol dehalogenase, an altered at least predominantly (R,R)-1,2-dichloro-6,7-epoxyheptane providing halohydrocarbon-haloalcohol dehalogenase, an altered at least predominantly (S,S)-1,2-dichloro-6,7-epoxyheptane providing halohydrocarbon-haloalcohol dehalogenase, an altered at least predominantly (R,S)-1,2-dichloro-6,7-epoxyheptane providing halohydrocarbon-haloalcohol dehalogenase, an altered at least predominantly (S,R)-1,2-dichloro-6,7-epoxyheptane providing halohydrocarbon-haloalcohol dehalogenase, an altered at least predominantly (R,R)-1,2-dichloro-7,8-epoxyoctane providing halohydrocarbon-haloalcohol dehalogenase, an altered at least predominantly (S,S)-1,2-dichloro-7,8-epoxyoctane providing halohydrocarbon-haloalcohol dehalogenase, an altered at least predominantly (R,S)-1,2-dichloro-7,8-epoxyoctane providing halohydrocarbon-haloalcohol dehalogenase, an altered at least predominantly (S,R)-1,2-dichloro-7,8-epoxyoctane providing halohydrocarbon-haloalcohol dehalogenase, an altered at least predominantly (S)-1-chloro-2,3-epoxypropane providing halohydrocarbon-haloalcohol dehalogenase, an altered at least predominantly (S)-1,2-epoxypropane providing halohydrocarbon-haloalcohol dehalogenase, an altered at least predominantly (R,R)-2,3-dichloro-1,4-butanediol providing halohydrocarbon-haloalcohol dehalogenase, an altered at least predominantly (S,S)-2,3-dichloro-1,4-butanediol providing halohydrocarbon-haloalcohol dehalogenase, an altered at least predominantly (R,S)-2,3-dichloro-1,4-butanediol providing halohydrocarbon-haloalcohol dehalogenase, an altered at least predominantly (R,R)-2,4-dichloro-1,5-pentanediol providing halohydrocarbon-haloalcohol dehalogenase, an altered at least predominantly (S,S)-2,4-dichloro-1,5-pentanediol providing halohydrocarbon-haloalcohol dehalogenase, an altered at least predominantly (R,S)-2,4-dichloro-1,5-pentanediol providing halohydrocarbon-haloalcohol dehalogenase, an altered at least predominantly (R,R)-2,5-dichloro-1,6-hexanediol providing halohydrocarbon-haloalcohol dehalogenase, an altered at least predominantly (S,S)-2,5-dichloro-1,6-hexanediol providing halohydrocarbon-haloalcohol dehalogenase, an altered at least predominantly (R,S)-2,5-dichloro-1,6-hexanediol providing halohydrocarbon-haloalcohol dehalogenase, an altered at least predominantly (R,R)-2,6-dichloro-1,7-heptanediol providing halohydrocarbon-haloalcohol dehalogenase, an altered at least predominantly (S,S)-2,6-dichloro-1,7-heptanediol providing halohydrocarbon-haloalcohol dehalogenase, an altered at least predominantly (R,S)-2,6-dichloro-1,7-heptanediol providing halohydrocarbon-haloalcohol dehalogenase, an altered at least predominantly (R,R)-2,7-dichloro-1,8-octanediol providing halohydrocarbon-haloalcohol dehalogenase, an altered at least predominantly (S,S)-2,7-dichloro-1,8-octanediol providing halohydrocarbon-haloalcohol dehalogenase, an altered at least predominantly (R,S)-2,7-dichloro-1,8-octanediol providing halohydrocarbon-haloalcohol dehalogenase, an altered 2,3-dihydoxypropanenitrile providing halohydrocarbon-haloalcohol dehalogenase, an altered 3,4-dihydoxybutanenitrile providing halohydrocarbon-haloalcohol dehalogenase, an altered 2,3-dihydoxypropanamide providing halohydrocarbon-haloalcohol dehalogenase, an altered 3,4-dihydoxybutanamide providing halohydrocarbon-haloalcohol dehalogenase, an altered 2,3-dihydoxypropanoic acid providing halohydrocarbon-haloalcohol dehalogenase, an altered 3,4-dihydoxybutanoic acid providing halohydrocarbon-haloalcohol dehalogenase, an altered at least predominantly (R)-2,3-dihydoxypropanenitrile providing halohydrocarbon-haloalcohol dehalogenase, an altered at least predominantly (S)-2,3-dihydoxypropanenitrile providing halohydrocarbon-haloalcohol dehalogenase, an altered at least predominantly (R)-3,4-dihydoxybutanenitrile providing halohydrocarbon-haloalcohol dehalogenase, an altered at least predominantly (S)-3,4-dihydoxybutanenitrile providing halohydrocarbon-haloalcohol dehalogenase, an altered at least predominantly (R)-2,3-dihydoxypropanamide providing halohydrocarbon-haloalcohol dehalogenase, an altered at least predominantly (S)-2,3-dihydoxypropanamide providing halohydrocarbon-haloalcohol dehalogenase, an altered at least predominantly (R)-3,4-dihydoxybutanamide providing halohydrocarbon- haloalcohol dehalogenase, an altered at least predominantly (S)-3,4-dihydoxybutanamide providing halohydrocarbon-haloalcohol dehalogenase, an altered at least predominantly (R)-2,3-dihydoxypropanoic acid providing halohydrocarbon-haloalcohol dehalogenase, an altered at least predominantly (S)-2,3-dihydoxypropanoic acid providing halohydrocarbon-haloalcohol dehalogenase, an altered at least predominantly (R)-3,4-dihydoxybutanoic acid providing halohydrocarbon-haloalcohol dehalogenase, an altered at least predominantly (S)-3,4-dihydoxybutanoic acid providing halohydrocarbon-haloalcohol dehalogenase, an altered 2,3-epoxy-1-propanenitrile providing halohydrocarbon-haloalcohol dehalogenase, an altered 3,4-epoxy-1-butanenitrile providing halohydrocarbon-haloalcohol dehalogenase, an altered 2,3-epoxy-1-propanamide providing halohydrocarbon-haloalcohol dehalogenase, an altered 3,4-epoxy-1-butanamide providing halohydrocarbon-haloalcohol dehalogenase, an altered 2,3-epoxy-1-propanoic acid providing halohydrocarbon-haloalcohol dehalogenase, an altered 3,4-epoxy-1-butanoic acid providing halohydrocarbon-haloalcohol dehalogenase, an altered at least predominantly (R)-2,3-epoxy-1-propanenitrile providing halohydrocarbon-haloalcohol dehalogenase, an altered at least predominantly (S)-2,3-epoxy-1-propanenitrile providing halohydrocarbon-haloalcohol dehalogenase, an altered at least predominantly (R)-3,4-epoxy-1-butanenitrile providing halohydrocarbon-haloalcohol dehalogenase, an altered at least predominantly (S)-3,4-epoxy-1-butanenitrile providing halohydrocarbon-haloalcohol dehalogenase, an altered at least predominantly (R)-2,3-epoxy-1-propanamide providing halohydrocarbon-haloalcohol dehalogenase, an altered at least predominantly (S)-2,3-epoxy-1-propanamide providing halohydrocarbon-haloalcohol dehalogenase, an altered at least predominantly (R)-3,4-epoxy-1-butanamide providing halohydrocarbon-haloalcohol dehalogenase, an altered at least predominantly (S)-3,4-epoxy-1-butanamide providing halohydrocarbon-haloalcohol dehalogenase, an altered at least predominantly (R)-2,3-epoxy-1-propanoic acid providing halohydrocarbon-haloalcohol dehalogenase, an altered at least predominantly (S)-2,3-epoxy-1-propanoic acid providing halohydrocarbon-haloalcohol dehalogenase, an altered at least predominantly (R)-3,4-epoxy-1-butanoic acid providing halohydrocarbon-haloalcohol dehalogenase, an altered at least predominantly (S)-3,4-epoxy-1-butanoic acid providing halohydrocarbon-haloalcohol dehalogenase, an altered alkyl 2,3-dihydroxypropanoate providing halohydrocarbon-haloalcohol dehalogenase, an altered alkyl 3,4-dihydroxybutanoate providing halohydrocarbon-haloalcohol dehalogenase, an altered alkyl 2,3-epoxy-1-propanoate providing halohydrocarbon-haloalcohol dehalogenase, an altered alkyl 3,4-epoxy-1-butanoate providing halohydrocarbon-haloalcohol dehalogenase, an altered at least predominantly (R)-alkyl 2,3-dihydroxypropanoate providing halohydrocarbon-haloalcohol dehalogenase, an altered at least predominantly (R)-alkyl 3,4-dihydroxybutanoate providing halohydrocarbon-haloalcohol dehalogenase, an altered at least predominantly (S)-alkyl 2,3-dihydroxypropanoate providing halohydrocarbon-haloalcohol dehalogenase, an altered at least predominantly (S)-alkyl 3,4-dihydroxybutanoate providing halohydrocarbon-haloalcohol dehalogenase, an altered at least predominantly (R)-alkyl 2,3-epoxy-1-propanoate providing halohydrocarbon-haloalcohol dehalogenase, an altered at least predominantly (R)-alkyl 3,4-epoxy-1-butanoate providing halohydrocarbon-haloalcohol dehalogenase, an altered at least predominantly (S)-alkyl 2,3-epoxy-1-propanoate providing halohydrocarbon-haloalcohol dehalogenase, an altered at least predominantly (S)-alkyl 3,4-epoxy-1-butanoate providing halohydrocarbon-haloalcohol dehalogenase, an altered 2,3-dihydroxybutanenitrile providing halohydrocarbon-haloalcohol dehalogenase, an altered 2,3-dihydroxybutanamide providing halohydrocarbon-haloalcohol dehalogenase, an altered 2,3-dihydroxybutanoic acid providing halohydrocarbon-haloalcohol dehalogenase, an altered alkyl 2,3-dihydroxybutanoate providing halohydrocarbon-haloalcohol dehalogenase, an altered 2,3-epoxy-1-butanenitrile providing halohydrocarbon-haloalcohol dehalogenase, an altered 2,3-epoxy-1-butanamide providing halohydrocarbon-haloalcohol dehalogenase, an altered 2,3-epoxy-1-butanoic acid providing halohydrocarbon-haloalcohol dehalogenase, an altered alkyl 2,3-epoxy-1-butanoate providing halohydrocarbon-haloalcohol dehalogenase, an altered at least predominantly (R,R)-2,3-dihydroxybutanenitrile providing halohydrocarbon-haloalcohol dehalogenase, an altered at least predominantly (R,R)-2,3-dihydroxybutanamide providing halohydrocarbon-haloalcohol dehalogenase, an altered at least predominantly (R,R)-2,3-dihydroxybutanoic acid providing halohydrocarbon-haloalcohol dehalogenase, an altered at least predominantly (R,R)-alkyl 2,3-dihydroxybutanoate providing halohydrocarbon-haloalcohol dehalogenase, an altered at least predominantly (S,S)-2,3-dihydroxybutanenitrile providing halohydrocarbon-haloalcohol dehalogenase, an altered at least predominantly (S,S)-2,3-dihydroxybutanamide providing halohydrocarbon-haloalcohol dehalogenase, an altered at least predominantly (S,S)-2,3-dihydroxybutanoic acid providing halohydrocarbon-haloalcohol dehalogenase, an altered at least predominantly (S,S)-alkyl 2,3-dihydroxybutanoate providing halohydrocarbon-haloalcohol dehalogenase, an altered at least predominantly (R,S)-2,3-dihydroxybutanenitrile providing halohydrocarbon-haloalcohol dehalogenase, an altered at least predominantly (R,S)-2,3-dihydroxybutanamide providing halohydrocarbon-haloalcohol dehalogenase, an altered at least predominantly (R,S)-2,3-dihydroxybutanoic acid providing halohydrocarbon-haloalcohol dehalogenase, an altered at least predominantly (R,S)-alkyl 2,3-dihydroxybutanoate providing halohydrocarbon-haloalcohol dehalogenase, an altered at least predominantly (S,R)-2,3-dihydroxybutanenitrile providing halohydrocarbon-haloalcohol dehalogenase, an altered at least predominantly (S,R)-2,3-dihydroxybutanamide providing halohydrocarbon-haloalcohol dehalogenase, an altered at least predominantly (S,R)-2,3-dihydroxybutanoic acid providing halohydrocarbon-haloalcohol dehalogenase, an altered at least predominantly (S,R)-alkyl 2,3-dihydroxybutanoate providing halohydrocarbon-haloalcohol dehalogenase, an altered at least predominantly (R,R)-2,3-epoxy-1-butanenitrile providing halohydrocarbon-haloalcohol dehalogenase, an altered at least predominantly (R,R)-2,3-epoxy-1-butanamide providing halohydrocarbon-haloalcohol dehalogenase, an altered at least predominantly (R,R)-2,3-epoxy-1-butanoic acid providing halohydrocarbon-haloalcohol dehalogenase, an altered at least predominantly (R,R)-alkyl 2,3-epoxy-1-butanoate providing halohydrocarbon-haloalcohol dehalogenase, an altered at least predominantly (S,S)-2,3-epoxy-1-butanenitrile providing halohydrocarbon-haloalcohol dehalogenase, an altered at least predominantly (S,S)-2,3-epoxy-1-butanamide providing halohydrocarbon-haloalcohol dehalogenase, an altered at least predominantly (S,S)-2,3-epoxy-1-butanoic acid providing halohydrocarbon-haloalcohol dehalogenase, an altered at least predominantly (S,S)-alkyl 2,3-epoxy-1-butanoate providing halohydrocarbon-haloalcohol dehalogenase, an altered at least predominantly (R,S)-2,3-epoxy-1-butanenitrile providing halohydrocarbon-haloalcohol dehalogenase, an altered at least predominantly (R,S)-2,3-epoxy-1-butanamide providing halohydrocarbon-haloalcohol dehalogenase, an altered at least predominantly (R,S)-2,3-epoxy-1-butanoic acid providing halohydrocarbon-haloalcohol dehalogenase, an altered at least predominantly (R,S)-alkyl 2,3-epoxy-1-butanoate providing halohydrocarbon-haloalcohol dehalogenase, an altered at least predominantly (S,R)-2,3-epoxy-1-butanenitrile providing halohydrocarbon-haloalcohol dehalogenase, an altered at least predominantly (S,R)-2,3-epoxy-1-butanamide providing halohydrocarbon-haloalcohol dehalogenase, an altered at least predominantly (S,R)-2,3-epoxy-1-butanoic acid providing halohydrocarbon-haloalcohol dehalogenase, an altered at least predominantly (S,R)-alkyl 2,3-epoxy-1-butanoate providing halohydrocarbon-haloalcohol dehalogenase, an altered at least predominantly (R,R)-2,3-epoxy-1-butanenitrile providing halohydrocarbon-haloalcohol dehalogenase, an altered at least predominantly (R,R)-2,3-epoxy-1-butanamide providing halohydrocarbon-haloalcohol dehalogenase, an altered at least predominantly (R,R)-2,3-epoxy-1-butanoic acid providing halohydrocarbon-haloalcohol dehalogenase, an altered at least predominantly (R,R)-alkyl 2,3-epoxy-1-butanoate providing halohydrocarbon-haloalcohol dehalogenase, an altered at least predominantly (S,S)-2,3-epoxy-1-butanenitrile providing halohydrocarbon-haloalcohol dehalogenase, an altered at least predominantly (S,S)-2,3-epoxy-1-butanamide providing halohydrocarbon-haloalcohol dehalogenase, an altered at least predominantly (S,S)-2,3-epoxy-1-butanoic acid providing halohydrocarbon-haloalcohol dehalogenase, an altered at least predominantly (S,S)-alkyl 2,3-epoxy-1-butanoate providing halohydrocarbon-haloalcohol dehalogenase, an altered at least predominantly (R,S)-2,3-epoxy-1-butanenitrile providing halohydrocarbon-haloalcohol dehalogenase, an altered at least predominantly (R,S)-2,3-epoxy-1-butanamide providing halohydrocarbon-haloalcohol dehalogenase, an altered at least predominantly (R,S)-2,3-epoxy-1-butanoic acid providing halohydrocarbon-haloalcohol dehalogenase, an altered at least predominantly (R,S)-alkyl 2,3-epoxy-1-butanoate providing halohydrocarbon-haloalcohol dehalogenase, an altered at least predominantly (S,R)-2,3-epoxy-1-butanenitrile providing halohydrocarbon-haloalcohol dehalogenase, an altered at least predominantly (S,R)-2,3-epoxy-1-butanamide providing halohydrocarbon-haloalcohol dehalogenase, an altered at least predominantly (S,R)-2,3-epoxy-1-butanoic acid providing halohydrocarbon-haloalcohol dehalogenase, an altered at least predominantly (S,R)-alkyl 2,3-epoxy-1-butanoate providing halohydrocarbon-haloalcohol dehalogenase, an altered 2-chloro-3,4-dihydroxybutanenitrile providing halohydrocarbon-haloalcohol dehalogenase, an altered 2-bromo-3,4-dihydroxybutanenitrile providing halohydrocarbon-haloalcohol dehalogenase, an altered 2-chloro-3,4-dihydroxybutanamide providing halohydrocarbon-haloalcohol dehalogenase, an altered 2-bromo-3,4-dihydroxybutanamide providing halohydrocarbon-haloalcohol dehalogenase, an altered 2-chloro-3,4-dihydroxybutanoic acid providing halohydrocarbon-haloalcohol dehalogenase, an altered 2-bromo-3,4-dihydroxybutanoic acid providing halohydrocarbon-haloalcohol dehalogenase, an altered alkyl 2-chloro-3,4-dihydroxybutanoate providing halohydrocarbon-haloalcohol dehalogenase, an altered alkyl 2-bromo-3,4-dihydroxybutanoate providing halohydrocarbon-haloalcohol dehalogenase, an altered 3-chloro-2,4-dihydroxybutanenitrile providing halohydrocarbon-haloalcohol dehalogenase, an altered 3-bromo-2,4-dihydroxybutanenitrile providing halohydrocarbon-haloalcohol dehalogenase, an altered 3-chloro-2,4-dihydroxybutanamide providing halohydrocarbon-haloalcohol dehalogenase, an altered 3-bromo-2,4-dihydroxybutanamide providing halohydrocarbon-haloalcohol dehalogenase, an altered 3-chloro-2,4-dihydroxybutanoic acid providing halohydrocarbon-haloalcohol dehalogenase, an altered 3-bromo-2,4-dihydroxybutanoic acid providing halohydrocarbon-haloalcohol dehalogenase, an altered alkyl 3-chloro-2,4-dihydroxybutanoate providing halohydrocarbon-haloalcohol dehalogenase, an altered alkyl 3-bromo-2,4-dihydroxybutanoate providing halohydrocarbon-haloalcohol dehalogenase, an altered 4-chloro-2,3-dihydroxybutanenitrile providing halohydrocarbon-haloalcohol dehalogenase, an altered 4-bromo-2,3-dihydroxybutanenitrile providing halohydrocarbon-haloalcohol dehalogenase, an altered 4-chloro-2,3-dihydroxybutanamide providing halohydrocarbon-haloalcohol dehalogenase, an altered 4-bromo-2,3-dihydroxybutanamide providing halohydrocarbon-haloalcohol dehalogenase, an altered 4-chloro-2,3-dihydroxybutanoic acid providing halohydrocarbon-haloalcohol dehalogenase, an altered 4-bromo-2,3-dihydroxybutanoic acid providing halohydrocarbon-haloalcohol dehalogenase, an altered alkyl 4-chloro-2,3-dihydroxybutanoate providing halohydrocarbon-haloalcohol dehalogenase, an altered alkyl 4-bromo-2,3-dihydroxybutanoate providing halohydrocarbon-haloalcohol dehalogenase, an altered at least predominantly (R,R)-2-chloro-3,4-dihydroxybutanenitrile providing halohydrocarbon-haloalcohol dehalogenase, an altered at least predominantly (R,R)-2-bromo-3,4-dihydroxybutanenitrile providing halohydrocarbon-haloalcohol dehalogenase, an altered at least predominantly (R,R)-2-chloro-3,4-dihydroxybutanamide providing halohydrocarbon-haloalcohol dehalogenase, an altered at least predominantly (R,R)-2-bromo-3,4-dihydroxybutanamide providing halohydrocarbon-haloalcohol dehalogenase, an altered at least predominantly (R,R)-2-chloro-3,4-dihydroxybutanoic acid providing halohydrocarbon-haloalcohol dehalogenase, an altered at least predominantly (R,R)-2-bromo-3,4-dihydroxybutanoic acid providing halohydrocarbon-haloalcohol dehalogenase, an altered at least predominantly (R,R)-alkyl 2-chloro-3,4-dihydroxybutanoate providing halohydrocarbon-haloalcohol dehalogenase, an altered at least predominantly (R,R)-alkyl 2-bromo-3,4-dihydroxybutanoate providing halohydrocarbon-haloalcohol dehalogenase, an altered at least predominantly (R,R)-3-chloro-2,4-dihydroxybutanenitrile providing halohydrocarbon-haloalcohol dehalogenase, an altered at least predominantly (R,R)-3-bromo-2,4-dihydroxybutanenitrile providing halohydrocarbon-haloalcohol dehalogenase, an altered at least predominantly (R,R)-3-chloro-2,4-dihydroxybutanamide providing halohydrocarbon-haloalcohol dehalogenase, an altered at least predominantly (R,R)-3-bromo-2,4-dihydroxybutanamide providing halohydrocarbon-haloalcohol dehalogenase, an altered at least predominantly (R,R)-3-chloro-2,4-dihydroxybutanoic acid providing halohydrocarbon-haloalcohol dehalogenase, an altered at least predominantly (R,R)-3-bromo-2,4-dihydroxybutanoic acid providing halohydrocarbon-haloalcohol dehalogenase, an altered at least predominantly (R,R)-alkyl 3-chloro-2,4-dihydroxybutanoate providing halohydrocarbon-haloalcohol dehalogenase, an altered at least predominantly (R,R)-alkyl 3-bromo-2,4-dihydroxybutanoate providing halohydrocarbon-haloalcohol dehalogenase, an altered at least predominantly (R,R)-4-chloro-2,3-dihydroxybutanenitrile providing halohydrocarbon-haloalcohol dehalogenase, an altered at least predominantly (R,R)-4-bromo-2,3-dihydroxybutanenitrile providing halohydrocarbon-haloalcohol dehalogenase, an altered at least predominantly (R,R)-4-chloro-2,3-dihydroxybutanamide providing halohydrocarbon-haloalcohol dehalogenase, an altered at least predominantly (R,R)-4-bromo-2,3-dihydroxybutanamide providing halohydrocarbon-haloalcohol dehalogenase, an altered at least predominantly (R,R)-4-chloro-2,3-dihydroxybutanoic acid providing halohydrocarbon-haloalcohol dehalogenase, an altered at least predominantly (R,R)-4-bromo-2,3-dihydroxybutanoic acid providing halohydrocarbon-haloalcohol dehalogenase, an altered at least predominantly (R,R)-alkyl 4-chloro-2,3-dihydroxybutanoate providing halohydrocarbon-haloalcohol dehalogenase, an altered at least predominantly (R,R)-alkyl 4-bromo-2,3-dihydroxybutanoate providing halohydrocarbon-haloalcohol dehalogenase, an altered at least predominantly (S,S)-2-chloro-3,4-dihydroxybutanenitrile providing halohydrocarbon-haloalcohol dehalogenase, an altered at least predominantly (S,S)-2-bromo-3,4-dihydroxybutanenitrile providing halohydrocarbon-haloalcohol dehalogenase, an altered at least predominantly (S,S)-2-chloro-3,4-dihydroxybutanamide providing halohydrocarbon-haloalcohol dehalogenase, an altered at least predominantly (S,S)-2-bromo-3,4-dihydroxybutanamide providing halohydrocarbon-haloalcohol dehalogenase, an altered at least predominantly (S,S)-2-chloro-3,4-dihydroxybutanoic acid providing halohydrocarbon-haloalcohol dehalogenase, an altered at least predominantly (S,S)-2-bromo-3,4-dihydroxybutanoic acid providing halohydrocarbon-haloalcohol dehalogenase, an altered at least predominantly (S,S)-alkyl 2-chloro-3,4-dihydroxybutanoate providing halohydrocarbon-haloalcohol dehalogenase, an altered at least predominantly (S,S)-alkyl 2-bromo-3,4-dihydroxybutanoate providing halohydrocarbon-haloalcohol dehalogenase, an altered at least predominantly (S,S)-3-chloro-2,4-dihydroxybutanenitrile providing halohydrocarbon-haloalcohol dehalogenase, an altered at least predominantly (S,S)-3-bromo-2,4-dihydroxybutanenitrile providing halohydrocarbon-haloalcohol dehalogenase, an altered at least predominantly (S,S)-3-chloro-2,4-dihydroxybutanamide providing halohydrocarbon-haloalcohol dehalogenase, an altered at least predominantly (S,S)-3-bromo-2,4-dihydroxybutanamide providing halohydrocarbon-haloalcohol dehalogenase, an altered at least predominantly (S,S)-3-chloro-2,4-dihydroxybutanoic acid providing halohydrocarbon-haloalcohol dehalogenase, an altered at least predominantly (S,S)-3-bromo-2,4-dihydroxybutanoic acid providing halohydrocarbon-haloalcohol dehalogenase, an altered at least predominantly (S,S)-alkyl 3-chloro-2,4-dihydroxybutanoate providing halohydrocarbon-haloalcohol dehalogenase, an altered at least predominantly (S,S)-alkyl 3-bromo-2,4-dihydroxybutanoate providing halohydrocarbon-haloalcohol dehalogenase, an altered at least predominantly (S,S)-4-chloro-2,3-dihydroxybutanenitrile providing halohydrocarbon-haloalcohol dehalogenase, an altered at least predominantly (S,S)-4-bromo-2,3-dihydroxybutanenitrile providing halohydrocarbon-haloalcohol dehalogenase, an altered at least predominantly (S,S)-4-chloro-2,3-dihydroxybutanamide providing halohydrocarbon-haloalcohol dehalogenase, an altered at least predominantly (S,S)-4-bromo-2,3-dihydroxybutanamide providing halohydrocarbon-haloalcohol dehalogenase, an altered at least predominantly (S,S)-4-chloro-2,3-dihydroxybutanoic acid providing halohydrocarbon-haloalcohol dehalogenase, an altered at least predominantly (S,S)-4-bromo-2,3-dihydroxybutanoic acid providing halohydrocarbon-haloalcohol dehalogenase, an altered at least predominantly (S,S)-alkyl 4-chloro-2,3-dihydroxybutanoate providing halohydrocarbon-haloalcohol dehalogenase, an altered at least predominantly (S,S)-alkyl 4-bromo-2,3-dihydroxybutanoate providing halohydrocarbon-haloalcohol dehalogenase, an altered at least predominantly (R,S)-2-chloro-3,4-dihydroxybutanenitrile providing halohydrocarbon-haloalcohol dehalogenase, an altered at least predominantly (R,S)-2-bromo-3,4-dihydroxybutanenitrile providing halohydrocarbon-haloalcohol dehalogenase, an altered at least predominantly (R,S)-2-chloro-3,4-dihydroxybutanamide providing halohydrocarbon-haloalcohol dehalogenase, an altered at least predominantly (R,S)-2-bromo-3,4-dihydroxybutanamide providing halohydrocarbon-haloalcohol dehalogenase, an altered at least predominantly (R,S)-2-chloro-3,4-dihydroxybutanoic acid providing halohydrocarbon-haloalcohol dehalogenase, an altered at least predominantly (R,S)-2-bromo-3,4-dihydroxybutanoic acid providing halohydrocarbon-haloalcohol dehalogenase, an altered at least predominantly (R,S)-alkyl 2-chloro-3,4-dihydroxybutanoate providing halohydrocarbon-haloalcohol dehalogenase, an altered at least predominantly (R,S)-alkyl 2-bromo-3,4-dihydroxybutanoate providing halohydrocarbon-haloalcohol dehalogenase, an altered at least predominantly (R,S)-3-chloro-2,4-dihydroxybutanenitrile providing halohydrocarbon-haloalcohol dehalogenase, an altered at least predominantly (R,S)-3-bromo-2,4-dihydroxybutanenitrile providing halohydrocarbon-haloalcohol dehalogenase, an altered at least predominantly (R,S)-3-chloro-2,4-dihydroxybutanamide providing halohydrocarbon-haloalcohol dehalogenase, an altered at least predominantly (R,S)-3-bromo-2,4-dihydroxybutanamide providing halohydrocarbon-haloalcohol dehalogenase, an altered at least predominantly (R,S)-3-chloro-2,4-dihydroxybutanoic acid providing halohydrocarbon-haloalcohol dehalogenase, an altered at least predominantly (R,S)-3-bromo-2,4-dihydroxybutanoic acid providing halohydrocarbon-haloalcohol dehalogenase, an altered at least predominantly (R,S)-alkyl 3-chloro-2,4-dihydroxybutanoate providing halohydrocarbon-haloalcohol dehalogenase, an altered at least predominantly (R,S)-alkyl 3-bromo-2,4-dihydroxybutanoate providing halohydrocarbon-haloalcohol dehalogenase, an altered at least predominantly (R,S)-4-chloro-2,3-dihydroxybutanenitrile providing halohydrocarbon-haloalcohol dehalogenase, an altered at least predominantly (R,S)-4-bromo-2,3-dihydroxybutanenitrile providing halohydrocarbon-haloalcohol dehalogenase, an altered at least predominantly (R,S)-4-chloro-2,3-dihydroxybutanamide providing halohydrocarbon-haloalcohol dehalogenase, an altered at least predominantly (R,S)-4-bromo-2,3-dihydroxybutanamide providing halohydrocarbon-haloalcohol dehalogenase, an altered at least predominantly (R,S)-4-chloro-2,3-dihydroxybutanoic acid providing halohydrocarbon-haloalcohol dehalogenase, an altered at least predominantly (R,S)-4-bromo-2,3-dihydroxybutanoic acid providing halohydrocarbon-haloalcohol dehalogenase, an altered at least predominantly (R,S)-alkyl 4-chloro-2,3-dihydroxybutanoate providing halohydrocarbon-haloalcohol dehalogenase, an altered at least predominantly (R,S)-alkyl 4-bromo-2,3-dihydroxybutanoate providing halohydrocarbon-haloalcohol dehalogenase, an altered at least predominantly (S,R)-2-chloro-3,4-dihydroxybutanenitrile providing halohydrocarbon-haloalcohol dehalogenase, an altered at least predominantly (S,R)-2-bromo-3,4-dihydroxybutanenitrile providing halohydrocarbon-haloalcohol dehalogenase, an altered at least predominantly (S,R)-2-chloro-3,4-dihydroxybutanamide providing halohydrocarbon-haloalcohol dehalogenase, an altered at least predominantly (S,R)-2-bromo-3,4-dihydroxybutanamide providing halohydrocarbon-haloalcohol dehalogenase, an altered at least predominantly (S,R)-2-chloro-3,4-dihydroxybutanoic acid providing halohydrocarbon-haloalcohol dehalogenase, an altered at least predominantly (S,R)-2-bromo-3,4-dihydroxybutanoic acid providing halohydrocarbon-haloalcohol dehalogenase, an altered at least predominantly (S,R)-alkyl 2-chloro-3,4-dihydroxybutanoate providing halohydrocarbon-haloalcohol dehalogenase, an altered at least predominantly (S,R)-alkyl 2-bromo-3,4-dihydroxybutanoate providing halohydrocarbon-haloalcohol dehalogenase, an altered at least predominantly (S,R)-3-chloro-2,4-dihydroxybutanenitrile providing halohydrocarbon-haloalcohol dehalogenase, an altered at least predominantly (S,R)-3-bromo-2,4-dihydroxybutanenitrile providing halohydrocarbon-haloalcohol dehalogenase, an altered at least predominantly (S,R)-3-chloro-2,4-dihydroxybutanamide providing halohydrocarbon-haloalcohol dehalogenase, an altered at least predominantly (S,R)-3-bromo-2,4-dihydroxybutanamide providing halohydrocarbon-haloalcohol dehalogenase, an altered at least predominantly (S,R)-3-chloro-2,4-dihydroxybutanoic acid providing halohydrocarbon-haloalcohol dehalogenase, an altered at least predominantly (S,R)-3-bromo-2,4-dihydroxybutanoic acid providing halohydrocarbon-haloalcohol dehalogenase, an altered at least predominantly (S,R)-alkyl 3-chloro-2,4-dihydroxybutanoate providing halohydrocarbon-haloalcohol dehalogenase, an altered at least predominantly (S,R)-alkyl 3-bromo-2,4-dihydroxybutanoate providing halohydrocarbon-haloalcohol dehalogenase, an altered at least predominantly (S,R)-4-chloro-2,3-dihydroxybutanenitrile providing halohydrocarbon-haloalcohol dehalogenase, an altered at least predominantly (S,R)-4-bromo-2,3-dihydroxybutanenitrile providing halohydrocarbon-haloalcohol dehalogenase, an altered at least predominantly (S,R)-4-chloro-2,3-dihydroxybutanamide providing halohydrocarbon-haloalcohol dehalogenase, an altered at least predominantly (S,R)-4-bromo-2,3-dihydroxybutanamide providing halohydrocarbon-haloalcohol dehalogenase, an altered at least predominantly (S,R)-4-chloro-2,3-dihydroxybutanoic acid providing halohydrocarbon-haloalcohol dehalogenase, an altered at least predominantly (S,R)-4-bromo-2,3-dihydroxybutanoic acid providing halohydrocarbon-haloalcohol dehalogenase, an altered at least predominantly (S,R)-alkyl 4-chloro-2,3-dihydroxybutanoate providing halohydrocarbon-haloalcohol dehalogenase, an altered at least predominantly (S,R)-alkyl 4-bromo-2,3-dihydroxybutanoate providing halohydrocarbon-haloalcohol dehalogenase, an altered 4-chloro-2,3-epoxybutanenitrile providing halohydrocarbon-haloalcohol dehalogenase, an altered 4-bromo-2,3-epoxybutanenitrile providing halohydrocarbon-haloalcohol dehalogenase, an altered 4-chloro-2,3-epoxybutanamide providing halohydrocarbon-haloalcohol dehalogenase, an altered 4-bromo-2,3-epoxybutanamide providing halohydrocarbon-haloalcohol dehalogenase, an altered 4-chloro-2,3-epoxybutanoic acid providing halohydrocarbon-haloalcohol dehalogenase, an altered 4-bromo-2,3-epoxybutanoic acid providing halohydrocarbon-haloalcohol dehalogenase, an altered alkyl 4-chloro-2,3-epoxybutanoate providing halohydrocarbon-haloalcohol dehalogenase, an altered alkyl 4-bromo-2,3-epoxybutanoate providing halohydrocarbon-haloalcohol dehalogenase, an altered 2-chloro-3,4-epoxybutanenitrile providing halohydrocarbon-haloalcohol dehalogenase, an altered 2-bromo-3,4-epoxybutanenitrile providing halohydrocarbon-haloalcohol dehalogenase, an altered 2-chloro-3,4-epoxybutanamide providing halohydrocarbon-haloalcohol dehalogenase, an altered 2-bromo-3,4-epoxybutanamide providing halohydrocarbon-haloalcohol dehalogenase, an altered 2-chloro -3,4-epoxybutanoic acid providing halohydrocarbon-haloalcohol dehalogenase, an altered 2-bromo-3,4-epoxybutanoic acid providing halohydrocarbon-haloalcohol dehalogenase, an altered alkyl 2-chloro-3,4-epoxybutanoate providing halohydrocarbon-haloalcohol dehalogenase, an altered alkyl 2-bromo-3,4-epoxybutanoate providing halohydrocarbon-haloalcohol dehalogenase, an altered at least predominantly (R,R)-4-chloro-2,3-epoxybutanenitrile providing halohydrocarbon-haloalcohol dehalogenase, an altered at least predominantly (R,R)-4-bromo-2,3-epoxybutanenitrile providing halohydrocarbon-haloalcohol dehalogenase, an altered at least predominantly (R,R)-4-chloro-2,3-epoxybutanamide providing halohydrocarbon-haloalcohol dehalogenase, an altered at least predominantly (R,R)-4-bromo-2,3-epoxybutanamide providing halohydrocarbon-haloalcohol dehalogenase, an altered at least predominantly (R,R)-4-chloro-2,3-epoxybutanoic acid providing halohydrocarbon-haloalcohol dehalogenase, an altered at least predominantly (R,R)-4-bromo-2,3-epoxybutanoic acid providing halohydrocarbon-haloalcohol dehalogenase, an altered at least predominantly (R,R)-alkyl 4-chloro -2,3-epoxybutanoate providing halohydrocarbon-haloalcohol dehalogenase, an altered at least predominantly (R,R)-alkyl 4-bromo-2,3-epoxybutanoate providing halohydrocarbon-haloalcohol dehalogenase, an altered at least predominantly (R,R)-2-chloro-3,4-epoxybutanenitrile providing halohydrocarbon-haloalcohol dehalogenase, an altered at least predominantly (R,R)-2-bromo-3,4-epoxybutanenitrile providing halohydrocarbon-haloalcohol dehalogenase, an altered at least predominantly (R,R)-2-chloro-3,4-epoxybutanamide providing halohydrocarbon-haloalcohol dehalogenase, an altered at least predominantly (R,R)-2-bromo-3,4-epoxybutanamide providing halohydrocarbon-haloalcohol dehalogenase, an altered at least predominantly (R,R)-2-chloro-3,4-epoxybutanoic acid providing halohydrocarbon-haloalcohol dehalogenase, an altered at least predominantly (R,R)-2-bromo-3,4-epoxybutanoic acid providing halohydrocarbon-haloalcohol dehalogenase, an altered at least predominantly (R,R)-alkyl 2-chloro-3,4-epoxybutanoate providing halohydrocarbon-haloalcohol dehalogenase, an altered at least predominantly (R,R)-alkyl 2-bromo-3,4-epoxybutanoate providing halohydrocarbon-haloalcohol dehalogenase, an altered at least predominantly (S,S)-4-chloro-2,3-epoxybutanenitrile providing halohydrocarbon-haloalcohol dehalogenase, an altered at least predominantly (S,S)-4-bromo-2,3-epoxybutanenitrile providing halohydrocarbon-haloalcohol dehalogenase, an altered at least predominantly (S,S)-4-chloro-2,3-epoxybutanamide providing halohydrocarbon-haloalcohol dehalogenase, an altered at least predominantly (S,S)-4-bromo-2,3-epoxybutanamide providing halohydrocarbon-haloalcohol dehalogenase, an altered at least predominantly (S,S)-4-chloro-2,3-epoxybutanoic acid providing halohydrocarbon-haloalcohol dehalogenase, an altered at least predominantly (S,S)-4-bromo-2,3-epoxybutanoic acid providing halohydrocarbon-haloalcohol dehalogenase, an altered at least predominantly (S,S)-alkyl 4-chloro-2,3-epoxybutanoate providing halohydrocarbon-haloalcohol dehalogenase, an altered at least predominantly (S,S)-alkyl 4-bromo-2,3-epoxybutanoate providing halohydrocarbon-haloalcohol dehalogenase, an altered at least predominantly (S,S)-2-chloro-3,4-epoxybutanenitrile providing halohydrocarbon-haloalcohol dehalogenase, an altered at least predominantly (S,S)-2-bromo-3,4-epoxybutanenitrile providing halohydrocarbon-haloalcohol dehalogenase, an altered at least predominantly (S,S)-2-chloro-3,4-epoxybutanamide providing halohydrocarbon-haloalcohol dehalogenase, an altered at least predominantly (S,S)-2-bromo-3,4-epoxybutanamide providing halohydrocarbon-haloalcohol dehalogenase, an altered at least predominantly (S,S)-2-chloro-3,4-epoxybutanoic acid providing halohydrocarbon-haloalcohol dehalogenase, an altered at least predominantly (S,S)-2-bromo-3,4-epoxybutanoic acid providing halohydrocarbon-haloalcohol dehalogenase, an altered at least predominantly (S,S)-alkyl 2-chloro-3,4-epoxybutanoate providing halohydrocarbon-haloalcohol dehalogenase, an altered at least predominantly (S,S)-alkyl 2-bromo-3,4-epoxybutanoate providing halohydrocarbon-haloalcohol dehalogenase, an altered at least predominantly (R,S)-4-chloro-2,3-epoxybutanenitrile providing halohydrocarbon-haloalcohol dehalogenase, an altered at least predominantly (R,S)-4-bromo-2,3-epoxybutanenitrile providing halohydrocarbon-haloalcohol dehalogenase, an altered at least predominantly (R,S)-4-chloro-2,3-epoxybutanamide providing halohydrocarbon-haloalcohol dehalogenase, an altered at least predominantly (R,S)-4-bromo-2,3-epoxybutanamide providing halohydrocarbon-haloalcohol dehalogenase, an altered at least predominantly (R,S)-4-chloro-2,3-epoxybutanoic acid providing halohydrocarbon-haloalcohol dehalogenase, an altered at least predominantly (R,S)-4-bromo-2,3-epoxybutanoic acid providing halohydrocarbon-haloalcohol dehalogenase, an altered at least predominantly (R,S)-alkyl 4-chloro-2,3-epoxybutanoate providing halohydrocarbon-haloalcohol dehalogenase, an altered at least predominantly (R,S)-alkyl 4-bromo-2,3-epoxybutanoate providing halohydrocarbon-haloalcohol dehalogenase, an altered at least predominantly (R,S)-2-chloro-3,4-epoxybutanenitrile providing halohydrocarbon-haloalcohol dehalogenase, an altered at least predominantly (R,S)-2-bromo-3,4-epoxybutanenitrile providing halohydrocarbon-haloalcohol dehalogenase, an altered at least predominantly (R,S)-2-chloro-3,4-epoxybutanamide providing halohydrocarbon-haloalcohol dehalogenase, an altered at least predominantly (R,S)-2-bromo-3,4-epoxybutanamide providing halohydrocarbon-haloalcohol dehalogenase, an altered at least predominantly (R,S)-2-chloro-3,4-epoxybutanoic acid providing halohydrocarbon-haloalcohol dehalogenase, an altered at least predominantly (R,S)-2-bromo-3,4-epoxybutanoic acid providing halohydrocarbon-haloalcohol dehalogenase, an altered at least predominantly (R,S)-alkyl 2-chloro-3,4-epoxybutanoate providing halohydrocarbon-haloalcohol dehalogenase, an altered at least predominantly (R,S)-alkyl 2-bromo-3,4-epoxybutanoate providing halohydrocarbon-haloalcohol dehalogenase, an altered at least predominantly (S,R)-4-chloro-2,3-epoxybutanenitrile providing halohydrocarbon-haloalcohol dehalogenase, an altered at least predominantly (S,R)-4-bromo-2,3-epoxybutanenitrile providing halohydrocarbon-haloalcohol dehalogenase, an altered at least predominantly (S,R)-4-chloro-2,3-epoxybutanamide providing halohydrocarbon-haloalcohol dehalogenase, an altered at least predominantly (S,R)-4-bromo-2,3-epoxybutanamide providing halohydrocarbon-haloalcohol dehalogenase, an altered at least predominantly (S,R)-4-chloro-2,3-epoxybutanoic acid providing halohydrocarbon-haloalcohol dehalogenase, an altered at least predominantly (S,R)-4-bromo-2,3-epoxybutanoic acid providing halohydrocarbon-haloalcohol dehalogenase, an altered at least predominantly (S,R)-alkyl 4-chloro-2,3-epoxybutanoate providing halohydrocarbon-haloalcohol dehalogenase, an altered at least predominantly (S,R)-alkyl 4-bromo-2,3-epoxybutanoate providing halohydrocarbon-haloalcohol dehalogenase, an altered at least predominantly (S,R)-2-chloro-3,4-epoxybutanenitrile providing halohydrocarbon-haloalcohol dehalogenase, an altered at least predominantly (S,R)-2-bromo-3,4-epoxybutanenitrile providing halohydrocarbon-haloalcohol dehalogenase, an altered at least predominantly (S,R)-2-chloro-3,4-epoxybutanamide providing halohydrocarbon-haloalcohol dehalogenase, an altered at least predominantly (S,R)-2-bromo-3,4-epoxybutanamide providing halohydrocarbon-haloalcohol dehalogenase, an altered at least predominantly (S,R)-2-chloro-3,4-epoxybutanoic acid providing halohydrocarbon-haloalcohol dehalogenase, an altered at least predominantly (S,R)-2-bromo-3,4-epoxybutanoic acid providing halohydrocarbon-haloalcohol dehalogenase, an altered at least predominantly (S,R)-alkyl 2-chloro-3,4-epoxybutanoate providing halohydrocarbon-haloalcohol dehalogenase, an altered at least predominantly (S,R)-alkyl 2-bromo-3,4-epoxybutanoate providing halohydrocarbon-haloalcohol dehalogenase, and the like.

An “altered halohydrocarbon-haloalcohol-halopolyol dehalogenase” can include, e.g., an altered 2,3-epoxy-1-propanol providing halohydrocarbon-haloalcohol-halopolyol dehalogenase, an altered 1,2,3,4-diepoxybutane providing halohydrocarbon-haloalcohol-halopolyol dehalogenase, an altered 1,2,4,5-diepoxypentane providing halohydrocarbon-haloalcohol-halopolyol dehalogenase, an altered 1,2,5,6-diepoxyhexane providing halohydrocarbon-haloalcohol-halopolyol dehalogenase, an altered 1,2,6,7-diepoxyheptane providing halohydrocarbon-haloalcohol-halopolyol dehalogenase, an altered 1,2,7,8-diepoxyoctane providing halohydrocarbon-haloalcohol-halopolyol dehalogenase, an altered at least predominantly (R)-2,3-epoxy-1-propanol providing halohydrocarbon-haloalcohol-halopolyol dehalogenase, an altered at least predominantly (S)-2,3-epoxy-1-propanol providing halohydrocarbon-haloalcohol-halopolyol dehalogenase, an altered at least predominantly (R,R)-1,2,3,4-diepoxybutane providing halohydrocarbon-haloalcohol-halopolyol dehalogenase, an altered at least predominantly (S,S)-1,2,3,4-diepoxybutane providing halohydrocarbon-haloalcohol-halopolyol dehalogenase, an altered at least predominantly (R,S)-1,2,3,4-diepoxybutane providing halohydrocarbon-haloalcohol-halopolyol dehalogenase, an altered at least predominantly (R,R)-1,2,4,5-diepoxypentane providing halohydrocarbon-haloalcohol-halopolyol dehalogenase, an altered at least predominantly (S,S)-1,2,4,5-diepoxypentane providing halohydrocarbon-haloalcohol-halopolyol dehalogenase, an altered at least predominantly (R,S)-1,2,4,5-diepoxypentane providing halohydrocarbon-haloalcohol-halopolyol dehalogenase, an altered at least predominantly (R,R)-1,2,5,6-diepoxyhexane providing halohydrocarbon-haloalcohol-halopolyol dehalogenase, an altered at least predominantly (S,S)-1,2,5,6-diepoxyhexane providing halohydrocarbon-haloalcohol-halopolyol dehalogenase, an altered at least predominantly (R,S)-1,2,5,6-diepoxyhexane providing halohydrocarbon-haloalcohol-halopolyol dehalogenase, an altered at least predominantly (R,R)-1,2,6,7-diepoxyheptane providing halohydrocarbon-haloalcohol-halopolyol dehalogenase, an altered at least predominantly (S,S)-1,2,6,7-diepoxyheptane providing halohydrocarbon-haloalcohol-halopolyol dehalogenase, an altered at least predominantly (R,S)-1,2,6,7-diepoxyheptane providing halohydrocarbon-haloalcohol-halopolyol dehalogenase, an altered at least predominantly (R,R)-1,2,7,8-diepoxyoctane providing halohydrocarbon-haloalcohol-halopolyol dehalogenase, an altered at least predominantly (S,S)-1,2,7,8-diepoxyoctane providing halohydrocarbon-haloalcohol-halopolyol dehalogenase, an altered at least predominantly (R,S)-1,2,7,8-diepoxyoctane providing halohydrocarbon-haloalcohol-halopolyol dehalogenase, an altered 4-hydroxy-2,3-epoxybutanenitrile providing halohydrocarbon-haloalcohol-halopolyol dehalogenase, an altered 4-hydroxy-2,3-epoxybutanamide providing halohydrocarbon-haloalcohol-halopolyol dehalogenase, an altered 4-hydroxy-2,3-epoxybutanoic acid providing halohydrocarbon-haloalcohol-halopolyol dehalogenase, an altered alkyl 4-hydroxy-2,3-epoxybutanoate providing halohydrocarbon-haloalcohol-halopolyol dehalogenase, an altered 2-hydroxy-3,4-epoxybutanenitrile providing halohydrocarbon-haloalcohol-halopolyol dehalogenase, an altered 2-hydroxy-3,4-epoxybutanamide providing halohydrocarbon-haloalcohol-halopolyol dehalogenase, an altered 2-hydroxy-3,4-epoxybutanoic acid providing halohydrocarbon-haloalcohol-halopolyol dehalogenase, an altered alkyl 2-hydroxy-3,4-epoxybutanoate providing halohydrocarbon-haloalcohol-halopolyol dehalogenase, an altered at least predominantly (R,R)-4-hydroxy-2,3-epoxybutanenitrile providing halohydrocarbon-haloalcohol-halopolyol dehalogenase, an altered at least predominantly (R,R)-4-hydroxy-2,3-epoxybutanamide providing halohydrocarbon-haloalcohol-halopolyol dehalogenase, an altered at least predominantly (R,R)-4-hydroxy-2,3-epoxybutanoic acid providing halohydrocarbon-haloalcohol-halopolyol dehalogenase, an altered at least predominantly (R,R)-alkyl 4-hydroxy-2,3-epoxybutanoate providing halohydrocarbon-haloalcohol-halopolyol dehalogenase, an altered at least predominantly (R,R)-2-hydroxy-3,4-epoxybutanenitrile providing halohydrocarbon-haloalcohol-halopolyol dehalogenase, an altered at least predominantly (R,R)-2-hydroxy-3,4-epoxybutanamide providing halohydrocarbon-haloalcohol-halopolyol dehalogenase, an altered at least predominantly (R,R)-2-hydroxy-3,4-epoxybutanoic acid providing halohydrocarbon-haloalcohol-halopolyol dehalogenase, an altered at least predominantly (R,R)-alkyl 2-hydroxy-3,4-epoxybutanoate providing halohydrocarbon-haloalcohol-halopolyol dehalogenase, an altered at least predominantly (S,S)-4-hydroxy-2,3-epoxybutanenitrile providing halohydrocarbon-haloalcohol-halopolyol dehalogenase, an altered at least predominantly (S,S)-4-hydroxy-2,3-epoxybutanamide providing halohydrocarbon-haloalcohol-halopolyol dehalogenase, an altered at least predominantly (S,S)-4-hydroxy-2,3-epoxybutanoic acid providing halohydrocarbon-haloalcohol-halopolyol dehalogenase, an altered at least predominantly (S,S)-alkyl 4-hydroxy-2,3-epoxybutanoate providing halohydrocarbon-haloalcohol-halopolyol dehalogenase, an altered at least predominantly (S,S)-2-hydroxy-3,4-epoxybutanenitrile providing halohydrocarbon-haloalcohol-halopolyol dehalogenase, an altered at least predominantly (S,S)-2-hydroxy-3,4-epoxybutanamide providing halohydrocarbon-haloalcohol-halopolyol dehalogenase, an altered at least predominantly (S,S)-2-hydroxy-3,4-epoxybutanoic acid providing halohydrocarbon-haloalcohol-halopolyol dehalogenase, an altered at least predominantly (S,S)-alkyl 2-hydroxy-3,4-epoxybutanoate providing halohydrocarbon-haloalcohol-halopolyol dehalogenase, an altered at least predominantly (R,S)-4-hydroxy-2,3-epoxybutanenitrile providing halohydrocarbon-haloalcohol-halopolyol dehalogenase, an altered at least predominantly (R,S)-4-hydroxy-2,3-epoxybutanamide providing halohydrocarbon-haloalcohol-halopolyol dehalogenase, an altered at least predominantly (R,S)-4-hydroxy-2,3-epoxybutanoic acid providing halohydrocarbon-haloalcohol-halopolyol dehalogenase, an altered at least predominantly (R,S)-alkyl 4-hydroxy-2,3-epoxybutanoate providing halohydrocarbon-haloalcohol-halopolyol dehalogenase, an altered at least predominantly (R,S)-2-hydroxy-3,4-epoxybutanenitrile providing halohydrocarbon-haloalcohol-halopolyol dehalogenase, an altered at least predominantly (R,S)-2-hydroxy-3,4-epoxybutanamide providing halohydrocarbon-haloalcohol-halopolyol dehalogenase, an altered at least predominantly (R,S)-2-hydroxy-3,4-epoxybutanoic acid providing halohydrocarbon-haloalcohol-halopolyol dehalogenase, an altered at least predominantly (R,S)-alkyl 2-hydroxy-3,4-epoxybutanoate providing halohydrocarbon-haloalcohol-halopolyol dehalogenase, an altered at least predominantly (S,R)-4-hydroxy-2,3-epoxybutanenitrile providing halohydrocarbon-haloalcohol-halopolyol dehalogenase, an altered at least predominantly (S,R)-4-hydroxy-2,3-epoxybutanamide providing halohydrocarbon-haloalcohol-halopolyol dehalogenase, an altered at least predominantly (S,R)-4-hydroxy-2,3-epoxybutanoic acid providing halohydrocarbon-haloalcohol-halopolyol dehalogenase, an altered at least predominantly (S,R)-alkyl 4-hydroxy-2,3-epoxybutanoate providing halohydrocarbon-haloalcohol-halopolyol dehalogenase, an altered at least predominantly (S,R)-2-hydroxy-3,4-epoxybutanenitrile providing halohydrocarbon-haloalcohol-halopolyol dehalogenase, an altered at least predominantly (S,R)-2-hydroxy-3,4-epoxybutanamide providing halohydrocarbon-haloalcohol-halopolyol dehalogenase, an altered at least predominantly (S,R)-2-hydroxy-3,4-epoxybutanoic acid providing halohydrocarbon-haloalcohol-halopolyol dehalogenase, an altered at least predominantly (S,R)-alkyl 2-hydroxy-3,4-epoxybutanoate providing halohydrocarbon-haloalcohol-halopolyol dehalogenase, and the like.

An “altered haloalcohol-halopolyol dehalogenase” can include, e.g., an altered 2,3-epoxy-1-propanol providing haloalcohol-halopolyol dehalogenase, an altered 1,2,3,4-diepoxybutane providing haloalcohol-halopolyol dehalogenase, an altered 1,2,4,5-diepoxypentane providing haloalcohol-halopolyol dehalogenase, an altered 1,2,5,6-diepoxyhexane providing haloalcohol-halopolyol dehalogenase, an altered 1,2,6,7-diepoxyheptane providing haloalcohol-halopolyol dehalogenase, an altered 1,2,7,8-diepoxyoctane providing haloalcohol-halopolyol dehalogenase, an altered (R)-2,3-epoxy-1-propanol providing haloalcohol-halopolyol dehalogenase, an altered (S)-2,3-epoxy-1-propanol providing haloalcohol-halopolyol dehalogenase, an altered (R,R)-1,2,3,4-diepoxybutane providing haloalcohol-halopolyol dehalogenase, an altered (S,S)-1,2,3,4-diepoxybutane providing haloalcohol-halopolyol dehalogenase, an altered (R,S)-1,2,3,4-diepoxybutane providing haloalcohol-halopolyol dehalogenase, an altered (R,R)-1,2,4,5-diepoxypentane providing haloalcohol-halopolyol dehalogenase, an altered (S,S)-1,2,4,5-diepoxypentane providing haloalcohol-halopolyol dehalogenase, an altered (R,S)-1,2,4,5-diepoxypentane providing haloalcohol-halopolyol dehalogenase, an altered (R,R)-1,2,5,6-diepoxyhexane providing haloalcohol-halopolyol dehalogenase, an altered (S,S)-1,2,5,6-diepoxyhexane providing haloalcohol-halopolyol dehalogenase, an altered (R,S)-1,2,5,6-diepoxyhexane providing haloalcohol-halopolyol dehalogenase, an altered (R,R)-1,2,6,7-diepoxyheptane providing haloalcohol-halopolyol dehalogenase, an altered (S,S)-1,2,6,7-diepoxyheptane providing haloalcohol-halopolyol dehalogenase, an altered (R,S)-1,2,6,7-diepoxyheptane providing haloalcohol-halopolyol dehalogenase, an altered (R,R)-1,2,7,8-diepoxyoctane providing haloalcohol-halopolyol dehalogenase, an altered (S,S)-1,2,7,8-diepoxyoctane providing haloalcohol-halopolyol dehalogenase, an altered (R,S)-1,2,7,8-diepoxyoctane providing haloalcohol-halopolyol dehalogenase, an altered 4-hydroxy-2,3-epoxybutanenitrile providing haloalcohol-halopolyol dehalogenase, an altered 4-hydroxy-2,3-epoxybutanamide providing haloalcohol-halopolyol dehalogenase, an altered 4-hydroxy-2,3-epoxybutanoic acid providing haloalcohol-halopolyol dehalogenase, an altered alkyl 4-hydroxy-2,3-epoxybutanoate providing haloalcohol-halopolyol dehalogenase, an altered 2-hydroxy-3,4-epoxybutanenitrile providing haloalcohol-halopolyol dehalogenase, an altered 2-hydroxy-3,4-epoxybutanamide providing haloalcohol-halopolyol dehalogenase, an altered 2-hydroxy-3,4-epoxybutanoic acid providing haloalcohol-halopolyol dehalogenase, an altered alkyl 2-hydroxy-3,4-epoxybutanoate providing haloalcohol-halopolyol dehalogenase, an altered at least predominantly (R,R)-4-hydroxy-2,3-epoxybutanenitrile providing haloalcohol-halopolyol dehalogenase, an altered at least predominantly (R,R)-4-hydroxy-2,3-epoxybutanamide providing haloalcohol-halopolyol dehalogenase, an altered at least predominantly (R,R)-4-hydroxy-2,3-epoxybutanoic acid providing haloalcohol-halopolyol dehalogenase, an altered at least predominantly (R,R)-alkyl 4-hydroxy-2,3-epoxybutanoate providing haloalcohol-halopolyol dehalogenase, an altered at least predominantly (R,R)-2-hydroxy-3,4-epoxybutanenitrile providing haloalcohol-halopolyol dehalogenase, an altered at least predominantly (R,R)-2-hydroxy-3,4-epoxybutanamide providing haloalcohol-halopolyol dehalogenase, an altered at least predominantly (R,R)-2-hydroxy-3,4-epoxybutanoic acid providing haloalcohol-halopolyol dehalogenase, an altered at least predominantly (R,R)-alkyl 2-hydroxy-3,4-epoxybutanoate providing haloalcohol-halopolyol dehalogenase, an altered at least predominantly (S,S)-4-hydroxy-2,3-epoxybutanenitrile providing haloalcohol-halopolyol dehalogenase, an altered at least predominantly (S,S)-4-hydroxy-2,3-epoxybutanamide providing haloalcohol-halopolyol dehalogenase, an altered at least predominantly (S,S)-4-hydroxy-2,3-epoxybutanoic acid providing haloalcohol-halopolyol dehalogenase, an altered at least predominantly (S,S)-alkyl 4-hydroxy-2,3-epoxybutanoate providing haloalcohol-halopolyol dehalogenase, an altered at least predominantly (S,S)-2-hydroxy-3,4-epoxybutanenitrile providing haloalcohol-halopolyol dehalogenase, an altered at least predominantly (S,S)-2-hydroxy-3,4-epoxybutanamide providing haloalcohol-halopolyol dehalogenase, an altered at least predominantly (S,S)-2-hydroxy-3,4-epoxybutanoic acid providing haloalcohol-halopolyol dehalogenase, an altered at least predominantly (S,S)-alkyl 2-hydroxy-3,4-epoxybutanoate providing haloalcohol-halopolyol dehalogenase, an altered at least predominantly (R,S)-4-hydroxy-2,3-epoxybutanenitrile providing haloalcohol-halopolyol dehalogenase, an altered at least predominantly (R,S)-4-hydroxy-2,3-epoxybutanamide providing haloalcohol-halopolyol dehalogenase, an altered at least predominantly (R,S)-4-hydroxy-2,3-epoxybutanoic acid providing haloalcohol-halopolyol dehalogenase, an altered at least predominantly (R,S)-alkyl 4-hydroxy-2,3-epoxybutanoate providing haloalcohol-halopolyol dehalogenase, an altered at least predominantly (R,S)-2-hydroxy-3,4-epoxybutanenitrile providing haloalcohol-halopolyol dehalogenase, an altered at least predominantly (R,S)-2-hydroxy-3,4-epoxybutanamide providing haloalcohol-halopolyol dehalogenase, an altered at least predominantly (R,S)-2-hydroxy-3,4-epoxybutanoic acid providing haloalcohol-halopolyol dehalogenase, an altered at least predominantly (R,S)-alkyl 2-hydroxy-3,4-epoxybutanoate providing haloalcohol-halopolyol dehalogenase, an altered at least predominantly (S,R)-4-hydroxy-2,3-epoxybutanenitrile providing haloalcohol-halopolyol dehalogenase, an altered at least predominantly (S,R)-4-hydroxy-2,3-epoxybutanamide providing haloalcohol-halopolyol dehalogenase, an altered at least predominantly (S,R)-4-hydroxy-2,3-epoxybutanoic acid providing haloalcohol-halopolyol dehalogenase, an altered at least predominantly (S,R)-alkyl 4-hydroxy-2,3-epoxybutanoate providing haloalcohol-halopolyol dehalogenase, an altered at least predominantly (S,R)-2-hydroxy-3,4-epoxybutanenitrile providing haloalcohol-halopolyol dehalogenase, an altered at least predominantly (S,R)-2-hydroxy-3,4-epoxybutanamide providing haloalcohol-halopolyol dehalogenase, an altered at least predominantly (S,R)-2-hydroxy-3,4-epoxybutanoic acid providing haloalcohol-halopolyol dehalogenase, and an altered at least predominantly (S,R)-alkyl 2-hydroxy-3,4-epoxybutanoate providing haloalcohol-halopolyol dehalogenase, and the like.

An “altered first haloalcohol isomer providing halohydrocarbon-second haloalcohol isomer providing halohydrocarbon dehalogenase” can include, e.g., an altered 2-halo-1-propanol providing halohydrocarbon-1-halo-2-propanol providing halohydrocarbon dehalogenase, an altered 2-chloro-1-propanol providing halohydrocarbon-1-chloro-2-propanol providing halohydrocarbon dehalogenase.

An “altered epoxide providing halohydrocarbon-haloalcohol isomer dehalogenase” can include, e.g., an altered 1,2-epoxypropane providing halohydrocarbon-haloalcohol isomer dehalogenase, an altered (R)-1,2-epoxypropane providing halohydrocarbon-haloalcohol isomer dehalogenase, an altered (S)-1,2-epoxypropane providing halohydrocarbon-haloalcohol isomer dehalogenase, and the like.

An “altered epoxide providing first haloalcohol isomer-epoxide providing second haloalcohol isomer dehalogenase” can include, e.g., an altered 1,2-epoxypropane providing 2-chloro-1-propanol-1,2-epoxypropane providing 1-chloro-2-propanol dehalogenase, an altered (R)-1,2-epoxypropane providing 2-chloro-1-propanol-1,2-epoxypropane providing 1-chloro-2-propanol dehalogenase, an altered (S)-1,2-epoxypropane providing 2-chloro-1-propanol-1,2-epoxypropane providing 1-chloro-2-propanol dehalogenase, and the like.

An “altered haloepoxide dehalogenase” can include, e.g., an altered 2,3-epoxy-1-propanol providing haloepoxide dehalogenases and the like.

An “altered epoxide hydrolase” can include, e.g., an altered 1,3-propanediol providing epoxide hydrolase, an altered (R)-1,2-propanediol providing epoxide hydrolase, an altered (S)-1,2-propanediol providing epoxide hydrolase, an altered (R)-2,3-epoxy-1-propanol providing epoxide hydrolase, an altered (S)-2,3-epoxy-1-propanol providing epoxide hydrolase, and the like.

An “altered haloepoxide dehalogenase-epoxide hydrolase” can include, e.g., an altered 1,3-propanediol providing haloepoxide dehalogenase-epoxide hydrolase, an altered at least predominantly (R)-1,2-propanediol providing haloepoxide dehalogenase-epoxide hydrolase, an altered at least predominantly (S)-1,2-propanediol providing haloepoxide dehalogenase-epoxide hydrolase, an altered at least predominantly (R)-2,3-epoxy-1-propanol providing haloepoxide dehalogenase-epoxide hydrolase, an altered at least predominantly (S)-2,3-epoxy-1-propanol providing haloepoxide dehalogenase-epoxide hydrolase, and the like.

A “set” refers to a collection of at least two molecule or sequence types.

Two nucleic acid sequences “correspond” when they have the same sequence, or when one nucleic acid sequence is a subsequence of the other, or when one sequence is derived, by natural or artificial manipulation.

A “DNAse enzyme” is an enzyme that catalyzes the cleavage of DNA, in vitro or in vivo. Many varieties of DNAse enzymes are well characterized, e.g., in Berger and Kimmel, Guide to Molecular Cloning Techniques, Methods in Enzymology volume 152 Academic Press, Inc., San Diego, Calif.; Sambrook et al., Molecular Cloning—A Laboratory Manual (2nd Ed.), Vol. 1-3, Cold Spring Harbor Laboratory, Cold Spring Harbor, N.Y., 1989 and Current Protocols in Molecular Biology, F. M. Ausubel et al., eds., Current Protocols, a joint venture between Greene Publishing Associates, Inc. and John Wiley & Sons, Inc., (supplemented through 1999), and many are commercially available.

Nucleic acids are “elongated” in a reaction that incorporates additional nucleotides, or analogs thereof, into the nucleic acid sequence. The reaction is typically catalyzed by a polymerase, e.g., a DNA polymerase. Nucleic acid fragments are “ligated” or joined together in a reaction typically catalyzed by, e.g., a ligase.

A set of “fragmented” nucleic acids results from the cleavage of at least one parental nucleic acid, e.g., enzymatically or chemically, or by providing subsequences of parental sequences in any other manner, including partially elongating a complementary sequence with a polymerase or utilizing any synthetic format.

A “full-length protein” is a protein with substantially the same sequence domains as a corresponding protein encoded by a natural gene. Such a protein can have altered sequences relative to the corresponding naturally encoded gene, e.g., due to recombination and selection, but unless specified to the contrary, is typically at least about 95% the length of the naturally encoded gene.

Nucleic acids “hybridize” when complementary single strands of nucleic acid pair to give a double-stranded nucleic acid sequence. Hybridization occurs due to a variety of well-characterized forces, including hydrogen bonding, solvent exclusion, and base stacking, which allow one to control the probability of hybridization by controlling external/experimental conditions. An extensive guide to nucleic hybridization may be found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” Elsevier, N.Y.”

A “library” is a set of polynucleotides. The set can be pooled, or can be individually accessible. The polynucleotides may comprise DNA, RNA or combinations thereof.

Nucleic acid sequences “overlap” when they possess at least one complementary subsequence.

Two nucleic acids “recombine” when sequences from each of the two nucleic acids are combined to form a chimeric progeny nucleic acid.

The terms “efficient catalyzation” or “efficient conversion” refer to one or more of various improved or otherwise novel enzymatic properties resulting from one or more cycles of directed molecular breeding, i.e., recursive sequence recombination, including, e.g., the ability of an altered enzyme to catalyze a novel reaction pathway or pathways, the ability of a single altered enzyme to catalyze multi-step reaction pathways, an altered enzyme's specific activity, an altered enzyme's selectivity, an altered enzyme's lack of susceptibility to feedback or other inhibition, an altered enzyme's pH tolerance, an altered enzyme's pH range of tolerance, an altered enzyme's enhanced Vₘₐₓ, stereoselective transformation, and the like.

The term “shuffling” is used herein to indicate recombination between non-identical sequences, in some embodiments shuffling may include crossover via homologous recombination or via non-homologous recombination, such as via cre/lox and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

### BRIEF DESCRIPTION OF THE DRAWING

FIG. 1 depicts various halohydrocarbon reaction pathways and the different classes of dehalogenases that can catalyze specific steps in the pathways.

FIG. 2 shows intermediate isomer reaction pathways including the various dehalogenases that can catalyze the particular reaction pathway steps.

FIG. 3 depicts a ring opening reaction pathway including the enzyme classes capable of catalyzing the reaction steps.

FIG. 4 provides representative C2-C3 substrates and dehalogenase reactions.

FIG. 5 provides representative C4 substrates.

FIGS. 6A-C provide representative C5 substrates.

FIGS. 7A-C provide representative C6 substrates.

### DETAILED DISCUSSION OF THE INVENTION

Halocarbon and halohydrocarbon compounds are pervasive chemical species found in the environment both as products, or at least by-products, of many naturally occurring pathways and of various synthetic processes, including certain industrial-scale chemical processes. Examples of the latter class include flame retardants, organic solvents, insecticides, fungicides, herbicides, dielectrics, degreasing agents, and intermediates employed in the synthesis of numerous other compounds. Furthermore, many halohydrocarbons are classified as recalcitrant molecules depending upon the number, type, and position of halogen substituents. Fetzner, S. and Lingens, F. (1994) “Bacterial Dehalogenases: Biochemistry, Genetics, and Biotechnological Application,” Microbiol. Rev. 58:641-685. In general, the increasing recalcitrance of carbon-halogen bonds directly correlates with the increased electronegativity of the particular substituent and polyhalogenated compounds are often less readily degradable than corresponding halogenated substances with one or few substituents. Id. For additional information relating to various aspects of recalcitrant halohydrocarbons see, e.g., Slater, J. H. “Microbial Dehalogenation of Haloaliphatic Compounds,” in Biochemistry of Microbial Degradation, Ratledge C., Ed., Kluwer Academic Publishers, Dordrecht, 379-421 (1994); Pries, F. et al. (1994) “Degradation of Halogenated Aliphatic Compounds: The Role of Adaptation,” FEMS Microbiol. Rev. 15:279-295; and Fantroussi, S. E. et al. (1998) “Anaerobic Dechlorinating Bacteria,” Biotechnol. Prog. 14:167-188.

The isolation of bacteria and fungi that grow on halogenated alkanoic acids led to the discovery of dehalogenases. Jensen, H. L. (1951) “Decomposition of Chlorosubstituted Aliphatic Acids by Soil Bacteria,” Can. J. Microbiol. 3:151-164 and Jensen, H. L. (1957) “Decomposition of Chloroorganic Acids by Fungi,” Nature 180:1416. Since then, many other microbial enzyme systems have also been discovered which catalyze the cleavage of carbon-halogen bonds. Janssen, D. B. et al. (1994) “Genetics and Biochemistry of Dehalogenating Enzymes,” Annu. Rev. Microbiol. 48:163-191; Slater, J. H. (1997) “Microbial Dehalogenation of Halogenated Alkanoic Acids, Alcohols and Alkanes,” Adv. Microbiol. Physiol. 38:133-176; van Agteren, M. H. et al. Handbook on Biodegradation and Biological Treatment of Hazardous Organic Compounds Dordrecht: Kluwer Academic Publishers (1998); and Fantroussi, S. E. et al. (1998) “Anaerobic Dechlorinating Bacteria,” Biotechnol. Prog. 14:167-188.

Many have sought to develop dehalogenating enzyme systems using either whole-cell microbial catalysts or ex vivo enzymes for bioremediation in addition to other chemical processing applications. Dehalogenating enzymes and the reactions that they are known to catalyze are discussed further in, e.g., Krooshof, G. H. (1998) “Kinetic Analysis and X-ray Structure of Haloalkane Dehalogenase with a Modified Halide-Binding Site,” Biochemistry 37:15013-15023; Holloway, P. et al. (1998) “Alteration of the Substrate Range of Haloalkane Dehalogenase by Site-Directed Mutagenesis,” Biotechnol. Bioeng. 59:520-523; Assis, H. M. S. et al. (1998) “Synthesis of Chiral Epihalohydrins Using Haloalcohol Dehalogenase A from Arthrobacter erithii H10a,” Enzyme Microbiol. Technol. 22:545-551; Kasai, N. et al. (1998) “Chiral C3 Epoxidases and Halohydrins: Their Preparation and Synthetic Application,” J. Mol. Catal. B: Enzymatic 4:237-252; Swanson, P. E. (1999) “Dehalogenases Applied to Industrial-Scale Biocatalysis,” Curr. Opin. Biotechnol. 10:365-369; Uhlig, H. et al. “Industrial Uses of Enzymes,” in Enzymes in Industry: Production and Applications, Gerhartz W., Ed., VCH Publishers, New York, 77-149 (1990); Nakamura, T. et al. (1993) “Production of (R)-3-Chloro-1,2-Propanediol from Prochiral 1,3-Dichloro-2-Propanol by Cornybacterium sp. Strain N-1074,” Appl. Environ. Microbiol. 59:227-230; Slater, J. B. et al. (1997) “Microbial Dehalogenation of Halogenated Alkanoic Acids, Alcohols and Alkanes,” Adv. Microbiol. Physiol. 38:133-176; and the references therein.

The present invention utilizes various directed molecular evolution methods to provide altered genes that encode altered hydrolases (e.g., artificially evolved hydrolases, recursively recombined hydrolases, recombinant hydrolases, chimeric hydrolases, mutant hydrolases, and the like) with, e.g., improved activities, the ability to catalyze specific reactions, including one-step or multi-step pathways (e.g., single-pot multi-step chemical transformations), and the like. As mentioned, the altered hydrolases can include altered dehalogenases. The invention also relates to methods of screening the resulting altered libraries for these and other attributes.

In one embodiment, the invention provides altered dehalogenase enzymes that have multiple or improved activities for use in the dehalogenation of 1,2,3-trichloropropane to provide 1-chloro-2,3-epoxypropane (i.e., epichlorohydrin), halohydrins, or 2,3-epoxy-1-propanol (i.e., glycidol). Altered enzymes are also provided which catalyze, e.g., the dehalogenation of 1,2-dichloropropane to yield 1,2-epoxypropane (i.e., propylene oxide). The invention also relates to altered enzymes that can be used, e.g., to generate 2-propen-1-ol (i.e., allyl alcohol) or 1,3-propanediol from 3-chloro-1-propene (i.e., allyl chloride). Other altered enzymes can dehalogenate, e.g., 1,2-dichloroethane (i.e., dichloroethylene) to provide 2-chloro-1-ethanol (i.e., ethylene glycol) or ethylene oxide. Ethylene oxide can subsequently be converted by hydrolysis to ethylene glycol which, in turn, can be used as a monomer to make polyethers. The invention also provides altered enzymes that can, e.g., convert environmentally recalcitrant compounds, including 1,2-dichloroethane (i.e., dichloroethylene), 1,2-dibromo-3-chloropropane, and the like to commercially useful compounds.

The present invention also includes altered enzymes for use in dehalogenating many other halohydrocarbons or halocarbons. For example, various haloacids (e.g., α-haloacids, β-haloacids, and the like), halonitriles, haloamides, haloesters, and the like can serve as precursors or monomers for the production of many other materials, such as polyesters, lactic acid and other hydroxy acids (e.g., α-haloacids, β-haloacids, etc.), and the like. Additionally, many products including, e.g., specific enantiomers of hydroxy propanoic and butanoic acids, can also function as highly valuable chiral intermediates for pharmaceutical and agrochemical applications. Optionally, the haloacids, halonitriles, haloamides, haloesters, and the like of the present invention can be interconverted before or after being dehalogenated. To illustrate, a haloacid can be derived from a halonitrile prior to being converted to a hydroxy acid by an altered dehalogenase. The hydroxy acid can also be obtained from a hydroxy nitrile following dehalogenation of a halonitrile by an altered dehalogenase. These interconversions can be accomplished using, e.g., nitrile hydratases, nitrilases, and the like.

As mentioned, the reaction pathways of the present invention can, e.g., be enantioselective or enantiospecific which in many cases can yield commercially valuable compounds. The significance of optically active compounds has been increasingly recognized in many diverse industries including the fields of pharmaceuticals, agrochemicals, perfumery, and liquid crystals. Stinson, S. C. (1995) Chem. Eng. News 9:44. For example, optically active epoxides and certain epoxide precursors (e.g., halohydrins) are important compounds in organic synthesis, because the epoxy ring is very reactive with nucleophiles and easily yields asymmetric alcohols. Kasai, N. et al. (1998), supra.

Optically active epichlorohydrin, glycidol, 2,3-dichloro-1-propanol, 3-chloro-1,2-propanediol, or their derivatives have a compact skeleton of glycerol and have broad potential for synthesis. For example, optically active epichlorohydrin can be used as an intermediate for the synthesis of chiral pharmaceuticals (e.g., β-adrenergic blockers, platelet-activating factor, L-camitine, vitamins, pheromones, antibiotics, and the like), epoxy resins, epoxy rubbers, adhesives, paints, agrochemicals, and ferro-electric liquid crystals. Kasai, N. et al. (1992) “Isolation of (S)-2,3-dichloro-1-propanol Assimilating Bacterium, its Characterization, and its Use in Preparation of (R)-2,3-dichloro-1-propanol and (S)-epichlorohydrin,” J. Ind. Microbiol. 10:37-43. Optically active glycidol can function as an important C-3 building block for chiral pharmaceuticals such as antibiotics, β-adrenergic blockers, and cardiovascular drugs. Id.

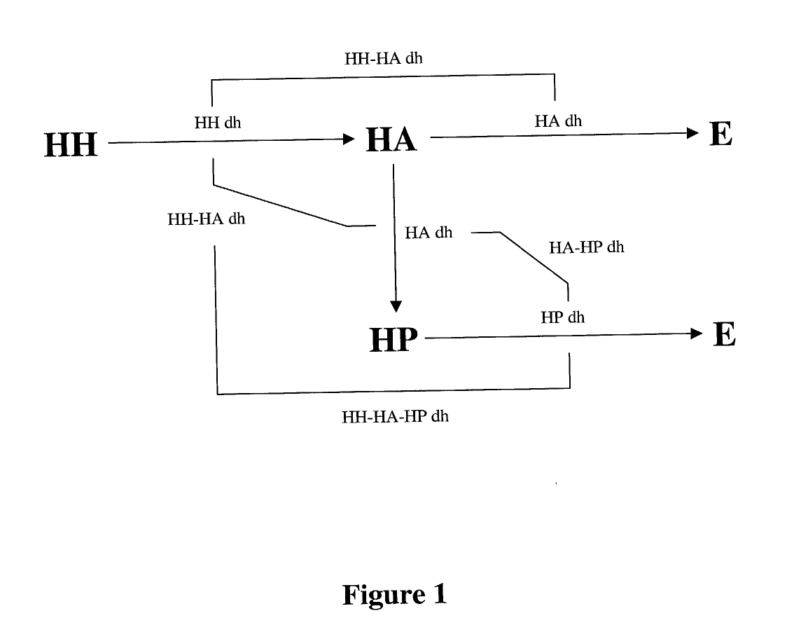

FIGS. 1 through 3, provide an overview of some of the reaction pathways included in the present invention in which altered hydrolases can catalyze single or multiple steps. In certain cases, altered hydrolases can be involved in fewer than all steps (e.g., one step) in a multiple step reaction pathway. FIG. 1 depicts various halohydrocarbon reaction pathways. For example, a halohydrocarbon (HH) like 1,2,3-trichloropropane can be converted to a haloalcohol (HA) such as 2,3-dichloro-1-propanol, which in turn can be converted, in this case, either to a haloepoxide (HE) such as 1-chloro-2,3-epoxypropane, or to a halopolyol (HP) such as 2-chloro-1,3-propanediol. The HP can, e.g., also be converted to an epoxide (E) (e.g., 2,3-epoxy-1-propanol). Although not depicted in FIG. 1, halocarbons can also be converted to similar products in comparable reaction pathways that use analogous enzymes (e.g., altered halocarbon dehalogenases).

The enzymes applicable to these pathways include an altered halohydrocarbon dehalogenase (HH dh) which can catalyze, e.g., the conversion of the HH to the HA. (FIG. 1). Altered haloalcohol dehalogenases (HA dh) can, e.g., catalyze conversions, such as, HA to HE or HA to HP. Altered halohydrocarbon-haloalcohol dehalogenases (HH-HA dh) can catalyze multi-step conversions, e.g., HH to HE and HH to HP, both of which can occur through an intermediate HA. Other enzymes include altered halopolyol dehalogenases (HP dh) which can, e.g., catalyze the conversion of HP to E. Enzymes like altered haloalcohol-halopolyol dehalogenases (HA-HP dh) can catalyze, e.g., the multi-step conversion of HA to E through an intermediate HP. Additionally, altered halohydrocarbon-haloalcohol-halopolyol dehalogenases (HH-HA-HP dh) can be used to catalyze, e.g., the multi-step conversion from HH to E through an intermediate HA and an intermediate HP.

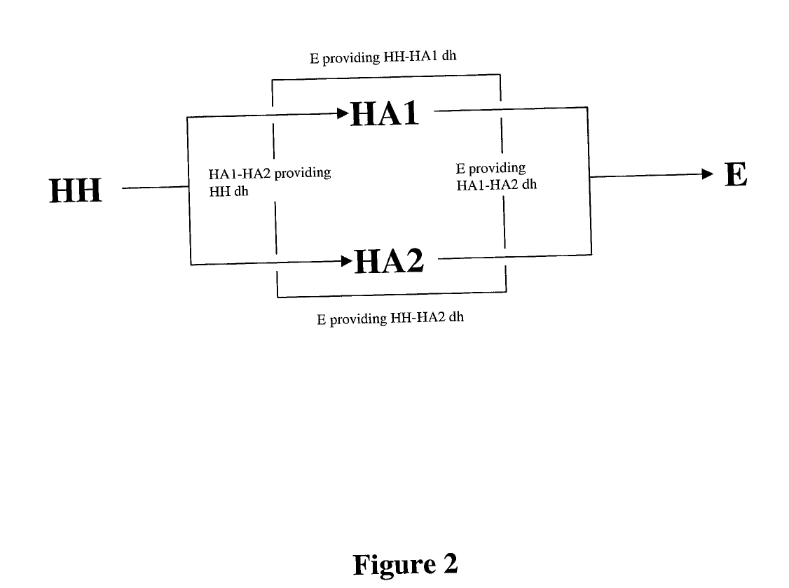

FIG. 2 relates to intermediate isomer reaction pathways. For example, a halohydrocarbon (HH) such as 1,2-dichloropropane can be converted to a first haloalcohol isomer (HA1) like 2-chloro-1-propanol and to a second haloalcohol isomer (HA2) such as 1-chloro-2-propanol. The haloalcohol isomers can subsequently be converted to an epoxide (E) such as 1,2-epoxypropane. Enzymes applicable to this pathway, include an altered first haloalcohol isomer providing halohydrocarbon-second haloalcohol isomer providing halohydrocarbon dehalogenase (HA1-HA2 providing HH dh) which can catalyze the conversion of the HH to the mixture of HA1 and HA2. An altered epoxide providing first haloalcohol isomer-epoxide providing second haloalcohol isomer dehalogenase (E providing HA1-HA2 dh) can catalyze, e.g., the conversion of the mixture of HA1 and HA2 to the E. In multi-step reactions along these pathways, altered epoxide providing halohydrocarbon-haloalcohol isomer dehalogenases (e.g., E providing HH-HA1 dh and E providing HH-HA2 dh) can catalyze, e.g., the conversion of the HH to the E through each path's respective haloalcohol intermediate (i.e., HA1 and HA2). Although not depicted in FIG. 2, halocarbons can also be converted to similar products in comparable reaction pathways that utilize analogous enzymes (e.g., altered halocarbon dehalogenases).

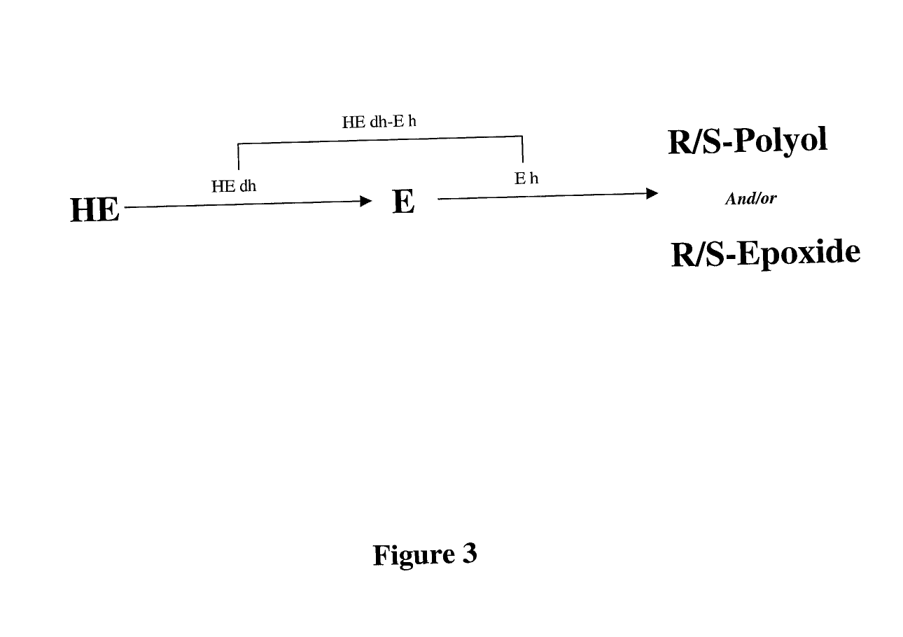

FIG. 3 shows certain ring opening reaction pathways. In one reaction, a haloepoxide (HE) such as 1-chloro-2,3-epoxypropane can be dehalogenated to form an epoxide (E) like 2,3-epoxy-1-propanol. Thereafter, the ring of E can be opened to yield a polyol (e.g., 1,3-propanediol, 1,2-propanediol, etc.) or a particular enantiomer of the epoxide. As indicated, the ring opening reactions can be enantioselective or even enantiospecific to yield the R-polyol, S-polyol, R-epoxide, and/or S-epoxide. An altered haloepoxide dehalogenase (HE dh) can catalyze, e.g., the conversion of HE to E, while an altered epoxide hydrolase (E h) can, e.g., open the epoxide ring to yield the enantiomers. Optionally, an altered haloepoxide dehalogenase-epoxide hydrolase (HE dh-E h) can be used to catalyze, e.g., the multi-step conversion of HE to the enantiomers. This latter conversion can occur through an intermediate epoxide.

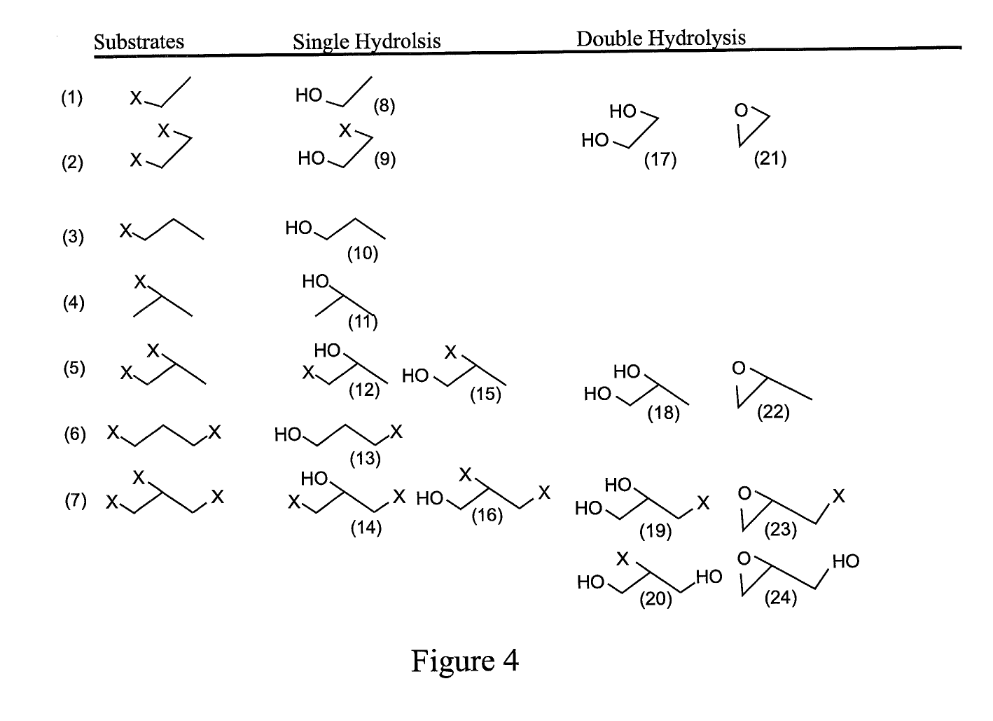

Preferred reagents for use with the methods disclosed herein are provided in FIGS. 4-7C, in which X represents a leaving group. The C2-C6 series shown in FIGS. 4-7C is representative, but not comprehensive in its listing of preferred substrates. For example, preferred reagents also typically include monomers or functional groups that include 1-20 carbons and have at least one monohalogenated carbon. Further, preferred substrates also include monomers and polymers in which the chemical entities described in the C2-C6 series (see, FIGS. 4-7C) occur as functional groups either within main polymer backbones or as appendages of more complex (e.g., aromatic, block monomer) entities. Additionally, preferred X groups include any electrophilic leaving group, such as halogens, carboxylate and carboxylate esters, methoxy, thiomethoxy, and other alkoxy and thioalkoxy groups. Particularly preferred leaving groups include chlorides and bromides. However, X can also include a hydroxy group, e.g., when there is at least one other non-hydroxy leaving group in the target molecule (see, e.g., the representative C2-C3 reactions shown in FIG. 4).

In particular, FIG. 4 provides representative C2-C3 substrates and dehalogenase reactions. It should be noted that essentially any hydrolysis product (e.g., single hydrolysis and double hydrolysis products depicted in FIG. 4) with at least one remaining leaving group (e.g., —X) is potentially a substrate for an altered hydrolase disclosed herein. Specifically, FIG. 4 illustrates potential products derived from two sequential hydrolysis reactions. It ignores the potential for the second hydrolysis step (i.e., reactions in which single hydrolysis products are further hydrolyzed to form the representative double hydrolysis products, as shown) to be an intramolecular hydrolysis, to form, e.g., an epoxide. Although specific stereochemistry is not depicted in FIG. 4 (e.g., at C2 of certain single hydrolysis products), as mentioned the present invention includes stereoselective and stereospecific enzymes, and reaction pathways.

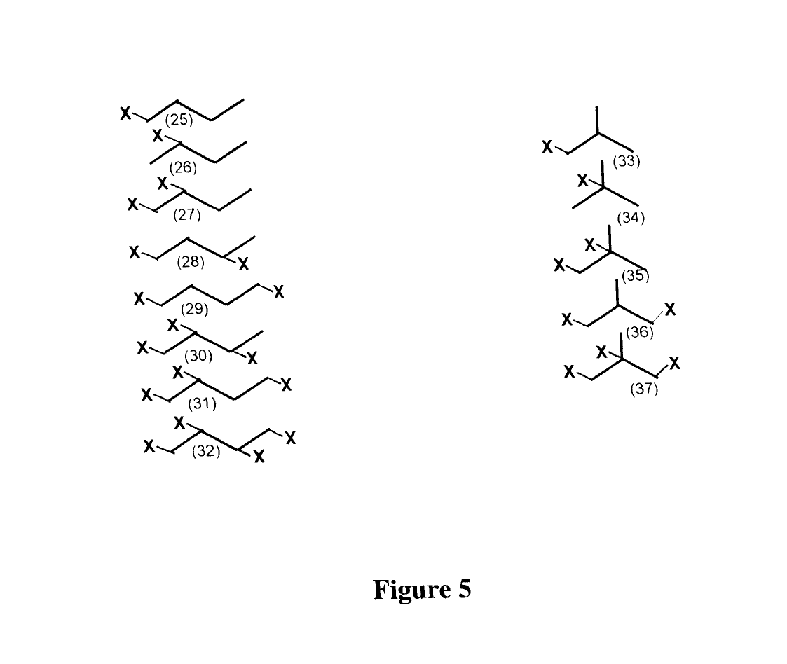

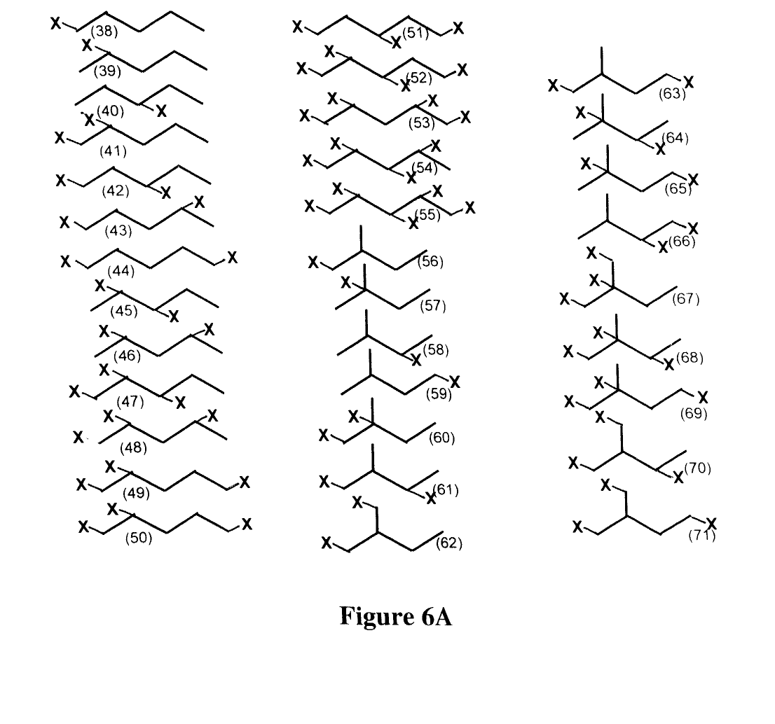

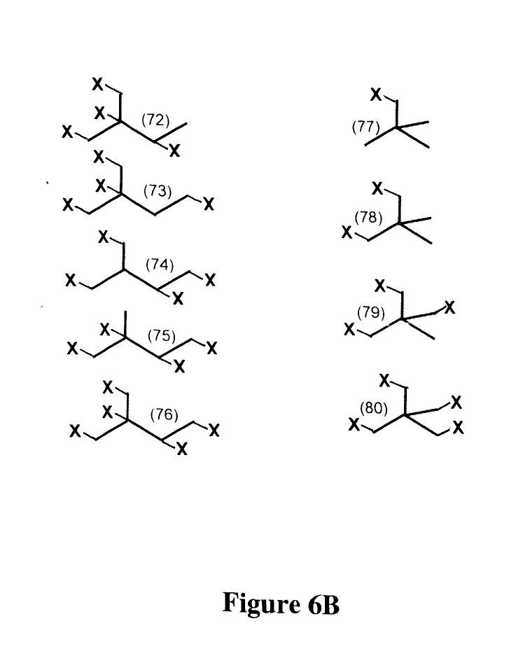

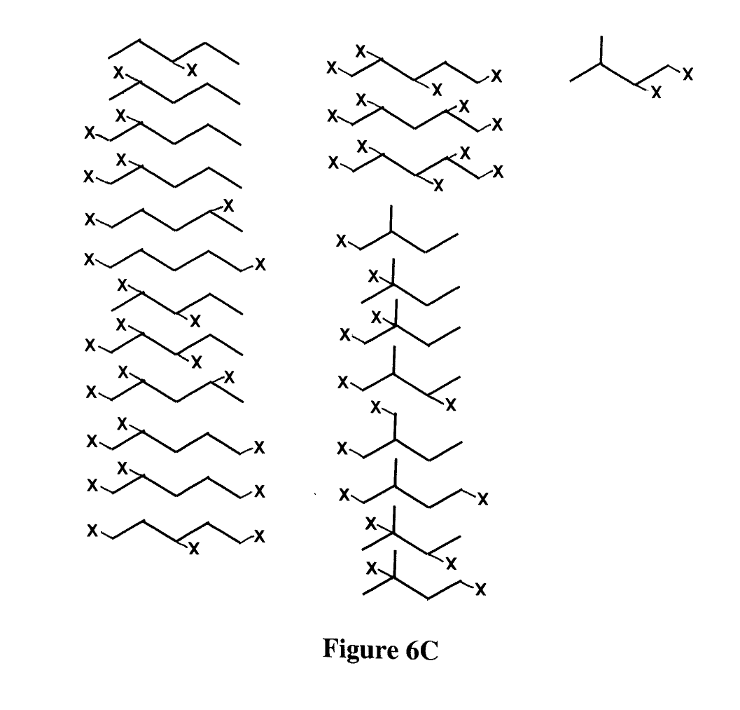

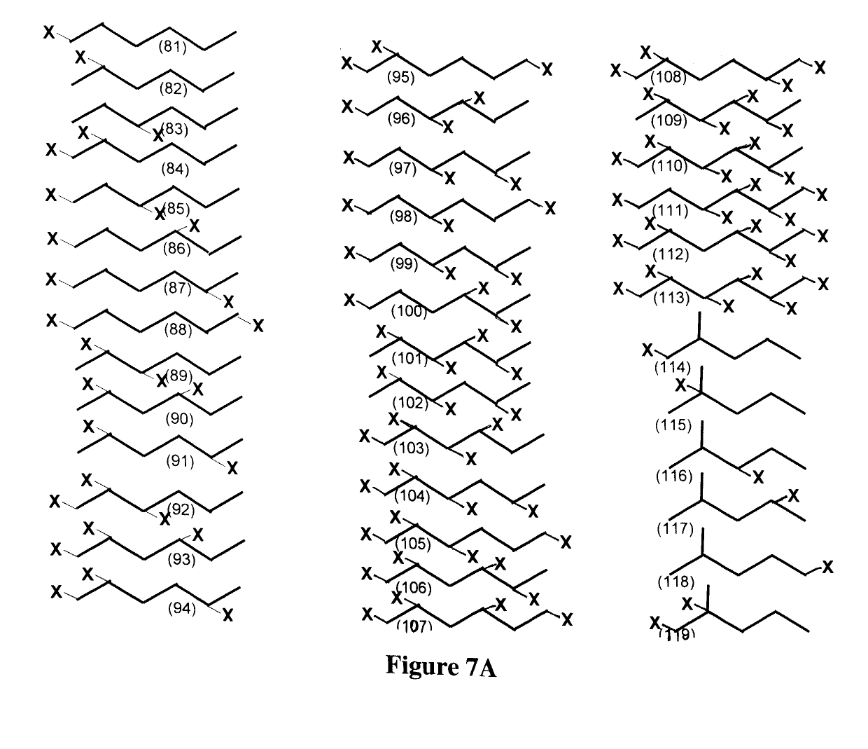

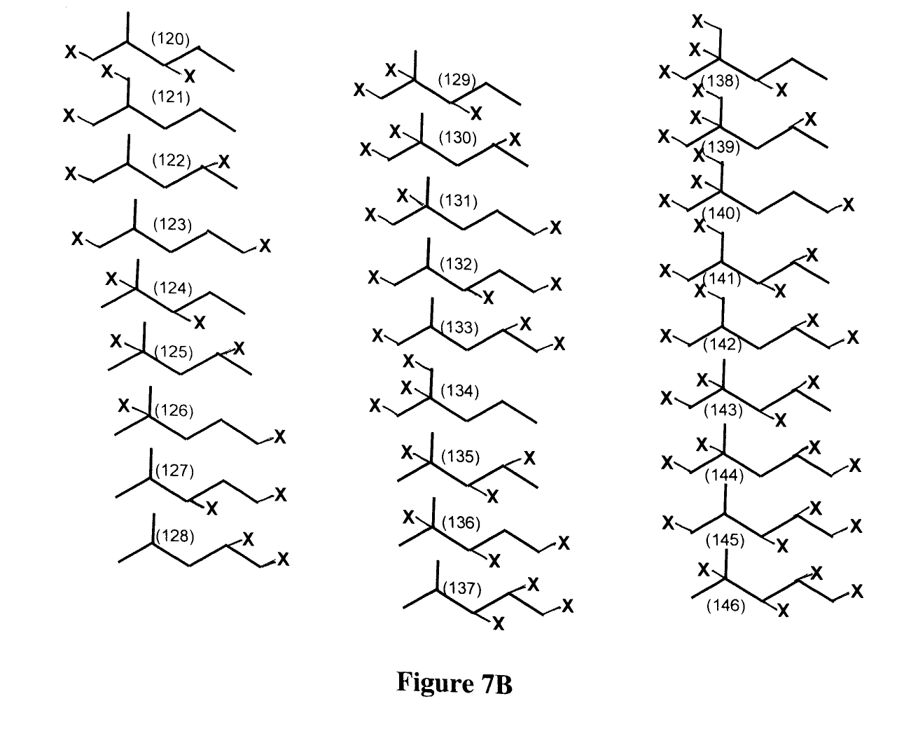

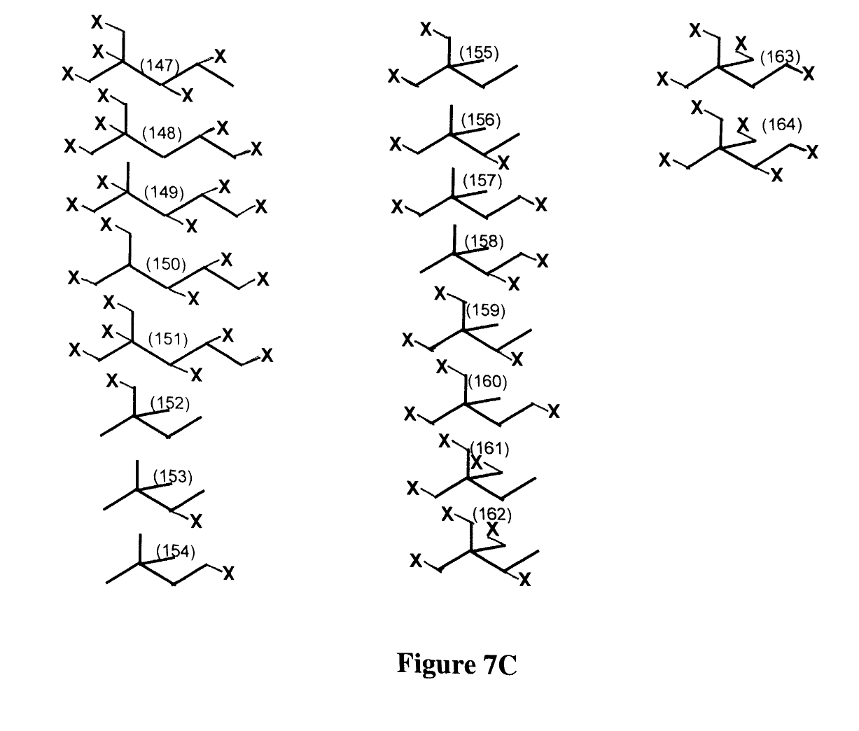

FIG. 5 provides representative C4 halohydrocarbon substrates, such as 2-halobutane and 1,2,3,4-tetrahalobutane, among others. FIGS. 6A-C provide many different representative C5 halohydrocarbon substrates including 1,3-dihalopentane, 1,3,5-trihalopentane, etc. FIGS. 7A-C provide assorted representative C6 halohydrocarbon substrates including, inter alia, 2-halohexane and 2-halo-2-methylhexane. Again, although stereochemistry is not depicted in these figures, the invention does provide stereoselective and stereospecific enzymes, and reaction pathways relating to these compounds.

The C2-C6 series of FIGS. 4-7C illustrate noncyclic, saturated alkane entities. The list of preferred substrates also includes unsaturated C1-C20 substrates in which the carbon-halogen bond to be hydrolyzed occurs at a singly bonded or partially double-bonded carbon. The list of preferred substrates also includes halogenated C1-C20 molecules that include one or more carboxylate, amide, ester, or nitrile functionalities at a terminal position in the target molecule. In such molecules, altered dehalogenases are generated by, e.g., selecting genes from the list of known haloacid, haloalkane, and/or haloalcohol dehalogenases (described further, below). Such dehalogenases are typically altered (e.g., artificially evolved, recombined, mutated, etc.) to efficiently hydrolyze the haloacids, halonitriles, haloamides, or haloesters in a series of structurally related compounds to their derivative hydroxy compounds. Any of these classes are optionally converted (e.g., enzymatically or otherwise) to hydroxy acids or esters by chemistry well represented in the art. For example, 3-hydroxybutyric acid can be derived directly by dehalogenation from 3-chlorobutyric acid, or alternately, through hydration and dehalogenation of 3-chloro-1-cyanopropane. The generation of dehalogenase to work upon any of these intermediates is contemplated in this disclosure.

As mentioned, the compounds shown in the C2-C6 compound series (FIGS. 4-7C) illustrate a representative sampling of substrates and substituent groups encountered in claimed processes and dehalogenase catalyst activities. Of the substrates shown, compounds 5, 11-13, 15, 19, and 23-24 (in FIG. 4); 26-28, 31, 34, 35, and 37 (in FIG. 5); 39-43 and most of the compounds in the series 45-80, 82-87, 89-90, 92-113, 115-151,153, and 156-164 (in FIGS. 6A-7C) have at least one stereochemical center. In addition, compounds such as 7, 14, 20, 32 and 36 (in FIGS. 4 and 5 are prochiral. For each chiral center, the present invention includes the screening and selection of dehalogenases that selectively accept or catalyze conversion at either the R or S centers as well as those which catalyze substantial conversion at both R and S stereocenters. For each prochiral compound, the present invention includes the screening of dehalogenases which accept the prochiral substrates, but yield substantially pure R or S stereoisomers from the prochiral substrates, or which yield a substantially racemic mixture from such substrates.

In particular, the chiral compounds, discussed above, typically have significant industrial importance. For example, the chiral isomers in the numbered series of FIGS. 4-7C that are important in the generation of pharmaceuticals, pharmaceutical intermediates, and high-performance materials include compounds 7, 14, 16, 19, 23, and 24 (in FIG. 4) among others. In addition, a large number of acid, nitrile and ester containing substrates with halogens at the alpha or beta positions can be dehalogenated to form R or S enantiomers of hydroxy acids such as those described, as follows:

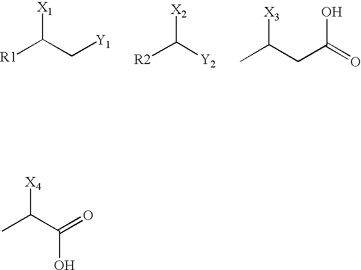

wherein R1 and R2 (and R3 and R4, below) are independently selected from: —H, —CH₃, —CH₂CH₃, —(CH₂)ₙCH₃, —C₆H₅, —C₆H₄X⁵, —C₆H₄Y³, and —((CH₂)ₑ(CHX⁶)_{f}(CH₂)_{g})ₕM¹, wherein n comprises an integer selected from and including 0 to and including 100; wherein X¹, X², X³, X⁴, X⁵, and X⁶ (and X⁷, below) are independently selected from: —F, —Cl, —Br, —I, and —CF₃; wherein Y¹, Y², and Y³ (and Y⁴, below) are independently selected from: —CN, —CONR³, —COOR⁴, —CONH₂, and —COOH; wherein e, f, g, and h comprise integers independently selected from and including 0 to and including 100; and, wherein M² is selected from: —H, —CH₂X⁷, —CH₃, and —Y⁴.

The following provides details regarding various aspects of the methods of converting various reactants (e.g., halocarbons, halohydrocarbons, haloalcohols, halopolyols, etc.) to particular products (e.g., haloalcohols, halopolyols, epoxides, etc.) using different altered dehalogenases and/or other altered hydrolases. It also provides specific information pertaining to the methods of shuffling nucleic acids and of selecting or screening encoded enzymatic products, and to kits and integrated systems.

Enzymatically Catalyzed Reaction Pathways

Overview of Reactions

In general, the present invention includes methods of converting an organic compound, a halocarbon, or a first halohydrocarbon to a product comprising incubating the organic compound, the halocarbon, or the first halohydrocarbon and at least one altered hydrolase (e.g., an altered halocarbon dehalogenase, an altered halohydrocarbon dehalogenase, etc.), in which the altered hydrolase converts the organic compound, the halocarbon, or the first halohydrocarbon to the product. Various hydrocarbon and halocarbon reactants and derivatives are discussed above.

The first halohydrocarbon optionally includes a compound having the formula:

M¹((CH₂)ₐ(CHX¹)b(CH₂)_{c})_{d}M²

wherein a, b, c, and d comprise integers independently selected from and including 0 to and including 100; wherein M¹ and M² are independently selected from: —H, —CH₂X², —CH₃, —R¹, —CN, —CONHR², —CONR³R⁴, —COOR⁵, —CONH₂, and —COOH; wherein R¹, R², R³, R⁴, and R⁵ are independently selected from: —H, —CH₃, —CH₂CH₃, —(CH₂)ₙCH₃, —C₆H₅, —C₆H₄X³, —C₆H₄Y, and —((CH₂)ₑ(CHX⁴)_{f}(CH₂)_{g})ₕM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³ is selected from: —H, —CH₂X⁵, —CH₃, —R¹⁰, —CN, —CONHR¹¹, —CONR¹²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶; wherein nn comprises an integer selected from and including 0 to and including 100; and, wherein X¹, X², X³, X⁴, X⁵, and X⁶ are independently selected from: —F, —Cl, —Br, and —I (—Cl and —Br are generally preferred). In certain embodiments d is about 0 and b is at least about 1, or e.g., at least about 2. In other embodiments h is about 0 and f is at least about 1.

Alternatively, the first halohydrocarbon includes a compound having the formula:

M¹((CQ¹Q²)ₐ(CHX¹)_{b}(CQ³Q⁴)_{c})_{d}M²

wherein a, b, c, and d comprise integers independently selected from and including 0 to and including 100; wherein M¹ and M² are independently selected from: —H, —CH₂X², —CH₃, —CN, —CONHR¹, —CONR²R³, —COOR⁴, —CONH₂, —COOH, and —((CH₂)ₑ(CHX³)_{f}(CH₂)_{g})ₕM³; wherein e, f, g, and h comprise integers independently selected from and including 0 to and including 100; wherein R¹, R², R³, and R⁴ are independently selected from: —H, —CH₃, —CH₂CH₃, —(CH₂)ₙₙCH₃, —C₆H₅, —C₆H₄, —C₆H₄Y, and —((CH₂)ᵢ(CHX⁵)ⱼ(CH₂)ₖ)ₗM⁴; wherein nn comprises an integer selected from and including 0 to and including 100; wherein Y is selected from: —CN, —CONHR⁵, —CONR⁶R⁷, —COOR⁸, —CONH₂, —COOH; wherein i, j, k, and l comprise integers independently selected from and including 0 to and including 100; wherein Q¹ is selected from: —H and —((CH₂)ₘ(CHX⁶)ₙ(CH₂)ₒ)ₚM⁵; wherein m, n, o, and p comprise integers independently selected from and including 0 to and including 100; wherein Q² is selected from: —H and —((CH₂)_{q}(CHX⁷)ᵣ(CH₂)ₛ)ₜM⁶; wherein q, r, s, and t comprise integers independently selected from and including 0 to and including 100; wherein Q³ is selected from: —H and —((CH₂)ᵤ(CHX⁸)ᵥ(CH₂)_{w})ₓM⁷; wherein u, v, w, and x comprise integers independently selected from and including 0 to and including 100; wherein Q⁴ is selected from: —H and —((CH₂)_{y}(CHX⁹)_{z}(CH₂)ₐₐ)_{bb}M⁸; wherein y, z, aa, and bb comprise integers independently selected from and including 0 to and including 100; wherein M³, M⁴, M⁵, M⁶, M⁷, and M⁸ are independently selected from: —H, —CH₂X¹⁰, —CH₃, —R⁹, —CN, —CONHR¹⁰, —CONR¹¹R¹², —COOR¹³, —CONH₂, and —COOH; wherein R⁵, R⁶, R⁷, R⁸, R⁹, R¹⁰, R¹¹, R¹², and R¹³ are independently selected from: —H, —CH₃, —CH₂CH₃, —(CH₂)ₙₙₙCH₃, —C₆H₅, —C₆H₄X¹¹; wherein nnn comprises an integer selected from and including 0 to and including 100;and wherein X¹, X², X³, X⁴, X⁵, X⁶, X⁷, X⁸, X⁹, X¹⁰, and X¹¹ are independently selected from: —F, —Cl, —Br, and —I (—Cl and —Br are generally preferred). In certain embodiments, d is about 0 and b is at least about 1, or e.g., at least about 2; h is about 0 and f is at least about 1; 1 is about 0 and j is at least about 1; p is about 0 and n is at least about 1; t is about 0 and r is at least about 1; x is about 0 and v is at least about 1; and/or bb is about 0 and z is at least about 1. In alternative embodiments, at least one of Q¹, Q², Q³, and Q⁴ include one or more halogens, in which at least one of the one or more halogens is hydrolyzed upon conversion of M¹((CQ¹Q²)ₐ(CHX¹)_{b}(CQ³Q⁴)_{c})_{d}M² to the at least one product.

The product optionally includes a compound having the formula:

M¹((CH₂)ₐ(CHX¹)_{b-c}(CHOH)_{c}(CH₂)_{d})ₑM²

wherein a, b, c, d, and e comprise integers independently selected from and including 0 to and including 100; wherein M¹ and M² are independently selected from: —H, —CH₂X², —CH₃, —R¹, —CN, —CONHR², —CONR³R⁴, —COOR⁵, —CONH₂, and —COOH; wherein R¹, R², R³, R⁴, and R⁵ are independently selected from: —H, —CH₃, —CH₂CH₃, —(CH₂)ₙCH₃, —C₆H₅, —C₆H₄X³, —C₆H⁴Y, and —((CH₂)_{f}(CHX⁴)_{g}(CH₂)ₕ)ᵢM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³is selected from: —H, —CH₂X⁵, —CH₃, —R¹⁰, —CN, —CONHR¹¹, —CONR¹²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶; wherein nn comprises an integer selected from and including 0 to and including 100; and, wherein X¹, X², X³, X⁴, X⁵, and X⁶ are independently selected from: —F, —Cl, —Br, and —I (—Cl and —Br are generally preferred). In some embodiments, e is about 0 and b-c is at least about 1, or e.g., at least about 2; i is about 0 and f is at least about 1; and/or c is at most equal to b.

In alternative embodiments, the product includes a compound having the formula:

M¹((CQ¹Q²)ₐ(CHX¹)_{b-c}(CHOH)_{c}(CQ³Q⁴)_{d})ₑM²

wherein a, b, c, d, and e comprise integers independently selected from and including 0 to and including 100; wherein M¹ and M² are independently selected from: —H, —CH₂X², —CH₃, —CN, —CONHR¹, —CONR², R³, —COOR⁴, —CONH₂, —COOH, and —((CH₂)_{f}(CHX³)_{g}(CH₂)ₕ)ᵢM³; wherein f, g, h, and i comprise integers independently selected from and including 0 to and including 100; wherein R¹, R², R³, and R⁴ are independently selected from: —H, —CH₃, —CH₂CH₃, —(CH₂)ₙₙCH₃, —C₆H₅, —C₆H₄X⁴, —C₆H₄Y, and —((CH₂)ⱼ(CHX⁵)ₖ(CH₂)ₗ)ₘM⁴; wherein nn comprises an integer selected from and including 0 to and including 100; wherein Y is selected from: —CN, —CONHR⁵, —CONR⁶R⁷, —COOR⁸, —CONH₂, —COOH; wherein j, k, l, and m comprise integers independently selected from and including 0 to and including 100; wherein Q¹ is selected from: —H and —((CH₂)ₙ(CHX⁶)ₒ(CH₂)ₚ)_{q}M⁵; wherein n, o, p, and q comprise integers independently selected from and including 0 to and including 100; wherein Q² is selected from: —H and —((CH₂)ᵣ(CHX⁷)ₛ(CH₂)ₜ)ᵤM⁶; wherein r, s, t, and u comprise integers independently selected from and including 0 to and including 100; wherein Q³ is selected from: —H and —((CH₂)ᵥ(CHX⁸)_{w}(CH₂)ₓ)_{y}M⁷; wherein v, w, x, and y comprise integers independently selected from and including 0 to and including 100; wherein Q⁴ is selected from: —H and —((CH₂)_{z}(CHX⁹)ₐₐ(CH₂)_{bb})_{cc}M⁸; wherein z, aa, bb, and cc comprise integers independently selected from and including 0 to and including 100; wherein M³, M⁴, M⁵, M⁶, M⁷, and M⁸ are independently selected from: —H, —CH₂X¹⁰, —CH₃, —R⁹, —CN, —CONHR¹⁰, —CONR¹¹R¹², —COOR¹³, —CONH₂, and —COOH; wherein R⁵, R⁶, R⁷, R⁸, R⁹, R¹⁰, R¹¹, R¹², and R¹³ are independently selected from: —H, —CH₃, —CH₂CH₃, —(CH₂)ₙₙₙCH₃, —C₆H₅, —C₆H₄X¹¹; wherein nnn comprises an integer selected from and including 0 to and including 100;and wherein X¹, X², X³, X⁴, X⁵, X⁶, X⁷, X⁸, X⁹, X¹⁰, and X¹¹ are independently selected from: —F, —Cl, —Br, and —I (—Cl and —Br are generally preferred). In certain embodiments, e is about 0 and b-c is at least about 1, e.g., at least about 2; i is about 0 and g is at least about 1; m is about 0 and k is at least about 1; q is about 0 and o is at least about 1; u is about 0 and s is at least about 1; y is about 0 and w is at least about 1; cc is about 0 and aa is at least about 1; and/or c is at most equal to b.

Halohydrocarbon Reaction Pathways

The present invention relates to methods of converting, e.g., a first halohydrocarbon (e.g., an acyclic or cyclic halohydrocarbon) to a first product (e.g., a first hydrocarbon, a second halohydrocarbon, a first haloalcohol (e.g., a halohydrin), to a first polyol (e.g., a diol, a triol, etc.), a first halopolyol, a first haloepoxide, a first epoxide, or to an alcohol (e.g., an α-hydroxy acid, a β-hydroxy acid, etc.). As mentioned, the methods can include incubating the first halohydrocarbon and an altered halohydrocarbon dehalogenase in which the altered halohydrocarbon dehalogenase converts the first halohydrocarbon to the first product. Methods of providing altered dehalogenases and other hydrolases are described, infra. The first halohydrocarbon can optionally be at least enantioselectively (or enantiospecifically) converted to the first product. In this portion of the disclosure, for the sake of concision, reagents (i.e., reactants, intermediates, products, etc.) and enzymes are often referenced only according to the general classes (e.g., halohydrocarbon, haloalcohol, epoxide, altered halopolyol dehalogenase, etc.) to which they belong. More specific examples of preferred reagents and enzymes are provided, supra.

If a first haloalcohol is provided by the conversion of the halohydrocarbon, the first haloalcohol can thereafter optionally be converted to a second product (e.g., a second hydrocarbon, a third halohydrocarbon, a second haloalcohol, a second polyol, a second halopolyol, a second epoxide, a second haloepoxide, and the like). The methods involve incubating the first haloalcohol and an altered haloalcohol dehalogenase in which the altered haloalcohol dehalogenase converts the first haloalcohol to the second product (e.g., to the second epoxide, to the second polyol (e.g., 1,2-ethanediol, 1,3-propanediol, and the like), to the second halopolyol, etc.). Thereafter, the second epoxide (e.g., ethylene oxide) can be hydrolyzed to provide a third polyol (e.g., 1,2-ethanediol). Furthermore, the second polyol (e.g., 1,2-ethanediol) can optionally be incubated with a fourth halohydrocarbon (e.g., 1,2-dichloroethane) to provide a polyether.

These reaction pathways can also include converting the first halopolyol to a second epoxide. This conversion includes incubating the first halopolyol and an altered halopolyol dehalogenase in which the altered halopolyol dehalogenase converts the first halopolyol to the second epoxide. Additionally, the first halopolyol can be at least enantioselectively (or enantiospecifically) converted to the second epoxide. Preferred enantiomers of the second epoxide are discussed further, supra.

The present invention also includes a multi-step method of converting a first halohydrocarbon to a product (e.g., a hydrocarbon, a second halohydrocarbon, a haloalcohol, a first polyol, a first halopolyol, a first epoxide, a haloepoxide first, and the like). The method includes incubating the first halohydrocarbon and the altered halohydrocarbon-haloalcohol dehalogenase in which the altered halohydrocarbon-haloalcohol dehalogenase converts the first halohydrocarbon to the product. The conversion of the first halohydrocarbon to the first epoxide or to the first polyol can occur through an intermediate haloalcohol. Preferred reagents and altered halohydrocarbon-haloalcohol dehalogenases are discussed, supra. According to this method, the first halohydrocarbon can be at least enantioselectively converted to the product or the intermediate haloalcohol. Preferred predominant enantiomers of the first epoxide, of the first halopolyol, and of the intermediate haloalcohol that can be provided by these reaction pathways are listed, supra. Furthermore, the method can optionally include hydrolyzing the first epoxide (e.g., ethylene oxide) to provide a second polyol (e.g., 1,2-ethanediol). Additionally, the method can also optionally include incubating the first polyol (e.g., 1,2-ethanediol) with a third halohydrocarbon (e.g., 1,2-dichloroethane) to provide a polyether.

The invention also includes a multi-step method of converting a halohydrocarbon to an epoxide utilizing an altered halohydrocarbon-haloalcohol-halopolyol dehalogenase. The method includes incubating the halohydrocarbon and an altered halohydrocarbon-haloalcohol-halopolyol dehalogenase in which the altered halohydrocarbon-haloalcohol-halopolyol dehalogenase converts the halohydrocarbon to the epoxide. The conversion of the halohydrocarbon to the epoxide can occur through an intermediate haloalcohol and an intermediate halopolyol. Furthermore, the halohydrocarbon can be at least enantioselectively converted to the epoxide. Preferred halohydrocarbons, epoxides, predominant epoxide enantiomers, intermediate haloalcohols, intermediate halopolyols, and altered halohydrocarbon-haloalcohol-halopolyol dehalogenases are all described, supra.

When a first halohydrocarbon is converted to a first haloalcohol in accordance with the methods described above, the invention further includes a method of converting the first haloalcohol to a second epoxide. The method includes incubating the first haloalcohol and an altered haloalcohol-halopolyol dehalogenase in which the altered haloalcohol-halopolyol dehalogenase converts the first haloalcohol to the second epoxide. This conversion can occur through an intermediate halopolyol. Additionally, the first haloalcohol can be at least enantioselectively converted to the epoxide. Preferred first haloalcohols, intermediate halopolyols, epoxides, predominant epoxide enantiomers, and altered haloalcohol-halopolyol dehalogenases are discussed, supra.

If an altered halohydrocarbon-haloalcohol dehalogenase converts a first halohydrocarbon to a first halopolyol as provided by the methods described above, the invention also includes a method of converting the first halopolyol to a second epoxide. The method can include incubating the first halopolyol and an altered halopolyol dehalogenase in which the altered halopolyol dehalogenase converts the first halopolyol to the second epoxide. Optionally, the first halopolyol can be at least enantioselectively converted to the second epoxide. Preferred halopolyols, epoxides, predominant epoxide enantiomers, and halopolyol dehalogenases are discussed further, supra.

Haloalcohol Reaction Pathways

The present invention also relates to methods of converting a haloalcohol (e.g., a halohydrin) to a product (e.g., a hydrocarbon, a first halohydrocarbon, a second haloalcohol, a first polyol, a first halopolyol, an epoxide, a haloepoxide, and the like). The method includes incubating the haloalcohol and an altered haloalcohol dehalogenase in which the altered haloalcohol dehalogenase converts the haloalcohol to the product. The first halopolyol obtained by these methods can also optionally be converted to a second epoxide, by incubating the first halopolyol and an altered halopolyol dehalogenase in which the altered halopolyol dehalogenase converts the first halopolyol to the second epoxide. If the first halopolyol is further converted to a second epoxide, the conversion can occur at least enantioselectively or optionally, enantiospecifically. Preferred predominant epoxide enantiomers are listed, supra.

The haloalcohol can optionally be at least enantioselectively converted to the first epoxide. Preferred predominant enantiomers of the first epoxide are listed, supra. However, these and other epoxide enantiomers can also be enantiospecifically provided. A haloalcohol can also be at least enantioselectively (or optionally enantiospecifically) converted to a first halopolyol. A list of preferred predominant enantiomers of the first halopolyol is provided, supra.

Once the first epoxide (e.g., ethylene oxide) is produced, it can optionally be hydrolyzed to provide a second polyol (e.g., 1,2-ethanediol). In one embodiment of the present invention, the first polyol (e.g., 1,2-ethanediol) can optionally be incubated with a second halohydrocarbon (e.g., 1,2-dichloroethane) to produce a polyether.

The present invention also includes a multi-step method of converting a haloalcohol to an epoxide using an altered haloalcohol-halopolyol dehalogenase. The method can include incubating the haloalcohol and an altered haloalcohol-halopolyol dehalogenase in which the altered haloalcohol-halopolyol dehalogenase converts the haloalcohol to the epoxide. The conversion of the haloalcohol to the epoxide can occur through an intermediate halopolyol. Optionally, the haloalcohol can be at least enantioselectively converted to the epoxide. Preferred reagents including haloalcohols, intermediate halopolyols, epoxides, and predominant epoxide enantiomers are all listed, supra. Preferred altered haloalcohol-halopolyol dehalogenases are also discussed, supra.

Halopolyol Reaction Pathways

The present invention includes a method of converting a halopolyol to an epoxide. The method includes incubating the halopolyol and an altered halopolyol dehalogenase in which the altered halopolyol dehalogenase converts the halopolyol to the epoxide. The halopolyol can optionally be at least enantioselectively converted to the epoxide. Preferred predominant enantiomers of the epoxide as well as other reagents and altered halopolyol dehalogenases are all discussed, supra.

Intermediate Isomer Reaction Pathways

The present invention includes a method of converting a plurality of a halohydrocarbon (e.g., 1,2-dichloropropane) to at least one first haloalcohol (e.g., 2-chloro-1-propanol) isomer and to at least one second haloalcohol isomer (e.g., 1-chloro-2-propanol). The method can include incubating the plurality of the halohydrocarbon and an altered first haloalcohol isomer providing halohydrocarbon-second haloalcohol isomer providing halohydrocarbon dehalogenase. In so doing, the altered first haloalcohol isomer providing halohydrocarbon-second haloalcohol isomer providing halohydrocarbon dehalogenase can convert the plurality of the halohydrocarbon to the first haloalcohol isomer and the second haloalcohol isomer. A preferred altered first haloalcohol isomer providing halohydrocarbon-second haloalcohol isomer providing halohydrocarbon dehalogenase includes, e.g., an altered 2-chloro-1-propanol providing halohydrocarbon-1-chloro-2-propanol providing halohydrocarbon dehalogenase.

When the plurality of a halohydrocarbon is converted to at least one first haloalcohol isomer (e.g., 2-chloro-1-propanol) and to at least one second haloalcohol isomer (e.g., 1-chloro-2-propanol) in accordance with the method described above, the invention also includes a method of converting the first haloalcohol isomer and the second haloalcohol isomer to an epoxide (e.g., 1,2-epoxypropane). The method includes incubating the first haloalcohol isomer and the second haloalcohol isomer and an altered epoxide providing first haloalcohol isomer-epoxide providing second haloalcohol isomer dehalogenase. In so doing, the altered epoxide providing first haloalcohol isomer-epoxide providing second haloalcohol isomer dehalogenase can convert the first haloalcohol isomer and the second haloalcohol isomer to the epoxide. The plurality of a halohydrocarbon can also be at least enantioselectively converted to the at least one epoxide, in which the predominant enantiomer of the epoxide can include, e.g., (R)-1,2-epoxypropane, (S)-1,2-epoxypropane, and the like. Preferred altered epoxide providing first haloalcohol isomer-epoxide providing second haloalcohol isomer dehalogenases can include, e.g., an altered 1,2-epoxypropane providing 2-chloro-1-propanol-1,2-epoxypropane providing 1-chloro-2-propanol dehalogenase, an altered (R)-1,2-epoxypropane providing 2-chloro-1-propanol-1,2-epoxypropane providing 1-chloro-2-propanol dehalogenase, an altered (S)-1,2-epoxypropane providing 2-chloro-1-propanol-1,2-epoxypropane providing 1-chloro-2-propanol dehalogenase, and the like.

The invention also includes a multi-step method of converting a halohydrocarbon (e.g., 1,2-dichloropropane) to at least one epoxide (e.g., 1,2-epoxypropane) using an altered epoxide providing halohydrocarbon-haloalcohol isomer dehalogenase. The method can involve incubating the halohydrocarbon and an altered epoxide providing halohydrocarbon-haloalcohol isomer dehalogenase in which the altered epoxide providing halohydrocarbon-haloalcohol isomer dehalogenase converts the halohydrocarbon to the epoxide. The conversion of the halohydrocarbon to the epoxide can occur through an intermediate haloalcohol isomer (e.g., 2-chloro-1-propanol, 1-chloro-2-propanol, and the like). Additionally, the halohydrocarbon can be at least enantioselectively converted to the epoxide. Preferred predominant enantiomers of the epoxide include, e.g., (R)-1,2-epoxypropane and (S)-1,2-epoxypropane. Preferred altered epoxide providing halohydrocarbon-haloalcohol isomer dehalogenases can include, e.g., an altered 1,2-epoxypropane providing halohydrocarbon-haloalcohol isomer dehalogenase, an altered (R)-1,2-epoxypropane providing halohydrocarbon-haloalcohol isomer dehalogenase, an altered (S)-1,2-epoxypropane providing halohydrocarbon-haloalcohol isomer dehalogenase, and the like.

The present invention also provides a method of converting a mixture that includes a first haloalcohol isomer (e.g., 2-chloro-1-propanol) and a second haloalcohol isomer (e.g., 1-chloro-2-propanol) to an epoxide (e.g., 1,2-epoxypropane) utilizing an altered epoxide providing first haloalcohol isomer-epoxide providing second haloalcohol isomer dehalogenase. The method includes incubating the mixture and an altered epoxide providing first haloalcohol isomer-epoxide providing second haloalcohol isomer dehalogenase. In so doing, the altered epoxide providing first haloalcohol isomer-epoxide providing second haloalcohol isomer dehalogenase can convert the mixture to the epoxide. Furthermore, the mixture can be at least enantioselectively converted to the epoxide, in which case a predominant enantiomer of the epoxide can include, e.g., (R)-1,2-epoxypropane, (S)-1,2-epoxypropane, and the like. Preferred altered epoxide providing first haloalcohol isomer-epoxide providing second haloalcohol isomer dehalogenases can include, e.g., an altered 1,2-epoxypropane providing 2-chloro-1-propanol-1,2-epoxypropane providing 1-chloro-2-propanol dehalogenase, an altered (R)-1,2-epoxypropane providing 2-chloro-1-propanol-1,2-epoxypropane providing 1-chloro-2-propanol dehalogenase, an altered (S)-1,2-epoxypropane providing 2-chloro-1-propanol-1,2-epoxypropane providing 1-chloro-2-propanol dehalogenase, and the like.

Ring Opening Reaction Pathways

The present invention includes a method of converting a haloepoxide (e.g., 1-chloro-2,3-epoxypropane) to an epoxide (e.g., 2,3-epoxy-1-propanol). The method includes incubating the haloepoxide and an altered haloepoxide dehalogenase in which the altered haloepoxide dehalogenase converts the haloepoxide to the epoxide. Preferred altered haloepoxide dehalogenases include, e.g., an altered 2,3-epoxy-1-propanol providing haloepoxide dehalogenases and the like.

When the altered haloepoxide dehalogenase converts the haloepoxide (e.g., 1-chloro-2,3-epoxypropane) to an epoxide (e.g., 2,3-epoxy-1-propanol) in accordance with the method described above, the invention further includes a method in which the epoxide can be converted to a polyol or to an enantiomer of the epoxide. The method includes incubating the epoxide and an altered epoxide hydrolase in which the altered epoxide hydrolase can convert the epoxide to the polyol (e.g., 1,3-propanediol, (R)-1,2-propanediol, (S)-1,2-propanediol, and the like) or to the enantiomer of the epoxide (e.g., (R)-2,3-epoxy-1-propanol, (S)-2,3-epoxy-1-propanol, and the like). Preferred altered epoxide hydrolases can include, e.g., an altered 1,3-propanediol providing epoxide hydrolase, an altered (R)-1,2-propanediol providing epoxide hydrolase, an altered (S)-1,2-propanediol providing epoxide hydrolase, an altered (R)-2,3-epoxy-1-propanol providing epoxide hydrolase, an altered (S)-2,3-epoxy-1-propanol providing epoxide hydrolase, and the like.

The invention also includes a single-step method of converting an epoxide (e.g., 2,3-epoxy-1-propanol) to a polyol (e.g., 1,3-propanediol, (R)-1,2-propanediol, (S)-1,2-propanediol, and the like) or to an enantiomer of the epoxide (e.g., (R)-2,3-epoxy-1-propanol, (S)-2,3-epoxy-1-propanol, and the like). This method can include incubating the epoxide and an altered epoxide hydrolase in which the altered epoxide hydrolase converts the epoxide to the polyol or to the enantiomer of the epoxide. Preferred altered epoxide hydrolases can include, e.g., an altered 1,3-propanediol providing epoxide hydrolase, an altered (R)-1,2-propanediol providing epoxide hydrolase, an altered (S)-1,2-propanediol providing epoxide hydrolase, an altered (R)-2,3-epoxy-1-propanol providing epoxide hydrolase, an altered (S)-2,3-epoxy-1-propanol providing epoxide hydrolase, and the like.

The present invention includes another method of converting a haloepoxide (e.g., 1-chloro-2,3-epoxypropane) to a polyol (e.g., 1,3-propanediol, (R)-1,2-propanediol, (S)-1,2-propanediol, and the like) or to an enantiomer of an epoxide (e.g., (R)-2,3-epoxy-1-propanol, (S)-2,3-epoxy-1-propanol, and the like) utilizing an altered haloepoxide dehalogenase-epoxide hydrolase. The method includes incubating the haloepoxide and an altered haloepoxide dehalogenase-epoxide hydrolase in which the altered haloepoxide dehalogenase-epoxide hydrolase converts the haloepoxide to the polyol or to the enantiomer of the epoxide. The conversion can occur through an intermediate epoxide (e.g., 2,3-epoxy-1-propanol). Furthermore, preferred altered haloepoxide dehalogenase-epoxide hydrolase can include, e.g., an altered 1,3-propanediol providing haloepoxide dehalogenase-epoxide hydrolase, an altered at least predominantly (R)-1,2-propanediol providing haloepoxide dehalogenase-epoxide hydrolase, an altered at least predominantly (S)-1,2-propanediol providing haloepoxide dehalogenase-epoxide hydrolase, an altered at least predominantly (R)-2,3-epoxy-1-propanol providing haloepoxide dehalogenase-epoxide hydrolase, an altered at least predominantly (S)-2,3-epoxy-1-propanol providing haloepoxide dehalogenase-epoxide hydrolase, and the like.

Hydrolase Alteration Methodologies

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property, e.g., an ability to efficiently catalyze any of the reactions or pathways described herein. This can include identifying any activity that can be detected, for example, in an automated or automatable format, by any of the assays in the art. A variety of related (or even unrelated) properties can be evaluated, in serial or in parallel, at the discretion of the practitioner.

The altered hydrolases, including the altered dehalogenases, of the present invention can be derived using many different techniques. For example, chimeric enzymes including identifiable components derived from two or more parental sequences can be utilized. Altered enzymes can additionally be developed using various mutagenic methods, such as cassette mutagenesis, site-directed mutagenesis, chemical mutagenesis, error-prone PCR, site-saturation mutagenesis, ensemble mutagenesis, recursive ensemble mutagenesis, and the like. These and other techniques for creating diversity are well-known and suitable for the methods of the present invention. Dehalogenase and/or other hydrolase encoding polynucleotides can also optionally be used as substrates for a variety of recombination (e.g., recursive sequence recombination) reactions. A variety of such reactions are known, including those developed by the inventor and co-workers.

The following publications describe a variety of recursive recombination procedures and/or methods which can be incorporated into such procedures: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library ImLmunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION” by del Cardayre et al., filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

The following exemplify some of the different types of preferred formats for diversity generation in the context of the present invention, including, e.g., certain recombination based diversity generation formats.

Nucleic acids can be recombined in vitro by any of a variety of techniques discussed in the references above, including, e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants are described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al. , filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids that correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579). Extensive details regarding in silico recombination methods are found in these applications. This methodology is generally applicable to the present invention in providing for recombination of hydrolase encoding sequences in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “SINGLE-STRANDED NUCLEIC ACID TEMPLATE-MEDIATED RECOMBINATION AND NUCLEIC ACID FRAGMENT ISOLATION” by Affholter, U.S. Ser. No. 09/656,549, filed Sep. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences that hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11: 1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g., a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above. “Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Overview of Diversity Generation Methods

The present invention includes methods of generating diversity in a population of hydrolase nucleic acids. The methods generally include altering at least one nucleotide of one or more members of the population of hydrolase nucleic acids using at least one modification technique, and selecting or screening altered members of the population of hydrolase nucleic acids. The methods also optionally include repeating the altering and the selecting or screening steps at least once. Modification techniques are typically selected from, e.g., recombination, oligonucleotide-directed mutagenesis, site-directed mutagenesis, error-prone PCR, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, site saturation mutagenesis, ensemble mutagenesis, recursive ensemble mutagenesis, chimeric nucleic acid multimer creation, or the like.

The selecting or screening step of these methods typically includes selecting or screening at least one altered member for an efficient catalyzation of a reaction of interest by an encoded altered hydrolase. Reactions of interest include, e.g., a conversion of at least one organic compound to at least one product; a conversion of at least one halocarbon to at least one product; a conversion of at least one first halohydrocarbon to at least one hydrocarbon, to at least one second halohydrocarbon, to at least one alcohol, to at least one haloalcohol, to at least one polyol, to at least one halopolyol, to at least one epoxide, or to at least one haloepoxide; a conversion of at least one first haloalcohol to at least one hydrocarbon, to at least one halohydrocarbon, to at least one second haloalcohol, to at least one polyol, to at least one halopolyol, to at least one epoxide, or to at least one haloepoxide; a conversion of at least one first halohydrocarbon to at least one hydrocarbon, to at least one halohydrocarbon, to at least one polyol, to at least one halopolyol, to at least one epoxide, to at least one haloepoxide, or to at least one haloalcohol; a conversion of at least one halopolyol to at least one epoxide; a conversion of at least one haloalcohol to at least one epoxide; a conversion of at least one halohydrocarbon to at least one epoxide; a conversion of at least one halohydrocarbon to at least one of a first haloalcohol isomer and at least one of a second haloalcohol isomer; a conversion of a mixture comprising at least one of a first haloalcohol isomer and at least one of a second haloalcohol isomer to at least one epoxide; a conversion of at least one haloepoxide to at least one epoxide; a conversion at least one epoxide to at least one polyol or to at least one enantiomer of the epoxide; a conversion of at least one haloepoxide to at least one polyol or to at least one enantiomer of an epoxide; or the like.

The population of hydrolase nucleic acids optionally includes various types of nucleic acids. These include, e.g., a population of halocarbon dehalogenase nucleic acids; a population of halohydrocarbon dehalogenase nucleic acids; a population of haloalcohol dehalogenase nucleic acids; a population of halopolyol dehalogenase nucleic acids; a population of first haloalcohol isomer providing halohydrocarbon dehalogenase nucleic acids; a population of second haloalcohol isomer providing halohydrocarbon dehalogenase nucleic acids; a population of haloalcohol isomer dehalogenase nucleic acids; a population of epoxide providing first haloalcohol isomer dehalogenase nucleic acids; a population of epoxide providing second haloalcohol isomer dehalogenase nucleic acids; a population of haloepoxide dehalogenase nucleic acids; a population of epoxide hydrolase nucleic acids; or the like.

The methods for generating diversity also typically include expressing the altered members of the population of hydrolase nucleic acids to provide at least one altered hydrolase. Optionally, the methods include introducing the altered members of the population of hydrolase nucleic acids into at least one cell in which the introduced altered members are expressed to provide an altered hydrolase. The altered hydrolases of the present invention typically include, e.g., an altered halocarbon dehalogenase; an altered halohydrocarbon dehalogenase; an altered haloalcohol dehalogenase; an altered halohydrocarbon-haloalcohol dehalogenase; an altered halopolyol dehalogenase; an altered haloalcohol-halopolyol dehalogenase; an altered halohydrocarbon-haloalcohol-halopolyol dehalogenase; an altered first haloalcohol isomer providing halohydrocarbon-second haloalcohol isomer providing halohydrocarbon dehalogenase; an altered epoxide providing halohydrocarbon-haloalcohol isomer dehalogenase; an altered epoxide providing first haloalcohol isomer-epoxide providing second haloalcohol isomer dehalogenase; an altered haloepoxide dehalogenase; an altered epoxide hydrolase; an altered haloepoxide dehalogenase-epoxide hydrolase; or the like.

Overview of Hydrolase Shuffling Methods

The present invention includes methods of providing a population of altered hydrolase nucleic acids that can include hybridizing a set of hydrolase nucleic acid fragments (e.g., overlapping fragments). The population of hydrolase altered nucleic acids can thereafter be provided by elongating or ligating the set of hybridized hydrolase nucleic acid fragments. The population of altered hydrolase nucleic acids derived in this manner are typically expressed to provide an altered hydrolase. The altered hydrolase nucleic acids are also optionally introduced into a cell (e.g., an organism), in which the introduced altered hydrolase nucleic acids can be expressed to provide an altered hydrolase (e.g., an altered dehalogenase or the like).

Once a population of altered hydrolase nucleic acids has been obtained, as described above, the population of altered hydrolase nucleic acids is optionally denatured. The denatured population of altered hydrolase nucleic acids can be rehybridized, and the rehybridized population of altered hydrolase nucleic acids can be extended to provide a population of further altered hydrolase nucleic acids. Thereafter, the steps of denaturing, rehybridizing, and extending are optionally repeated at least once. Furthermore, the population of further altered hydrolase nucleic acids can be selected or screened for efficient catalyzation by encoded altered hydrolases of at least one reactant to at least one product. The population of further altered hydrolase nucleic acids can also be expressed to provide a further altered hydrolase.

Altered Halocarbon Dehalogenases

The present invention includes methods of providing a population of altered halocarbon dehalogenase nucleic acids, which includes hybridizing a set of halocarbon dehalogenase nucleic acid fragments (e.g., synthesized oligonucleotides and nuclease digested nucleic acids) which can overlap, and elongating or ligating the set of hybridized halocarbon dehalogenase nucleic acid fragments. The population of altered halocarbon dehalogenase nucleic acids can thereafter be expressed to provide an altered halocarbon dehalogenase. Optionally, the altered halocarbon dehalogenase nucleic acids can be introduced into a cell (e.g., an organism) in which the introduced altered halocarbon dehalogenase nucleic acids can be expressed to provide an altered halocarbon dehalogenase.

The invention also includes optionally denaturing the population of altered halocarbon dehalogenase nucleic acids, rehybridizing the denatured population of altered halocarbon dehalogenase nucleic acids, and extending the rehybridized population of altered halocarbon dehalogenase nucleic acids to provide a population of further altered halocarbon dehalogenase nucleic acids. The steps of denaturing, rehybridizing, and extending can also be repeated at least once. The population of further altered halocarbon dehalogenase nucleic acids can also be selected or screened for an efficient catalyzation by an encoded altered halocarbon dehalogenase of a halocarbon to a product. For example, selection or screening typically includes incubating an altered halocarbon dehalogenase with a halocarbon, such as any of those described herein, and detecting one or more detectable signals indicative of enzyme activity, such as a product concentration or the like. Suitable selection and screening methods are described further below. Additionally, the population of further altered halocarbon dehalogenase nucleic acids can be expressed to provide at least one further altered halocarbon dehalogenase.

Altered Halohydrocarbon Dehalogenases

The present invention relates to methods for generating altered halohydrocarbon dehalogenases. The methods include providing a population of altered halohydrocarbon dehalogenase nucleic acids by hybridizing a set of halohydrocarbon dehalogenase nucleic acid fragments (e.g., synthesized oligonucleotides and nuclease digested nucleic acids) which can overlap, and elongating or ligating the set of hybridized halohydrocarbon dehalogenase nucleic acid fragments. Thereafter, the population of altered halohydrocarbon dehalogenase nucleic acids can be expressed to provide an altered halohydrocarbon dehalogenase. The altered halohydrocarbon dehalogenase nucleic acids are also optionally introduced into a cell (e.g., an organism), in which the introduced altered halohydrocarbon dehalogenase nucleic acids can be expressed to provide an altered halohydrocarbon dehalogenase.

Once a population of altered halohydrocarbon dehalogenase nucleic acids has been obtained, as described above, it is optionally recursively altered. These methods include denaturing the population of altered halohydrocarbon dehalogenase nucleic acids, rehybridizing the denatured population of altered halohydrocarbon dehalogenase nucleic acids, and extending the rehybridized population of altered halohydrocarbon dehalogenase nucleic acids to provide a population of further altered halohydrocarbon dehalogenase nucleic acids. Optionally, the steps of denaturing, rehybridizing, and extending can be repeated at least once. Additionally, the population of further altered halohydrocarbon dehalogenase nucleic acids can be selected or screened for an efficient catalyzation by an encoded altered halohydrocarbon dehalogenase of a first halohydrocarbon to a haloalcohol, to a second halohydrocarbon, or to a polyol. For example, selection or screening generally includes incubating an altered halohydrocarbon dehalogenase with a halohydrocarbon, such as any of those described herein, and detecting one or more detectable signals indicative of enzyme activity (e.g., a product concentration, etc.). Suitable selection and screening methods are described further below. The population of further altered halohydrocarbon dehalogenase nucleic acids can also be expressed to provide a further altered halohydrocarbon dehalogenase.

Altered Haloalcohol Dehalogenases

The invention also relates to a method of providing a population of altered haloalcohol dehalogenase nucleic acids that includes hybridizing a set of haloalcohol dehalogenase nucleic acid fragments (e.g., synthesized oligonucleotides and nuclease digested nucleic acids) which can overlap, and elongating or ligating the set of hybridized haloalcohol dehalogenase nucleic acid fragments. The population of altered haloalcohol dehalogenase nucleic acids can thereafter be expressed to provide an altered haloalcohol dehalogenase. Optionally, the altered haloalcohol dehalogenase nucleic acids can be introduced into a cell (e.g., an organism) in which the introduced altered haloalcohol dehalogenase nucleic acids can be expressed to provide an altered haloalcohol dehalogenase.

The present invention also optionally includes denaturing the population of altered haloalcohol dehalogenase nucleic acids, rehybridizing the denatured population of altered haloalcohol dehalogenase nucleic acids, and extending the rehybridized population of altered haloalcohol dehalogenase nucleic acids to provide a population of further altered haloalcohol dehalogenase nucleic acids. The steps of denaturing, rehybridizing, and extending can also be repeated at least once. The population of further altered haloalcohol dehalogenase nucleic acids can also be selected or screened for an efficient catalyzation by an encoded altered haloalcohol dehalogenase of a first haloalcohol to an epoxide, to a polyol, or to a second haloalcohol. To further illustrate, selection or screening typically includes incubating an altered haloalcohol dehalogenase with a haloalcohol, such as any of those described herein, and detecting one or more detectable signals indicative of enzyme activity, such as a product concentration or the like. Suitable selection and screening methods are described further below. Additionally, the population of further altered haloalcohol dehalogenase nucleic acids can be expressed to provide at least one further altered haloalcohol dehalogenase.

Altered Halohydrocarbon-haloalcohol Dehalogenases

The present invention includes methods for creating altered halohydrocarbon-haloalcohol dehalogenases. The methods include providing a population of altered halohydrocarbon-haloalcohol dehalogenase nucleic acids by hybridizing a set of halohydrocarbon dehalogenase nucleic acid fragments or haloalcohol dehalogenase nucleic acid fragments or both (e.g., synthesized oligonucleotides and nuclease digested nucleic acids) which can overlap, and elongating or ligating the set of hybridized halohydrocarbon dehalogenase nucleic acid fragments or haloalcohol dehalogenase nucleic acid fragments or both. Thereafter, the population of altered halohydrocarbon-haloalcohol dehalogenase nucleic acids can be expressed to provide an altered halohydrocarbon-haloalcohol dehalogenase. The altered halohydrocarbon-haloalcohol dehalogenase nucleic acids are also optionally introduced into a cell (e.g., an organism) in which the introduced altered halohydrocarbon-haloalcohol dehalogenase nucleic acids can be expressed to provide an altered halohydrocarbon-haloalcohol dehalogenase.

The invention also includes optionally denaturing the population of altered halohydrocarbon-haloalcohol dehalogenase nucleic acids, rehybridizing the denatured population of altered halohydrocarbon-haloalcohol dehalogenase nucleic acids, and extending the rehybridized population of altered halohydrocarbon-haloalcohol dehalogenase nucleic acids to provide a population of further altered halohydrocarbon-haloalcohol dehalogenase nucleic acids. The steps of denaturing, rehybridizing, and extending can also be repeated. Additionally, the population of further altered halohydrocarbon-haloalcohol dehalogenase nucleic acids can be selected or screened for an efficient catalyzation by an encoded altered halohydrocarbon-haloalcohol dehalogenase of a halohydrocarbon to an epoxide, to a polyol, or to a haloalcohol. For example, selection or screening generally includes incubating an altered halohydrocarbon-haloalcohol dehalogenase with a halohydrocarbon, such as any of those described herein, and detecting one or more detectable signals indicative of enzyme activity, such as a product concentration or the like. Suitable selection and screening methods are described further below. The population of further altered halohydrocarbon-haloalcohol dehalogenase nucleic acids is also optionally expressed to provide a further altered halohydrocarbon-haloalcohol dehalogenase.

Altered Halopolyol Dehalogenases

The present invention relates to methods for generating altered halopolyol dehalogenases. These methods include providing a population of altered halopolyol dehalogenase nucleic acids by hybridizing a set of halopolyol dehalogenase nucleic acid fragments (e.g., synthesized oligonucleotides and nuclease digested nucleic acids) which can overlap, and elongating or ligating the set of hybridized halopolyol dehalogenase nucleic acid fragments. Thereafter, the population of altered halopolyol dehalogenase nucleic acids can be expressed to provide an altered halopolyol dehalogenase. The altered halopolyol dehalogenase nucleic acids are also optionally introduced into a cell (e.g., an organism) in which the introduced altered halopolyol dehalogenase nucleic acids can be expressed to provide an altered halopolyol dehalogenase.

Once a population of altered halopolyol dehalogenase nucleic acids has been obtained, as described above, it is optionally recursively altered. These methods include denaturing the population of altered halopolyol dehalogenase nucleic acids, rehybridizing the denatured population of altered halopolyol dehalogenase nucleic acids, and extending the rehybridized population of altered halopolyol dehalogenase nucleic acids to provide a population of further altered halopolyol dehalogenase nucleic acids. Optionally, the steps of denaturing, rehybridizing, and extending can be repeated one or more times. Additionally, the population of further altered halopolyol dehalogenase nucleic acids can be selected or screened for an efficient catalyzation by an encoded altered halopolyol dehalogenase of a halopolyol to an epoxide. To further illustrate, selection or screening typically includes incubating an altered halopolyol dehalogenase with a halopolyol, such as any of those described herein, and detecting one or more detectable signals indicative of enzyme activity, such as a product concentration or the like. Suitable selection and screening methods are described further below. The population of further altered halopolyol dehalogenase nucleic acids can also be expressed to provide a further altered halopolyol dehalogenase.

Altered Haloalcohol-halopolyol Dehalogenases

The invention also includes a method of providing a population of altered haloalcohol-halopolyol dehalogenase nucleic acids. The method includes hybridizing a set of haloalcohol dehalogenase nucleic acid fragments or halopolyol dehalogenase nucleic acid fragments or both (e.g., synthesized oligonucleotides and nuclease digested nucleic acids) which can overlap, and elongating or ligating the set of hybridized haloalcohol dehalogenase nucleic acid fragments or halopolyol dehalogenase nucleic acid fragments or both. The population of altered haloalcohol-halopolyol dehalogenase nucleic acids can also be expressed to provide an altered haloalcohol-halopolyol dehalogenase. Optionally, the altered haloalcohol-halopolyol dehalogenase nucleic acids can be introduced into a cell (e.g., an organism) in which the introduced altered haloalcohol-halopolyol dehalogenase nucleic acids can be expressed to provide an altered haloalcohol-halopolyol dehalogenase.

After a population of altered haloalcohol-halopolyol dehalogenase nucleic acids has been obtained, as described above, the population is optionally iteratively altered. These methods include denaturing the population of altered haloalcohol-halopolyol dehalogenase nucleic acids, rehybridizing the denatured population of altered haloalcohol-halopolyol dehalogenase nucleic acids, and extending the rehybridized population of altered haloalcohol-halopolyol dehalogenase nucleic acids to provide a population of further altered haloalcohol-halopolyol dehalogenase nucleic acids. Thereafter, the steps of denaturing, rehybridizing, and extending can be repeated one or more times. The population of further altered haloalcohol-halopolyol dehalogenase nucleic acids can also be selected or screened for an efficient catalyzation by an encoded altered haloalcohol-halopolyol dehalogenase of a haloalcohol to an epoxide. For example, selection or screening generally includes incubating an altered haloalcohol-halopolyol dehalogenase with a haloalcohol, such as those described herein, and detecting one or more detectable signals indicative of enzyme activity, such as a product concentration or the like. Suitable selection and screening methods are described further below. Additionally, the population of further altered haloalcohol-halopolyol dehalogenase nucleic acids can be expressed to provide a further altered haloalcohol-halopolyol dehalogenase.

Altered Halohydrocarbon-haloalcohol-halopolyol Dehalogenases

The invention also relates to a method of providing a population of altered halohydrocarbon-haloalcohol-halopolyol dehalogenase nucleic acids. The method includes hybridizing a set of halohydrocarbon dehalogenase nucleic acid fragments or haloalcohol dehalogenase nucleic acid fragments or halopolyol dehalogenase nucleic acid fragments or any combination thereof (e.g., synthesized oligonucleotides and nuclease digested nucleic acids) which can overlap, and elongating or ligating the set of hybridized halohydrocarbon dehalogenase nucleic acid fragments or haloalcohol dehalogenase nucleic acid fragments or halopolyol dehalogenase nucleic acid fragments or any combination thereof. The population of altered halohydrocarbon-haloalcohol-halopolyol dehalogenase nucleic acids is optionally expressed to provide an altered halohydrocarbon-haloalcohol-halopolyol dehalogenase. Additionally, the altered halohydrocarbon-haloalcohol-halopolyol dehalogenase nucleic acids can be introduced into a cell (e.g., an organism) in which the introduced altered halohydrocarbon-haloalcohol-halopolyol dehalogenase nucleic acids can be expressed to provide an altered halohydrocarbon-haloalcohol-halopolyol dehalogenase.

The invention also includes optionally denaturing the population of altered halohydrocarbon-haloalcohol-halopolyol dehalogenase nucleic acids, rehybridizing the denatured population of altered halohydrocarbon-haloalcohol-halopolyol dehalogenase nucleic acids, and extending the rehybridized population of altered halohydrocarbon-haloalcohol-halopolyol dehalogenase nucleic acids to provide a population of further altered halohydrocarbon-haloalcohol-halopolyol dehalogenase nucleic acids. The steps of denaturing, rehybridizing, and extending can also be repeated at least once. In addition, the population of further altered halohydrocarbon-haloalcohol-halopolyol dehalogenase nucleic acids can be selected or screened for an efficient catalyzation by an encoded altered halohydrocarbon-haloalcohol-halopolyol dehalogenase of a halohydrocarbon to an epoxide. To further illustrate, selection or screening generally includes incubating an altered halohydrocarbon-haloalcohol-halopolyol dehalogenase with a particular halohydrocarbon, such as any of those described herein, and detecting one or more detectable signals indicative of enzyme activity, such as a product concentration or the like. Suitable selection and screening methods are described further below. The population of further altered halohydrocarbon-haloalcohol-halopolyol dehalogenase nucleic acids can also be expressed to provide a further altered halohydrocarbon-haloalcohol-halopolyol dehalogenase.

Altered First Haloalcohol Isomer Providing Halohydrocarbon-second Haloalcohol Isomer Providing Halohydrocarbon Dehalogenases

The present invention also relates to methods for providing altered first haloalcohol isomer providing halohydrocarbon-second haloalcohol isomer providing halohydrocarbon dehalogenases. The methods include providing population of altered first haloalcohol isomer providing halohydrocarbon-second haloalcohol isomer providing halohydrocarbon dehalogenase nucleic acids by hybridizing a set of first haloalcohol isomer providing halohydrocarbon dehalogenase nucleic acid fragments or second haloalcohol isomer providing halohydrocarbon dehalogenase nucleic acid fragments or both (e.g., synthesized oligonucleotides and nuclease digested nucleic acids) which can overlap, and elongating or ligating the set of hybridized first haloalcohol isomer providing halohydrocarbon dehalogenase nucleic acid fragments or second haloalcohol isomer providing halohydrocarbon dehalogenase nucleic acid fragments or both. Thereafter, the population of altered first haloalcohol isomer providing halohydrocarbon-second haloalcohol isomer providing halohydrocarbon dehalogenase nucleic acids can be expressed to provide an altered first haloalcohol isomer providing halohydrocarbon-second haloalcohol isomer providing halohydrocarbon dehalogenase. Moreover, the altered first haloalcohol isomer providing halohydrocarbon-second haloalcohol isomer providing halohydrocarbon dehalogenase nucleic acids can be introduced into a cell (e.g., an organism) in which the introduced altered first haloalcohol isomer providing halohydrocarbon-second haloalcohol isomer providing halohydrocarbon dehalogenase nucleic acids can be expressed to provide at least one altered first haloalcohol isomer providing halohydrocarbon-second haloalcohol isomer providing halohydrocarbon dehalogenase.

Once a population of altered first haloalcohol isomer providing halohydrocarbon-second haloalcohol isomer providing halohydrocarbon dehalogenase nucleic acids has been obtained, it is optionally recursively altered. This can include denaturing the population of altered first haloalcohol isomer providing halohydrocarbon-second haloalcohol isomer providing halohydrocarbon dehalogenase nucleic acids, rehybridizing the denatured population of altered first haloalcohol isomer providing halohydrocarbon-second haloalcohol isomer providing halohydrocarbon dehalogenase nucleic acids, and extending the rehybridized population of altered first haloalcohol isomer providing halohydrocarbon-second haloalcohol isomer providing halohydrocarbon dehalogenase nucleic acids to provide a population of further altered first haloalcohol isomer providing halohydrocarbon-second haloalcohol isomer providing halohydrocarbon dehalogenase nucleic acids. The steps of denaturing, rehybridizing, and extending are optionally repeated one or more times. The population of further altered first haloalcohol isomer providing halohydrocarbon-second haloalcohol isomer providing halohydrocarbon dehalogenase nucleic acids can also be selected or screened for an efficient catalyzation by an encoded altered first haloalcohol isomer providing halohydrocarbon-second haloalcohol isomer providing halohydrocarbon dehalogenase of a halohydrocarbon to a first haloalcohol isomer and a second haloalcohol isomer. To further illustrate, selection or screening generally includes incubating an altered first haloalcohol isomer providing halohydrocarbon-second haloalcohol isomer providing halohydrocarbon dehalogenase with a halohydrocarbon, such as any of those described herein, and detecting one or more detectable signals indicative of enzyme activity, such as a product concentration or the like. Suitable selection and screening methods are described further below. In addition, the population of further altered first haloalcohol isomer providing halohydrocarbon-second haloalcohol isomer providing halohydrocarbon dehalogenase nucleic acids can be expressed to provide a further altered first haloalcohol isomer providing halohydrocarbon-second haloalcohol isomer providing halohydrocarbon dehalogenase.

Altered Epoxide Providing Halohydrocarbon-haloalcohol Isomer Dehalogenases

The invention also relates to a method of providing a population of altered epoxide providing halohydrocarbon-haloalcohol isomer dehalogenase nucleic acids. The method includes hybridizing a set of halohydrocarbon dehalogenase nucleic acid fragments or haloalcohol isomer dehalogenase nucleic acid fragments or both (e.g., synthesized oligonucleotides and nuclease digested nucleic acids) which can overlap, and elongating or ligating the set of hybridized halohydrocarbon dehalogenase nucleic acid fragments or haloalcohol isomer dehalogenase nucleic acid fragments or both. The method also includes expressing the population of altered epoxide providing halohydrocarbon-haloalcohol isomer dehalogenase nucleic acids to provide an altered epoxide providing halohydrocarbon-haloalcohol isomer dehalogenase. Optionally, the altered epoxide providing halohydrocarbon-haloalcohol isomer dehalogenase nucleic acids can be introduced into a cell (e.g., an organism) in which introduced altered epoxide providing halohydrocarbon-haloalcohol isomer dehalogenase nucleic acids can be expressed to provide an altered epoxide providing halohydrocarbon-haloalcohol isomer dehalogenase.

After a population of altered epoxide providing halohydrocarbon-haloalcohol isomer dehalogenase nucleic acids has been obtained, as described above, the population is optionally iteratively altered. This can include denaturing the population of altered epoxide providing halohydrocarbon-haloalcohol isomer dehalogenase nucleic acids, rehybridizing the denatured population of altered epoxide providing halohydrocarbon-haloalcohol isomer dehalogenase nucleic acids, and extending the rehybridized population of altered epoxide providing halohydrocarbon-haloalcohol isomer dehalogenase nucleic acids to provide a population of further altered epoxide providing halohydrocarbon-haloalcohol isomer dehalogenase nucleic acids. The steps of denaturing, rehybridizing, and extending are optionally repeated one or more times. The population of further altered epoxide providing halohydrocarbon-haloalcohol isomer dehalogenase nucleic acids can also be selected or screened for an efficient catalyzation by an encoded altered epoxide providing halohydrocarbon-haloalcohol isomer dehalogenase of a halohydrocarbon to an epoxide. For example, selection or screening typically includes incubating an altered epoxide providing halohydrocarbon-haloalcohol isomer dehalogenase with a particular halohydrocarbon, such as any of those described herein, and detecting one or more detectable signals indicative of enzyme activity, such as a product concentration or the like. Suitable selection and screening methods are described further below. Additionally, the population of further altered epoxide providing halohydrocarbon-haloalcohol isomer dehalogenase nucleic acids can be expressed to provide a further altered epoxide providing halohydrocarbon-haloalcohol isomer dehalogenase.

Altered Epoxide Providing First Haloalcohol Isomer-epoxide Providing Second Haloalcohol Isomer Dehalogenase

The present invention includes methods for generating altered epoxide providing first haloalcohol isomer-epoxide providing second haloalcohol isomer dehalogenases. The methods include providing a population of altered epoxide providing first haloalcohol isomer-epoxide providing second haloalcohol isomer dehalogenase nucleic acids by hybridizing a set of epoxide providing first haloalcohol isomer dehalogenase nucleic acid fragments or epoxide providing second haloalcohol isomer dehalogenase nucleic acid fragments or both (e.g., synthesized oligonucleotides and nuclease digested nucleic acids) which can overlap, and elongating or ligating the set of hybridized epoxide providing first haloalcohol isomer dehalogenase nucleic acid fragments or epoxide providing second haloalcohol isomer dehalogenase nucleic acid fragments or both. The population of altered epoxide providing first haloalcohol isomer-epoxide providing second haloalcohol isomer dehalogenase nucleic acids can also be expressed to provide an altered epoxide providing first haloalcohol isomer-epoxide providing second haloalcohol isomer dehalogenase. Optionally, the altered epoxide providing first haloalcohol isomer-epoxide providing second haloalcohol isomer dehalogenase nucleic acids can be introduced into a cell (e.g., an organism) in which the introduced altered epoxide providing first haloalcohol isomer-epoxide providing second haloalcohol isomer dehalogenase nucleic acids can be expressed to provide an altered epoxide providing first haloalcohol isomer-epoxide providing second haloalcohol isomer dehalogenase.

Once a population of altered epoxide providing first haloalcohol isomer-epoxide providing second haloalcohol isomer dehalogenase nucleic acids has been provided, as described above, it is optionally recursively altered. This can include denaturing the population of altered epoxide providing first haloalcohol isomer-epoxide providing second haloalcohol isomer dehalogenase nucleic acids, rehybridizing the denatured population of altered epoxide providing first haloalcohol isomer-epoxide providing second haloalcohol isomer dehalogenase nucleic acids, and extending the rehybridized population of altered epoxide providing first haloalcohol isomer-epoxide providing second haloalcohol isomer dehalogenase nucleic acids to provide a population of further altered epoxide providing first haloalcohol isomer-epoxide providing second haloalcohol isomer dehalogenase nucleic acids. The steps of denaturing, rehybridizing, and extending can also optionally be repeated one or more times. In addition, the population of further altered epoxide providing first haloalcohol isomer-epoxide providing second haloalcohol isomer dehalogenase nucleic acids can also be selected or screened for an efficient catalyzation by an encoded altered epoxide providing first haloalcohol isomer-epoxide providing second haloalcohol isomer dehalogenase of a mixture of a first and a second haloalcohol isomer to an epoxide. To further illustrate, selection or screening typically includes incubating an altered epoxide providing first haloalcohol isomer-epoxide providing second haloalcohol isomer dehalogenase with a mixture of haloalcohol isomers, such as isomers of any of the haloalcohols described herein, and detecting one or more detectable signals indicative of enzyme activity, such as a product (e.g., a desired epoxide) concentration or the like. Suitable selection and screening methods are described further below. Additionally, the population of further altered epoxide providing first haloalcohol isomer-epoxide providing second haloalcohol isomer dehalogenase nucleic acids can be expressed to provide a further altered epoxide providing first haloalcohol isomer-epoxide providing second haloalcohol isomer dehalogenase.

Altered Haloepoxide Dehalogenases

The present invention also relates to a method of providing a population of altered haloepoxide dehalogenase nucleic acids that includes hybridizing a set of haloepoxide dehalogenase nucleic acid fragments (e.g., synthesized oligonucleotides and nuclease digested nucleic acids) which can overlap, and elongating or ligating the set of hybridized haloepoxide dehalogenase nucleic acid fragments. The population of altered haloepoxide dehalogenase nucleic acids can also be expressed to provide an altered haloepoxide dehalogenase. Optionally, the altered haloepoxide dehalogenase nucleic acids can be introduced into a cell (e.g., an organism) in which the introduced altered haloepoxide dehalogenase nucleic acids can be expressed to provide an altered haloepoxide dehalogenase.

The invention also includes optionally denaturing the population of altered haloepoxide dehalogenase nucleic acids, rehybridizing the denatured population of altered haloepoxide dehalogenase nucleic acids, and extending the rehybridized population of altered haloepoxide dehalogenase nucleic acids to provide a population of further altered haloepoxide dehalogenase nucleic acids. The steps of denaturing, rehybridizing, and extending are also optionally repeated one or more times. In addition, the population of further altered haloepoxide dehalogenase nucleic acids can be selected or screened for an efficient catalyzation by an encoded altered haloepoxide dehalogenase of a haloepoxide to an epoxide. For example, selection or screening generally includes incubating an altered haloepoxide dehalogenase with a haloepoxide, such as any of those described herein, and detecting one or more detectable signals indicative of enzyme activity, such as a product concentration or the like. Suitable selection and screening methods are described further below. The population of further altered haloepoxide dehalogenase nucleic acids can also be expressed to provide a further altered haloepoxide dehalogenase.

Altered Epoxide Hydrolases

The present invention also relates to methods for generating altered epoxide hydrolases. The methods include providing a population of altered epoxide hydrolase nucleic acids by hybridizing a set of epoxide hydrolase nucleic acid fragments (e.g., synthesized oligonucleotides and nuclease digested nucleic acids) and elongating the set of hybridized epoxide hydrolase nucleic acid fragments. The population of altered epoxide hydrolase nucleic acids can also be expressed to provide an altered epoxide hydrolase. Optionally, the altered epoxide hydrolase nucleic acids can be introduced into a cell (e.g., an organism) in which the introduced altered epoxide hydrolase nucleic acids can be expressed to provide an altered epoxide hydrolase.

After a population of altered epoxide hydrolase nucleic acids has been obtained, as described above, the population can also be iteratively altered. This can include denaturing the population of altered epoxide hydrolase nucleic acids, rehybridizing the denatured population of altered epoxide hydrolase nucleic acids, and extending the rehybridized population of altered epoxide hydrolase epoxide hydrolase nucleic acids to provide a population of further altered epoxide hydrolase nucleic acids. The steps of denaturing, rehybridizing, and extending are optionally repeated at least once. Moreover, the population of further altered epoxide hydrolase nucleic acids can be selected or screened for an efficient catalyzation by an encoded altered epoxide hydrolase of an epoxide to a polyol or to an enantiomer of the epoxide. To further illustrate, selection or screening generally includes incubating an altered epoxide hydrolase with an epoxide, such as any of those described herein, and detecting one or more detectable signals indicative of enzyme activity, such as a product concentration or the like. Suitable selection and screening methods are described further below. The population of further altered epoxide hydrolase nucleic acids can also be expressed to provide a further altered epoxide hydrolase.

Altered Haloepoxide Dehalogenase-epoxide Hydrolases

The invention also relates to a method of providing a population of altered haloepoxide dehalogenase-epoxide hydrolase nucleic acids that includes hybridizing a set of haloepoxide dehalogenase nucleic acid fragments or epoxide hydrolase nucleic acid fragments or both (e.g., synthesized oligonucleotides and nuclease digested nucleic acids) which can overlap, and elongating or ligating the set of hybridized haloepoxide dehalogenase nucleic acid fragments or epoxide hydrolase nucleic acid fragments or both. Thereafter, the population of altered haloepoxide dehalogenase-epoxide hydrolase nucleic acids can be expressed to provide an altered haloepoxide dehalogenase-epoxide hydrolase. Optionally, the altered haloepoxide dehalogenase-epoxide hydrolase nucleic acids can be introduced into a cell (e.g., an organism) in which the introduced altered haloepoxide dehalogenase-epoxide hydrolase nucleic acids can be expressed to provide an altered haloepoxide dehalogenase-epoxide hydrolase.

Once a population of altered haloepoxide dehalogenase-epoxide hydrolase nucleic acids has been obtained, the population is optionally recursively altered. This can include denaturing the population of altered haloepoxide dehalogenase-epoxide hydrolase, rehybridizing the denatured population of altered haloepoxide dehalogenase-epoxide hydrolase nucleic acids, and extending the rehybridized population of altered haloepoxide dehalogenase-epoxide hydrolase nucleic acids to provide a population of further altered haloepoxide dehalogenase-epoxide hydrolase nucleic acids. The steps of denaturing, rehybridizing, and extending are optionally repeated one or more times to further evolve the enzyme. Additionally, the population of further altered haloepoxide dehalogenase-epoxide hydrolase nucleic acids can be selected or screened for an efficient catalyzation by an encoded altered haloepoxide dehalogenase-epoxide hydrolase of a haloepoxide to a polyol or to an enantiomer of an epoxide. For example, selection or screening typically includes incubating an altered haloepoxide dehalogenase-epoxide hydrolase with a haloepoxide, such as any of those described herein, and detecting one or more detectable signals indicative of enzyme activity, such as a product concentration or the like. Suitable selection and screening methods are described further below. The population of further altered haloepoxide dehalogenase-epoxide hydrolase nucleic acids can also be expressed to provide a further altered haloepoxide dehalogenase-epoxide hydrolase.

Target Dehalogenase and Other Hydrolase Gene Sequence Preparation

Virtually any set of dehalogenase or other hydrolase nucleic acid fragments can be recombined by the methods described in this disclosure to produce new altered variants having the activities noted throughout. As such, no attempt is made to identify all of the known nucleic acids. Certain preferred sets of dehalogenase or other hydrolase nucleic acid fragments (e.g., synthesized oligonucleotides and/or nuclease digested nucleic acids) can be selected or otherwise derived from one or more of: accession number AJ012627, accession number AF060871, accession number AF017179, accession number L49435, accession number M26950, accession number D14594, accession number CAB01264, accession number A28028, accession number AL079353, accession number AL022121, accession number AL123456, accession number AL035636, accession number AE002084, accession number AE000513, accession number AL137165, accession number AL133252, accession number AL132707, accession number AJ005843, accession number AL079345, accession number AL031124, accession number AF043240, accession number X84038, accession number M81691, and the like. Relevant reference information pertaining to these sequences includes the following:

(1) Accession number AJ012627 corresponds to Mycobacterium sp. DhaAf gene, strain GP1. Poelarends, G. J. et al. (1999) “Degradation of 1,2-Dibromoethane by Mycobacterium sp.,” J. Bacteriol. 181(7):2050-2058;

(2) Accession numbers AF060871, AF017179, and L49435 correspond to Rhodococcus rhodochrous plasmid pRTL1 insertion sequence IS2112 putative transposase (tnpA), putative site specific recombinase (invA), haloalkane dehalogenase (dhaA), hypothetical alcohol dehydrogenase, and hypothetical aldehyde dehydrogenase precursor, genes, complete cds. Kulakova, A. N. et al. (1997) “The Plasmid-Located Haloalkane Dehalogenase Gene from Rhodococcus rhodochrous NCIMB 13064,” Microbiology 143(Pt 1): 109-115 and Kulakov, L. A. et al. (1999) “Characterization of IS2112, A New Insertion Sequence from Rhodococcus, and its Relationship with Mobile Elements Belonging to the IS110 Family,” Microbiology 145(3):561-568;

(3) Accession number M26950 corresponds to X. autotrophicus haloalkane dehalogenase (dhlA) gene, complete cds. Janssen, D. B. et al. (1989) “Cloning of 1,2-dichloroethane degradation genes of Xanthobacter autotrophicus GJ10 and Expression and Sequencing of the dhlA Gene,” J. Bacteriol. 171(12):6791-6799;

(4) Accession number D14594 corresponds to Sphingomonas paucimobilis linB gene for 1,3,4,6-tetrachloro-1,4-cyclohexadiene hydrolase, complete cds. Nagata, Y. et al. (1993) “Cloning and Sequencing of a Dehalogenase Gene Encoding an Enzyme with Hydrolase activity Involved in the Degradation of Gamma-Hexachlorocyclohexane in Pseudomonas paucimobilis,” J. Bacteriol. 175(20):6403-6410;

(5) Accession number CAB01264 is derived from Mycobacterium tuberculosis. Cole, S. T. et al. (1998) “Deciphering the Biology of Mycobacterium tuberculosis from the Complete Genome Sequence,” Nature 393(6685):537-544;

(6) Accession number A28028 is derived from R. reniformis luciferase cDNA. WO 92/15673-A (1992) “CLONING AND EXPRESSION OF RENILLA LUCIFERASE;”

(7) Accession number AL079353 corresponds to Streptomyces coelicolor cosmid H17. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(8) Accession numbers AL022121 and AL123456 correspond to Mycobacterium tuberculosis H37Rv complete genome; segment 155/162 Cole, S. T. et al. (1998) “Deciphering the Biology of Mycobacterium tuberculosis from the Complete Genome Sequence,” Nature 393(6685):537-544;

(9) Accession number AL035636 corresponds to Streptomyces coelicolor cosmid H5. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(10) Accession numbers AE002084 and AE000513 correspond to Deinococcus radiodurans R1 section 221 or 229 of the complete chromosome 1. White, 0. et al. (1999) “Genome Sequence of the Radioresistant Bacterium Deinococcus radiodurans R1,” Science 286:1571-1577;

(11) Accession number AL137165 corresponds to Streptomyces coelicolor cosmid F42. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(12) Accession number AL133252 corresponds to Streptomyces coelicolor cosmid E46. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(13) Accession number AL132707 corresponds to Streptomyces coelicolor cosmid F51. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(14) Accession number AJ005843 corresponds to Burkholderia cepacia gene encoding cryptic haloacid dehalogenase 1. Tsang, J. S. and Sam, L. (1999) “Cloning and Characterization of a Cryptic Haloacid Dehalogenase from Burkholderia cepacia MBA4,” J. Bacteriol. 181(19):6003-6009;

(15) Accession number AL079345 corresponds to Streptomyces coelicolor cosmid E68. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(16) Accession number AL031124 corresponds to Streptomyces coelicolor cosmid IC2. Redenbach, M. et al. (1996) “A Set of Ordered Cosmids and a Detailed Genetic and Physical Map for the 8 Mb Streptomyces coelicolor A3(2) Chromosome,” Mol. Microbiol. 21(1):77-96;

(17) Accession number AF043240 corresponds to Ancylobacter aquaticus haloacid dehalogenase gene, partial cds. Fortin, N. et al. “Molecular Analysis of Bacterial Isolates and Total Community DNA from Kraft Pulp Mill Effluent Treatment Systems,” unpublished;

(18) Accession number X84038 corresponds to Xanthobacter autotrophicus insertion element and dhlB gene. van der Ploeg, J. et al. (1995) “Adaptation of Xanthobacter autotrophicus GJ10 to Bromoacetate due to Activation and Mobilization of the Haloacetate Dehalogenase Gene by Insertion Element IS1247,” J. Bacteriol. 177(5):1348-1356; and,

(19) Accession number M81691 corresponds to Xanthobacter autotrophicus haloacid dehalogenase (dhlB) gene, complete cds. van der Ploeg, J. et al. (1991) “Characterization of the Haloacid Dehalogenase from Xanthobacter autotrophicus GJ10 and Sequencing of the dhlB Gene,” J. Bacteriol. 173:7925-7933.

In general, target sequences can be prepared using various methods or combinations thereof, including certain DNA synthetic techniques (e.g., mononucleotide- and/or trinucleotide-based synthesis, reverse-transcription, etc.), DNA amplification, nuclease digestion, etc. Searchable sequence information is available from nucleic acid databases can be utilized during the nucleic acid sequence selection and/or design processes. Genbank®, Entrez®, EMBL, DDBJ, GSDB, NDB and the NCBI are examples of public database/search services that can be accessed. These databases are generally available via the internet or on a contract basis from a variety of companies specializing in genomic information generation and/or storage. These and other helpful resources are readily available and known to those of skill.

The sequence of a polynucleotide to be used in any of the methods of the present invention can also be readily determined using techniques well-known to those of skill, including Maxam-Gilbert, Sanger Dideoxy, and Sequencing by Hybridization methods. For general descriptions of these processes consult, e.g., Stryer, L., Biochemistry (4^{th} Ed.) W.H. Freeman and Company, New York, 1995 (Stryer) and Lewin, B. Genes VI Oxford University Press, Oxford, 1997 (Lewin). See also, Maxam, A. M. and Gilbert, W. (1977) “A New Method for Sequencing DNA,” Proc. Natl. Acad. Sci. 74:560-564, Sanger, F. et al. (1977) “DNA Sequencing with Chain-Terminating Inhibitors,” Proc. Natl. Acad. Sci. 74:5463-5467, Hunkapiller, T. et al. (1991) “Large-Scale and Automated DNA Sequence Determination,” Science 254:59-67, and Pease, A. C. et al. (1994) “Light-Generated Oligonucleotide Arrays for Rapid DNA Sequence Analysis,” Proc. Natl. Acad. Sci. 91:5022-5026. See also, Berger, Sambrook, and Ausubel, supra.

When shuffling homologous nucleic acids, e.g., from a dehalogenase or another hydrolase family, the present invention optionally includes aligning homologous nucleic acid sequences or regions of similarity. For example, in one aspect, the invention relates to a method of recombining at least two parental nucleic acids. In an embodiment of this method, the composition of nucleic acids to be recombined is provided by aligning homologous nucleic acid sequences to select conserved regions of sequence identity and regions of sequence diversity. Dehalogenase and/or other hydrolase fragments can, e.g., then synthesized to correspond to at least one region of sequence diversity. Similarly, an aspect of the invention can include deriving the sequences of a second set of dehalogenase and/or other hydrolase fragments from first round selected nucleic acids by aligning those first round sequences to identify regions of identity and regions of diversity.

In the processes of sequence comparison and homology determination, one sequence, e.g., one fragment or subsequence of a gene sequence to be recombined, can be used as a reference against which other test nucleic acid sequences are compared. This comparison can be accomplished with the aid of a sequence comparison instruction set, i.e., algorithm, or by visual inspection. When an algorithm is employed, test and reference sequences are input into a computer, subsequence coordinates are designated, as necessary, and sequence algorithm program parameters are specified. The algorithm then calculates the percent sequence identity for the test nucleic acid sequence(s) relative to the reference sequence, based on the specified program parameters. Among other things, a sequence comparison algorithm can provide sets of nucleic acid sequences to be synthesized and used to facilitate the recombination process. Integrated systems that are relevant to the invention are discussed further, infra.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of sequence similarity over the comparison window) generated by the various methods is selected.

The term sequence identity means that two polynucleotide sequences are identical (i.e., on a nucleotide-by-nucleotide basis) over a window of comparison. The term “percentage of sequence identity” is calculated by comparing two optimally aligned sequences over the window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

As applied to polypeptides, the term substantial identity means that two peptide sequences, when optimally aligned, such as by the programs GAP or BESTFIT using default gap weights (described in detail below), share at least about 80 percent sequence identity, preferably at least about 90 percent sequence identity, more preferably at least about 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

A preferred example of an algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also, W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., 1977, Nuc. Acids Res. 25: 3389-3402 and Altschul et al., 1990, J. Mol. Biol. 215: 403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. U.S.A. 89: 10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Natl. Acad. Sci. U.S.A. 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35:351-360. The method used is similar to the method described by Higgins & Sharp, 1989, CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12: 387-395).

Another preferred example of an algorithm that is suitable for multiple DNA and amino acid sequence alignments is the CLUSTALW program (Thompson, J. D. et al., 1994, Nucl. Acids. Res. 22: 4673-4680). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, 1992, Proc. Natl. Acad. Sci. U.S.A. 89: 10915-10919).

Target Sequence Acquisition

After sequence information has been obtained as described above, that information can be used to design and synthesize target nucleic acid sequences corresponding to, e.g., dehalogenase or other hydrolase nucleic acid fragments (e.g., for the oligonucleotide and in silico shuffling approaches noted above. These sequences can be synthesized utilizing various solid-phase strategies involving mononucleotide and/or trinucleotide-based phosphoramidite coupling chemistry. In these approaches, nucleic acid sequences are synthesized by the sequential addition of activated monomers and/or trimers to an elongating polynucleotide chain. See, e.g., Caruthers, M. H. et al. (1992) Meth. Enzymol. 211:3-20.

In the formats involving trimers, trinucleotide phosphoramidites representing codons for all 20 amino acids are used to introduce entire codons into the growing oligonucleotide sequences being synthesized. The details on synthesis of trinucleotide phosphoramidites, their subsequent use in oligonucleotide synthesis, and related issues are described in, e.g., Virnekäs, B., et al. (1994) Nucleic Acids Res., 22, 5600-5607, Kayushin, A. L. et al. (1996) Nucleic Acids Res., 24, 3748-3755, Huse, U.S. Pat. No. 5,264,563 “PROCESS FOR SYNTHESIZING OLIGONUCLEOTIDES WITH RANDOM CODONS,” Lyttle et al., U.S. Pat. No. 5,717,085 “PROCESS FOR PREPARING CODON AMIDITES,” Shortle et al., U.S. Pat. No. 5,869,644 “SYNTHESIS OF DIVERSE AND USEFUL COLLECTIONS OF OLIGONUCLEOTIDES,” Greyson, U.S. Pat. No. 5,789,577 “METHOD FOR THE CONTROLLED SYNTHESIS OF POLYNUCLEOTIDE MIXTURES WHICH ENCODE DESIRED MIXTURES OF PEPTIDES,” and Huse, WO 92/06176 “SURFACE EXPRESSION LIBRARIES OF RANDOMIZED PEPTIDES.”

The chemistry involved in these synthetic methods is known by those of skill. In general, they utilize phosphoramidite solid-phase chemical synthesis in which the 3′ ends of nucleic acid substrate sequences are covalently attached to a solid support, e.g., controlled pore glass. The 5′ protecting groups can be, e.g., a triphenylmethyl group, such as, dimethoxyltrityl (DMT) or monomethyoxytrityl, a carbonyl-containing group, such as, 9-fluorenylmethyloxycarbonyl (FMOC) or levulinoyl, an acid-clearable group, such as, pixyl, a fluoride-cleavable alkylsilyl group, such as, tert-butyl dimethylsilyl (T-BDMSi), triisopropyl silyl, or trimethylsilyl. The 3′ protecting groups can be, e.g., β-cyanoethyl groups.

These formats can optionally be performed in an integrated automated synthesizer system that automatically performs the synthetic steps. See also, Integrated Systems, infra. This aspect includes inputting character string information into a computer, the output of which then directs the automated synthesizer to perform the steps necessary to synthesize the desired nucleic acid sequences. Automated synthesizers are available from many commercial suppliers including PE Biosystems and Beckman Instruments, Inc.

To further ensure that target gene sequences, e.g., dehalogenase or other hydrolase nucleic acid sequences are ultimately obtained, certain techniques can be utilized following DNA synthesis. For example, gel purification is one method that can be used to purify synthesized oligonucleotides. High-performance liquid chromatography can be similarly employed. Furthermore, translational coupling can be used to assess gene functionality, e.g., to test whether full-length sequences such as full-length altered dehalogenases or full-length altered hydrolases are generated. In this process, the translation of a reporter protein, e.g., green fluorescent protein or β-galactosidase is coupled to that of the target gene product. This enables one to distinguish, e.g., full-length enzyme sequences from those that contain deletions or frame shifts. The subsequent selection of desired traits or properties of target gene sequences is discussed further, supra.

In lieu of synthesizing the desired sequences, essentially any nucleic acid can optionally be custom ordered from any of a variety of commercial sources, such as The Midland Certified Reagent Company (mcrc@oligos.com), The Great American Gene Company (www.genco.com), ExpressGen, Inc. (www.expressgen.com), Operon Technologies, Inc. (www.operon.com), and many others.

Target nucleic acid sequences, e.g., dehalogenase gene sequences can be derived from expression products, e.g., mRNAs expressed from genes within a cell of a plant or other organism. A number of techniques are available for detecting RNAs. For example, northern blot hybridization is widely used for RNA detection, and is generally taught in a variety of standard texts on molecular biology, including Current Protocols in Molecular Biology, F. M. Ausubel et al., eds., Current Protocols, a joint venture between Greene Publishing Associates, Inc. and John Wiley & Sons, Inc., (supplemented through 1999) (Ausubel), Sambrook et al., Molecular Cloning—A Laboratory Manual (2nd Ed.), Vol. 1-3, Cold Spring Harbor Laboratory, Cold Spring Harbor, N.Y., 1989 (Sambrook), and Berger and Kimmel, Guide to Molecular Cloning Techniques, Methods in Enzymology volume 152 Academic Press, Inc., San Diego, Calif. (Berger). Furthermore, one of skill will appreciate that essentially any RNA can be converted into a double stranded DNA using a reverse transcriptase enzyme and a polymerase. See, Ausubel, Sambrook and Berger. Messenger RNAs can be detected by converting, e.g., mRNAs into cDNAs, which are subsequently detected in, e.g., a standard “Southern blot” format.

Examples of techniques sufficient to direct persons of skill through in vitro amplification methods, useful, e.g., for amplifying synthesized dehalogenase or other hydrolase nucleic acid sequences, include the polymerase chain reaction (PCR), the ligase chain reaction (LCR), Qβ-replicase amplification, and other RNA polymerase mediated techniques (e.g., NASBA). These techniques are found in Ausubel, Sambrook, and Berger, as well as in Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al. (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids, e.g., full-length dehalogenase or other hydrolase nucleic acid sequences, by PCR are summarized in Cheng et al. (1994) Nature 369: 684-685 and the references therein, in which PCR amplicons of up to 40 kb are generated.

In one preferred method, assembled sequences are checked, e.g., for incorporation of specific dehalogenase or other hydrolase nucleic acid subsequences. This can be done by cloning and sequencing the nucleic acids, and/or by restriction digestion, e.g., as essentially taught in Ausubel, Sambrook, and Berger, supra. In addition, sequences can be PCR amplified and sequenced directly. Thus, in addition to, e.g., Ausubel, Sambrook, Berger, and Innis, additional PCR sequencing methodologies are also particularly useful. For example, direct sequencing of PCR generated amplicons by selectively incorporating boronated nuclease resistant nucleotides into the amplicons during PCR and digestion of the amplicons with a nuclease to produce sized template fragments has been performed (Porter et al. (1997) Nucleic Acids Res. 25(8): 1611-1617).

Introduction of Altered Dehalogenase/Hydrolase Nucleic Acid Sequences into the Cells or Organisms of Interest

In certain embodiments of the present invention, altered dehalogenase or other hydrolase nucleic acid sequences are introduced into the cells of particular organisms of interest. There are several well-known methods of introducing target nucleic acids into, e.g., bacterial cells, any of which may be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention.

Bacteria are typically grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress Expression System™ from Qiagen). The isolated and purified plasmids are then further manipulated to produce other plasmids.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC.

Additional basic procedures for sequencing, cloning and other aspects of molecular biology and underlying theoretical considerations are also found in Watson et al. (1992) Recombinant DNA Second Edition Scientific American Books, NY. Furthermore, a wide variety of cloning kits and associated products are commercially available from, e.g., Pharmacia Biotech, Stratagene, Sigma-Aldrich Co., Novagen, Inc., Fermentas, and 5 Prime→3 Prime, Inc.

Selection of a Desired Trait or Property

There are various “breedable” properties for which the biocatalysts of the present invention can be selected including assorted kinetic constants, stability, selectivity, inhibition profiles, altered substrate specificity, increased enantioselectivity, increased activity, increased gene expression, activity under diverse environmental conditions (i.e., increased thermostability, increased activity in various organic solvents, pH tolerance, etc.), ability to catalyze reactions of interest, and the like. Generally, one or more recombination cycle(s) is/are optionally followed by at least one cycle of selection for molecules having one or more of these or other desired traits or properties.

In particular, in one embodiment, the selecting or screening includes incubating expression products of the altered or recombined members of the one or more populations of hydrolase nucleic acids with at least one halohydrocarbon that includes a compound having the formula:

M¹((CH₂)ₐ(CHX¹)_{b}(CH₂)_{c})_{d}M²

wherein a, b, c, and d comprise integers independently selected from and including 0 to and including 100; wherein M¹ and M² are independently selected from: —H, —CH₂X², —CH₃, —R , —CN, —CONHR², —CONR³R⁴, —COOR⁵, —CONH₂, and —COOH; wherein R¹, R², R³, R⁴, and R⁵ are independently selected from: —H—CH₃, —CH₂CH₃, —(CH₂)ₙCH₃, —C₆H₅, —C₆H₄X³, —C₆H₄Y, and —((CH₂)ₑ(CHX⁴)_{f}(CH₂)_{g})ₕM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³is selected from: —H, —CH₂X⁵, —CH₃, —R¹⁰, —CN, —CONHR¹¹, —CONR¹²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶; wherein nn comprises an integer selected from and including 0 to and including 100; and, wherein X¹, X², X³, X⁴, X⁵, and X⁶ are independently selected from: —F, —Cl, —Br, and —I; and detecting a detectable signal which indicates an activity of the expression products. Furthermore, in certain of these embodiments, d is about 0 and b is at least about 1 and/or h is about 0 and f is at least about 1. Optionally, encoded polypeptides of members of the one or more populations of hydrolase nucleic acids include hydrolase activities other than halogenated aliphatic dehalogenase activities.

If a recombination cycle is performed in vitro, the products of recombination, i.e., recombinant or altered nucleic acids, are sometimes introduced into cells before the selection step. Recombinant nucleic acids can also be linked to an appropriate vector or to other regulatory sequences before selection. Alternatively, products of recombination generated in vitro are sometimes packaged in viruses (e.g., bacteriophage) before selection. If recombination is performed in vivo, recombination products may sometimes be selected in the cells in which recombination occurred. In other applications, recombinant segments are extracted from the cells, and optionally packaged as viruses or other vectors, before selection.

The nature of selection depends on what trait or property is to be acquired or for which improvement is sought. It is not usually necessary to understand the molecular basis by which particular recombination products have acquired new or improved traits or properties relative to the starting substrates. For instance, a gene has many component sequences, each having a different intended role (e.g., coding sequences, regulatory sequences, targeting sequences, stability-conferring sequences, subunit sequences and sequences affecting integration). Each of these component sequences are optionally varied and recombined simultaneously. Selection is then performed, for example, for recombinant products that have an increased ability to confer activity upon a cell without the need to attribute such improvement to any of the individual component sequences of the vector.

Depending on the particular protocol used to select for a desired trait or property, initial round(s) of screening can sometimes be performed using bacterial cells due to high transfection efficiencies and ease of culture. However, yeast, fungal or other eukaryotic systems may also be used for library expression and screening when bacterial expression is not practical or desired. Similarly, other types of selection that are not amenable to screening in bacterial or simple eukaryotic library cells, are performed in cells selected for use in an environment close to that of their intended use. Final rounds of screening are optionally performed in the precise cell type of intended use.

When further improvement in a trait is sought, at least one and usually a collection of recombinant products surviving a first round of screening/selection are optionally subject to a further round of recombination. These recombinant products can be recombined with each other or with exogenous segments representing the original substrates or further variants thereof. Again, recombination can proceed in vitro or in vivo. If the previous screening step identifies desired recombinant products as components of cells, the components can be subjected to further recombination in vivo, or can be subjected to further recombination in vitro, or can be isolated before performing a round of in vitro recombination. Conversely, if the previous selection step identifies desired recombinant products in naked form or as components of viruses, these segments can be introduced into cells to perform a round of in vivo recombination. The second round of recombination, irrespective how performed, generates additionally recombined products which encompass more diversity than is present in recombinant products resulting from previous rounds.

The second round of recombination may be followed by still further rounds of screening/selection according to the principles discussed for the first round. The stringency of selection can be increased between rounds. Also, the nature of the screen and the trait or property being selected may be varied between rounds if improvement in more than one trait or property is sought. Additional rounds of recombination and screening can then be performed until the recombinant products have sufficiently altered to acquire the desired new or improved trait or property.

Multiple cycles of recombination can be performed to increase library diversity before a round of selection is performed. Alternately, where the library is diverse, multiple rounds of selection can be performed prior to recombination methods.

In the context of the present invention, a variety of related (or even unrelated) properties can be selected for using any available assay. For example, screening assays for altered dehalogenase or other altered hydrolase activity can be performed, e.g., by detecting hydronium ions or halide ions liberated upon hydrolysis of, e.g., carbon-halogen bonds in reactant or substrate molecules. Other suitable techniques can include alcohol dehydrogenase-linked enzyme assays, fluorescence resonance energy transfer (FRET) assays, gas chromatography mass spectroscopy (GCMS) analysis, and nuclear magnetic resonance (NMR) (e.g., to detect reaction products, such as the alcohols, polyols, epoxides, or the like described herein).

For example, pH changes caused by the release of protons can serve as an indicator of enzymatic activity. These changes can be detected using fluorescent or visible pH indicators that undergo discernable color changes over the functional pH range of the particular enzyme. Various visible pH indicators are available which can be used for purposes of the present invention including thymol blue, m-cresol purple, phenol red, cresol red, bromothymol blue. Appropriate fluorescent pH indicators can include 1,4-naphthol sulfonic acid, α-napthol sulfonic acid, 3,6-dioxyphthalic dinitrile, orcinaurine, and coumaric. In vitro pH indicators, e.g., multiple parallel pH probes can be used to directly detect pH changes.

As mentioned, the release of halide ions can also serve as an indicator of dehalogenase activity. In this embodiment, a halide-sensitive fluorescent dye (e.g., lucigenin) can be included in the reaction mixture to be assayed. The halide-sensitive dye will be quenched upon contact with halide ion and thereby decrease the measured fluorescence. For example, the conversion of 1,2,3-trichloropropane to 1-chloro-2,3-epoxypropane catalyzed by an altered 1-chloro-2,3-epoxypropane providing halohydrocarbon-haloalcohol dehalogenase or any other dehalogenation reaction, disclosed herein, is optionally monitored by, e.g., lucigenin quenching. Alternatively, a halide ion sensitive probe (e.g., a halide-selective electrode) can be used to detect halide ion liberation.

Enzyme activity can also be assessed using a coupled enzyme system to detect the product molecules. For example, the dehalogenation of halogenated alkanes yields alcohols, many of which can serve as substrates for alcohol dehydrogenase enzymes. In turn, alcohol dehydrogenase activity can be monitored by detecting NADH disappearance. This coupling format is well-known and many alcohol dehydrogenase enzymes are readily available commercially.

Integrated Systems

The present invention provides computers, computer readable media and integrated systems comprising character strings corresponding to altered dehalogenases and other hydrolases or to their encoding nucleic acids. These sequences can be manipulated by in silico shuffling methods, or by standard sequence alignment (also discussed, supra), or word processing software.

For example, different types of similarity and considerations of various stringency and character string length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among four principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). Examples of software packages with GOs for calculating sequence similarity include BLAST and BLAST 2.0, which can be adapted to the present invention by inputting character strings corresponding to the sequences herein. An additional example of a useful sequence alignment algorithm is PILEUP. These and other sequence alignment algorithms are described further, supra.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting character strings corresponding to altered dehalogenases or other hydrolases (or coding nucleic acids), e.g., altered by the methods herein. For example, the integrated systems can include the foregoing software having the appropriate character string information, e.g., used in conjunction with a user interface (e.g., a GUI in a standard operating system such as a Windows, Macintosh or LINUX system) to manipulate strings of characters. As noted, specialized alignment programs such as BLAST or PILEUP can also be incorporated into the systems of the invention for alignment of nucleic acids or proteins (or corresponding character strings).

Integrated systems for analysis in the present invention typically include a digital computer with software for aligning or manipulating dehalogenase or other hydrolase sequences, as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visual basic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of dehalogenase or other hydrolase nucleic acid sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the system to carry out any desired operation, e.g., nucleic acid sequence alignment, nucleic acid synthesis, etc.

In one aspect, the computer system is used to perform in silico shuffling of character strings that correspond to dehalogenase and/or other hydrolase nucleic acid fragments. A variety of such methods are set forth in “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118854) and “METHODS FOR MAKING CHARACTER STRINGS, POLYNUCLEOTIDES & POLYPEPTIDES HAVING DESIRED CHARACTERISTICS” by Selifonov and Stemmer, filed Oct. 12, 1999 (U.S. Ser. No. 09/416,375). In brief, genetic operators are used in genetic algorithms to change given sequences, e.g., by mimicking genetic events such as mutation, recombination, death and the like. Multi-dimensional analysis to optimize sequences can also be performed in the computer system, e.g., as described in the '375 application.

A digital system can also instruct an oligonucleotide synthesizer to synthesize dehalogenase or other hydrolase oligonucleotides, e.g., used for gene reconstruction or recombination, or to order dehalogenase or other hydrolase oligonucleotides from commercial sources (e.g., by printing appropriate order forms or by linking to an order form on the internet).

The digital system can also include output elements for controlling nucleic acid synthesis (e.g., based upon a sequence or an alignment of an altered dehalogenase or other hydrolase as herein), i.e., an integrated system of the invention optionally includes an oligonucleotide synthesizer or an oligonucleotide synthesis controller for synthesizing dehalogenase or other hydrolase nucleic acid fragments. The system can include other operations which occur downstream from an alignment or other operation performed using a character string corresponding to a sequence herein, e.g., as noted above with reference to assays.

Kits and Assay Systems

The present invention also provides kits and assay systems for performing any of the altered enzyme catalyzed reactions described herein. For example, a kit optionally includes a set of instructions for practicing the methods described herein; assay components that include altered enzymes, or cells that include altered enzymes, or both, and one or more reagents; and a container for packaging the set of instructions and the assay components. The assay system generally includes a set of instructions for practicing the methods disclosed herein, and a reaction vessel (e.g., a bioreactor or fermentor) that includes altered enzymes, or cells that include the altered enzymes, or both. The reaction vessel optionally also includes, e.g., a reagent inlet; a reagent outlet; a sparging line, an impeller, or the like. The altered enzymes of the assay system typically include immobilized altered enzymes, or altered enzymes free in solution, or both. As further alternatives, the cells of the assay system optionally include immobilized cells, or cells free in solution, or both.

The kits and assays systems of the invention include altered enzymes that are selected from, e.g., an altered halocarbon dehalogenase, an altered halohydrocarbon dehalogenase, an altered haloalcohol dehalogenase, an altered halopolyol dehalogenase, an altered haloepoxide dehalogenase, an altered epoxide hydrolase, an altered halohydrocarbon-haloalcohol dehalogenase, an altered haloalcohol-halopolyol dehalogenase, an altered halohydrocarbon-haloalcohol-halopolyol dehalogenase, an altered haloepoxide dehalogenase-epoxide hydrolase, an altered epoxide providing halohydrocarbon-haloalcohol isomer dehalogenase, an altered first haloalcohol isomer providing halohydrocarbon-second haloalcohol isomer providing halohydrocarbon dehalogenase, an altered epoxide providing first haloalcohol isomer-epoxide providing second haloalcohol isomer dehalogenase, and the like.

As mentioned, the reaction vessel of the assay system optionally includes altered dehalogenases and/or other hydrolases that are immobilized, e.g., on a solid support. Either covalent and/or non-covalent immobilization methods can be used in the present invention depending upon the particular application. Covalent approaches typically utilize reactive groups present on various amino acid side chains to attach enzymes to polymeric or inorganic supports. Indirect attachment can also be accomplished using various bifunctional cross-linking agents. Non-covalent enzyme immobilization can involve adsorptive, ionic (e.g., chelate- or chelant-mediated), or bioaffinity support (e.g., biotin-avidin) interactions. Non-covalent attachment can also be achieved using gel-entrapment or microencapsulation.

A wide variety of organic and inorganic polymers, both natural and synthetic, can be employed as the material for the solid support. Factors to consider when selecting an appropriate support include its biocompatibility, durability, and cost. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that can be appropriate depending on the assay include ceramics, metals, metalloids, semiconductive materials, cements and the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

Immobilized altered dehalogenases and/or other hydrolases can be included as assay components of the kits and assay systems of the present invention to catalyze the conversion selected reactants to one or more products, e.g., the conversion of recalcitrant halogenated compounds to useful products. In certain embodiments, reactants can be added to a reaction mixture in solution, e.g., as in batch method approach, or into the liquid stream of a continuous feed process.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of generating diversity in one or more populations of hydrolase nucleic acids, the method comprising:
altering at least one nucleotide of one or more members of the one or more populations of hydrolase nucleic acids using at least one modification technique, wherein encoded polypeptides of each of the one or more members comprise abilities to catalyze at least two different reactions or at least two steps in a multiple step reaction pathway; or,
recombining two or more members of the one or more populations of hydrolase nucleic acids, wherein encoded polypeptides of each of the two or more members comprise abilities to catalyze at least one different reaction than other members of the one or more populations of hydrolase nucleic acids; or,
recombining one or more members from at least a first population of hydrolase nucleic acids, which one or more members encode one or more polypeptides comprising abilities to catalyze at least one step in a multiple step reaction pathway and recombining one or more members from at least a second population of hydrolase nucleic acids, which one or more members encode one or more polypeptides comprising abilities to catalyze at least one different step in the multiple step reaction pathway; and,
selecting or screening at least one altered or recombined member of the one or more populations of hydrolase nucleic acids, thereby generating diversity in the one or more populations of hydrolase nucleic acids; and,
repeating the altering step, or at least one of the recombining steps, and the selecting or screening step at least once.

2. The method of claim 1, wherein the selecting or screening, comprises:
incubating expression products of the altered or recombined members of the one or more populations of hydrolase nucleic acids with at least one halohydrocarbon comprising a compound having the formula:
M¹ ((CH₂)ₐ(CHX¹)_{b}(CH₂)_{c})_{d}M²
wherein a, b, c, and d comprise integers independently selected from and including 0 to and including 100; wherein M¹ and M² are independently selected from: —H, —CH₂X², —CH₃, —R¹, —CN, —CONHR², —CONR³R⁴, —COOR⁵, —CONH₂, and —COOH; wherein R¹, R², R³, R⁴, and R⁵ are independently selected from: —H, —CH₃, —CH₂CH₃, —(CH₂)ₙCH₃, —C₆H₅, —C₆H₄X³, —C₆H₄Y, and —((CH₂)ₑ(CHX⁴)_{f}(CH₂)_{g})ₕM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³ is selected from: —H, —CH₂X⁵, —CH₃, —R¹⁰, —CN, —CONHR¹¹, —CONR¹²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶; wherein nn comprises an integer selected from and including 0 to and including 100; and, wherein X¹, X², X³, X⁴, X⁵, and x are independently selected from: —F, —Cl, —Br, and —I; and,
detecting at least one detectable signal which indicates at least one activity of the expression products.

3. The method of claim 2, wherein d is about 0 and b is at least about 1.

4. The method of claim 2, wherein h is about 0 and f is at least about 1.

5. The method of claim 1, wherein the one or more populations of hydrolase nucleic acids comprise one or more of: a population of halocarbon dehalogenase nucleic acids; a population of halohydrocarbon dehalogenase nucleic acids; a population of haloalcohol dehalogenase nucleic acids; a population of halopolyol dehalogenase nucleic acids; a population of first haloalcohol isomer providing halohydrocarbon dehalogenase nucleic acids; a population of second haloalcohol isomer providing halohydrocarbon dehalogenase nucleic acids; a population of haloalcohol isomer dehalogenase nucleic acids; a population of epoxide providing first haloalcohol isomer dehalogenase nucleic acids; a population of epoxide providing second haloalcohol isomer dehalogenase nucleic acids; a population of haloepoxide dehalogenase nucleic acids; or a population of epoxide hydrolase nucleic acids.

6. The method of claim 1, wherein the one or more populations of hydrolase nucleic acids comprise one or more of: accession number AJ012627, accession number AF060871, accession number AF017179, accession number L49435, accession number M26950, accession number D14594, accession number CAB01264, accession number AL079353, accession number AL022121, accession number AL123456, accession number AL035636, accession number AE002084, accession number AE000513, accession number AL137165, accession number AL133252, accession number AL132707, accession number AJ005843, accession number AL079345, accession number AL031124, accession number AF043240, accession number X84038, accession number M81691, or accession number A28028.

7. The altered or recombined members made by the method of claim 1.

8. The method of claim 1, wherein the at least one modification technique is selected from the group consisting of: recombination, recursive sequence recombination, oligonucleotide-directed mutagenesis, site-directed mutagenesis, error-prone PCR, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, site saturation mutagenesis, ensemble mutagenesis, recursive ensemble mutagenesis, and chimeric nucleic acid multimer creation.

9. The method of claim 1, wherein encoded polypeptides of members of the one or more populations of hydrolase nucleic acids comprise hydrolase activities other than halogenated aliphatic dehalogenase activities.

10. The method of claim 1, the selecting or screening step comprising selecting or screening at least one altered or recombined member for an efficient catalyzation of at least one reaction of interest by an encoded altered hydrolase.

11. The method of claim 10, wherein the at least one reaction of interest is selected from the group consisting of: a conversion of at least one organic compound to at least one product; a conversion of at least one halocarbon to at least one product; a conversion of at least one first halohydrocarbon to at least one hydrocarbon, to at least one second halohydrocarbon, to at least one alcohol, to at least one haloalcohol, to at least one polyol, to at least one halopolyol, to at least one epoxide, or to at least one haloepoxide; a conversion of at least one first haloalcohol to at least one hydrocarbon, to at least one halohydrocarbon, to at least one second haloalcohol, to at least one polyol, to at least one halopolyol, to at least one epoxide, or to at least one haloepoxide; a conversion of at least one first halohydrocarbon to at least one hydrocarbon, to at least one halohydrocarbon, to at least one polyol, to at least one halopolyol, to at least one epoxide, to at least one haloepoxide, or to at least one haloalcohol; a conversion of at least one halopolyol to at least one epoxide; a conversion of at least one haloalcohol to at least one epoxide; a conversion of at least one halohydrocarbon to at least one epoxide; a conversion of at least one halohydrocarbon to at least one of a first haloalcohol isomer and at least one of a second haloalcohol isomer; a conversion of a mixture comprising at least one of a first haloalcohol isomer and at least one of a second haloalcohol isomer to at least one epoxide; a conversion of at least one haloepoxide to at least one epoxide; a conversion at least one epoxide to at least one polyol or to at least one enantiomer of the epoxide; and a conversion of at least one haloepoxide to at least one polyol or to at least one enantiomer of an epoxide.

12. The method of claim 1, further comprising introducing one or more of the altered or recombined members of the one or more populations of hydrolase nucleic acids into at least one cell, wherein the one or more introduced altered or recombined members are expressed to provide at least one altered or recombined hydrolase.

13. The method of claim 12, wherein the at least one altered or recombined hydrolase is selected from the group consisting of: an altered or recombined halocarbon dehalogenase; an altered or recombined halohydrocarbon dehalogenase; an altered or recombined haloalcohol dehalogenase; an altered or recombined halohydrocarbon-haloalcohol dehalogenase; an altered or recombined halopolyol dehalogenase; an altered or recombined haloalcohol-halopolyol dehalogenase; an altered or recombined halohydrocarbon-haloalcohol-halopolyol dehalogenase; an altered or recombined first haloalcohol isomer providing halohydrocarbon-second haloalcohol isomer providing halohydrocarbon dehalogenase; an altered or recombined epoxide providing halohydrocarbon-haloalcohol isomer dehalogenase; an altered or recombined epoxide providing first haloalcohol isomer-epoxide providing second haloalcohol isomer dehalogenase; an altered or recombined haloepoxide dehalogenase; an altered or recombined epoxide hydrolase; and an altered or recombined haloepoxide dehalogenase-epoxide hydrolase.

14. The method of claim 12, wherein the at least one cell is an organism.

15. The at least one cell made by the method of claim 12.

16. The method of claim 1, further comprising expressing the altered or recombined members of the one or more populations of hydrolase nucleic acids to provide at least one altered or recombined hydrolase.

17. The method of claim 16, wherein the at least one altered or recombined hydrolase is selected from the group consisting of: an altered or recombined halocarbon dehalogenase; an altered or recombined halohydrocarbon dehalogenase; an altered or recombined haloalcohol dehalogenase; an altered or recombined halohydrocarbon-haloalcohol dehalogenase; an altered or recombined halopolyol dehalogenase; an altered or recombined haloalcohol-halopolyol dehalogenase; an altered or recombined halohydrocarbon-haloalcohol-halopolyol dehalogenase; an altered or recombined first haloalcohol isomer providing halohydrocarbon-second haloalcohol isomer providing halohydrocarbon dehalogenase; an altered or recombined epoxide providing halohydrocarbon-haloalcohol isomer dehalogenase; an altered or recombined epoxide providing first haloalcohol isomer-epoxide providing second haloalcohol isomer dehalogenase; an altered or recombined haloepoxide dehalogenase; an altered or recombined epoxide hydrolase; and an altered or recombined haloepoxide dehalogenase-epoxide hydrolase.

18. The at least one altered or recombined hydrolase made by the method of claim 16.

19. A method of converting at least one organic compound, at least one halocarbon, or at least one first halohydrocarbon to at least one product, comprising: incubating the at least one organic compound, the at least one halocarbon, or the at least one first halohydrocarbon and the at least one altered or recombined hydrolase of claim 18, wherein the at least one altered or recombined hydrolase converts the at least one organic compound, the at least one halocarbon, or the at least one first halohydrocarbon to the at least one product.

20. The method of claim 19, wherein the at least one first halohydrocarbon comprises a compound having the formula:
M¹.((CH₂)ₐ(CHX¹)_{b}(CH₂)_{c})_{d}M²
wherein a, b, c, and d comprise integers independently selected from and including 0 to and including 100; wherein M¹ and M² are independently selected from: —H, —CH₂X², —CH₃, —R¹, —CN, —CONHR², —CONR³R⁴, —COOR⁵, —CONH₂, and —COOH; wherein R¹, R², R³, R⁴, and R⁵ are independently selected from: —H, —CH₃, —CH₂CH₃, —(CH₂)ₙCH₃, —C₆H₅, —C₆H₄X³, —C₆H₄Y, and —((CH₂)ₑ(CHX⁴)_{f}(CH₂)_{g})ₕM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³ is selected from: —H, —CH₂X⁵, —CH₃, —R¹⁰, —CN, —CONHR¹¹, —CONR¹²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶); wherein nn comprises an integer selected from and including 0 to and including 100; and, wherein X¹, X², X³, X⁴, X⁵, and X⁶ are independently selected from: —F, —Cl, —Br, and —I.

21. The method of claim 20, wherein d is about 0 and b is at least about 1.

22. The method of claim 20, wherein h is about 0 and f is at least about 1.

23. The method of claim 19, wherein the at least one first halohydrocarbon comprises a compound having the formula:
M¹((CQ¹Q²)ₐ(CHX¹)_{b}(CQ³Q⁴)_{c})_{d}M²
wherein a, b, c, and d comprise integers independently selected from and including 0 to and including 100; wherein M¹ and M² are independently selected from: —H, —CH₂X², CH₃, —CN, —CONHR¹, —CONR²R³, —COOR⁴, —CONH₂, —COOH, and —((CH₂)ₑ(CHX³)_{f}(CH₂)_{g})ₕM³; wherein e, f, g, and h comprise integers independently selected from and including 0 to and including 100; wherein R¹, R², R³, and R⁴ are independently selected from: —H, —CH₃, —CH₂CH₃, —(CH₂)ₙₙCH₃, —C₆H₅, —C₆H₄X⁴, —C₆H₄Y, and —((CH₂)ᵢ(CHX⁵)ⱼ(CH₂)ₖ)ₗM⁴; wherein nn comprises an integer selected from and including 0 to and including 100; wherein Y is selected from: —CN, —CONHR⁵, —CONR⁶R⁷, —COOR⁸, —CONH₂, —COOH; wherein i, j, k, and l comprise integers independently selected from and including 0 to and including 100; wherein Q¹ is selected from: —H and —((CH₂)ₘ(CHX⁶)ₙ(CH₂)ₒ)ₚM⁵; wherein m, n, o, and p comprise integers independently selected from and including 0 to and including 100; wherein Q² is selected from: -H and —((CH₂)_{q}(CHX⁷)ᵣ(CH₂)ₛ)ₜM⁶; wherein q, r, s, and t comprise integers independently selected from and including 0 to and including 100; wherein Q³ is selected from: —H and —((CH₂)ᵤ(CHX⁸)ᵥ(CH₂)_{w})ₓM⁷; wherein u, v, w, and x comprise integers independently selected from and including 0 to and including 100; wherein Q⁴ is selected from: —H and —((CH₂)_{y}(CHX⁹)_{z}(CH₂)ₐₐ)_{bb}M⁸; wherein y, z, aa, and bb comprise integers independently selected from and including 0 to and including 100; wherein M³, M⁴, M⁵, M⁶, M⁷, and M⁸ are independently selected from: —H, —CH₂X¹⁰, —CH₃, —R⁹, —CN, —CONHR¹⁰, —CONR¹¹R¹², —COOR¹³, —CONH₂ and —COOH; wherein R⁵, R⁶, R⁷, R⁸, R⁹, R¹⁰, R¹¹, R¹², and R¹³ are independently selected from: —H, —CH₃, —CH₂CH₃, —(CH₂)ₙₙₙCH₃, —C₆H₅, —C₆H₄X¹¹; wherein nnn comprises an integer selected from and including 0 to and including 100;and wherein X¹, X², X³, X⁴, X⁵, X⁶, X⁷, X⁸, X⁹, X¹⁰, and X¹¹ are independently selected from: —F, —Cl, —Br, and —I.

24. The method of claim 23, wherein d is about 0 and b is at least about 1.

25. The method of claim 23, wherein h is about 0 and f is at least about 1.

26. The method of claim 23, wherein l is about 0 and j is at least about 1.

27. The method of claim 23, wherein p is about 0 and n is at least about 1.

28. The method of claim 23, wherein t is about 0 and r is at least about 1.

29. The method of claim 23, wherein x is about 0 and v is at least about 1.

30. The method of claim 23, wherein bb is about 0 and z is at least about 1.

31. The method of claim 23, wherein at least one of Q¹, Q², Q³, and Q⁴ comprises one or more halogens, wherein at least one of the one or more halogens is hydrolyzed upon conversion of M¹((CQ¹Q²)ₐ(CHX¹)_{b}(CQ³Q⁴)_{c})_{d}M² to the at least one product.

32. The method of claim 19, wherein the at least one product comprises a compound having the formula:
M¹((CH₂)ₐ(CHX¹)_{b-c}(CHOH)_{c}(CH₂)_{d})ₑM²
wherein a, b, c, d, and e comprise integers independently selected from and including 0 to and including 100; wherein Ml and M² are independently selected from: —H, —CH₂X², —CH₃, —R¹, —CN, —CONHR², —CONR³R⁴, —COOR⁵, —CONH₂, and —COOH; wherein R¹, R², R³, R⁴, and R⁵ are independently selected from: —H, —CH₃, —CH₂CH₃, —CH₂)ₙCH₃, —C₆H₅, —C₆H₄X³, —C₆H₄Y, and —((CH₂)_{f}(CHX⁴)_{g}(CH₂)ₕ)ᵢM³; wherein n comprises an integer selected from and including 0 to and including 100; wherein e, f, g, and h comprise integers independently selected from and including 0 to and including 100; wherein Y is selected from: —CN, —CONHR⁶, —CONR⁷R⁸, —COOR⁹, —CONH₂, and —COOH; wherein M³ is selected from: —H, —CH₂X⁵, —CH₃, —R¹⁰, —CN, —CONHR¹¹, —CONR²R¹³, —COOR¹⁴, —CONH₂, and —COOH; wherein R⁶, R⁷, R⁸, R⁹, R¹⁰, R¹¹, R¹², R¹³, and R¹⁴ are independently selected from: —H, —CH₃, —CH₂CH₃, —(CH₂)ₙₙCH₃, —C₆H₅, —C₆H₄X⁶; wherein nn comprises an integer selected from and including 0 to and including 100; and, wherein X¹, X², X³, X⁴, X⁵, and X⁶ are independently selected from: —F, —Cl, —Br, and —I.

33. The method of claim 32, wherein e is about 0 and b-c is at least about 1.

34. The method of claim 32, wherein i is about 0 and f is at least about 1.

35. The method of claim 32, wherein c is at most equal to b.

36. The method of claim 19, wherein the at least one product comprises a compound having the formula:
M¹((CQ¹Q²)ₐ(CHX¹)_{b-c}(CHOH)_{c}(CQ³Q⁴)_{d})ₑM²
wherein a, b, c, d, and e comprise integers independently selected from and including 0 to and including 100; wherein M¹ and M² are independently selected from: —H, —CH₂X², —CH₃, —CN, —CONHR¹, —CONR²R³, —COOR⁴, —CONH₂, —COOH, and —((CH₂)_{f}(CHX³)_{g}(CH₂)ₕ)ᵢM³; wherein f, g, h, and i comprise integers independently selected from and including 0 to and including 100; wherein R¹, R², R³, and R⁴ are independently selected from: —H, —CH₃, —CH₂CH₃, —(CH₂)ₙₙCH₃, —C₆H₅, —C₆H₄X⁴, —C₆H₄Y, and —((CH₂)ⱼ(CHX⁵)ₖ(CH₂)ₗ)ₘM⁴; wherein nn comprises an integer selected from and including 0 to and including 100; wherein Y is selected from: —CN, —CONHR⁵, —CONR⁶R⁷, —COOR⁸, —CONH₂, —COOH; wherein j, k, l, and m comprise integers independently selected from and including 0 to and including 100; wherein Q¹ is selected from: —H and —((CH₂)ₙ(CHX⁶)ₒ(CH₂)ₚ)_{q}M⁵; wherein n, o, p, and q comprise integers independently selected from and including 0 to and including 100; wherein Q² is selected from: —H and —((CH₂)ᵣ(CHX⁷)ₛ(CH₂)ₜ)ᵤM⁶; wherein r, s, t, and u comprise integers independently selected from and including 0 to and including 100; wherein Q³ is selected from: —H and —((CH₂)ᵥ(CHX⁸)_{w}(CH₂)ₓ)_{y}M⁷; wherein v, w, x, and y comprise integers independently selected from and including 0 to and including 100; wherein Q⁴ is selected from: —H and —((CH₂)_{z}(CHX⁹)ₐₐ(CH₂)_{bb})_{cc}M⁸; wherein z, aa, bb, and cc comprise integers independently selected from and including 0 to and including 100; wherein M³, M⁴, M⁵, M⁶, M⁷, and M⁸ are independently selected from: —H, —CH₂X¹⁰, —CH₃, —R⁹, —CN, —CONHR¹⁰, —CONR¹¹R¹², —COOR¹³, —CONH₂, and —COOH; wherein R⁵, R⁶, R⁷, R⁸, R⁹, R¹⁰, R¹¹, R¹², and R¹³ are independently selected from: —H, —CH₃, —CH₂CH₃, —(CH₂)ₙₙₙCH₃, —C₆H₅, —C₆H₄X¹¹; wherein nnn comprises an integer selected from and including 0 to and including 100;and wherein X¹, X², X³, X⁴, X⁵, X⁶, X⁷, X⁸, X⁹, X¹⁰, and X¹¹ are independently selected from: —F, —Cl, —Br, and —I.

37. The method of claim 36, wherein e is about 0 and b-c is at least about 1.

38. The method of claim 36, wherein i is about 0 and g is at least about 1.

39. The method of claim 36, wherein m is about 0 and k is at least about 1.

40. The method of claim 36, wherein q is about 0 and o is at least about 1.

41. The method of claim 36, wherein u is about 0 and s is at least about 1.

42. The method of claim 36, wherein y is 0 and w is at least 1.

43. The method of claim 36, wherein cc is about 0 and aa is at least about 1.

44. The method of claim 36, wherein c is at most equal to b.

45. The method of claim 19, wherein the at least one organic compound, the at least one halocarbon, or the at least one first halohydrocarbon comprises at least one prochiral or chiral center; or,
wherein the at least one organic compound, the at least one halocarbon, or the at least one first halohydrocarbon is converted to the at least one product enantioselectively; or,
wherein the at least one organic compound, the at least one halocarbon, or the at least one first halohydrocarbon is converted to the at least one product enantiospecifically.

46. The method of claim 19, wherein the at least one organic compound, the at least one halocarbon, or the at least one first halohydrocarbon comprises a component of one or more mixtures.

47. The method of claim 19, wherein the at least one organic compound, the at least one halocarbon, or the at least one first halohydrocarbon comprises from about 1 to about 100 carbon atoms.

48. The method of claim 47, wherein the carbon atoms or one or more subset of the carbon atoms comprise a straight chain structure, a branched structure, a double bond, a triple bond, or a ring structure.

49. The method of claim 19, wherein the at least one organic compound, the at least one halocarbon, or the at least one first halohydrocarbon is converted to the at least one product through a multiple step reaction pathway.

50. The method of claim 49, wherein the multiple step reaction pathway comprises at least one intermediate; or,
wherein each step in the multiple step reaction pathway occurs sequentially in a single reaction vessel, wherein the multiple step reaction pathway lacks intervening processing steps; or,
wherein at least one step in the multiple step reaction pathway occurs in a reaction vessel distinct from other reaction steps; or,
wherein the at least one altered or recombined hydrolase is involved in fewer than all steps in the multiple step reaction pathway; or,
wherein the at least one altered or recombined hydrolase catalyzes more than one reaction in the multiple step reaction pathway.

51. The method of claim 19, wherein the at least one first halohydrocarbon comprises one or more of: a cyclic halohydrocarbon, an acyclic halohydrocarbon, a haloalkane, a haloalkene, a haloalkyne, a haloalkyl nitrile, a haloalkyl amide, a haloalkyl carboxylic acid, or a haloalkyl carboxylic acid esters.

52. The method of claim 51,wherein the haloalkyl carboxylic acid comprises an α-haloacid or a β-haloacid.

53. The method of claim 52, wherein the α-haloacid or β-haloacid comprises a pharmaceutical precursor.

54. The method of claim 19, wherein the at least one product comprises one or more of: a second halohydrocarbon, a hydrocarbon, a haloalcohol, an alcohol, a halopolyol, a polyol, a haloepoxide, or an epoxide.

55. The method of claim 54, wherein the at least one alcohol is an α-hydroxy acid or a β-hydroxy acid.

56. The method of claim 19, wherein the at least one altered or recombined hydrolase is selected from the group consisting of: an artificially evolved hydrolase, a recursively recombined hydrolase, a recombinant hydrolase, a chimeric hydrolase, and a mutant hydrolase.

57. The method of claim 19, wherein the at least one altered or recombined hydrolase comprises at least one altered or recombined dehalogenase.

58. The method of claim 19, wherein the at least one altered or recombined hydrolase is selected from the group consisting of: an altered or recombined halocarbon dehalogenase, an altered or recombined halohydrocarbon dehalogenase, an altered or recombined haloalcohol dehalogenase, an altered or recombined haloalcohol-halopolyol dehalogenase, an altered or recombined halopolyol dehalogenase, an altered or recombined halohydrocarbon-haloalcohol dehalogenase, an altered or recombined halohydrocarbon-haloalcohol-halopolyol dehalogenase, an altered or recombined first haloalcohol isomer providing halohydrocarbon-second haloalcohol isomer providing halohydrocarbon dehalogenase, an altered or recombined epoxide providing first haloalcohol isomer-epoxide providing second haloalcohol isomer dehalogenase, an altered or recombined epoxide providing halohydrocarbon-haloalcohol isomer dehalogenase, an altered or recombined haloepoxide dehalogenase, an altered or recombined epoxide hydrolase, and an altered or recombined epoxide hydrolase.

59. The method of claim 19, further comprising expressing the at least one altered or recombined hydrolase in at least one heterologous host organism.

60. The at least one heterologous host organism made by the method of claim 59.

61. A method of converting at least one first halohydrocarbon to at least one first product, the method comprising:
incubating the at least one first halohydrocarbon and the at least one altered or recombined hydrolase of claim 18, wherein the at least one altered or recombined hydrolase comprises at least one altered or recombined halohydrocarbon dehalogenase, wherein the at least one altered or recombined halohydrocarbon dehalogenase converts the at least one first halohydrocarbon to the at least one first product.

62. The method of claim 61, wherein the at least one first product is selected from the group consisting of: a first hydrocarbon, a second halohydrocarbon, an alcohol, a first haloalcohol, a first polyol, a first halopolyol, a first epoxide, and a first haloepoxide.

63. The method of claim 62, wherein the at least one first haloalcohol is converted to at least one second product, the method further comprising:
incubating the at least one first haloalcohol and at least one altered or recombined haloalcohol dehalogenase, wherein the at least one altered or recombined haloalcohol dehalogenase converts the at least one first haloalcohol to the at least one second product.

64. The method of claim 63, wherein the at least one second product is selected from the group consisting of: a second hydrocarbon, a third halohydrocarbon, a second haloalcohol, a second polyol, a second halopolyol, a second epoxide, and a second haloepoxide.

65. The method of claim 64, further comprising hydrolyzing the at least one second epoxide to provide at least one third polyol; or,
further comprising incubating the at least one second polyol with at least one fourth halohydrocarbon to provide at least one polyether.

66. The method of claim 62, wherein the at least one first haloalcohol is converted to at least one second epoxide, the method further comprising:
incubating the at least one first haloalcohol and at least one altered or recombined haloalcohol-halopolyol dehalogenase, wherein the at least one altered or recombined haloalcohol-halopolyol dehalogenase converts the at least one first haloalcohol to the at least one second epoxide.

67. The method of claim 66, wherein the at least one first haloalcohol is converted to the at least one second epoxide through an intermediate halopolyol.

68. The method of claim 62, wherein the at least one first halopolyol is converted to at least one second epoxide, the method comprising:
incubating the at least one first halopolyol and at least one altered or recombined halopolyol dehalogenase, wherein the at least one altered or recombined halopolyol dehalogenase converts the at least one first halopolyol to the at least one second epoxide.

69. A method of converting at least one first haloalcohol to at least one product, the method comprising:
incubating the at least one first haloalcohol and the at least one altered or recombined hydrolase of claim 18, wherein the at least one altered or recombined hydrolase comprises at least one altered or recombined haloalcohol dehalogenase, wherein the at least one altered or recombined haloalcohol dehalogenase converts the at least one first haloalcohol to the at least one product.

70. The method of claim 69, wherein the at least one product is selected from the group consisting of: a hydrocarbon, a first halohydrocarbon, a second haloalcohol, a first polyol, a first halopolyol, a first epoxide, and a haloepoxide.

71. The method of claim 70, further comprising hydrolyzing the at least one first epoxide to provide at least one second polyol; or, further comprising incubating the at least one first polyol with at least one second halohydrocarbon to produce at least one polyether.

72. The method of claim 70, wherein the at least one first halopolyol is converted to at least one second epoxide, the method further comprising:
incubating the at least one first halopolyol and at least one altered or recombined halopolyol dehalogenase, wherein the at least one altered or recombined halopolyol dehalogenase converts the at least one first halopolyol to the at least one second epoxide.

73. A method of converting at least one first halohydrocarbon to at least one product, the method comprising:
incubating the at least one first halohydrocarbon and the at least one altered or recombined hydrolase of claim 18, wherein the at least one altered or recombined hydrolase comprises at least one altered or recombined halohydrocarbon-haloalcohol dehalogenase, wherein the at least one altered or recombined halohydrocarbon-haloalcohol dehalogenase converts the at least one first halohydrocarbon to the at least one product.

74. The method of claim 73, wherein the at least one product is selected from the group consisting of: a hydrocarbon, a second halohydrocarbon, a haloalcohol, a first polyol, a first halopolyol, a first epoxide, and a haloepoxide.

75. The method of claim 74, further comprising hydrolyzing the at least one first epoxide to provide at least one second polyol; or,
further comprising incubating the at least one first polyol with at least one third halohydrocarbon to provide at least one polyether; or,
wherein at least one first halohydrocarbon is converted to the at least one first epoxide or to the at least one first polyol through an intermediate haloalcohol.

76. The method of claim 74, wherein the at least one first halopolyol is converted to at least one second epoxide, the method further comprising:
incubating the at least one first halopolyol and at least one altered or recombined halopolyol dehalogenase, wherein the at least one altered or recombined halopolyol dehalogenase converts the at least one first halopolyol to the at least one second epoxide.

77. A method of converting at least one halopolyol to at least one epoxide, the method comprising:
incubating the at least one halopolyol and the at least one altered or recombined hydrolase of claim 18, wherein the at least one altered or recombined hydrolase comprises at least one altered or recombined halopolyol dehalogenase, wherein the at least one altered or recombined halopolyol dehalogenase converts the at least one halopolyol to the at least one epoxide.

78. A method of converting at least one haloalcohol to at least one epoxide, the method comprising:
incubating the at least one haloalcohol and the at least one altered or recombined hydrolase of claim 18, wherein the at least one altered or recombined hydrolase comprises at least one altered or recombined haloalcohol-halopolyol dehalogenase, wherein the at least one altered or recombined haloalcohol-halopolyol dehalogenase converts the at least one haloalcohol to the at least one epoxide.

79. The method of claim 78, wherein the at least one haloalcohol is converted to the at least one epoxide through an intermediate halopolyol.

80. A method of converting at least one halohydrocarbon to at least one epoxide, the method comprising:
incubating the at least one halohydrocarbon and the at least one altered or recombined hydrolase of claim 18, wherein the at least one altered or recombined hydrolase comprises at least one altered or recombined halohydrocarbon-haloalcohol-halopolyol dehalogenase, wherein the at least one altered or recombined halohydrocarbon-haloalcohol-halopolyol dehalogenase converts the at least one halohydrocarbon to the at least one epoxide.

81. The method of claim 80, wherein the at least one halohydrocarbon is converted to the at least one epoxide through at least one intermediate haloalcohol and at least one intermediate halopolyol.

82. A method of converting a plurality of a halohydrocarbon to at least one of a first haloalcohol isomer and at least one of a second haloalcohol isomer, the method comprising:
incubating the plurality of the halohydrocarbon and the at least one altered or recombined hydrolase of claim 18, wherein the at least one altered or recombined hydrolase comprises at least one altered or recombined first haloalcohol isomer providing halohydrocarbon-second haloalcohol isomer providing halohydrocarbon dehalogenase, wherein the at least one altered or recombined first haloalcohol isomer providing halohydrocarbon-second haloalcohol isomer providing halohydrocarbon dehalogenase converts the plurality of the halohydrocarbon to the at least one of the first haloalcohol isomer and the at least one of the second haloalcohol isomer.

83. The method of claim 82, wherein the at least one of the first haloalcohol isomer and the at least one of the second haloalcohol isomer are converted to at least one epoxide, the method further comprising:
incubating the at least one of the first haloalcohol isomer and the at least one of the second haloalcohol isomer and at least one altered or recombined epoxide providing first haloalcohol isomer-epoxide providing second haloalcohol isomer dehalogenase, wherein the at least one altered or recombined epoxide providing first haloalcohol isomer-epoxide providing second haloalcohol isomer dehalogenase converts the at least one of the first haloalcohol isomer and the at least one of the second haloalcohol isomer to the at least one epoxide.

84. A method of converting a plurality of a halohydrocarbon to at least one epoxide, the method comprising:
incubating the plurality of the halohydrocarbon and the at least one altered or recombined hydrolase of claim 18, wherein the at least one altered or recombined hydrolase comprises at least one altered or recombined epoxide providing halohydrocarbon-haloalcohol isomer dehalogenase, wherein the at least one altered or recombined epoxide providing halohydrocarbon-haloalcohol isomer dehalogenase converts the plurality of the halohydrocarbon to the at least one epoxide.

85. The method of claim 84, wherein the plurality of the halohydrocarbon is converted to the at least one epoxide through at least one intermediate haloalcohol isomer.

86. A method of converting a mixture comprising at least one of a first haloalcohol isomer and at least one of a second haloalcohol isomer to at least one epoxide, the method comprising:
incubating the mixture comprising the at least one of the first haloalcohol isomer and the at least one of the second haloalcohol isomer and the at least one altered or recombined hydrolase of claim 18, wherein the at least one altered or recombined hydrolase comprises at least one altered or recombined epoxide providing first haloalcohol isomer-epoxide providing second haloalcohol isomer dehalogenase, wherein the at least one altered or recombined epoxide providing first haloalcohol isomer-epoxide providing second haloalcohol isomer dehalogenase converts the mixture comprising the at least one of the first haloalcohol isomer and the at least one of the second haloalcohol isomer to the at least one epoxide.

87. A method of converting at least one haloepoxide to at least one epoxide, the method comprising:
incubating the at least one haloepoxide and the at least one altered or recombined hydrolase of claim 18, wherein the at least one altered or recombined hydrolase comprises at least one altered or recombined haloepoxide dehalogenase, wherein the at least one altered or recombined haloepoxide dehalogenase converts the at least one haloepoxide to the at least one epoxide.

88. The method of claim 87, wherein the at least one epoxide is converted to at least one polyol or to at least one enantiomer of the epoxide, the method further comprising:
incubating the at least one epoxide and at least one altered or recombined epoxide hydrolase, wherein the at least one altered or recombined epoxide hydrolase converts the at least one epoxide to the at least one polyol or to the at least one enantiomer of the epoxide.

89. A method of converting at least one epoxide to at least one polyol or to at least one enantiomer of the epoxide, the method comprising:
incubating the at least one epoxide and the at least one altered or recombined hydrolase of claim 18, wherein the at least one altered or recombined hydrolase comprises at least one altered or recombined epoxide hydrolase, wherein the at least one altered or recombined epoxide hydrolase converts the at least one epoxide to the at least one polyol or to the at least one enantiomer of the epoxide.

90. A method of converting at least one haloepoxide to at least one polyol or to at least one enantiomer of an epoxide, the method comprising:
incubating the at least one haloepoxide and the at least one altered or recombined hydrolase of claim 18, wherein the at least one altered or recombined hydrolase comprises at least one altered or recombined haloepoxide dehalogenase-epoxide hydrolase, wherein the at least one altered or recombined haloepoxide dehalogenase-epoxide hydrolase converts the at least one haloepoxide to the at least one polyol or to the at least one enantiomer of the epoxide.

91. The method of claim 90, wherein the at least one haloepoxide is converted to the at least one polyol or to the at least one enantiomer of the epoxide through an intermediate epoxide.

92. A method of providing a population of altered hydrolase nucleic acids, the method comprising:
hybridizing at least one set of hydrolase nucleic acid fragments; and,
elongating or ligating the hybridized hydrolase nucleic acid fragments, thereby providing the population of altered hydrolase nucleic acids.

93. The method of claim 92, wherein the at least one set of hydrolase nucleic acid fragments comprises one or more of: a set of halocarbon dehalogenase nucleic acid fragments; a set of halohydrocarbon dehalogenase nucleic acid fragments; a set of haloalcohol dehalogenase nucleic acid fragments; a set of halopolyol dehalogenase nucleic acid fragments; a set of first haloalcohol isomer providing halohydrocarbon dehalogenase nucleic acid fragments; a set of second haloalcohol isomer providing halohydrocarbon dehalogenase nucleic acid fragments; a set of haloalcohol isomer dehalogenase nucleic acid fragments; a set of epoxide providing first haloalcohol isomer dehalogenase nucleic acid fragments; a set of epoxide providing second haloalcohol isomer dehalogenase nucleic acid fragments; a set of haloepoxide dehalogenase nucleic acid fragments; and a set of epoxide hydrolase nucleic acid fragments.

94. The method of claim 92, wherein the at least one set of hydrolase nucleic acid fragments overlap.

95. The method of claim 92, wherein the at least one set of hydrolase nucleic acid fragments is selected from one or more of: accession number AJ012627, accession number AF060871, accession number AF017179, accession number L49435, accession number M26950, accession number D14594, accession number CAB01264, accession number AL079353, accession number AL022121, accession number AL123456, accession number AL035636, accession number AE002084, accession number AE000513, accession number AL137165, accession number AL133252, accession number AL132707, accession number AJ005843, accession number AL079345, accession number AL031124, accession number AF043240, accession number X84038, accession number M81691, and accession number A28028.

96. The method of claim 92, wherein the at least one set of hydrolase nucleic acid fragments comprises one or more of: synthesized oligonucleotides and nuclease digested nucleic acids.

97. The population of altered hydrolase nucleic acids made by the method of claim 92.

98. The method of claim 92, further comprising expressing the population of altered hydrolase nucleic acids to provide at least one altered hydrolase.

99. The method of claim 98, wherein the at least one altered hydrolase is selected from the group consisting of: an altered halocarbon dehalogenase; an altered halohydrocarbon dehalogenase; an altered haloalcohol dehalogenase; an altered halohydrocarbon-haloalcohol dehalogenase; an altered halopolyol dehalogenase; an altered haloalcohol-halopolyol dehalogenase; an altered halohydrocarbon-haloalcohol-halopolyol dehalogenase; an altered first haloalcohol isomer providing halohydrocarbon-second haloalcohol isomer providing halohydrocarbon dehalogenase; an altered epoxide providing halohydrocarbon-haloalcohol isomer dehalogenase; an altered epoxide providing first haloalcohol isomer-epoxide providing second haloalcohol isomer dehalogenase; an altered haloepoxide dehalogenase; an altered epoxide hydrolase; and an altered haloepoxide dehalogenase-epoxide hydrolase.

100. The at least one altered hydrolase made by the method of claim 98.

101. The method of claim 92, further comprising introducing one or more of the altered hydrolase nucleic acids into at least one cell, wherein the one or more introduced altered hydrolase nucleic acids are expressed to provide at least one altered hydrolase.

102. The method of claim 101, wherein the at least one altered hydrolase is selected from the group consisting of: an altered halocarbon dehalogenase; an altered halohydrocarbon dehalogenase; an altered haloalcohol dehalogenase; an altered halohydrocarbon-haloalcohol dehalogenase; an altered halopolyol dehalogenase; an altered haloalcohol-halopolyol dehalogenase; an altered halohydrocarbon-haloalcohol-halopolyol dehalogenase; an altered first haloalcohol isomer providing halohydrocarbon-second haloalcohol isomer providing halohydrocarbon dehalogenase; an altered epoxide providing halohydrocarbon-haloalcohol isomer dehalogenase; an altered epoxide providing first haloalcohol isomer-epoxide providing second haloalcohol isomer dehalogenase; an altered haloepoxide dehalogenase; an altered epoxide hydrolase; and an altered haloepoxide dehalogenase-epoxide hydrolase.

103. The method of claim 101, wherein the at least one cell is an organism.

104. The at least one cell made by the method of claim 101.

105. The method of claim 92, the method further comprising:
denaturing the population of altered hydrolase nucleic acids;
rehybridizing the denatured population of altered hydrolase nucleic acids;
extending the rehybridized population of altered hydrolase nucleic acids to provide a population of further altered hydrolase nucleic acids; and, optionally:
repeating the denaturing, rehybridizing, and extending steps at least once; and, optionally:
selecting or screening one or more members of the population of further altered hydrolase nucleic acids for an efficient catalyzation of at least one reaction of interest by an encoded altered hydrolase.

106. The method of claim 105, wherein the at least one reaction of interest is selected from the group consisting of: a conversion of at least one organic compound to at least one product; a conversion of at least one halocarbon to at least one product; a conversion of at least one first halohydrocarbon to at least one hydrocarbon, to at least one second halohydrocarbon, to at least one alcohol, to at least one haloalcohol, to at least one polyol, to at least one halopolyol, to at least one epoxide, or to at least one haloepoxide; a conversion of at least one first haloalcohol to at least one hydrocarbon, to at least one halohydrocarbon, to at least one second haloalcohol, to at least one polyol, to at least one halopolyol, to at least one epoxide, or to at least one haloepoxide; a conversion of at least one first halohydrocarbon to at least one hydrocarbon, to at least one halohydrocarbon, to at least one polyol, to at least one halopolyol, to at least one epoxide, to at least one haloepoxide, or to at least one haloalcohol; a conversion of at least one halopolyol to at least one epoxide; a conversion of at least one haloalcohol to at least one epoxide; a conversion of at least one halohydrocarbon to at least one epoxide; a conversion of at least one halohydrocarbon to at least one of a first haloalcohol isomer and at least one of a second haloalcohol isomer; a conversion of a mixture comprising at least one of a first haloalcohol isomer and at least one of a second haloalcohol isomer to at least one epoxide; a conversion of at least one haloepoxide to at least one epoxide; a conversion at least one epoxide to at least one polyol or to at least one enantiomer of the epoxide; and a conversion of at least one haloepoxide to at least one polyol or to at least one enantiomer of an epoxide.

107. The population of further altered hydrolase nucleic acids made by the method of claim 105.

108. The method of claim 105, further comprising expressing the population of further altered hydrolase nucleic acids to provide at least one further altered hydrolase.

109. The method of claim 108, wherein the at least one further altered hydrolase is selected from the group consisting of: a further altered halocarbon dehalogenase; a further altered halohydrocarbon dehalogenase; a further altered haloalcohol dehalogenase; a further altered halohydrocarbon-haloalcohol dehalogenase; a further altered halopolyol dehalogenase; a further altered haloalcohol-halopolyol dehalogenase; a further altered halohydrocarbon-haloalcohol-halopolyol dehalogenase; a further altered first haloalcohol isomer providing halohydrocarbon-second haloalcohol isomer providing halohydrocarbon dehalogenase; a further altered epoxide providing halohydrocarbon-haloalcohol isomer dehalogenase; a further altered epoxide providing first haloalcohol isomer-epoxide providing second haloalcohol isomer dehalogenase; a further altered haloepoxide dehalogenase; and a further altered haloepoxide dehalogenase-epoxide hydrolase.

110. The at least one further altered hydrolase made by the method of claim 108.

111. A kit for performing at least one altered enzyme catalyzed reaction comprising one or more of: a set of instructions for practicing one or more methods disclosed herein; at least one assay component comprising at least one altered enzyme or at least one cell comprising the at least one altered enzyme or both, and at least one reagent; or at least one container for packaging the set of instructions and the at least one assay component.

112. The kit of claim 111, wherein the at least one altered enzyme is selected from the group consisting of: an altered halocarbon dehalogenase, an altered halohydrocarbon dehalogenase, an altered haloalcohol dehalogenase, an altered halopolyol dehalogenase, an altered haloepoxide dehalogenase, an altered epoxide hydrolase, an altered halohydrocarbon-haloalcohol dehalogenase, an altered haloalcohol-halopolyol dehalogenase, an altered epoxide providing halohydrocarbon-haloalcohol isomer dehalogenase, an altered halohydrocarbon-haloalcohol-halopolyol dehalogenase, an altered haloepoxide dehalogenase-epoxide hydrolase, an altered first haloalcohol isomer providing halohydrocarbon-second haloalcohol isomer providing halohydrocarbon dehalogenase, and an altered epoxide providing first haloalcohol isomer-epoxide providing second haloalcohol isomer dehalogenase.

113. An assay system for performing at least one altered enzyme catalyzed reaction comprising a set of instructions for practicing one or more methods disclosed herein, and at least one reaction vessel comprising at least one altered enzyme, or at least one cell comprising the at least one altered enzyme, or both.

114. The assay system of claim 113, wherein the at least one reaction vessel further comprises one or more of: a reagent inlet, a reagent outlet, a sparging line, or an impeller.

115. The assay system of claim 113, wherein the at least one altered enzyme comprises at least one immobilized altered enzyme, or at least one altered enzyme free in solution, or both.

116. The assay system of claim 113, wherein the at least one cell comprises at least one immobilized cell, or at least one cell free in solution, or both.

117. The assay system of claim 113, wherein the at least one altered enzyme is selected from the group consisting of: an altered halocarbon dehalogenase, an altered halohydrocarbon dehalogenase, an altered haloalcohol dehalogenase, an altered halopolyol dehalogenase, an altered haloepoxide dehalogenase, an altered epoxide hydrolase, an altered halohydrocarbon-haloalcohol dehalogenase, an altered haloalcohol-halopolyol dehalogenase, an altered epoxide providing halohydrocarbon-haloalcohol isomer dehalogenase, an altered halohydrocarbon-haloalcohol-halopolyol dehalogenase, an altered haloepoxide dehalogenase-epoxide hydrolase, an altered first haloalcohol isomer providing halohydrocarbon-second haloalcohol isomer providing halohydrocarbon dehalogenase, and an altered epoxide providing first haloalcohol isomer-epoxide providing second haloalcohol isomer dehalogenase.
